(19) United States
(12) Patent Application Publication
(10) Pub. No.: US 2022/0071989 A1
(43) Pub. Date: Mar. 10, 2022

(54) METHODS OF TREATING NEGATIVE SYMPTOMS OF SCHIZOPHRENIA USING DEUTERATED DEXTROMETHORPHAN AND QUINIDINE

(71) Applicant: AVANIR PHARMACEUTICALS, INC., Aliso Viego, CA (US)

(72) Inventor: Sanjay DUBÉ, Aliso Viejo, CA (US)

(21) Appl. No.: 17/477,924

(22) Filed: Sep. 17, 2021

Related U.S. Application Data
(63) Continuation of application No. PCT/US2020/023205, filed on Mar. 17, 2020.
(60) Provisional application No. 62/820,142, filed on Mar. 18, 2019.

(51) Int. Cl. A61K 31/485; A61K 31/49; A61K 45/06; A61P 25/18

(52) U.S. Cl. CPC A61K 31/485; A61P 25/18; A61K 45/06; A61K 31/49

(57) ABSTRACT

The present disclosure relates to methods of treating negative symptoms of schizophrenia. The methods include administering to a patient having schizophrenia deuterated [d6]-dextromethorphan hydrobromide in combination with quinidine sulfate. Compositions useful for treating negative symptoms of schizophrenia are also disclosed.

This application claims the benefit of priority to U.S. Provisional Patent Application No. 62/820,142, filed Mar. 18, 2019, which is incorporated herein by reference in its entirety.

The present disclosure relates to the treatment of negative symptoms of schizophrenia. The present disclosure provides methods of treating negative symptoms of schizophrenia in a patient having schizophrenia by administering effective amounts of deuterated [d6]-dextromethorphan hydrobromide and quinidine sulfate.

Schizophrenia is a severe mental disorder that has a prevalence of approximately 1% of the world's population and is a leading cause of disability (Switaj et al. BMC Psychiatry. 2012; 12(1):193; World Health Organization. Mental Health: Schizophrenia. 2012). Onset of the illness is usually during adolescence or early adulthood and tends to begin earlier in men. Schizophrenia rarely occurs in children, but awareness of childhood-onset schizophrenia is increasing (World Health Organization. Mental Health: Schizophrenia. 2012).

Symptoms of schizophrenia are typically described as “positive” or “negative.” Positive symptoms include delusions, disturbances in the process of thinking, hallucinations, incoherence, hostility, and impulsive behaviors. Negative symptoms include deficits in motivation and emotional expressiveness, blunted affect, avolition, lack of motivation, apathy, and lack of desire to form social relationships. Negative symptoms can also manifest as severe expressive deficits in both verbal and non-verbal communication. This impairment in communication skills can cause severe functional deficits that result in diminished adaptive prosocial behaviors, social isolation, and withdrawal (Del-Monte et al. Psychia Res. 2013; 210:29-35; Adamczyk et al. Schizophr Res. 2016; 176(2-3):331-339). Furthermore, patients suffering from expression deficits may turn away from social interactions and become increasingly withdrawn or isolated, further adding to the severity of their illness. Communication is a key feature of an individual's ability to integrate into society—to form and keep relationships, to go to school, to find and maintain employment (Del-Monte et al. Psychia Res. 2013; 210:29-35; Adamczyk et al. Schizophr Res. 2016; 176(2-3):331-339). It is believed that the deficits in communication are a core feature of negative symptoms and key contributors to the long-term poor outcomes for patients.

Negative symptoms are estimated to affect between 20% and 40% of individuals with schizophrenia (Pai, Nitte Univ J Health Sci. 2015; 5(2):104-115). Nearly 60% of stable outpatients with schizophrenia have at least one negative symptom, and 41% have two or more negative symptoms (Bobes et al. J Clin Psychiatry. 2010; 71(3):280-6). Negative symptoms can impact the course of schizophrenia and account for much of patients' long-term morbidity (Fervaha et al. Eur Psychiatry. 2014; 29(7):449-55; Rabinowitz et al. Schizophr Res. 2012; 137(1-3):147-150; Sicras-Mainar et al. BMC Psychiatry. 2014; 14:225). Patients with more prominent negative symptoms of schizophrenia have consistently shown worse functional outcomes (Alphs et al. Schizophr Bull. 2006; 32(2):225-230; Barnes et al. Health Technol Assess. 2016; 20(29); Blanchard et al. Schizophr Res. 2005; 77(2-3):151-165; Milev et al. Am J Psychiatry. 2005; 162(3):495-506; Ho et al. Am J Psychiatry. 1998; 155(9):1196-1201). Negative symptoms are also associated with greater reductions in quality of life and greater family/caregiver burdens than positive symptoms (Gonfrier et al. J Nutr Health Aging. 2012; 16(2):134-137; Kirkpatrick and Fischer, Schizophr Bull. 2006; 32(2):246-249; Rofail et al. Qual Life Res. 2016; 25(1):201-211; Velligan et al. Schizophr Res. 1997; 25(1):21-31; Mantovani et al. Trends Psychiatry Psychother. 2016; 38(2):96-99). As such, negative symptoms of schizophrenia represent an important component of illness management and a clinically important target for pharmacologic treatment (Laughren and Levin, Schizophr Bull. 2006; 32(2):220-222; Marder et al. Schizophren Bull. 2011; 37(2):250-254; Foussias, Schizophr Bull. 2010; 36(2):359-369; Strauss et al. J Psychiatr Res. 2013; 47(6):783-790). Treatments that decrease the severity of these expression and communication deficits have the potential to fundamentally improve patients' functional ability and quality of life.

The International Society for Central Nervous System Clinical Trials and Methodology (ISCTM) Workshop panel considers use of the subscales of the Positive and Negative Syndrome Scale (PANSS, e.g., negative factors score) and the Negative Symptom Assessment-16 (NSA-16) scale as reliable and valid measures of negative symptoms, with the severity of these being reflective of patients' functional impairment (Marder et al. Schizophren Bull. 2011; 37(2):250-254; Daniel et al. Clin Schizophr Relat Psychoses. 2011; 5(2):87-94; Velligan et al. Psychiatry Res. 2009; 169(2):97-100). Furthermore, a 2009 ISCTM consensus statement expressed preference for the PANSS negative factors (e.g., the PANSS Marder negative factors) over the original PANSS negative subscale, as the original subscale includes N5, difficulty with abstract thinking and N7, stereotyped thinking, which are felt to be outside the accepted domains of negative symptoms of schizophrenia (Laughren and Levin, Schizophr Bull. 2006; 32(2):220-222).

Multiple interventions for the treatment of negative symptoms of schizophrenia have been the subject of investigation, including pharmacological strategies using dextromethorphan (Remington et al. Curr Treat Options Psychiatry. 2016; 3:133-150; Veerman et al. Drugs. 2017; 77(13):1423-1459; Lee et al. J Psychiatr Res. 2015; 69:50-56). “The array and diversity of strategies currently under investigation highlight the lack of evidence-based treatments and our limited understanding regarding negative symptoms underlying etiology and pathophysiology” (Remington et al. Curr Treat Options Psychiatry. 2016; 3:133-150 at 134). “There is insufficient evidence at present to support a specific treatment for negative symptoms.” Id. at 144. “This is despite a tremendous increase in the topic, as well as studies where negative symptoms are the identified primary outcome.” Id. There is currently no U.S. Food and Drug Administration (FDA)-approved treatment for negative symptoms of schizophrenia.

Thus, there remains an unmet need for a safe and effective pharmacologic intervention for treating negative symptoms of schizophrenia. “This is undoubtedly driven, at least in part, by evidence that negative symptoms play a critical role in functional decline that is not necessarily addressed with adequate control of positive symptoms” (Remington et al. Curr Treat Options Psychiatry. 2016; 3:133-150 at 145). An effective treatment for patients with negative symptoms of schizophrenia could profoundly improve patients' mental health, quality of life, caregiver burden, and reduce healthcare costs.

This disclosure provides, in some embodiments, methods of treating negative symptoms in patients with schizophrenia using deuterated [d6]-dextromethorphan, or a salt thereof, in combination with quinidine, or a salt thereof. In some embodiments, this disclosure provides methods of treating negative symptoms in patients with schizophrenia using deuterated [d6]-dextromethorphan hydrobromide (d6-DM) in combination with quinidine sulfate (Q). As used herein, the term “d6-DM” means deuterated [d6]-dextromethorphan hydrobromide. As used herein, the term “Q” means quinidine sulfate.

More specifically, in some embodiments, the present disclosure provides a method of specifically treating negative symptoms of schizophrenia in a patient having schizophrenia, comprising administering to the patient therapeutically effective amounts of d6-DM and Q. In some embodiments, the d6-DM is administered in a 27 mg to 54 mg dose twice daily and the Q is administered in a 4 mg to 7.5 mg dose twice daily. In some embodiments, the d6-DM is administered in a 30 mg to 45 mg dose twice daily and the Q is administered in a 4 mg to 6 mg dose twice daily. In some embodiments, the d6-DM is administered in a 34 mg to 42.63 mg dose twice daily and the Q is administered in a 4.9 mg dose twice daily. In some embodiments, the d6-DM is administered in a 34 mg dose twice daily and the Q is administered in a 4.9 mg dose twice daily. In some embodiments, the d6-DM is administered in a 42.63 mg dose twice daily and the Q is administered in a 4.9 mg dose twice daily.

In some embodiments, the present disclosure provides a method of treating negative symptoms of schizophrenia in a patient having schizophrenia and having clinically stable positive symptoms, comprising administering to the patient therapeutically effective amounts of d6-DM and Q.

In some embodiments, the present disclosure provides a method of treating negative symptoms of schizophrenia in a patient having schizophrenia, comprising administering to the patient therapeutically effective amounts of d6-DM and Q, wherein the patient has not had an inpatient psychiatric hospitalization within 4 months prior to treatment.

In some embodiments, the present disclosure provides a method of treating negative symptoms of schizophrenia in a patient having schizophrenia, comprising administering to the patient therapeutically effective amounts of d6-DM and Q, wherein the patient has not had a psychiatric hospital admission or acute exacerbation within 6 months prior to treatment.

In some embodiments, the present disclosure provides a method of treating negative symptoms of schizophrenia in a patient having schizophrenia, comprising administering to the patient therapeutically effective amounts of d6-DM and Q, wherein the patient has been assessed as having a score of less than or equal to 4 on the Positive and Negative Syndrome Scale (PANSS) items of delusions, hallucinations, and hostility. In some embodiments, the patient has been assessed as having a score of greater than or equal to 4 on any two, or greater than or equal to 5 on any one, of the PANSS items of blunted affect (N1), emotional withdrawal (N2), passive/apathetic social withdrawal (N4), and lack of spontaneity/flow of conversation (N6). In some embodiments, that patient has been assessed as having a total PANSS negative subscale score (N1 to N7) of greater than or equal to 18.

In some embodiments, the present disclosure provides a method of treating negative symptoms of schizophrenia in a patient having schizophrenia, comprising administering to the patient therapeutically effective amounts of d6-DM and Q, wherein the patient has been assessed as having a score of less than or equal to 4 on the Positive and Negative Syndrome Scale (PANSS) items of delusions, hallucinations, suspiciousness/persecution, and hostility. In some embodiments, the patient has been assessed as having a score of greater than or equal to 4 on any two, or greater than or equal to 5 on any one, of the PANSS items of blunted affect (N1), emotional withdrawal (N2), passive/apathetic social withdrawal (N4), and lack of spontaneity/flow of conversation (N6). In some embodiments, the patient has been assessed as having a total PANSS Marder negative factors score (N1: blunted effect; N2: emotional withdrawal; N3: poor rapport; N4: passive/apathetic social withdrawal; N6: lack of spontaneity/flow of conversation; G7: motor retardation; and G16: active social avoidance) of greater than or equal to 20.

In some embodiments, the present disclosure provides a method of specifically treating negative symptoms of schizophrenia in a patient having schizophrenia, comprising administering to the patient therapeutically effective amounts of d6-DM and Q, wherein the patient is being treated with an atypical antipsychotic. In some embodiments, the patient has been treated with the atypical antipsychotic for at least 3 months, the dose of the atypical antipsychotic has been stable for at least 1 month. In some embodiments, the patient has not had an inpatient psychiatric hospitalization within 4 months, prior to treatment with d6-DM and Q.

In some embodiments, the present disclosure provides a method of specifically treating negative symptoms of schizophrenia in a patient having schizophrenia, comprising administering to the patient therapeutically effective amounts of d6-DM and Q, wherein the patient is being treated with an antidepressant. In some embodiments, the patient has been treated with the antidepressant for at least 3 months, and the dose of the antidepressant has been stable for at least 1 month, prior to treatment with d6-DM and Q.

In some embodiments, the present disclosure provides a method of specifically treating negative symptoms of schizophrenia in a patient having schizophrenia, comprising administering to the patient therapeutically effective amounts of d6-DM and Q, wherein the patient is being treated with a hypnotic. In some embodiments, the dose of the hypnotic has been stable for at least 1 month prior to treatment with d6-DM and Q.

In some embodiments, the present disclosure provides a method of specifically treating negative symptoms of schizophrenia in a patient having schizophrenia, comprising administering to the patient therapeutically effective amounts of d6-DM and Q, wherein the patient is being treated with lorazepam up to a total dose of 2 mg per day for insomnia, anxiety, restlessness, or agitation. In some embodiments, the dose of the lorazepam has been stable for at least 1 month prior to treatment with d6-DM and Q.

In some embodiments, the present disclosure provides a method of specifically treating negative symptoms of schizophrenia in a patient having schizophrenia, comprising administering to the patient therapeutically effective amounts of d6-DM and Q, wherein the patient is not being treated with one or more monoamine oxidase inhibitors (MAOIs).

In some embodiments, the present disclosure provides a method of specifically treating negative symptoms of schizophrenia in a patient having schizophrenia, comprising administering to the patient therapeutically effective amounts of d6-DM and Q, wherein the patient is not being treated with clozapine.

In some embodiments, the present disclosure provides a method of specifically treating negative symptoms of schizophrenia in a patient having schizophrenia, comprising administering to the patient therapeutically effective amounts of d6-DM and Q, wherein the patient is not being treated with a benzodiazepine other than lorazepam.

In some embodiments, the present disclosure provides a method of specifically treating negative symptoms of schizophrenia in a patient having schizophrenia, comprising administering to the patient therapeutically effective amounts of d6-DM and Q, wherein the patient is not being treated with levodopa.

In some embodiments, the present disclosure provides a method of specifically treating negative symptoms of schizophrenia in a patient having schizophrenia, comprising administering to the patient therapeutically effective amounts of d6-DM and Q, wherein the patient is not being treated with a typical antipsychotic.

In some embodiments, the present disclosure provides a method of specifically treating negative symptoms of schizophrenia in a patient having schizophrenia, comprising administering to the patient therapeutically effective amounts of d6-DM and Q, wherein the patient is not being treated with an agent that:

(a) increases plasma levels of quinidine;

(b) is metabolized by CYP2D6;

(c) is related to quinidine;

(d) produces serotonin syndrome when co-administered with dextromethorphan;

(e) decreases plasma levels of dextromethorphan and quinidine;

(f) is clozapine; or

(g) is a typical antipsychotic.

In some embodiments, the present disclosure provides a method of specifically treating negative symptoms of schizophrenia in a patient having schizophrenia, comprising administering to the patient therapeutically effective amounts of d6-DM and Q, wherein the patient is not being treated with an anticholinergic medication.

In some embodiments, the present disclosure provides a method of specifically treating negative symptoms of schizophrenia in a patient having schizophrenia, comprising administering to the patient therapeutically effective amounts of d6-DM and Q, wherein the patient has not received electroconvulsive treatment, repetitive transcranial magnetic stimulation, or deep brain stimulation within one year prior to treatment.

In some embodiments, the present disclosure provides a method of specifically treating negative symptoms of schizophrenia in a patient having schizophrenia, comprising administering to the patient therapeutically effective amounts of d6-DM and Q, wherein the patient does not have myasthenia gravis.

In some embodiments, the present disclosure provides a method of specifically treating negative symptoms of schizophrenia in a patient having schizophrenia, comprising administering to the patient therapeutically effective amounts of d6-DM and Q, wherein the patient does not have schizoaffective disorder.

In some embodiments, the present disclosure provides a method of specifically treating negative symptoms of schizophrenia in a patient having schizophrenia, comprising administering to the patient therapeutically effective amounts of d6-DM and Q, wherein the patient does not have bipolar disorder.

In some embodiments, the present disclosure provides a method of specifically treating negative symptoms of schizophrenia in a patient having schizophrenia, comprising administering to the patient therapeutically effective amounts of d6-DM and Q, wherein the patient does not have a depressive disorder and/or a Calgary Depression Scale for Schizophrenia (CDSS) score of greater than or equal to 6.

In some embodiments, the present disclosure provides a method of specifically treating negative symptoms of schizophrenia in a patient having schizophrenia, comprising administering to the patient therapeutically effective amounts of d6-DM and Q, wherein the patient does not have a score of greater than 3 on the sum of eight items of the Simpson-Angus Scale (SAS): gait, arm dropping, shoulder shaking, elbow rigidity, wrist rigidity, leg pendulousness, head rotation, and glabella tap.

In some embodiments, the present disclosure provides a method of specifically treating negative symptoms of schizophrenia in a patient having schizophrenia, comprising administering to the patient therapeutically effective amounts of d6-DM and Q, wherein the patient does not have a concurrent clinically significant or unstable systemic disease, neurological disorder, cognitive disorder, neurodegenerative disorder, hepatic disorder, renal disorder, metabolic disorder, hematological disorder, immunological disorder, cardiovascular disorder, pulmonary disorder, or gastrointestinal disorder, as determined by the prescribing doctor.

In some embodiments, the present disclosure provides a method of specifically treating negative symptoms of schizophrenia in a patient having schizophrenia, comprising administering to the patient therapeutically effective amounts of d6-DM and Q, wherein the patient does not have a suicide risk. In some embodiments, suicide risk is determined by one or more of the following:

(a) judgment of the prescribing doctor;

(b) the patient answers yes on the Columbia Suicide Severity Rating Scale (C-SSRS Suicidal Ideation Item 4 (active suicidal ideation with some intent to act, without a specific plan) and the patient's most recent episode meeting this C-SSRS Item 4 occurred within six months;

(c) the patient answers yes on the C-SSRS Suicidal Behavior Item 5 (active suicidal ideation with specific plan and intent) and the patient's most recent episode meeting this C-SSRS Item 5 occurred within six months; and

(d) the patient answers yes on any of the 5 on the C-SSRS Suicidal Behavior Items (active attempt, interrupted attempt, aborted attempt, preparatory acts, or behavior) and the patient's most recent episode meeting any of these C-SSRS Items occurred within two years prior to treatment. For example, in some such embodiments, suicide risk is determined by all of (a), (b), (c), and (d). For example, in some such embodiments, suicide risk is determined by (a), (b), and (c). For example, in some such embodiments, suicide risk is determined by (a) and (b). For example, in some such embodiments, suicide risk is determined by any one of (a), (b), (c), or (d).

In some embodiments, the present disclosure provides a method of specifically treating negative symptoms of schizophrenia in a patient having schizophrenia, comprising administering to the patient therapeutically effective amounts of d6-DM and Q, wherein the patient does not have a cardiovascular history of any one or more of:

(a) history or evidence of complete heart block, ventricular tachycardia, presence of clinically significant premature ventricular contractions (PVCs) as evaluated by a central reader, QTc prolongation, or torsades de pointes;

(b) QTc using the Fridericia's formula (QTcF) greater than 450 msec for males and greater than 470 msec for females based on central review, unless due to ventricular pacing;

(c) family history of congenital QT interval prolongation syndrome; and

(d) history or presence of clinically significant syncope, orthostatic hypotension, or postural tachycardia. For example, in some such embodiments, the patient does not have a history of all of (a), (b), (c), and (d). For example, in some such embodiments, the patient does not have a history of (a), (b), and (c). For example, in some such embodiments, the patient does not have a history of (a) and (b). For example, in some such embodiments, does not have a history of any one of (a), (b), (c), or (d).

In some embodiments, the present disclosure provides a method of specifically treating negative symptoms of schizophrenia in a patient having schizophrenia, comprising administering to the patient therapeutically effective amounts of d6-DM and Q, wherein the patient does not have pseudoparkinsonism secondary to treatment with an antipsychotic.

In some embodiments, the present disclosure provides a method of specifically treating negative symptoms of schizophrenia in a patient having schizophrenia, comprising administering to the patient therapeutically effective amounts of d6-DM and Q, wherein the patient does not have a history of substance and/or alcohol abuse, but may use tobacco and/or nicotine products.

In some embodiments, the present disclosure provides a method of specifically treating negative symptoms of schizophrenia in a patient having schizophrenia, comprising administering to the patient therapeutically effective amounts of d6-DM and Q, wherein the patient does not use recreational or medicinal marijuana, as evidenced by a negative urine drug screen for cannabis.

In some embodiments, the present disclosure provides a method of specifically treating negative symptoms of schizophrenia in a patient having schizophrenia, comprising administering to the patient therapeutically effective amounts of d6-DM and Q, wherein the patient does not test positive for hepatitis B surface antigen, hepatitis C antibody, or HIV antibody.

In some embodiments, the present disclosure provides a method of specifically treating negative symptoms of schizophrenia in a patient having schizophrenia, comprising administering to the patient therapeutically effective amounts of d6-DM and Q, wherein during the first week of treatment, the d6-DM is administered in a 24 mg dose once daily and the Q is administered in a 4.9 mg dose once daily; during the second week of treatment, the d6-DM is administered in a 24 mg dose twice daily and the Q is administered in a 4.9 mg dose twice daily; and during the remainder of the treatment, the d6-DM is administered in a 34 mg dose twice daily and the Q is administered in a 4.9 mg dose twice daily.

In some embodiments, the present disclosure provides a method of specifically treating negative symptoms of schizophrenia in a patient having schizophrenia, comprising administering to the patient therapeutically effective amounts of d6-DM and Q, wherein during the first three days of treatment, the d6-DM is administered in a 28 mg dose once daily and the Q is administered in a 4.9 mg dose once daily; during the next four days of treatment, the d6-DM is administered in a 28 mg dose twice daily and the Q is administered in a 4.9 mg dose twice daily; and during the remainder of the treatment, the d6-DM is administered in a 42.63 mg dose twice daily and the Q is administered in a 4.9 mg dose twice daily.

In some embodiments of the methods disclosed herein, the patient is further administered an atypical antipsychotic other than clozapine.

In some embodiments of the methods disclosed herein, the patient is a male or female patient from 18 to 60 years of age.

In some embodiments of the methods disclosed herein, the patient is a female of childbearing potential. In some embodiments, the patient: (a) has a negative urine pregnancy test; (b) is not nursing or planning a pregnancy for the duration of treatment through 30 days after the last dose; and (c) is abstinent or willing to use a method of birth control prior to treatment, and to continue with the same method until 28 days after the last dose.

In some embodiments of the methods disclosed herein, the patient does not have hypersensitivity to dextromethorphan, quinidine, an opiate drug, d6-DM, 0, or any ingredient thereof.

In some embodiments of the methods disclosed herein, the patient does not have allergy or hypersensitivity to one or more medications.

In some embodiments of the methods disclosed herein, the patient does not have one or more clinically significant laboratory abnormalities, one or more safety values of clinical concern, or aspartate aminotransferase (AST) or alanine aminotransferase (ALT) levels greater than two times the upper limit of normal, as determined by the prescribing doctor.

In some embodiments of the methods disclosed herein, the patient has been diagnosed as having schizophrenia based on the Diagnostic and Statistical Manual of Mental Disorders (DSM) criteria for schizophrenia. In some embodiments, the diagnosis based on the DSM criteria has been confirmed by the Mini International Neuropsychiatric Interview (M.I.N.I.).

In some embodiments of the methods disclosed herein, the patient does not have schizoaffective disorder. In some embodiments, the patient does not have bipolar disorder.

In some embodiments of the methods disclosed herein, the treatment results in an at least 20% decrease in the PANSS Marder Negative Factor Score from baseline prior to treatment. In some embodiments, the treatment results in an at least 2 point decrease in the PANSS Marder Negative Factor Score from baseline prior to treatment.

The methods disclosed herein may also, optionally, include administration of the d6-DM and the Q in conjunction with other therapeutic agents, such as, for example, one or more therapeutic agents useful for the treatment of schizophrenia. In some embodiments, the d6-DM and the Q may be administered in combination with an atypical antipsychotic other than clozapine (e.g., a second-generation atypical antipsychotic drug (SGA)).

Also provided herein are therapeutic uses of d6-DM and Q. In some embodiments, the present disclosure provides d6-DM and Q for use in specifically treating negative symptoms of schizophrenia in a patient having schizophrenia. In some embodiments, the present disclosure provides the use of d6-DM and Q in specifically treating negative symptoms of schizophrenia in a patient having schizophrenia. In some embodiments, the present disclosure provides the use of d6-DM and Q in a method of manufacturing a medicament for specifically treating negative symptoms of schizophrenia in a patient having schizophrenia. Compositions useful for treating negative symptoms of schizophrenia are also provided.

In some embodiments, the present disclosure provides a medicament comprising a therapeutically effective amount of d6-DM for use in the treatment of negative symptoms of schizophrenia in a patient having schizophrenia, which is used in combination with a therapeutically effective amount of Q simultaneously, separately, or sequentially.

In some embodiments, the present disclosure provides a therapeutically effective amount of d6-DM for use in treating negative symptoms of schizophrenia in a patient having schizophrenia, characterized in that the d6-DM is administered in combination with a therapeutically effective amount of Q, wherein both medicaments are administered simultaneously, separately, or sequentially.

In some embodiments, the present disclosure provides a combination of a therapeutically effective amount of d6-DM and a therapeutically effective amount of Q for use in treating negative symptoms of schizophrenia in a patient having schizophrenia, wherein both medicaments are administered simultaneously, separately, or sequentially.

In some embodiments, the present disclosure provides a pharmaceutical composition comprising a therapeutically effective amount of d6-DM, which is used in combination with a therapeutically effective amount of Q simultaneously, separately, or sequentially, for treating negative symptoms of schizophrenia in a patient having schizophrenia.

### BRIEF DESCRIPTION OF THE DRAWINGS

FIG. 1 shows the chemical structure of deuterated [d6]-dextromethorphan hydrobromide (d6-DM).

FIG. 2 shows the study design and schedule of assessments in the study from Example 1. Study medication (active or placebo) was administered as 1 capsule in the morning and 1 capsule in the evening approximately 12 hours apart. M: Morning Dose (in mg d6-DM/mg Q); E: Evening Dose (in mg d6-DM/mg Q); *: Visit 4 (Day 43) stratification was based on treatment response criteria followed by Re-Randomization (1:1).

FIG. 3 shows disposition of patients in the modified intent-to-treat (mITT) population in the study from Example 1.

FIG. 4 shows NSA-16 Total Mean Scores by visit (sequential parallel comparison design (SPCD), mITT population) in the study from Example 1.

FIG. 5 shows PANSS Total Mean Scores by visit (SPCD, mITT population) in the study from Example 1.

FIG. 6 shows PANSS Negative Subscale Mean Scores by visit (SPCD, mITT population) in the study from Example 1.

FIG. 7 shows PANSS Marder Negative Factors Mean Scores by visit (SPCD, mITT population) in the study from Example 1.

FIG. 8 shows PANSS Prosocial Factors Mean Scores by visit (SPCD, mITT population) in the study from Example 1.

FIG. 9 shows NSA-16 Global Negative Symptoms Ratings by visit (SPCD, mITT population) in the study from Example 1.

FIG. 10 shows PGI-C Ratings of “Much” or “Very Much” Improved: Stage 1 (Baseline to Week 6) and Stage 2 (Week 6 to Week 12) in the study from Example 1.

### DETAILED DESCRIPTION OF ILLUSTRATIVE EMBODIMENTS

The following detailed description and examples illustrate certain embodiments of the present disclosure. Those of skill in the art will recognize that there are numerous variations and modifications of this disclosure that are encompassed by its scope. Accordingly, the description of certain embodiments should not be deemed to limit the scope of the present disclosure.

In order that the disclosure may be more readily understood, certain terms are defined throughout the detailed description. Unless defined otherwise herein, all scientific and technical terms used in connection with the present disclosure have the same meaning as commonly understood by those of ordinary skill in the art.

All references cited herein, including, but not limited to, published and unpublished applications, patents, and literature references, are incorporated herein by reference in their entirety and are hereby made a part of this specification. To the extent a cited reference conflicts with the disclosure herein, the specification shall control.

As used herein, the singular forms of a word also include the plural form, unless the context clearly dictates otherwise; as examples, the terms “a,” “an,” and “the” are understood to be singular or plural. By way of example, “an element” means one or more element. The term “or” shall mean “and/or” unless the specific context indicates otherwise.

The present disclosure, in some embodiments, provides methods of treating negative symptoms of schizophrenia in a patient having schizophrenia by administering to the patient deuterated [d6]-dextromethorphan hydrobromide (d6-DM) and quinidine sulfate (Q). Uses of d6-DM and Q, e.g., in treating negative symptoms of schizophrenia, are also provided. Compositions (e.g., pharmaceutical compositions) comprising d6-DM and Q are also disclosed and are useful in the therapeutic methods and uses described herein.

As used herein, the term “d6-DM” means deuterated [d6]-dextromethorphan hydrobromide. As used herein, the term “Q” means quinidine sulfate.

As used herein, the term “a patient having schizophrenia” means a patient that has been diagnosed as having schizophrenia.

“Negative symptoms of schizophrenia” include one or more of blunted affect, emotional withdrawal, poor rapport, passive/apathetic social withdrawal, lack of spontaneity and flow of conversation, motor retardation, active social avoidance, difficultly in abstract thinking, stereotyped thinking, alogia, asociality, anhedonia, and avolition. In some embodiments, the negative symptom of schizophrenia is blunted affect. In some embodiments, the negative symptom of schizophrenia is emotional withdrawal. In some embodiments, the negative symptom of schizophrenia is poor rapport. In some embodiments, the negative symptom of schizophrenia is passive/apathetic social withdrawal. In some embodiments, the negative symptom of schizophrenia is lack of spontaneity and flow of conversation. In some embodiments, the negative symptom of schizophrenia is motor retardation. In some embodiments, the negative symptom of schizophrenia is active social avoidance. In some embodiments, the negative symptom of schizophrenia is difficulty in abstract thinking. In some embodiments, the negative symptom of schizophrenia is stereotyped thinking. In some embodiments, the negative symptom of schizophrenia is alogia. In some embodiments, the negative symptom of schizophrenia is asociality. In some embodiments, the negative symptom of schizophrenia is anhedonia. In some embodiments, the negative symptom of schizophrenia is avolition.

In addition to negative symptoms, schizophrenia patients can also exhibit one or more positive symptoms. Exemplary positive symptoms of schizophrenia include delusions, conceptual disorganization, hallucinations, excitement, grandiosity, suspicious/persecution, and hostility.

As used herein, the term “treating negative symptoms of schizophrenia” means improving one or more negative symptoms of schizophrenia.

As used herein, the term “treating prosocial factors” means improving one or more prosocial factors.

As used herein, the term “specifically treating negative symptoms of schizophrenia” means improving one or more negative symptoms of schizophrenia irrespective of the effect on one or more of: any positive symptom of schizophrenia; depression; and any extrapyramidal symptom.

The term “therapeutically effective amounts of deuterated [d6]-dextromethorphan hydrobromide (d6-DM) and quinidine sulfate (Q)” refers to the amount of d6-DM and the amount of Q that are sufficient to improve one or more negative symptoms of schizophrenia when administered in combination. As used herein, the term “combination” applied to d6-DM and Q means a single pharmaceutical composition (formulation) comprising both d6-DM and Q or two separate pharmaceutical compositions (formulations), each comprising d6-DM or Q, to be administered in combination.

Administered “in combination” or “co-administration,” as used herein, refers to administration of d6-DM and Q simultaneously in one composition, or simultaneously in different compositions, or sequentially. For sequential administration to be considered administration “in combination” or “co-administration,” the d6-DM and the Q are administered separated by a time interval that permits the resultant beneficial effect for treating one or more negative symptoms of schizophrenia in a patient.

The term “patient” and “subject” means a human. In some embodiments, the patient is a human having schizophrenia.

In certain alternative embodiments, salt forms of deuterated [d6]-dextromethorphan other than hydrobromide and salt forms of quinidine other than sulfate may be used in the embodiments described herein.

Unless otherwise specified, the doses described herein refer to the hydrobromide and sulfate salt forms of deuterated [d6]-dextromethorphan and quinidine (i.e., d6-DM and Q), respectively. Based on such information, those skilled in the art can calculate corresponding dosages for the respective free-base forms of the active ingredient. A person of skill in the art can calculate the molecular weight for the salt of dextromethorphan and the molecular weight for free base of dextromethorphan and use the ratio to calculate appropriate dosages for the free base as well as for the salt.

### Therapeutic Methods

The present disclosure provides, in some embodiments, methods of treating negative symptoms of schizophrenia in a patient having schizophrenia, comprising administering to the patient therapeutically effective amounts of deuterated [d6]-dextromethorphan hydrobromide (d6-DM) and quinidine sulfate (Q).

In some embodiments, the specification provides a method of specifically treating negative symptoms of schizophrenia in a patient having schizophrenia, comprising administering to the patient therapeutically effective amounts of d6-DM and Q, wherein the d6-DM is administered in a 27 mg to 54 mg dose twice daily and the Q is administered in a 4 mg to 7.5 mg dose twice daily.

In some embodiments, the specification provides a method of specifically treating negative symptoms of schizophrenia in a patient having schizophrenia, comprising administering to the patient therapeutically effective amounts of d6-DM and Q, wherein the d6-DM is administered in a 30 mg to 45 mg dose twice daily and the Q is administered in a 4 mg to 6 mg dose twice daily.

In some embodiments, the present disclosure provides a method of specifically treating negative symptoms of schizophrenia in a patient having schizophrenia, comprising administering to the patient therapeutically effective amounts of d6-DM and Q, wherein the d6-DM is administered in a 34 mg to 42.63 mg dose twice daily and the Q is administered in a 4.9 mg dose twice daily. In some embodiments, the d6-DM is administered in a 34 mg dose twice daily and the Q is administered in a 4.9 mg dose twice daily. In some embodiments, the d6-DM is administered in a 42.63 mg dose twice daily and the Q is administered in a 4.9 mg dose twice daily.

In some embodiments, the present disclosure provides a method of treating negative symptoms of schizophrenia in a patient having schizophrenia and having clinically stable positive symptoms, comprising administering to the patient therapeutically effective amounts of d6-DM and Q.

In some embodiments, the present disclosure provides a method of treating negative symptoms of schizophrenia in a patient having schizophrenia, comprising administering to the patient therapeutically effective amounts of d6-DM and Q, wherein the patient has not had an inpatient psychiatric hospitalization within 4 months prior to treatment.

In some embodiments, the present disclosure provides a method of treating negative symptoms of schizophrenia in a patient having schizophrenia, comprising administering to the patient therapeutically effective amounts of d6-DM and Q, wherein the patient has not had a psychiatric hospital admission or acute exacerbation within 6 months prior to treatment.

In some embodiments, the present disclosure provides a method of treating negative symptoms of schizophrenia in a patient having schizophrenia, comprising administering to the patient therapeutically effective amounts of d6-DM and Q, wherein the patient has been assessed as having a score of less than or equal to 4 on the Positive and Negative Syndrome Scale (PANSS) items of delusions, hallucinations, and hostility. In some embodiments, the patient has been assessed as having a score of greater than or equal to 4 on any two, or greater than or equal to 5 on any one, of the PANSS items of blunted affect (N1), emotional withdrawal (N2), passive/apathetic social withdrawal (N4), and lack of spontaneity/flow of conversation (N6). In some embodiments, the patient has been assessed as having a total PANSS negative subscale score (N1 to N7) of greater than or equal to 18.

In some embodiments, the present disclosure provides a method of treating negative symptoms of schizophrenia in a patient having schizophrenia, comprising administering to the patient therapeutically effective amounts of d6-DM and Q, wherein the patient has been assessed as having a score of less than or equal to 4 on the Positive and Negative Syndrome Scale (PANSS) items of delusions, hallucinations, suspiciousness/persecution, and hostility. In some embodiments, the patient has been assessed as having a score of greater than or equal to 4 on any two, or greater than or equal to 5 on any one, of the PANSS items of blunted affect (N1), emotional withdrawal (N2), passive/apathetic social withdrawal (N4), and lack of spontaneity/flow of conversation (N6). In some embodiments, the patient has been assessed as having a total PANSS Marder negative factors score (N1: blunted effect; N2: emotional withdrawal; N3: poor rapport; N4: passive/apathetic social withdrawal; N6: lack of spontaneity/flow of conversation; G7: motor retardation; and G16: active social avoidance) of greater than or equal to 20.

In some embodiments, the present disclosure provides a method of specifically treating negative symptoms of schizophrenia in a patient having schizophrenia, comprising administering to the patient therapeutically effective amounts of d6-DM and Q, wherein the patient is being treated with an atypical antipsychotic, wherein the patient has been treated with the atypical antipsychotic for at least 3 months, the dose of the atypical antipsychotic has been stable for at least 1 month. In some embodiments, the patient has not had an inpatient psychiatric hospitalization within 4 months, prior to treatment with d6-DM and Q.

In some embodiments, the present disclosure provides a method of specifically treating negative symptoms of schizophrenia in a patient having schizophrenia, comprising administering to the patient therapeutically effective amounts of d6-DM and Q, wherein the patient is being treated with an antidepressant, wherein the patient has been treated with the antidepressant for at least 3 months, and the dose of the antidepressant has been stable for at least 1 month, prior to treatment with d6-DM and Q.

In some embodiments, the present disclosure provides a method of specifically treating negative symptoms of schizophrenia in a patient having schizophrenia, comprising administering to the patient therapeutically effective amounts of d6-DM and Q, wherein the patient is being treated with a hypnotic, wherein the dose of the hypnotic has been stable for at least 1 month prior to treatment with d6-DM and Q.

In some embodiments, the present disclosure provides a method of specifically treating negative symptoms of schizophrenia in a patient having schizophrenia, comprising administering to the patient therapeutically effective amounts of d6-DM and Q, wherein the patient is being treated with lorazepam up to a total dose of 2 mg per day for insomnia, anxiety, restlessness, or agitation, wherein the dose of the lorazepam has been stable for at least 1 month prior to treatment with d6-DM and Q.

In some embodiments of the methods disclosed herein, the patient is not being treated with certain additional therapeutic agents concomitantly with the d6-DM and the Q. In some embodiments, the patient has not taken certain additional therapeutic agent(s) within 2 weeks or 5 half-lives of the additional therapeutic agent(s), whichever is longer, prior to the start of treatment with the d6-DM and the Q.

In some embodiments, the present disclosure provides a method of specifically treating negative symptoms of schizophrenia in a patient having schizophrenia, comprising administering to the patient therapeutically effective amounts of d6-DM and Q, wherein the patient is not being treated with one or more monoamine oxidase inhibitors (MAOIs). Exemplary MAOIs include carbamazepine, cyproterone, hyperforin, oxcarbazepine, phenobarbital, phenytoin, rifampicin, and St. John's Wort.

In some embodiments, the present disclosure provides a method of specifically treating negative symptoms of schizophrenia in a patient having schizophrenia, comprising administering to the patient therapeutically effective amounts of d6-DM and Q, wherein the patient is not being treated with clozapine.

In some embodiments, the present disclosure provides a method of specifically treating negative symptoms of schizophrenia in a patient having schizophrenia, comprising administering to the patient therapeutically effective amounts of d6-DM and Q, wherein the patient is not being treated with a benzodiazepine other than lorazepam.

In some embodiments, the present disclosure provides a method of specifically treating negative symptoms of schizophrenia in a patient having schizophrenia, comprising administering to the patient therapeutically effective amounts of d6-DM and Q, wherein the patient is not being treated with levodopa.

In some embodiments, the present disclosure provides a method of specifically treating negative symptoms of schizophrenia in a patient having schizophrenia, comprising administering to the patient therapeutically effective amounts of d6-DM and Q, wherein the patient is not being treated with a typical antipsychotic. Exemplary typical antipsychotics include but are not limited to haloperidol, loxapine, thioridazine, molindone, thiothixene, fluphenazine, mesoridazine, trifluoperazine, perphenazine, and chlorpromazine.

In some embodiments, the present disclosure provides a method of specifically treating negative symptoms of schizophrenia in a patient having schizophrenia, comprising administering to the patient therapeutically effective amounts of d6-DM and Q, wherein the patient is not being treated with an agent that:

(a) increases plasma levels of quinidine;

(b) is metabolized by CYP2D6;

(c) is related to quinidine;

(d) produces serotonin syndrome when co-administered with dextromethorphan;

(e) decreases plasma levels of dextromethorphan and quinidine;

(f) is clozapine; or

(g) is a typical antipsychotic.

In some embodiments, the patient is not being treated with an agent that may increase plasma levels of quinidine. Exemplary agents that may increase plasma levels of quinidine include but are not limited to amiodarone, a carbonic anhydrase inhibitor, cimetidine, diltiazem, itraconazole, ketoconazole, a macrolide antibiotic, a protease inhibitor, and voriconazole. Non-limiting examples of macrolide antibiotics include erythromycin, azithromycin, clarithromycin, dirithromycin, and roxithromycin. Non-limiting examples of protease inhibitors include saquinavir, ritonavir, atazanavir, and indinavir.

In some embodiments, the patient is not being treated with an agent that is metabolized by CYP2D6 and may have increased plasma levels if co-administered with quinidine. Exemplary agents that are metabolized by CYP2D6 and may have increased plasma levels if co-administered with quinidine include but are not limited to dextromethorphan (over-the-counter or prescription), a tricyclic antidepressant (TCA), and atomoxetine. Non-limiting examples of TCAs include imipramine, desipramine, amitriptyline, and nortriptyline.

In some embodiments, the patient is not being treated with an agent that is related to quinidine. Exemplary agents that are related to quinidine include but are not limited to quinine and mefloquine.

In some embodiments, the patient is not being treated with an agent that might produce serotonin syndrome when co-administered with dextromethorphan. Exemplary agents that produce serotonin syndrome when co-administered with dextromethorphan include MAOIs. Non-limiting examples of MAOIs include carbamazepine, cyproterone, hyperforin, oxcarbazepine, phenobarbital, phenytoin, rifampicin, and St. John's Wort.

In some embodiments, the present disclosure provides a method of specifically treating negative symptoms of schizophrenia in a patient having schizophrenia, comprising administering to the patient therapeutically effective amounts of d6-DM and Q, wherein the patient is not being treated with an anticholinergic medication.

In some embodiments, the present disclosure provides a method of specifically treating negative symptoms of schizophrenia in a patient having schizophrenia, comprising administering to the patient therapeutically effective amounts of d6-DM and Q, wherein the patient has not received electroconvulsive treatment, repetitive transcranial magnetic stimulation, or deep brain stimulation within one year prior to treatment.

In some embodiments, the present disclosure provides a method of specifically treating negative symptoms of schizophrenia in a patient having schizophrenia, comprising administering to the patient therapeutically effective amounts of d6-DM and Q, wherein the patient does not have myasthenia gravis.

In some embodiments, the present disclosure provides a method of specifically treating negative symptoms of schizophrenia in a patient having schizophrenia, comprising administering to the patient therapeutically effective amounts of d6-DM and Q, wherein the patient does not have a depressive disorder and/or a Calgary Depression Scale for Schizophrenia (CDSS) score of greater than or equal to 6.

In some embodiments, the present disclosure provides a method of specifically treating negative symptoms of schizophrenia in a patient having schizophrenia, comprising administering to the patient therapeutically effective amounts of d6-DM and Q, wherein the patient does not have a score of greater than 3 on the sum of eight items of the Simpson-Angus Scale (SAS): gait, arm dropping, shoulder shaking, elbow rigidity, wrist rigidity, leg pendulousness, head rotation, and glabella tap.

In some embodiments, the present disclosure provides a method of specifically treating negative symptoms of schizophrenia in a patient having schizophrenia, comprising administering to the patient therapeutically effective amounts of d6-DM and Q, wherein the patient does not have a concurrent clinically significant or unstable systemic disease, neurological disorder, cognitive disorder, neurodegenerative disorder, hepatic disorder, renal disorder, metabolic disorder, hematological disorder, immunological disorder, cardiovascular disorder, pulmonary disorder, or gastrointestinal disorder, as determined by the prescribing doctor.

In some embodiments, the present disclosure provides a method of specifically treating negative symptoms of schizophrenia in a patient having schizophrenia, comprising administering to the patient therapeutically effective amounts of d6-DM and Q, wherein the patient does not have schizoaffective disorder.

In some embodiments, the present disclosure provides a method of specifically treating negative symptoms of schizophrenia in a patient having schizophrenia, comprising administering to the patient therapeutically effective amounts of d6-DM and Q, wherein the patient does not have bipolar disorder.

In some embodiments, the present disclosure provides a method of specifically treating negative symptoms of schizophrenia in a patient having schizophrenia, comprising administering to the patient therapeutically effective amounts of d6-DM and Q, wherein the patient does not have a suicide risk. In some embodiments, suicide risk is determined by one or more of the following:

(a) judgment of the prescribing doctor;

(b) the patient answers yes on the Columbia Suicide Severity Rating Scale (C-SSRS Suicidal Ideation Item 4 (active suicidal ideation with some intent to act, without a specific plan) and the patient's most recent episode meeting this C-SSRS Item 4 occurred within six months;

(c) the patient answers yes on the C-SSRS Suicidal Behavior Item 5 (active suicidal ideation with specific plan and intent) and the patient's most recent episode meeting this C-SSRS Item 5 occurred within six months; and

(d) the patient answers yes on any of the 5 on the C-SSRS Suicidal Behavior Items (active attempt, interrupted attempt, aborted attempt, preparatory acts, or behavior) and the patient's most recent episode meeting any of these C-SSRS Items occurred within two years prior to treatment. For example, in some such embodiments, suicide risk is determined by all of (a). (b), (c), and (d). For example, in some such embodiments, suicide risk is determined by (a), (b), and (c). For example, in some such embodiments, suicide risk is determined by (a) and (b). For example, in some such embodiments, suicide risk is determined by any one of (a), (b), (c), or (d).

In some embodiments, the present disclosure provides a method of specifically treating negative symptoms of schizophrenia in a patient having schizophrenia, comprising administering to the patient therapeutically effective amounts of d6-DM and Q, wherein the patient does not have a cardiovascular history of any one or more of:

(a) history or evidence of complete heart block, ventricular tachycardia, presence of clinically significant premature ventricular contractions (PVCs) as evaluated by a central reader. QTc prolongation, or torsades de pointes;

(b) QTc using the Fridericia's formula (QTcF) greater than 450 msec for males and greater than 470 msec for females based on central review, unless due to ventricular pacing;

(c) family history of congenital QT interval prolongation syndrome; and

(d) history or presence of clinically significant syncope, orthostatic hypotension, or postural tachycardia. For example, in some such embodiments, the patient does not have a history of all of (a), (b), (c), and (d). For example, in some such embodiments, the patient does not have a history of (a), (b), and (c). For example, in some such embodiments, the patient does not have a history of (a) and (b). For example, in some such embodiments, does not have a history of any one of (a), (b), (c), or (d)

In some embodiments, the present disclosure provides a method of specifically treating negative symptoms of schizophrenia in a patient having schizophrenia, comprising administering to the patient therapeutically effective amounts of d6-DM and Q, wherein the patient does not have pseudoparkinsonism secondary to treatment with an antipsychotic.

In some embodiments, the present disclosure provides a method of specifically treating negative symptoms of schizophrenia in a patient having schizophrenia, comprising administering to the patient therapeutically effective amounts of d6-DM and Q, wherein the patient does not have a history of substance and/or alcohol abuse, but may use tobacco and/or nicotine products.

In some embodiments, the present disclosure provides a method of specifically treating negative symptoms of schizophrenia in a patient having schizophrenia, comprising administering to the patient therapeutically effective amounts of d6-DM and Q, wherein the patient does not use recreational or medicinal marijuana, as evidenced by a negative urine drug screen for cannabis.

In some embodiments, the present disclosure provides a method of specifically treating negative symptoms of schizophrenia in a patient having schizophrenia, comprising administering to the patient therapeutically effective amounts of d6-DM and Q, wherein the patient does not test positive for hepatitis B surface antigen, hepatitis C antibody, or HIV antibody.

In some embodiments, the present disclosure provides a method of specifically treating negative symptoms of schizophrenia in a patient having schizophrenia, comprising administering to the patient therapeutically effective amounts of d6-DM and Q, wherein during the first week of treatment, the d6-DM is administered in a 24 mg dose once daily and the Q is administered in a 4.9 mg dose once daily; during the second week of treatment, the d6-DM is administered in a 24 mg dose twice daily and the Q is administered in a 4.9 mg dose twice daily; and during the remainder of the treatment, the d6-DM is administered in a 34 mg dose twice daily and the Q is administered in a 4.9 mg dose twice daily.

In some embodiments, the present disclosure provides a method of specifically treating negative symptoms of schizophrenia in a patient having schizophrenia, comprising administering to the patient therapeutically effective amounts of d6-DM and Q, wherein during the first three days of treatment, the d6-DM is administered in a 28 mg dose once daily and the Q is administered in a 4.9 mg dose once daily; during the next four days of treatment, the d6-DM is administered in a 28 mg dose twice daily and the Q is administered in a 4.9 mg dose twice daily; and during the remainder of the treatment, the d6-DM is administered in a 42.63 mg dose twice daily and the Q is administered in a 4.9 mg dose twice daily.

In some embodiments, the present disclosure provides a method of treating prosocial factors in a patient having schizophrenia, comprising administering to the patient therapeutically effective amounts of d6-DM and Q. Prosocial factors include the following PANSS factors: G16 (active social avoidance); N2 (emotional withdrawal); N4 (passive/apathetic social withdrawal); N7 (stereotyped thinking); P3 (hallucinatory behavior); and P6 (suspiciousness/persecution).

In some embodiments of the methods disclosed herein, the patient has been diagnosed as having schizophrenia based on the Diagnostic and Statistical Manual of Mental Disorders (DSM) criteria for schizophrenia. In some embodiments, the DSM criteria are the criteria set forth in the American Psychiatric Association's (2000) Diagnostic and Statistical Manual of Mental Disorders, 4^{th} Edition, Text Revision (DSM-IV-TR), which is incorporated herein by reference for the disclosure of such criteria. In some embodiments, the DSM criteria are the criteria set forth in the American Psychiatric Association's (2013) Diagnostic and Statistical Manual of Mental Disorders, 5^{th} Edition (DSM-V), which is incorporated herein by reference for the disclosure of such criteria.

In some embodiments, the patient's diagnosis of schizophrenia based on the DSM criteria has been confirmed by the Mini International Neuropsychiatric Interview (M.I.N.I.). The M.I.N.I. is a brief structured diagnostic interview for psychiatric disorders, including those in DSM-IV and DSM-5. In some embodiments, the M.I.N.I. used to confirm the diagnosis of schizophrenia is M.I.N.I. Version 6.0, based on the DSM-IV-TR criteria. In some embodiments, the M.I.N.I. used to confirm the diagnosis of schizophrenia is M.I.N.I. Version 7.0.2, based on the DSM-V criteria.

In some embodiments of the methods disclosed herein, the patient satisfies one, more than one, or all of the exemplary inclusion criteria described in Section 1.3.1 of the study from Example 1. In some embodiments, the patient satisfies one, more than one, or all of the exemplary inclusion criteria described in Section 2.1 of the study from Example 2.

In some embodiments of the methods disclosed herein, the patient does not have one or more of the exemplary exclusion criteria described in Section 1.3.2 of the study from Example 1. In some embodiments, the patient does not have one or more of the exemplary exclusion criteria described in Section 2.2 of the study from Example 2.

In some embodiments of the methods disclosed herein, the patient is a male or female patient from 18 to 60 years of age.

In some embodiments of the methods disclosed herein, the patient is a female of childbearing potential. In some embodiments, the patient: (a) has a negative urine pregnancy test; (b) is not nursing or planning a pregnancy for the duration of treatment through 30 days after the last dose; and (c) is abstinent or willing to use a method of birth control prior to treatment, and to continue with the same method until 28 days after the last dose.

In some embodiments of the methods disclosed herein, the patient does not have hypersensitivity to dextromethorphan, quinidine, an opiate drug, d6-DM, Q, or any ingredient thereof.

In some embodiments of the methods disclosed herein, the patient does not have allergy or hypersensitivity to one or more medications.

In some embodiments of the methods disclosed herein, the patient does not have one or more clinically significant laboratory abnormalities, one or more safety values of clinical concern, or aspartate aminotransferase (AST) or alanine aminotransferase (ALT) levels greater than two times the upper limit of normal, as determined by the prescribing doctor.

In some embodiments of the methods disclosed herein, the patient is administered the d6-DM and the Q irrespective of the patient's ALDH2 genotype.

Lee at al. (Psychiatr Res. 2015; 69:50-56) evaluated dextromethorphan as an add-on to treatment with the antipsychotic risperidone. In that study, Lee et al. assessed patients using the Positive and Negative Syndrome Scale (PANSS) and the Scale for the Assessment of Negative Symptoms (SANS). Id. at 52. No significant differences in the PANSS and SANS scores were reported between patients treated with risperidone and dextromethorphan and patients treated with risperidone and placebo. Id.

Lee at al. (Psychiatr Res. 2015; 69:50) also evaluated a subgroup of 13 patients that had the ALDH2*2 allele of the ALDH2 gene (7 administered risperidone and dextromethorphan; 6 administered risperidone and placebo). Id. at 52-53, including Table 2. The ALDH2 allele is carried by about 50% of the Asian population, but is rarely seen in Caucasians. Id. at 54. No significant difference in the two groups (dextromethorphan vs. placebo) was reported in the PANSS scores, including the PANSS negative symptom subscale, but a significant difference was reported in the total SANS scores. Id. at 52-53. According to Lee et al., these results indicate that the ALDH2 gene might affect changes in SANS total scores. Id. at 54. Lee et al. also mentioned several limitations of the study and questioned whether the results were applicable to Western populations. Id. It is not evident that any subsequent studies have further evaluated this subgroup of patients with the ALDH2*2 allele.

In studies described and exemplified herein, patients' ALDH2 genotype was not determined. A composition comprising d6-DM and Q (d6-DM/Q) was administered to treat negative symptoms of schizophrenia irrespective of the ALDH2 genotype of the patient (see, e.g., the study from Example 1; see also the study from Example 2).

In some embodiments of the methods disclosed herein, the patient is administered the d6-DM and the Q in conjunction with other therapeutic agents, such as, for example, one or more therapeutic agents known or identified for the treatment of schizophrenia.

In some embodiments of the methods disclosed herein, the patient is further administered an atypical antipsychotic other than clozapine. In some embodiments, the atypical antipsychotic is administered within the dose guidance from its U.S. package insert for the treatment of schizophrenia. In some embodiments, the atypical antipsychotic is an oral and long-acting intramuscular injectable. In some embodiments, the atypical antipsychotic is a second-generation atypical antipsychotic drug (SGA). Exemplary SGAs include but are not limited to olanzapine, risperidone, paliperidone, quetiapine, aripiprazole, and lurasidone. In some embodiments, the patient is not being treated with more than one SGA. In some embodiments, the patient is not being treated with more than one SGA with the exception of low dose quetiapine (e.g., up to 50 mg at night) for insomnia.

In some embodiments, the d6-DM is administered in a 24 mg, 28 mg, 34 mg, or 42.63 mg dose, e.g., once or twice daily. In some embodiments, the d6-DM is administered in a 24 mg dose, e.g., once or twice daily. In some embodiments, the d6-DM is administered in a 28 mg dose, e.g., once or twice daily. In some embodiments, the d6-DM is administered in a 34 mg dose, e.g., once or twice daily. In some embodiments, the d6-DM is administered in a 42.63 mg dose, e.g., once or twice daily. In some embodiments, the d6-DM is administered in a 34 mg dose twice daily. In some embodiments, the d6-DM is administered in a 42.63 mg dose twice daily.

In some embodiments, the Q is administered in a 4.9 mg dose, e.g., once or twice daily.

In some embodiments, the d6-DM and the Q are administered or used in a unit dosage form. In some embodiments, the unit dosage form includes 24 mg, 28 mg, 34 mg, or 42.63 mg of d6-DM and 4.9 mg of Q. In some embodiments, the unit dosage form includes 24 mg of d6-DM and 4.9 mg of Q. In some embodiments, the unit dosage form includes 28 mg of d6-DM and 4.9 mg of Q. In some embodiments, the unit dosage form includes 34 mg of d6-DM and 4.9 mg of Q. In some embodiments, the unit dosage form includes 42.63 mg of d6-DM and 4.9 mg of Q. In some embodiments, the unit dosage forms of the d6-DM and the Q are in the form of a tablet or a capsule. In some embodiments, the unit dosage forms of the d6-DM and the Q are in the form of a capsule.

In some embodiments, the d6-DM and the Q are administered or used in a combined dose, or in separate doses. In some embodiments, the separate doses are administered substantially simultaneously. In some embodiments, the combined dose of the d6-DM and the Q (or separate doses simultaneously administered) is administered once daily, twice daily, three times daily, four times daily, or more frequently. For example: 24 mg d6-DM and 4.9 mg Q per day provided in a single dose; 48 mg d6-DM and 9.8 mg Q per day provided in two doses, each dose containing 24 mg d6-DM and 4.9 mg Q; 68 mg d6-DM and 9.8 mg Q per day provided in two doses, each dose containing 34 mg d6-DM and 4.9 mg Q; 28 mg d6-DM and 4.9 mg Q per day provided in a single dose; 56 mg d6-DM and 9.8 mg Q per day provided in two doses, each dose containing 28 mg d6-DM and 4.9 mg Q; or 85.26 mg d6-DM and 9.8 mg Q per day provided in two doses, each dose containing 42.63 mg d6-DM and 4.9 mg Q. In some embodiments, the total amount of d6-DM and Q in a combined dose may be adjusted, depending upon the number of doses to be administered per day, so as to provide a suitable daily total dosage to the patient.

In some embodiments. 68 mg d6-DM and 9.8 mg Q per day are provided in two doses, each dose containing 34 mg d6-DM and 4.9 mg Q. In some embodiments, the two doses are administered 6, 8, 10, 12, 14, or 16 hours apart. In some embodiments, the two doses are administered 12 hours apart (e.g., morning and evening).

In some embodiments, 85.26 mg d6-DM and 9.8 mg Q per day are provided in two doses, each dose containing 42.63 mg d6-DM and 4.9 mg Q. In some embodiments, the two doses are administered about 6, about 8, about 10, about 12, about 14, or about 16 hours apart. In some embodiments, the two doses are administered about 12 hours apart (e.g., morning and evening).

In some embodiments, the treatment is initiated at a lower daily dose, for example 24 mg or 28 mg d6-DM in combination with 4.9 mg Q per day, and increased up to 85.26 mg d6-DM in combination with 9.8 mg Q per day.

For example, in some embodiments, during the first week of treatment, the d6-DM is administered in a 24 mg dose once daily and the Q is administered in a 4.9 mg dose once daily; during the second week of treatment, the d6-DM is administered in a 24 mg dose twice daily and the Q is administered in a 4.9 mg dose twice daily; and during the remainder of the treatment, the d6-DM is administered in a 34 mg dose twice daily and the Q is administered in a 4.9 mg dose twice daily.

In some embodiments, during the first three days of treatment, the d6-DM is administered in a 28 mg dose once daily and the Q is administered in a 4.9 mg dose once daily; during the next four days of treatment, the d6-DM is administered in a 28 mg dose twice daily and the Q is administered in a 4.9 mg dose twice daily; and during the remainder of the treatment, the d6-DM is administered in a 42.63 mg dose twice daily and the Q is administered in a 4.9 mg dose twice daily.

As will be apparent to those skilled in the art, dosages outside of these disclosed dosages and ranges may be administered in some cases. Further, it is noted that the ordinary skilled clinician or treating physician will know how and when to interrupt, adjust, or terminate therapy in consideration of individual response.

Oral administration can be employed for providing the patient with an effective dosage of d6-DM in combination with Q for treating negative symptoms of schizophrenia in a patient having schizophrenia. In some embodiments, the formulations can contain a combination of d6-DM and Q with pharmaceutically acceptable carriers or diluents known to those of skill in the art. In some embodiments, the d6-DM and the Q are administered orally. In some embodiments, the d6-DM and the Q are administered orally in a unit dosage form. In some embodiments, the unit dosage forms of the d6-DM and the Q are in the form of a capsule.

d6-DM and Q may be formulated as active ingredients in one or more pharmaceutical compositions. Such pharmaceutical compositions may also contain a pharmaceutically acceptable carrier, and optionally, other therapeutic ingredients.

Pharmaceutical compositions can be prepared in forms such as powders, capsules, tablets, suspensions, sachets, cachets, solutions, and elixirs. Carriers such as starches, sugars, microcrystalline cellulose, diluents, granulating agents, lubricants, binders, disintegrating agents, and the like can be used in oral solid preparations. In some embodiments, the compositions are prepared as oral solid preparations (such as powders, capsules, and tablets). In some embodiments, the compositions are prepared as oral liquid preparations. In some embodiments, the oral solid preparations are capsules or tablets. If desired, capsules or tablets can be coated by standard aqueous or nonaqueous techniques.

Pharmaceutical compositions suitable for oral administration can be provided as discrete units such as capsules, cachets, sachets, patches, tablets, and aerosol sprays, each containing predetermined amounts of the active ingredients, as powder or granules, or as a solution or a suspension in an aqueous liquid, a non-aqueous liquid, an oil-in-water emulsion, or a water-in-oil liquid emulsion. Such compositions can be prepared by any of the conventional methods of pharmacy, but the majority of the methods typically include the step of bringing into association the active ingredients with a carrier that constitutes one or more ingredients. In general, the compositions are prepared by uniformly and intimately admixing the active ingredients with liquid carriers, finely divided solid carriers, or both, and then, optionally, shaping the product into the desired presentation.

For example, a tablet can be prepared by compression or molding, optionally, with one or more additional ingredients. Compressed tablets can be prepared by compressing in a suitable machine the active ingredient in a free-flowing form such as powder or granules, optionally mixed with a binder, lubricant, inert diluent, surface active, or dispersing agent. Molded tablets can be made by molding, in a suitable machine, a mixture of the powdered compound moistened with an inert liquid diluent.

In some embodiments, the d6-DM and the Q are administered together in the form of a capsule. In some embodiments, the capsule comprising the d6-DM and the Q is an immediate release capsule. In some embodiments, the capsule is a hard gelatin capsule. In some embodiments, the capsule is size 3.

In some embodiments, each capsule contains 24 mg, 28 mg, 34 mg, or 42.63 mg of d6-DM and 4.9 mg of Q. In some embodiments, each capsule contains 24 mg of d6-DM and 4.9 mg of Q. In some embodiments, each capsule contains 28 mg of d6-DM and 4.9 mg of Q. In some embodiments, each capsule contains 34 mg of d6-DM and 4.9 mg of Q. In some embodiments, each capsule contains 42.63 mg of d6-DM and 4.9 mg of Q. In some embodiments, each capsule is administered once daily. In some embodiments, each capsule is administered twice daily.

In some embodiments, each capsule contains 34 mg of d6-DM and 4.9 mg of Q. and is administered twice daily.

In some embodiments, each capsule contains 42.63 mg of d6-DM and 4.9 mg of Q, and is administered twice daily.

In some embodiments, each capsule (or other composition comprising d6-DM and Q as active ingredients) also contains inactive ingredients. In some embodiments, the inactive ingredients may include croscarmellose sodium, microcrystalline cellulose, colloidal silicone dioxide, and/or magnesium stearate. In some embodiments, the inactive ingredients consist of or comprise croscarmellose sodium, microcrystalline cellulose, colloidal silicone dioxide, and magnesium stearate.

### Deuterated Dextromethorphan Hydrobromide

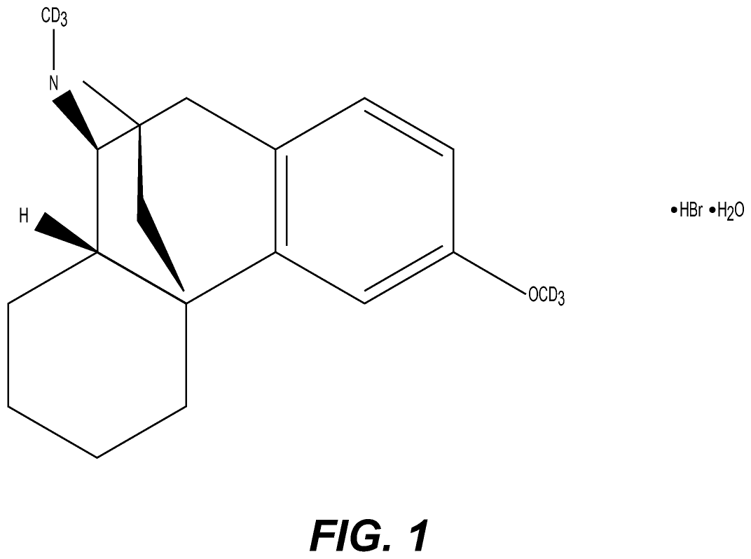

Dextromethorphan (DM) is the common name for (+)-3-methoxy-N-methylmorphinan, one of a class of molecules that are dextrorotatory analogs of morphine-like opioids. FIG. 1 shows the structure of deuterated [d6]-dextromethorphan hydrobromide (d6-DM), which is a deuterated isotope of DM in which deuterium replaces 6 hydrogen atoms at locations shown in FIG. 1.

In some embodiments, d6-DM is isolated or purified, e.g., d6-DM is present at a purity of at least 50% by weight (e.g., at least 55%, 60%, 65%, 70%, 75%, 80%, 85%, 90%, 95%, 97%, 98%, 98.5%, 99%, 99.5%, or 99.9%) of the total amount of isotopologues of d6-DM present, respectively. Thus, in some embodiments, a composition comprising d6-DM can include a distribution of isotopologues of the compound, provided at least 50% of the isotopologues by weight are d6-DM.

In some embodiments, any position in d6-DM designated as having D has a minimum deuterium incorporation of at least 80%, at least 85%, at least 87%, at least 90%, at least 95%, at least 97%, at least 99%, or at least 99.5%) at the designated position(s) in the d6-DM. Thus, in some embodiments, a composition comprising d6-DM can include a distribution of isotopologues of the compound, provided at least 80% of the isotopologues include a D at the designated position(s).

In some embodiments, d6-DM is substantially free of other isotopologues of the compound, e.g., less than 20%, less than 10%, less than 5%, less than 2%, less than 1%, or less than 0.5% of other isotopologues are present.

The synthesis of d6-DM can be readily achieved by synthetic chemists of ordinary skill. Relevant procedures and intermediates are disclosed, for instance in Kim et al. (Bioorg Med Chem Lett 2001, 11:1651) and Newman et al. (J Med Chem 1992, 35:4135).

A convenient method for synthesizing d6-DM according to some embodiments substitutes the appropriate deuterated intermediates and reagents in synthesis methods utilized for the preparation of dextromethorphan. These methods are described, for example, in U.S. Pat. No. 7,973,049, which is incorporated by reference in its entirety.

Additional methods of synthesizing d6-DM are within the means of chemists of ordinary skill in the art. Synthetic chemistry transformations and protecting group methodologies (protection and deprotection) useful in synthesizing d6-DM are known in the art and include, for example, those described in: Larock, Comprehensive Organic Transformations, VCH Publishers (1989); Greene et al. Protective Groups in Organic Synthesis, 3^{rd} Ed., John Wiley and Sons (1999); Fieser et al. Fieser and Fieser's Reagents for Organic Synthesis, John Wiley and Sons (1994); and Paquette, ed., Encyclopedia of Reagents for Organic Synthesis, John Wiley and Sons (1995); and subsequent editions thereof.

### Quinidine Sulfate

The present disclosure envisions the use of quinidine sulfate (Q) in combination with d6-DM. In most people (estimated to include about 90% of the general population in the U.S.), dextromethorphan undergoes extensive hepatic O-demethylation to dextrorphan that is catalyzed by CYP2D6 and is rapidly eliminated by the body (Ramachander et al. J. Pharm. Sci. 1977; 66(7):1047-1048; Vetticaden et al. Pharm. Res. 1989; 6(1):13-19). However, quinidine is a potent CYP2D6 inhibitor and has been particularly studied in this use (see, e.g., U.S. Pat. No. 5,206,248). The chemical structure of the sulfate salt form of quinidine, Q ((C₂₀H₂₄N₂O₂)₂.H₂SO₄.2H₂O), is as follows:

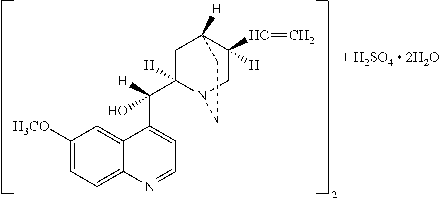

Quinidine administration can convert subjects with extensive dextromethorphan metabolizer phenotype to poor metabolizer phenotype (Inaba et al. Br. J. Clin. Pharmacol. 1986; 22:199-200).

### Exemplary Scales

In some embodiments of the methods disclosed herein, one or more scales described herein, or others known in the art, may be used. Exemplary scales include the Positive and Negative Syndrome Scale (PANSS), 16-Item Negative Symptom Assessment (NSA-16), Clinical Global Impression (CGI) Scales (e.g., Clinical Global Impression of Severity (CGI-S), Clinical Global Impression of Change (CGI-C)), Patient Global Impression of Change (PGI-C), Measurement and Treatment Research to Improve Cognition in Schizophrenia (MATRICS) Consensus Cognitive Battery (MCCB), Calgary Depression Scale for Schizophrenia (CDSS), and Effort Expenditure for Reward Task (EEfRT). In some embodiments one or more of the scales is used to assess a patient's baseline status before treatment. In some embodiments, one or more of the scales is used to assess a patient's progress at various points during treatment. In some embodiments, the patient's progress at one or more points during treatment is compared to a patient's baseline status before treatment.

Positive and Negative Syndrome Scale (PANSS)

The PANSS is a validated clinical scale that has been extensively used as a reliable and valid measure of the negative and positive symptoms of schizophrenia (Daniel, Schizophr Res. 2013; 150(2-3):343-345). The scale comprises 30 disparate items that collectively assess the positive and negative syndromes in schizophrenia, including their relationship to one another and to global psychopathology. Each is scored for “1” (absent) to “7” (extremely severe).

The established psychometric properties of the PANSS allow for the assessment of positive, negative, and general psychopathology as part of a categorical or dimensional perspective of schizophrenia (Kay, Schizophr Bull. 1987; 13(2):261-276; Kumari et al. J Addict Res Ther. 2017; 8(3)). Thus, different combinations of items are generally analyzed as factor structures to score specific aspects of the negative syndrome of schizophrenia.

The concept of a five-factor solution for the PANSS has been successfully used in clinical trials, and identifies five factors or dimensions of schizophrenia: 1) negative symptoms; 2) positive symptoms; 3) disorganized thought; 4) uncontrolled hostility/excitement; and 5) anxiety/depression (Lindenmayer et al. Psychopathology. 1995; 28(1):22-31; Marder et al. J Clin Psychiatry. 1997; 58(12):538-546). Marder investigated the five-factor solution in two controlled trials and found that risperidone produced significantly greater improvements than haloperidol on all five dimensions, with a particularly potent effect of risperidone vs. haloperidol on Factor 1 (negative symptoms). Factor 1, i.e., the PANSS Marder negative factors, has several aspects of improved content validity versus the negative subscale, meeting more consistently with the domains identified in the 2006 NIMH-MATRICS Consensus Statement (Kirkpatrick et al. Schizophr Bull. 2006; 32(2):214-219).

PANNS Marder Negative Factors Score

The PANSS Marder negative factors score is a reliable and validated measure of the negative symptoms of schizophrenia, and is comprised of the following 7 items of the 30-item PANSS:

Marder Negative Factors:
N1: Blunted affect
N2: Emotional withdrawal
N3: Poor rapport
N4: Passive/apathetic social withdrawal
N6: Lack of spontaneity and flow of conversation
G7: Motor retardation
G16: Active social avoidance

Each of the PANSS Marder negative factors correlates with one of the five main domains of negative symptoms (Kirkpatrick et al. Schizophr Bull. 2006; 32(2):214-219). PANSS item N1: blunted affect, correlates with Blunted affect; N6: lack of spontaneity and conversation flow, correlates with Alogia; and N4: passive/apathetic social withdrawal; G16: active social avoidance; and N3: poor rapport, are factors that correlate with Asociality. PANSS item N2: emotional withdrawal, correlates with Anhedonia; and G7: motor retardation, correlates with both Anhedonia and Avolition (Daniel, Schizophr Res. 2013; 150(2-3):343-345).

Two of the items in the PANSS Marder negative factors score (N4 and G16) are based solely on information obtained from an informant. In some embodiments, patients identify a reliable informant (e.g., case manager, social worker, family member) who spends sufficient time with them to be able to provide information to PANSS raters.

In some embodiments, one or more negative symptoms of schizophrenia is evaluated based on PANSS scores. In some embodiments, only PANSS scores are determined. In some embodiments, PANSS scores are determined in combination with one or more additional scales (e.g., any one or more of the exemplary scales described herein).

16-Item Negative Symptom Assessment (NSA-16)

The NSA-16 is considered a valid and reliable measure of the presence, severity, and range of negative symptoms associated with schizophrenia; it has high interrater and test-retest reliability across languages and cultures (Daniel, Schizophr Res. 2013; 150(2-3):343-345; Axelrod et al. J Psychiatr Res. 1993; 27(3):253-258). The NSA-16 uses a 5-factor model to describe negative symptoms: (1) communication, (2) emotion/affect, (3) social involvement, (4) motivation, and (5) retardation. These factors, assessed through a structured interview, are comprehensive and well-defined to help standardize assessment. As a truncated version of the 25-item NSA, the NSA-16 still captures the multidimensionality of negative symptoms but can be completed in approximately 15 to 20 minutes (Axelrod et al. J Psychiatr Res. 1993; 27(3):253-258). The NSA-4 (Alphs et al. Int J Methods Psychiatr Res. 2011; 20(2):e31-37) is comprised of the 4 NSA-16 items as follows: 1) restricted speech quantity, 2) emotion: reduced range, 3) reduced social drive, and 4) reduced interests, as well as an overall global rating of negative symptoms.

NSA global negative symptom score rates the overall severity of negative symptoms when defined as the absence or reduction of behaviors normally present in a healthy young person. In some embodiments, ratings do not depend on any specific item or items from the NSA or any other similar instrument. Instead, in some embodiments, ratings measure the rater's gestalt of the interview and are assessed following completion of the NSA-16 interview (Alphs et al. Int J Methods Psychiatr Res. 2011; 20(2):e31-37).

In some embodiments, one or more negative symptoms of schizophrenia is evaluated using the NSA-16. In some embodiments, the NSA-16 is used alone. In some embodiments, the NSA-16 is used in combination with one or more additional scales (e.g., any one or more of the exemplary scales described herein).

Clinical Global Impression (CGI) Scales

The CGI was developed to provide a brief, stand-alone assessment of the clinician's view of the patient's global functioning prior to and after initiating a treatment (Busner and Targum, Psychiatry (Edgmont). 2007; 4(7):28-37). The CGI provides an overall clinician-determined summary measure that takes into account all available information, including knowledge of the patient's history, psychosocial circumstances, symptoms, behavior, and the impact of the symptoms on the patient's ability to function. The CGI comprises 2 companion 1-item measures, the CGI-S(Severity) and CGI-C(Change).

Clinical Global Impression-Severity (CGI-S)

The CGI-S is a 7-point scale that requires the clinician to rate the severity of the patient's illness at the time of assessment, relative to the clinician's past experience with patients who have the same diagnosis (Guy, ECDEU Assessment Manual for Psychopharmacology. 1976:76-338). Considering total clinical experience, a patient is assessed on severity of mental illness at the time of rating 1, normal, not at all ill; 2, borderline mentally ill; 3, mildly ill; 4, moderately ill; 5, markedly ill; 6, severely ill; or 7, among the most extremely ill patients.

Clinical Global Impression-Change (CGI-C)

The CGI-C is a 7-point scale that requires the clinician to rate the change of the patient's condition at the time of assessment, relative to the clinician's past experience with the patient's condition at admission. Considering total clinical experience, a patient is assessed for change of mental illness as 1, Very much improved; 2. Much improved; 3, Minimally improved; 4, No change; 5, Minimally worse; 6, Much worse; or 7, Very much worse.

In some embodiments, one or more negative symptoms of schizophrenia is evaluated using the CGI (e.g., the CGI-S and/or CGI-C). In some embodiments, the CGI (e.g., the CGI-S and/or CGI-C) is used alone. In some embodiments, the CGI (e.g., the CGI-S and/or CGI-C) is used in combination with one or more additional scales (e.g., any one or more of the exemplary scales described herein).

Patient Global Impression-Severity (PGI-S)

The PGI-S is a 7-point (1-7), patient-rated scale used to assess the severity of the patient's schizophrenia as follows: 1) normal, not at all ill; 2) borderline ill; 3) mildly ill; 4) moderately ill; 5) markedly ill; 6) severely ill; 7) extremely ill.

In some embodiments, one or more negative symptoms of schizophrenia is evaluated using the PGI-S. In some embodiments, the PGI-S is used alone. In some embodiments, the PGI-S is used in combination with one or more additional scales (e.g., any one or more of the exemplary scales described herein).

Patient Global Impression-Change (PGI-C)

The PGI-C is a 7-point (1-7), patient-rated scale used to assess treatment response with respect to the patient's schizophrenia as follows: very much improved, much improved, minimally improved, no change, minimally worse, much worse, or very much worse.

In some embodiments, one or more negative symptoms of schizophrenia is evaluated using the PGI-C. In some embodiments, the PGI-C is used alone. In some embodiments, the PGI-C is used in combination with one or more additional scales (e.g., any one or more of the exemplary scales described herein).

Measurement and Treatment Research to Improve Cognition in Schizophrenia (MATRICS) Consensus Cognitive Battery (MCCB)

The MCCB is the standard tool for assessing cognitive change in trials of cognitive-enhancing agents in schizophrenia. The MCCB (Nuechterlein et al. Am J Psychiatry. 2008; 165(2):203-213) is intended to provide a relatively brief evaluation of key cognitive domains relevant to schizophrenia and related disorders. The MCCB includes 10 tests that measure 7 cognitive domains: Speed of Processing, Attention/Vigilance, Working Memory, Verbal Learning, Visual Learning, Reasoning, and Problem Solving and Social Cognition.

In some embodiments, cognitive domains are evaluated using the MCCB. In some embodiments, the MCCB is used alone. In some embodiments, the MCCB is used in combination with one or more additional scales (e.g., any one or more of the exemplary scales described herein).

Calgary Depression Scale for Schizophrenia (CDSS)

The CDSS is a 9-item scale derived from the Hamilton Depression Scale (Ham-D) that is designed to assess depression specifically in patients with schizophrenia (Addington et al. Schizophr Res. 1996; 19(2-3):205-12). Unlike the Ham-D, the CDSS does not contain depressive symptoms that overlap with negative symptoms of schizophrenia, such as anhedonia and social withdrawal. The CDSS has shown excellent psychometric properties. Each item on the scale is scored as 0, Absent; 1, Mild; 2, Moderate; or 3, Severe. The CDSS score is obtained by adding each of the item scores. A score above 6 has an 82% specificity and 85% sensitivity for predicting the presence of a major depressive episode.

In some embodiments, depression is evaluated using the CDSS. In some embodiments, the CDSS is used alone. In some embodiments, the CDSS is used in combination with one or more additional scales (e.g., any one or more of the exemplary scales described herein).

Effort Expenditure for Reward Task (EEfRT)

The Effort Expenditure for Rewards Task (EEfRT) (Treadway et al. PLoS One. 2009; 4(8):e6598) is a multi-trial computerized task in which patients are given an opportunity to choose between 2 tasks that differ in difficulty level and are associated with varying levels of monetary reward. This task examines probabilistic learning in response to variable reward schedules and effort expended (button pushing) for reward. Probability is manipulated in the EEfRT, because, like mobilization of effort, probability discounting appears to be highly predictive of negative symptoms. Additionally, the inclusion of a probability manipulation improves the overall ecological validity of the task, as most real-world choices that require motivation are usually associated with some level of uncertainty in the outcome. The EEfRT reliably measures drug effects on willingness to expend effort in relation to amount of reward or probability of reward. For example, amphetamine increased effort in response to low- and moderate probability rewards (Wardle et al. J Neurosci. 2011; 31(46):16597-16602). Whereas reward salience and behavioral response have been linked to dopamine release in the striatum, glutamatergic input to midbrain dopamine neurons via NMDA receptors is also required for reward conditioning (Stuber et al. Science. 2008; 321(5896):1690-1692). In some embodiments, the ratio of hard task choices with moderate probability reward is used as outcome measure for negative symptoms.

In some embodiments, one or more negative symptoms of schizophrenia is evaluated using the EEfRT. In some embodiments, the EEfRT is used alone. In some embodiments, the EEfRT is used in combination with one or more additional scales (e.g., any one or more of the exemplary scales described herein).

In some embodiments of the methods disclosed herein, one or more negative symptoms of schizophrenia is evaluated using the NSA-16 total score. In some embodiments, one or more negative symptoms of schizophrenia is evaluated using any one or more the PANSS total score; PANSS subscales (e.g., positive, negative, general psychopathology, Marder negative factors, excitement component, and/or prosocial factors); the NSA-16 factor domains; the NSA-16 global symptom/functioning score; NSA-16 individual items score; the NSA-4 score; the CGI-S score; the CGI-C score; the PGI-C score; and the EEfRT score. In some such embodiments, only one of the scales is used. In some such embodiments, all of the scales are used. In some such embodiments, combinations of two or more scales are used. In some embodiments, cognition is evaluated using the MCCB composite score. In some embodiments, depression is evaluated using the CDSS. In some embodiments, one or more negative symptoms of schizophrenia is evaluated using any one or more of the efficacy endpoints and/or scales described in the study from Example 1.

In some embodiments of the methods disclosed herein, one or more negative symptoms of schizophrenia is evaluated using the PANSS Marder negative factors score. In some embodiments, one or more negative symptoms of schizophrenia is evaluated using the NSA-16 global negative symptoms score. In some embodiments, one or more negative symptoms of schizophrenia is evaluated using the PGI-S score. In some embodiments, one or more negative symptoms of schizophrenia is evaluated using the PGI-C score. In some embodiments, one or more negative symptoms of schizophrenia is evaluated using the PANSS positive subscale and depression is evaluated using the CDSS. In some embodiments, one or more negative symptoms of schizophrenia is evaluated using any one or more of the efficacy endpoints and/or scales described in the study from Example 2.

In some embodiments of the methods disclosed herein, the patient has clinically stable positive symptoms. In some embodiments, the patient has been diagnosed as having clinically stable positive symptoms based on factors such as inpatient psychiatric hospitalization, psychiatric hospital admission or acute exacerbation, and/or a particular score on certain aspects of the Positive and Negative Syndrome Scale (PANSS).

In some embodiments, the PANSS positive subscale (P1-P7) indicates changes in psychotic symptoms, and includes P1: delusions; P2: conceptual disorganization; P3: hallucinatory behavior; P4: excitement; P5: grandiosity; P6: suspicious/persecution; and P7: hostility. In some embodiments, the patient has clinically stable positive symptoms based on the PANSS positive subscale.

### Clinical Study Design

In the clinical study in Example 1 below, the benefit of treating negative symptoms of schizophrenia by administering d6-DM and Q was assessed. That study included a placebo group and a group that was administered d6-DM and Q. Having a placebo group in this study was particularly informative, because high placebo responses are commonly observed in studies of psychiatric disorders (see, e.g., Fava et al. “The Problem of the Placebo Response in Clinical Trials for Psychiatric Disorders: Culprits, Possible Remedies, and a Novel Study Design Approach,” Psychother Psychosom. 2003; 72(3):115-127 at 115-116). A “placebo response represents an apparent improvement in the clinical condition of patients randomly assigned to placebo treatment . . . ” Id. at 116. Thus, to avoid ambiguity of whether improvement in patients is provided by a therapeutic benefit of the drug or due to a placebo response, a study involving administration of a drug for treatment of a psychiatric disorder should include a placebo group.

Also, the clinical study in Example 1 below considered certain other factors that can exacerbate negative symptoms of schizophrenia, such as positive symptoms, depression (evaluated with the Calgary Depression Scale for Schizophrenia), and extrapyramidal symptoms. After considering those other factors, the study concluded that the drug treatment was specifically treating the negative symptoms of schizophrenia rather than improving such other exacerbating symptoms. See, e.g., Kirkpatrick et al., “The NIMH-MATRICS Consensus Statement on Negative Symptoms,” Schizophrenia Bulletin, 32(2):214-219 (2006) (Kirkpatrick); Arango et al., “Pharmacological approaches to treating negative symptoms: A review of clinical trials,” Schizophrenia Research, 150(2-3):346-352 (2013). An ambiguity that arises when it is not clear if a drug has had a direct effect on negative symptoms or instead indirectly effected negative symptoms by improving exacerbating symptoms has been called a “pseudo-specificity problem.” Kirkpatrick at 216. “An improvement of negative symptoms in clinically stable patients, whose psychotic symptoms have been treated to a usual clinical standard and do not change significantly, would allow an unambiguous interpretation.” Id.

In the clinical study in Example 1 below, patients had low baseline PANSS positive scores and demonstrated little change during the study. Also, in the study in Example 1 below, patients had low baseline symptoms of depression (evaluated with the Calgary Depression Scale for Schizophrenia) and pyramidal symptoms and did not experience significant changes in these symptoms throughout the study. Such results support the conclusion that administration of d6-DM and Q specifically treated negative symptoms of schizophrenia.

The following examples provide illustrative embodiments of the disclosure. One of ordinary skill in the art will recognize the numerous modifications and variations that may be performed without altering the spirit or scope of the disclosure. Such modifications and variations are encompassed within the scope of the disclosure. The examples provided do not in any way limit the disclosure.

### EXAMPLES

### Example 1

A Multicenter, Randomized, Double-Blind, Placebo-Controlled, Study to Assess the Efficacy, Safety, and Tolerability of d6-DM/Q (Deuterated [d6]-Dextromethorphan Hydrobromide [d6-DM]/Quinidine Sulfate [Q]) as an Adjunctive Treatment for Patients with Schizophrenia

To evaluate the efficacy, safety, and tolerability of d6-DM/Q when taken as adjunctive therapy in patients with negative symptoms of schizophrenia, a randomized, placebo-controlled sequential parallel comparison design (SPCD) study was performed.

The patient population studied was enriched for negative symptoms of schizophrenia and the primary efficacy endpoint of the study was the 16-Item Negative Symptom Assessment (NSA-16) total score, a validated and widely-used measure of negative symptoms of schizophrenia (Daniel, Schizophr Res. 2013; 150(2-3):343-5; Axelrod et al. J Psychiatr Res. 1993; 27(3):253-8). The patient population studied had clinically stable positive symptoms treated with background second-generation atypical antipsychotic medication. d6-DM/Q was tested at doses of 34 mg d6-DM/4.9 mg Q (d6-DM/Q-34/4.9) twice daily (BID).

1 Investigational Plan

1.1 Overall Study Design

This was a Phase 2, multicenter, randomized, double-blind, placebo-controlled, SPCD study consisting of an up to 4-week screening period, a 12-week double-blind treatment period with 2 consecutive stages (Stage 1 and Stage 2), and a 5-day follow-up by telephone. The length of each patient's participation in the study was approximately 12 weeks with a maximum of 17 weeks including the screening phase and post study exit phone calls for 5 days.

Approximately 120 patients were planned to be enrolled at 15 centers in the U.S. Patients diagnosed with schizophrenia who were clinically stable, in a residual (non-acute) phase of illness, and who met all inclusion and none of the exclusion criteria were eligible for enrollment. In Stage 1, patients were randomized in a 1:2 (active:placebo) ratio to receive either d6-DM/Q or matching placebo capsules. Patients who were randomized to the d6-DM/Q group began receiving d6-DM 24 mg/Q 4.9 mg (d6-DM/Q-24/4.9) once daily (QD) on Day 1 for the first 7 days. Scheduled dose escalations occurred on Day 8 (to d6-DM/Q-24/4.9 BID and Day 14 to d6-DM/Q-34/4.9 BID whereas patients randomized to placebo received placebo BID in Stage 1.

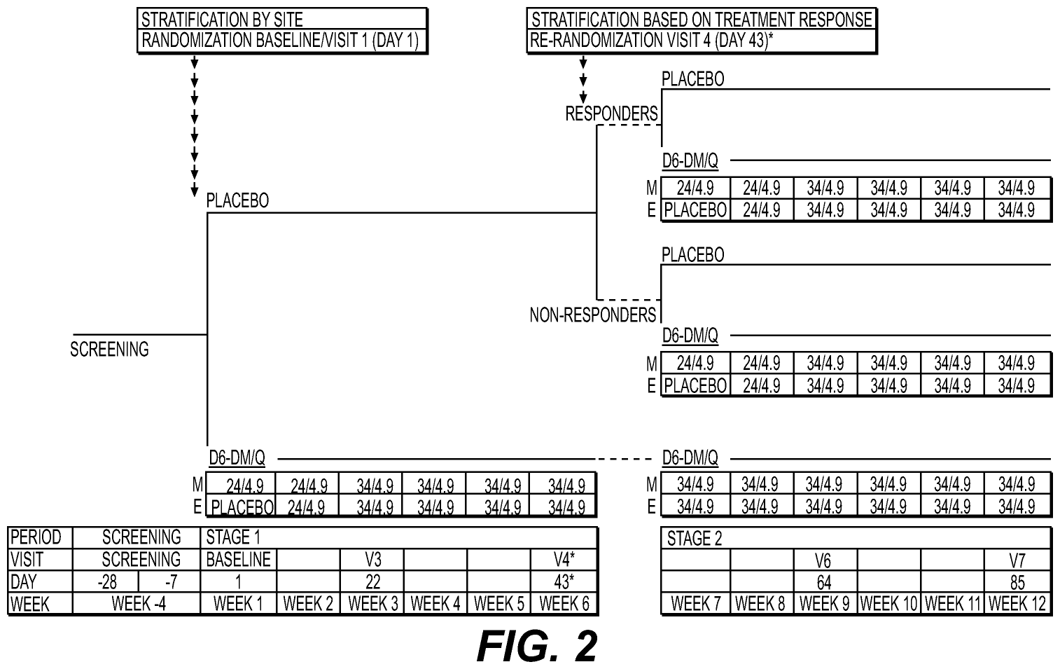

Patients who completed Stage 1 were eligible to participate in Stage 2. Patients who received d6-DM/Q in Stage 1 continued to receive d6-DM/Q 34/4.9 BID in Stage 2 with no further dose escalations. Patients who received placebo in Stage 1 were stratified into 1 of 2 treatment-response subgroups (responders vs. non-responders) at Stage 2 baseline (Visit 4 [Day 43]) and re-randomized in a 1:1 (active:placebo) ratio within each subgroup. Patients were considered responders if their percent of change in Positive and Negative Syndrome Scale (PANSS) total score decreased ≥20% from baseline, and those who did not meet this criterion were considered as non-responders. Patients who were re-randomized from placebo in Stage 1 to d6-DM/Q in Stage 2 began receiving d6-DM/Q in Stage 2 using the same dose escalation schedule used in Stage 1 whereas patients re-randomized to placebo received placebo BID for the entire duration of Stage 2. A schematic of the study is shown in FIG. 2.

Patients attended a screening visit up to 4 weeks (28 days) prior to the baseline visit to determine eligibility for the study. During the study, patients attended clinic visits at baseline/Visit 1 (Day 1), Week 2/Visit 2 (Day 15), Week 3/Visit 3 (Day 22), Week 6/Visit 4 (Day 43), Week 8/Visit 5 (Day 57), Week 9/Visit 6 (Day 64) and Week 12/Visit 7/Early Termination visit (Day 85) as outlined in Table 1. Patients also received telephone follow-up calls at Days 8 and 50 to inquire about adverse events (AEs) and study drug compliance. In addition, post study exit follow-up telephone calls were made daily on Days 86 to 90 to assess any changes in health (i.e., AEs) and medication (i.e., concomitant medications).

TABLE 1
Study Design and Schedule of Assessments

| | | | | | | | | | | | | |
|---|---|---|---|---|---|---|---|---|---|---|---|---|
| | | | | | | | | | | | Visit 7/ | |
| | Visit: | Screening | Baseline | Phone⁷ | Visit 2² | | Visit 4² | Phone⁷ | Visit 5² | Visit 6¹ | ET ^{2,4} | Post Exit |
| | Study Day: | Day −28 to −7 | Visit 1 | Day 8 | Day 15 | Day 22 | Day 43 | Day 50 | Day 57 | Day 64 | Day 85 | Phone⁷ |
| Procedure | Study Week: | Week −4 to −1 | Day 1 | Week 1 | Week 2 | Week 3 | Week 6 | Week 7 | Week 8 | Week 9 | Week 12 | Day 86-90 |

| | | | | | | | | | | | |
|---|---|---|---|---|---|---|---|---|---|---|---|
| Informed Consent Form signed | X | | | | | | | | | | |
| CTS Database | X | | | | | | | | | X | |
| Medical History | X | | | | | | | | | | |
| M.I.N.I. Exam | X | | | | | | | | | | |
| Inclusion and Exclusion | X | | | | | | | | | | |
| Randomization/ | | X | | | | X | | | | | |
| (re-randomization) | | | | | | | | | | | |
| Physical Examination | X | | | | | | | | | | |
| Resting 12-lead Electrocardiogram | X⁶ | X⁶ | | | | X⁶ | | | | X⁶ | |
| Chemistry, Hematology, and | X | | | | | X | | | | X | |
| Urinalysis³ | | | | | | | | | | | |
| Pregnancy Test³ | X | X | | | X | X | | | X | X | |
| Lab-CYP2 D6 | | X⁵ | | | | | | | | | |
| Plasma Antipsychotic Levels | X | | | | | X | | | | X | |
| PK and Additional Genotyping | | X⁵ | | | | X⁵ | | | | X⁵ | |
| Blood Samples | | | | | | | | | | | |
| Review of Adverse Events | | X | X | X | X | X | X | X | X | X | X |
| Concomitant Medications | X | X | X | X | X | X | X | X | X | X | X |
| Record Vital Signs/Weight⁹ | X | X | | | X | X | | X | X | X | |
| NSA-16 | X | X | | | X | X | | | X | X | |
| PANSS | X | X | | | | X | | | X | X | |
| MCCB¹⁰ | X | X | | | | X | | | | X | |
| CGI-S | | X | | | | X | | | | X | |
| CDSS | X | X | | | X | X | | | X | X | |
| CGI-C | | | | | | X | | | | X | |
| PGI-C | | | | | | X | | | | X | |
| RDoC Task (EEfRT) | | X | | | | X | | | | X | |
| Side Effects Scales | | X | | | | X | | | | X | |
| (AIMS, BAS, SAS) | | | | | | | | | | | |
| C-SSRS | X | X | | X | X | X | | X | X | X | |
| Smoking Cessation Question | | X | | | | X | | | | X | |
| Dispense Study Medication | | X | | | X | X | | | X | | |
| Dose in Clinic | | X | | X | X | X | | X | X | X | |
| Review and/or Return Unused | | | X⁸ | X | X | X | X⁸ | X | X | X | |
| Study Medication | | | | | | | | | | | |

AIMS = Abnormal Involuntary Movements Scale;
BAS = Barnes Akathisia Scale;
CDSS = Calgary Depression Scale for Schizophrenia;
CGI-C = Clinical Global Impression of Change;
CGI-S = Clinical Global Impression of Severity of Illness;
C-SSRS = Columbia Suicide Severity Rating Scale;
CTS = Clinical Trial Subject (database);
CYP2D6 = Cytochrome P450 isoenzyme 2D6;
EEfRT = Effort Expenditure for Reward Task;
MCCB = MATRICS Consensus Cognitive Battery;
M.I.N.I = Mini International Neuropsychiatric Interview;
NSA-16 = 16-Item Negative Symptom Assessment;
PANSS = Positive and Negative Syndrome Scale;
PGI-C = Patient Global Impression of Change.
¹Visits 3 and 6 had a +3-day window.
²Visits 2, 4, 5, and 7 had a ±3-day window.
³Urinary (beta-hCG) test were performed for all females regardless of childbearing potential (serum beta-hCG at screening only). Fasting glucose and lipids were measured at screening, Visits 4, and 7.
⁴ Final visit or Early Termination visit for patients who withdrew prior to study completion.
⁵PK blood draws were taken 2-3 hours' post-dose for Baseline and Visits 4 and 7. Blood samples for CYP2D6 genotyping were taken before dosing at Baseline. A one-time blood sample for additional genotyping may have been taken at any visit when blood was already being drawn.
⁶Electrocardiogram were performed prior to dosing and 2-3 hours post-dosing for Visits 1 and 4, Post-dose only for Visit 7. Triplicate ECGs at screening only.
⁷Telephone call had a +3-day window. Post study exit calls were made daily for 5 days to assess any changes in health (AEs)/concomitant medications.
⁸Patient were asked if they have taken their medications as directed during telephone calls at Days 8 and 50.
⁹Height was measured at screening only.
¹⁰The MCCB should have been conducted at approximately the same time of day (±2 hours) and preferably in the AM. After the screening visit, patients who used sedatives/hypnotics or benzodiazepine medications on a prn basis should not have taken any of these medications the day of, or the day before, the assessment of cognitive function by MCCB. Patients who were on stable dose regimens of sedatives/hypnotics or benzodiazepine medications were to take their medication as
prescribed.

1.2 Discussion of the Study Design, Including the Choice of Control Groups

A randomized, placebo-controlled, double-blind study design was adopted to reduce sources of bias inherent to studies of schizophrenia. In addition, an SPCD that stratified placebo-treated patients in Stage 2 according to their responder status at the end of Stage 1 was used to reduce the impact of a confounding placebo response. High placebo responses are commonly observed in studies of behavioral and psychiatric disorders and constitute a significant challenge for drug development in these indications (Fava et al. Psychother Psychosom. 2003; 72(3):115-127; Chen et al. Contemp Clin Trials. 2011; 32(4):592-604). The SPCD (Chen et al. Contemp Clin Trials. 2011; 32(4):592-604) selected for this study attempts to overcome these challenges by stratifying placebo-treated patients according to treatment response at the end of Stage 1 in order to reduce the detrimental impact of placebo response on signal detection. As a result, this design essentially comprised of two randomized trials run one after another with the expectation that signal detection would be enhanced by including only placebo non-responders in the primary analysis. The opportunity to compare patients who have taken drug or placebo for the entire duration of the trial (a built-in parallel study comparison) was retained with this design.

The 6-week durations for Stage 1 and Stage 2 were selected to ensure exposure to the targeted optimal dose of d6-DM/Q for at least 4 weeks and up to 10 weeks for patients randomized to d6-DM/Q in Stage 1. This treatment duration also allowed for sufficient time to observe a treatment response, which was expected to be within the first few weeks of study treatment, and to assess duration of response. A consensus group comprised of representatives from the National Institute of Mental Health, FDA, academic, and industry identified a 6 to 12-week duration of treatment as appropriate for designing studies of negative symptoms of schizophrenia (Laughren and Levin, Schizophr Bull. 2006; 32(2):220-222).

The safety assessments used in this study are standard in clinical research. The rating scales used to assess efficacy are well-established instruments that are clinically validated and have been widely used in clinical studies of schizophrenia and other psychiatric and behavioral disorders. The primary efficacy analysis was based on methods reviewed by Chen et al. and employed in previously conducted studies using SPCD (Chen et al. Contemp Clin Trials. 2011; 32(4):592-604). Calculation of the overall effect is based on a combination of the effects observed in Stage 1 and observed in Stage 2 for placebo non-responder stratum only (Fava et al. Psychother Psychosom. 2003; 72(3):115-127).

1.3 Selection of Study Population

1.3.1 Inclusion Criteria
1. Males and females 18 to 60 years of age, inclusive, at time of informed consent.
2. Patients who met DSM-IV-TR diagnostic criteria for schizophrenia using the Mini International Neuropsychiatric Interview (M.I.N.I.) version 6.0 (Appendix 11A) and who met DSM-IV-TR diagnostic criteria for residual schizophrenia.
3. Patients must have had a score of ≤4 on PANSS items of delusions, hallucinations, and hostility. Patients must have a PANSS score of ≥4 (moderate) on any 2, or ≥5 on any 1, of the following items of the PANSS: blunted affect, emotional withdrawal, passive/apathetic social withdrawal, or lack of spontaneity/flow of conversation and a total PANSS negative subscale score (N1 to N7) of ≥18 at screening and baseline.
4. If female of childbearing potential, patients' must
   a. have had a negative urine pregnancy test (all females regardless of childbearing potential are required to submit a pregnancy test), and
   b. not have been nursing or planning a pregnancy for the duration of the study through 30 days after the last dosing visit, and
   c. have been abstinent or willing to use a reliable method of birth control from the screening visit, and continue with the same method until 28 days after the last dosing visit
   Reliable methods of contraception that meet the study requirements were:
      Intrauterine device
      Vasectomized partner
      Surgical sterilization (have had uterus and/or both ovaries removed, and/or have had a bilateral tubal ligation)
      Hormonal contraceptives (estrogen-containing birth control pills, vaginal ring, patch, injections or implants)
      The use of 2 barrier methods of contraception (i.e., 2 of the following used together): male condom with intravaginal spermicide, diaphragm with spermicide; cervical cap with spermicide
   Note: The mini pill (micro-dosed progesterone preparations that do not contain estrogen) was not an acceptable form of contraception for this study.
   Females of childbearing potential who were abstinent could have enrolled in the study.
   A female was considered of childbearing potential unless she was post-menopausal (i.e., history compatible with menopause [i.e., reported lack of menses for ≥12 months] and no other biological/surgical cause).
   All male patients must have followed the same methods of birth control with partners of childbearing potential from the screening visit until 28 days after the last dosing visit, Visit 7/Early Termination visit (Day 85).
5. Patients currently receiving atypical antipsychotics (oral and long acting intramuscular injectables) within the dose guidance from the U.S. package insert for the treatment of schizophrenia disorder (e.g., second-generation antipsychotics [SGAs] like olanzapine, risperidone, paliperidone, quetiapine, aripiprazole and lurasidone) were eligible provided they had been treated with the medication for at least 3 months (90 days), the dose had been stable for at least 1 month (30 days) prior to the screening visit (includes no change from screening to baseline/Visit 1 [Day 1]), and there had been no inpatient psychiatric hospitalization in the past 4 months prior to screening.
6. Concomitant use of antidepressants such as selective serotonin reuptake inhibitors (SSRIs; e.g., fluoxetine, sertraline, citalopram), serotonin-norepinephrine reuptake inhibitors (SNRIs; e.g., venlafaxine, desvenlafaxine, duloxetine, vortoxetine, vilazodone) were allowed as long as patient was on an optimized dose for 3 months (90 days), provided the dose had been stable for at least 1 month (30 days) prior to baseline and the dose used was within the guidance from the U.S. label for that drug. Paroxetine, a CYP2D6 substrate, was allowed provided the dose did not exceed 10 mg/day.
7. Concomitant use of hypnotics at bedtime (e.g., eszopiclone, zolpidem, zaleplon, trazodone [up to 100 mg/day]) for the nighttime treatment of insomnia was allowed, provided the dose had been stable for at least 1 month (30 days) prior to baseline and remained stable throughout the study. Patients on lorazepam for anxiety, restlessness, or agitation prior to study entry should have remained on the same treatment regimen for the duration of the study. All other benzodiazepines were disallowed, except for lorazepam use for short term or prn treatment of insomnia and behavioral disturbances. The duration of dosing should not have exceeded 3 days in a 7-day period.
8. Patients who were capable, according to the Investigator, and had signed and received a copy of the patient informed consent form (ICF) after the nature and risks of study participation had been fully explained to them.
9. Patients must have had a reliable informant as deemed appropriate by the investigator.

1.3.2 Exclusion Criteria

Patients were not to be enrolled in the study if they met any of the following criteria:
1. Patients with myasthenia gravis.
2. Patients with current major depressive disorder at the screening visit and/or a Calgary Depression Scale for Schizophrenia (CDSS) score ≥6.
3. Patients with cardiovascular concerns at screening or baseline such as:
   a. History or evidence of complete heart block, ventricular tachycardia, presence of clinically significant premature ventricular contractions as evaluated by a central reader, QTc prolongation or torsades de pointes.
   b. QTc using the Fridericia's formula (QTcF) at screening >450 msec for males and >470 msec for females based on central review at the screening visit, unless due to ventricular pacing.
   c. Any family history of congenital QT interval prolongation syndrome.
   d. History or presence of clinically significant syncope, orthostatic hypotension or postural tachycardia.
4. Patients with known hypersensitivity to DM, Q, opiate drugs (codeine, etc.), or any other ingredient of the study medication.
5. Patients with history of allergy or hypersensitivity to several classes of medications.
6. Patients who had received DM co-administered with Q within 3 months (90 days) prior to baseline.
7. Patients with pseudoparkinsonism secondary to their ongoing antipsychotic medication based on PI judgement.
8. Patients treated with any typical antipsychotic within 3 months (90 days) prior to baseline. Typical antipsychotic medications were not allowed during the study.
9. Patients with a history of clozapine use within 3 months (90 days) prior to baseline. Clozapine was not allowed during the study.
10. Patients who were currently using anticholinergic medications or within 1 month (30 days) prior to baseline for treatment of AEs associated with antipsychotic medications.
11. Patients who were currently treated with monoamine oxidase inhibitors (MAOIs) or within 2 weeks prior to baseline.
12. Patients who had been taking prohibited concomitant medications per study protocol, within 2 weeks or 5 half-lives, whichever is longer, prior to baseline.
13. Patients with concurrent clinically significant or unstable systemic diseases that could confound the interpretation of the safety results of the study (e.g., malignancy [except skin basal-cell carcinoma or untreated prostate cancer], poorly controlled diabetes, poorly controlled hypertension, unstable pulmonary, renal or hepatic disease, unstable ischemic cardiac disease, dilated cardiomyopathy, or unstable valvular heart disease), cognitive and other neurodegenerative disorders. Some cases might have been evaluated individually with the Investigator and medical monitor.
14. Current suicide risk, as evidenced by any of the following:
   a. It is the judgment of the Investigator that the patient might have been at risk for suicide.
   b. The patient was rated a “yes” to question 4 or question 5 on the screening and baseline Columbia Suicide Severity Rating Scale (C-SSRS), and the most recent episode occurred within 6 months prior to screening and baseline.
   c. The patient had attempted suicide within 12 months prior to screening and baseline.
15. Patients who had an inpatient psychiatric hospitalization within 4 months of screening.
16. Patients with clinically significant laboratory abnormalities (hematology, chemistry, and urinalysis) or with safety values of potential clinical concern or with aspartate aminotransferase (AST) or alanine aminotransferase (ALT) >2×the upper limit of normal at the screening visit.
17. Patients who were currently participating in, or who had participated in other interventional (drug or device) clinical study within 30 days prior to baseline.
18. Unwilling or unable, in the opinion of the Investigator, to comply with study instructions.
19. Patients with a history of substance and/or alcohol abuse, not including cigarettes (cannabis use might have been allowed per Investigator discretion) within 6 months or dependence within 1 year prior to baseline.
20. Patients who had received electroconvulsive treatment, repetitive transcranial magnetic stimulation or deep brain stimulation in the past year prior to screening.
21. Patients who were found to be a virtually certain match in CTS database with a patient who had participated in another interventional drug or device study within 30 days prior to baseline.

1.3.3 Removal of Patients from Therapy or Assessment

Patients were advised verbally and in the ICF that they had the right to withdraw from the study at any time without prejudice or loss of benefits to which they were otherwise entitled, and were not obligated to provide the reason.

The Investigator or Sponsor may have discontinued a patient from the study for any of the following reasons:
In case of an inter-current illness, AE, other reasons concerning the health or well-being of the patient
In the case of lack of cooperation, non-compliance, protocol violation, or other administrative reasons.
Patients who presented a QTcF >500 msec (unless due to ventricular pacing) or a QTcF change from the pre-dose baseline ECG of >60 msec at any time after baseline. The QTcF values was recorded and assessed for clinical significance.
Patients who started a prohibited concomitant medication, psychotherapy, or somatic therapy (light therapy, repetitive transcranial magnetic stimulation, and other non-invasive brain stimulation techniques) during the study. The decision to withdraw the patient was made on a case-by-case basis in conjunction with the medical monitor.

Patients who withdrew prior to study completion for any reason were asked to return to the clinic to complete the Visit 7 (Early Termination) assessments. If a patient did not return for a scheduled visit, every effort was made to contact the patient. In any circumstance, every effort was made to document patient outcome, if possible.

If the patient withdrew from the study and consent for disclosure of further information was withdrawn, then no further evaluations were performed and no additional data was collected.

Patients who withdrew from the study were not replaced.

1.4 Treatments

1.4.1 Treatments Administered

Clinical study medication was provided as hard, blue-colored opaque gelatin capsules (size 3). Three different capsule strengths were provided, as follows:
d6-DM/Q-24/4.9 (d6-DM 24 mg/Q 4.9 mg)
d6-DM/Q-34/4.9 (d6-DM 34 mg/Q 4.9 mg)
d6-DM/Q Placebo, with the same excipients as the study medication

All medication used in this study were prepared, packaged, and labeled in accordance with Good Manufacturing Practice guidelines, ICH guidelines, GCP guidelines, and applicable laws and regulations.

1.4.2 Identity of Investigational Product(s)

d6-DM/Q and matching placebo were supplied as a solid oral dosage form (gelatin capsule). The composition of each investigational product is shown in Table 2.

TABLE 2
Composition of Study Medication

| | d6-DM/Q (d6- | d6-DM/Q (d6- | |
|---|---|---|---|
| | DM 24 mg/Q | DM 34 mg/Q | Placebo |
| Ingredient | 4.9 mg) (mg) | 4.9 mg) (mg) | (mg) |
| d6-dextromethorphan | 24.0 | 34.0 | 0 |
| hydrobromide | | | |
| Quinidine sulfate USP, EP | 4.9 | 4.9 | 0 |
| Croscarmellose sodium | 6.6 | 6.6 | 6.6 |
| NF, EP | | | |
| Microcrystalline cellulose | 181.2 | 171.2 | 210.1 |
| NF, EP | | | |
| Colloidal silicone dioxide | 2.2 | 2.2 | 2.2 |
| NF, EP | | | |
| Magnesium stearate NF, EP | 1.1 | 1.1 | 1.1 |
| Total | 220.0 | 220.0 | 220.0 |
| Capsule: Hard gelatin | 48.0 | 48.0 | 48.0 |
| capsules, opaque blue | | | |
| cap and body, size 3. | | | |
| (average weight) | | | |
| Total Weight | 268.0 | 268.0 | 268.0 |

EP = European Pharmacopoeia;
NF = National Formulary;
USP = United States Pharmacopoeia

The study medication was supplied as ready-packaged, blinded, pre-labeled, individually pre-packaged blister cards. Each blister card contained enough study medication to last for 3 weeks, i.e., 48 capsules of 1 of the 2 active study medications or placebo. Each 3-week blister card was clearly labeled to identify the morning and evening doses.

1.4.3 Method of Assigning Patients to Treatment Groups

Eligible patients were randomized in a 1:2 ratio of d6-DM/Q or matching placebo at Stage 1 baseline. Patients randomized to placebo at Stage 1 baseline were re-randomized at the beginning of Stage 2, in a 1:1 ratio to d6-DM/Q and placebo. The re-randomization was stratified by patient responder status (responder vs. non-responder). A responder was defined as a patient with ≥20% change from baseline (Stage 1) in PANSS total score. Patients assigned to placebo in Stage 1 who dropped out early in Stage 1 were also randomly assigned Stage 2 treatment in the same manner as the other placebo patients for statistical analysis purposes; their responder status was based on the measurements at their Early Termination visit.

Double-blinded study medication assignment adhered to a randomization scheme and was managed using Interactive Response Technology (IRT). Allocation was handled by an IRT, which dynamically assigned the randomization blocks to study centers as they were needed. Enrollment was centrally randomized across the study.

1.4.4 Selection of Doses in the Study

The doses selected for evaluation in this study were hypothesized to be potentially efficacious in the treatment of schizophrenia based on several in vitro studies of d6-DM assessing receptor pharmacology. The doses of d6-DM/Q were also expected to have a good safety and tolerability profile based on data from completed Phase 1 studies of d6-DM/Q. Therefore, the doses of d6-DM/Q used in this study, d6-DM/Q-24/4.9 and d6-DM/Q-34/4.9, were selected to provide an optimal benefit-risk ratio in this patient population.

Escalation to the higher dose of d6-DM/Q (d6-DM/Q-34/4.9) using a fixed titration scheme was implemented with the assumption that it may increase tolerability.

1.4.5 Selection and Timing of Dose for Each Patient

Patients self-administered the study medication approximately every 12 hours, orally with water (morning and evening), except on visit days when the morning dose of study medication was administered in the presence of site personnel.

Patients randomized to d6-DM/Q in Stage 1, took d6-DM/Q-24/4.9 QD in the morning and placebo in the evening during the first week (Day 1 to 7), d6-DM/Q-24/4.9 BID for the next 1 week (Day 8 to 13), and d6-DM/Q-34/4.9 BID for the remaining 10 weeks of the study (Day 14 to 85).

Patients randomized to placebo in Stage 1, took placebo BID for the 6-week duration of Stage 1 (Day 1 to 42). Those who were re-randomized to placebo continued taking placebo BID for the 6-week duration of Stage 2 (Day 43 to 85) and those who were re-randomized to d6-DM/Q took d6-DM/Q using the same dose escalation schedule in Stage 1, i.e., d6-DM/Q-24/4.9 QD in the morning and placebo in the evening during the first week of Stage 2 (Day 43 to 50), d6-DM/Q-24/4.9 BID for the next 1 week (Day 51 to 58), and d6-DM/Q-34/4.9 BID for the remaining 4 weeks of Stage 2 (Day 59 to 85).

1.4.6 Blinding

All study medication, including d6-DM/Q capsules and placebo capsules, were of identical appearance in order to maintain the integrity of the blind, including during dose escalation. Neither the Sponsor, patients, investigators, nor other study personnel were aware of a patient's treatment assignment. In the event that it became medically necessary to identify which treatment a patient had received, the blind could be broken. In that event, the investigator was to make all attempts to contact the medical monitor or representative to request the unblinding of a patient. The IRT manager was not required to be blinded, and he or she had access to the study medication list and the randomization code.

1.4.7 Prior and Concomitant Therapy

1.4.7.1 Allowed Concomitant Medications

Allowed concomitant medications were to be evaluated by the Investigator and discussed with the medical monitor as necessary to determine if there was any concern for use during the study.

Concomitant use of hypnotics at bedtime (e.g., eszopiclone, zolpidem, zaleplon, trazodone [up to 100 mg/day]) for the nighttime treatment of insomnia was allowed, provided the dose had been stable for at least 1 month prior to baseline and remained stable throughout the study. Patients on lorazepam for anxiety, restlessness, or agitation prior to study entry remained on the same treatment regimen for the duration of the study.

All other benzodiazepines were disallowed, except for lorazepam use for short term or prn treatment of insomnia and behavioral disturbances. Dosing was not to exceed 3 days in a 7-day period.

1.4.7.2 Prohibited Medications

Patients were not allowed to take any of the prohibited medications during the study or 2 weeks or 5 half-lives, whichever was longer, prior to the start of dosing on Day 1. Examples of the prohibited medications are listed in Table 3. At each visit, patients were queried as to whether they took any concomitant medications and, if so, the Investigator recorded the medications taken and the reasons for their use. After the screening visit, patients who were using sedatives/hypnotics or benzodiazepine medications on a prn basis were not allowed to take any of these medications the day of, or the day before, the assessment of cognitive function by MCCB. Patients who were on stable dose regimens of sedatives/hypnotics or benzodiazepine medications were to take their medication as prescribed.

TABLE 3
Prohibited Medications

| Classes | Prohibited Concomitant Medications |
|---|---|
| May increase Q plasma levels¹ | Amiodarone |
| | Carbonic anhydrase inhibitors |
| | Cimetidine |
| | Diltiazem |
| | Itraconazole |
| | Ketoconazole |
| | Macrolide antibiotics² |
| | Protease inhibitors³ |
| | Voriconazole |
| Metabolized by CYP2D6 and might | Dextromethorphan⁴ |
| have increased plasma levels if co- | TCA⁵ |
| administered with Q | Atomoxetine |
| Related to Q | Quinine |
| | Mefloquine |
| Might produce serotonin syndrome | MAOIs⁶ |
| when co-administered with DM | |
| Might decrease DM and Q plasma | Carbamazepine |
| levels | Cyproterone |
| | Hyperforin |
| | Oxcarbazepine |
| | Phenobarbital |
| | Phenytoin |
| | Rifampicin |
| | St. John's Wort |
| Other | Clozapine⁷ |
| Typical antipsychotic medications⁷ | |

These are examples of disallowed medications and not a comprehensive list.
CYP2D6 = cytochrome P450 isoenzyme 2D6;
DM = dextromethorphan;
MAOI = monoamine oxidase inhibitor;
Q = quinidine sulfate;
TCA = tricyclic antidepressants.
¹Topical preparations were permitted unless applied under occlusive dressing or other technique that was intended to increase systemic absorption.
²Examples included erythromycin, azithromycin, clarithromycin, dirithromycin, and roxithromycin.
³Examples included saquinavir, ritonavir, atazanavir, and indinavir.
⁴Over-the-counter and prescription.
⁵Examples included imipramine; desipramine, amitriptyline, and nortriptyline.
⁶Patients were to allow at least 14 days after stopping study medication before starting an MAOI.
⁷Not allowed during the study or within 3 months (90 days) prior to baseline.

1.5 Efficacy and Safety Variables

1.5.1 Efficacy and Safety Measurements Assessed and Flow Chart

The schedule of procedures and assessments is presented in Table 1.

1.5.1.1 Efficacy Measures

The primary efficacy measure was the 16-Item NSA-16 total score.

Secondary measures included scores from the PANSS, Clinical Global Impression of Severity (CGI-S), Clinical Global Impression of Change (CGI-C), Patient Global Impression of Change (PGI-C), MCCB, CDSS, EEfRT, and Smoking Cessation Question. Descriptions of the scales used to measure efficacy are provided below.

1.5.1.1.1 16-Item Negative Symptom Assessment (NSA-16)

The NSA-16 (Appendix 3A) is considered a valid and reliable measure of the presence, severity, and range of negative symptoms associated with schizophrenia; it has high interrater and test-retest reliability across languages and cultures (Daniel, Schizophr Res. 2013; 150(2-3):343-345; Axelrod et al. J Psychiatr Res. 1993; 27(3):253-258). The NSA-16 uses a 5-factor model to describe negative symptoms: (1) communication, (2) emotion/affect, (3) social involvement, (4) motivation, and (5) retardation. These factors, assessed through a structured interview, are comprehensive and well-defined to help standardize assessment. As a truncated version of the 25-item NSA, the NSA-16 still captures the multidimensionality of negative symptoms but can be completed in approximately 15 to 20 minutes (Axelrod et al. J Psychiatr Res. 1993; 27(3):253-258). The NSA-4 (Alphs et al. Int J Methods Psychiatr Res. 2011; 20(2):e31-37) is comprised of the 4 NSA-16 items as follows: 1) restricted speech quantity, 2) emotion: reduced range, 3) reduced social drive, and 4) reduced interests, as well as an overall global rating of negative symptoms.

The NSA-16 evaluation was performed at screening (Day −28 to Day −1), baseline/Visit 1 (Day 1), Visit 3 (Day 22), Visit 4 (Day 43), Visit 6 (Day 64) and Visit 7/Early Termination visit (Day 85).

1.5.1.1.2 Positive and Negative Syndrome Scale (PANSS)

The PANSS (Appendix 2) is a 30-item clinical scale that has been extensively used as a reliable and valid measure for negative symptom trials (Daniel, Schizophr Res. 2013; 150(2-3):343-345). Each item is scored for “1” (absent) to “7” (extremely severe). Subscales of the PANSS include:
Positive subscale (P1-P7),
Negative subscale (N1-N7),
General psychopathology subscale (G1-G16),
Prosocial factors (G16. active social avoidance, N2. emotional withdrawal, N4. passive/apathetic social withdrawal, N7. stereotyped thinking, P3. hallucinatory behavior, P6. suspiciousness/persecution),
Marder negative factors (N1. blunted affect, N2. emotional withdrawal, N3. poor rapport, N4. passive/apathetic social withdrawal, N6. lack of spontaneity and flow of conversation, G7. motor retardation, G16. active social avoidance),
Excitement component (P4. excitement, P7. hostility, G4. tension, G8. uncooperativeness, G14. poor impulse control).

The PANSS evaluation was performed at screening (Day −28 to Day −1), baseline/Visit 1 (Day 1), Visit 3 (Day 22), Visit 4 (Day 43), Visit 6 (Day 64) and Visit 7/Early Termination visit (Day 85).

1.5.1.1.3 Clinical Global Impression (CGI) Scales

The CGI was developed to provide a brief, stand-alone assessment of the clinician's view of the patient's global functioning prior to and after initiating a study medication (Busner and Targum, Psychiatry (Edgmont). 2007; 4(7):28-37). The CGI provides an overall clinician-determined summary measure that takes into account all available information, including knowledge of the patient's history, psychosocial circumstances, symptoms, behavior, and the impact of the symptoms on the patient's ability to function. The CGI comprises 2 companion 1-item measures, the CGI-S(Severity) and CGI-C(Change). The CGI forms can be completed in less than 1 minute by an experienced rater.

Clinical Global Impression-Severity (CGI-S)

The CGI-S is a 7-point scale that requires the clinician to rate the severity of the patient's illness at the time of assessment, relative to the clinician's past experience with patients who have the same diagnosis (Guy W. ECDEU Assessment Manual for Psychopharmacology. 1976:76-338). Considering total clinical experience, a patient is assessed on severity of mental illness at the time of rating 1, normal, not at all ill; 2, borderline mentally ill; 3, mildly ill; 4, moderately ill; 5, markedly ill; 6, severely ill; or 7, among the most extremely ill patients.

The CGI-S evaluation was performed at baseline/Visit 1 (Day 1), Visit 4 (Day 43) and Visit 7/Early Termination visit (Day 85).

Clinical Global Impression-Change (CGI-C)

The CGI-C is a 7-point scale that requires the clinician to rate the change of the patient's condition at the time of assessment, relative to the clinician's past experience with the patient's condition at admission. Considering total clinical experience, a patient is assessed for change of mental illness as 1, Very much improved; 2, Much improved; 3, Minimally improved; 4, No change; 5, Minimally worse; 6, Much worse; or 7, Very much worse.

The CGI-C evaluation was performed at Visit 4 (Day 43) and Visit 7/Early Termination visit (Day 85). At Day 43 (Visit 4), the CGI-C was completed to assess change from the baseline visit (Day 1). At Day 85 (Visit 7), the CGI-C was completed to assess change from Day 43 (Visit 4) and change from the baseline visit (Day 1).

1.5.1.1.4 Patient Global Impression-Change (PGI-C)

The PGI-C (Appendix 5A) is a 7-point (1-7), patient-rated scale used to assess treatment response as: very much improved, much improved, minimally improved, no change, minimally worse, much worse, or very much worse.

The PGI-C evaluation was performed at Visit 4 (Day 43) and Visit 7/Early Termination visit (Day 85).

1.5.1.1.5 Measurement and Treatment Research to Improve Cognition in Schizophrenia (MATRICS) Consensus Cognitive Battery (MCCB)

The MCCB is the standard tool for assessing cognitive change in trials of cognitive-enhancing agents in schizophrenia. The MCCB (Nuechterlein et al. Am J Psychiatry. 2008; 165(2):203-213) is intended to provide a relatively brief evaluation of key cognitive domains relevant to schizophrenia and related disorders. The MCCB includes 10 tests that measure 7 cognitive domains: Speed of Processing, Attention/Vigilance, Working Memory, Verbal Learning, Visual Learning, Reasoning, and Problem Solving and Social Cognition.

The MCCB evaluation was performed at screening (Day −28 to Day −1), baseline/Visit 1 (Day 1), Visit 4 (Day 43) and Visit 7/Early Termination visit (Day 85). The MCCB was to be conducted at approximately the same time of day (+/−2 hours) and preferably in the AM. Alternate versions of the battery were used at different visits to decrease the learning confound.

1.5.1.1.6 Calgary Depression Scale for Schizophrenia (CDSS)

The CDSS (Appendix 6) is a 9-item scale derived from the Hamilton Depression Scale (Ham-D) that is designed to assess depression specifically in patients with schizophrenia (Addington et al. Schizophr Res. 1996; 19(2-3):205-212). Unlike the Ham-D, the CDSS does not contain depressive symptoms that overlap with negative symptoms of schizophrenia, such as anhedonia and social withdrawal. The CDSS has shown excellent psychometric properties. Each item on the scale is scored as 0, Absent; 1, Mild; 2, Moderate; or 3, Severe. The CDSS score is obtained by adding each of the item scores. A score above 6 has an 82% specificity and 85% sensitivity for predicting the presence of a major depressive episode.

The CDSS evaluation was performed at screening (Day −28 to Day −1), baseline/Visit 1 (Day 1), Visit 3 (Day 22), Visit 4 (Day 43), Visit 6 (Day 64) and Visit 7/Early Termination visit (Day 85).

1.5.1.1.7 Effort Expenditure for Reward Task (EEfRT)

The Effort Expenditure for Rewards Task (EEfRT) (Treadway et al. PLoS One. 2009; 4(8):e6598) is a multi-trial computerized task in which participants are given an opportunity on each trial to choose between 2 tasks that differ in difficulty level and are associated with varying levels of monetary reward. This task examines probabilistic learning in response to variable reward schedules and effort expended (button pushing) for reward. Probability is manipulated in the EEfRT, because, like mobilization of effort, probability discounting appears to be highly predictive of negative symptoms. Additionally, the inclusion of a probability manipulation improves the overall ecological validity of the task, as most real-world choices that require motivation are usually associated with some level of uncertainty in the outcome. The EEfRT reliably measures drug effects on willingness to expend effort in relation to amount of reward or probability of reward. For example, amphetamine increased effort in response to low- and moderate probability rewards (Wardle et al. J Neurosci. 2011; 31(46):16597-16602). Whereas reward salience and behavioral response have been linked to dopamine release in the striatum, glutamatergic input to midbrain dopamine neurons via NMDA receptors is also required for reward conditioning (Stuber et al. Science. 2008; 321(5896):1690-1692). The ratio of hard task choices with moderate probability reward was used as outcome measure for negative symptoms.

The EEfRT evaluation was performed at baseline/Visit 1 (Day 1), Visit 4 (Day 43) and Visit 7/Early Termination visit (Day 85).

1.5.1.1.8 Smoking Cessation Question

A smoking cessation question was asked at baseline/Visit 1 (Day 1), Visit 4 (Day 43), and Visit 7/Early Termination visit (Day 85). At the baseline visit, patients were asked about their usage of tobacco (cigarettes); for example, never, current, former. They were then asked about the number of cigarettes and what frequency. At visit 4 and visit 7, subjects were asked if there had been any change in usage.

1.5.1.2 Efficacy Variables

The primary efficacy variable was the change from baseline/Visit 1 to Week 6/Visit 4 (Day 43, Stage 1) and from Week 6/Visit 4 (Day 43) to Week 12/Visit 7 (Day 85, Stage 2) on the NSA-16 total score analyzed based on the SPCD method using a weighted ordinary least square test statistics combining treatment effects from Stage 1 and 2.

The secondary efficacy variables included change from baseline to Week 6/Visit 4 (Day 43, Stage 1) and from Week 6/Visit 4 (Day 43) to Week 12/Visit 7 (Day 85, Stage 2) for the following efficacy measures:
PANSS total score
PANSS subscales (positive, negative, general psychopathology, Marder negative factors, excitement component, and prosocial factors)
NSA-16 (factor domains, global score, individual items, and NSA-4 factors)
Proportion of patients with ≥20% reduction in PANSS total score
MCCB composite score
CGI-S score
CGI-C scores (measures change at post baseline visits)
PGI-C scores (measures change at post baseline visits)
CDSS
EEfRT

1.5.1.3 Safety Measures

Safety was assessed by reported AEs, serious AEs (SAEs), physical examinations (scheduled for screening visit only), vital signs, weight, pregnancy tests, clinical laboratory assessments, and resting 12-lead ECGs. In addition, the following scales were used to assess safety:
Columbia Suicide Severity Rating Scale (C-SSRS)
Abnormal Involuntary Movements Scale (AIMS)
Barnes Akathisia Scale (BAS)
Simpson Angus Scale for Extrapyramidal Symptoms (SAS)

1.5.1.3.1 Adverse Events

An AE was defined as any untoward medical occurrence or unintended change (including physical, psychological, or behavioral) from the time the ICF was signed, including inter-current illness, which occurred during the course of a clinical trial after treatment was started, whether considered related to treatment or not. An AE is therefore any unfavorable and unintended sign (including an abnormal laboratory finding, for example), symptom, or disease temporally associated with the use of a medicinal product, whether or not considered related to the medicinal product.

Treatment-emergent adverse events (TEAEs) were defined as AEs that first occurred, or worsened, after the first dose of study medication and within 30 days after the permanent discontinuation of the study medication (first dose date on or before AE start date which was on or before the date of last dose+30 days). Changes associated with normal physiology that did not vary in frequency or magnitude from what was ordinarily anticipated clinically were not considered AEs (e.g., onset of menstruation occurring at a physiologically appropriate time). A deliberate or inadvertent administration of a treatment at a dose higher than specified in the protocol and higher than known therapeutic doses was considered an overdose. Overdoses were reported irrespective of outcome (even if toxic effects were not observed).

Any AE reported after a patient received the last dose of study medication and up until 30 days after receiving the last dose of study medication was followed up until resolution (patient's health returned to his/her baseline status or all variables returned to normal) or until stabilization of the event occurred (the investigators did not expect any further improvement or worsening of the event).

All AEs were graded on a 3-point scale and reported in detail as indicated on the electronic Case Report Form (eCRF) as follows:

| Rating | Definition |
|---|---|
| Mild | Easily tolerated, causing minimal discomfort and not |
| | interfering with normal everyday activities |
| Moderate | Sufficiently discomforting to interfere with normal |
| | everyday activities |
| Severe | Incapacitating and/or preventing normal everyday activities |

The relationship of each AE to study medication was determined by the investigators using the following explanations:
Not related: this category was applicable to those AEs that were clearly related to other factors such as the patient's clinical state, therapeutic interventions, or concomitant medications administered to the patient.
Unlikely related: this category was applicable to those AEs that were most likely produced by other factors such as the patient's clinical state, therapeutic interventions, or concomitant medications administered to the patient; and did not follow a known response pattern to the study medication.
Possibly related: this category was applicable to those AEs that followed a reasonable temporal sequence from the time of drug administration; and/or followed a known response pattern to the study medication; but could have been produced by other factors such as the patient's clinical state, therapeutic interventions, or concomitant medications administered to the patient.
Related: this category was applicable to those AEs that followed a reasonable temporal sequence from the time of drug administration; and followed a known response pattern to the study medication; and cannot be reasonably explained by other factors such as the patient's clinical state, therapeutic interventions, or concomitant medications administered to the patient.

1.5.1.3.2 Serious Adverse Events

An SAE was defined as any AE occurring at any dose that resulted in any of the following outcomes:
Death;
Life-threatening experience (one that placed the patient, in the view of the initial reporter, at immediate risk of death from the AE as it occurred; i.e., it did not include an AE that, had it occurred in a more severe form, might have caused death);
Persistent or significant disability/incapacity (disability was a substantial disruption of a person's ability to conduct normal life functions);
In-patient hospitalization or prolongation of hospitalization;
Congenital anomaly/birth defect.

Important medical events that did not result in death, were not life-threatening, or did not require hospitalization were considered an SAE when, based upon appropriate medical judgment, they jeopardized the patient or required medical or surgical intervention to prevent one of the outcomes listed in the definition. The medical monitor had to be contacted immediately (within 24 hours) for any SAE (including an abnormal laboratory test value).

A death that occurred during the study, or that came to the attention of the investigators within 30 days after stopping the treatment whether considered treatment-related or not, was to be reported. Pregnancy was not considered an AE or SAE, unless a complication occurred that met the requirements for an AE or SAE; however, it was reported on a pregnancy report form. The terms “cancer” and “overdose” were not considered SAEs unless the other criteria for SAE were met; however, cancer and overdose were reported as AEs.

1.5.1.3.3 Physical and Neurological Examination

Physical and neurological examinations were performed at screening (Day −28 to Day −1) and at the discretion of the Investigator at the subsequent visits. It included assessments of head, eyes, ears, nose, throat, lymph nodes, skin, extremities, respiratory, gastrointestinal, musculoskeletal, cardiovascular, and nervous systems. Physical and neurological examinations were performed by the same person each time, whenever possible.

Physical and neurological examination abnormalities determined by the investigators to be clinically significant at screening were recorded as medical history. Any clinically significant changes in physical and neurological examination findings from the screening examination were recorded as AEs.

1.5.1.3.4 Vital Signs and Weight

At screening (Day −28 to Day −1), orthostatic blood pressure (BP) and heart rate (HR) measurements were performed. Supine BP and HR were measured after a patient rested for at least 5 minutes in the supine position. Each measurement was taken and recorded twice. After the measurement of supine BP and HR, the patient stood still for up to 3 minutes and a single measurement of standing BP and HR was recorded within these 3 minutes of standing. Respiratory rate (breaths/minute), body temperature, height, and weight were also recorded.

Patients presenting with orthostatic hypotension (decrease of a ≥20 mm Hg in systolic blood pressure [SBP] or a ≥10 mm Hg in diastolic blood pressure [DBP] upon postural change from supine to standing measured within 3 minutes) and/or postural tachycardia (HR increase ≥30 beats per minute (bpm) from the supine measurements or HR ≥120 bpm on standing) met exclusion criteria 3.

At all subsequent visits, the supine/semi-recumbent systolic and diastolic BP and HR (beats/minute), after at least 5 minutes of rest, were taken and recorded twice. Respiratory rate (breaths/minute), body temperature, and weight were also recorded.

The vital signs and weight were captured at all visits as indicated in Table 1.

1.5.1.3.5 Pregnancy Test

All female patients of childbearing potential were instructed to use appropriate birth control methods for up to 4 weeks following the last dose of study medication.

Urine pregnancy tests (beta-hCG) were performed on all females regardless of childbearing potential at all clinic visits except at the screening visit where serum beta-hCG test was performed.

1.5.1.3.6 Clinical Laboratory Assessments

The clinical laboratory assessments which included blood chemistry, hematology, and urinalyses were performed at the visits presented in Table 1. The clinical laboratory assessments included:
Blood chemistry (calcium, magnesium, phosphorus, glucose, sodium, potassium, chloride, carbon dioxide, blood urea nitrogen, serum creatinine, uric acid, albumin, total bilirubin, alkaline phosphatase, lactate dehydrogenase, aspartate aminotransferase/serum glutamic oxaloacetic transaminase [AST/SGOT], alanine aminotransferase/serum glutamic pyruvic transaminase [ALT/SGPT], creatine kinase, gamma-glutamyl transferase, triglycerides, total protein, total cholesterol, and glycosylated hemoglobin [HbA1c, at screening and Visit 7]).
Hematology (red blood cell count, hemoglobin, hematocrit, white blood cell count, neutrophils, bands, lymphocytes, monocytes, eosinophils, basophils, platelet count, and morphology).
Urinalysis (pH, specific gravity, protein, glucose, ketones, bilirubin, urobilinogen, nitrites, leucocytes, and blood). Microscopic analysis is performed on those samples that are positive for blood, protein, leucocyte esterase or nitrates.
Urine screen for presence of alcohol and substances of abuse—pheneyelidine, PCP, benzodiazepines, cannabinoids, amphetamines, barbiturates, cocaine, and opiates. (screening visit only).
Thyroid function tests TSH, T3, T4 (screening visit only).
Plasma antipsychotic levels were measured at Screening, Week 6, and Week 12.

Any patients with clinically significant abnormal laboratory test results were required by the medical monitor to have a repeat test 1 week later or sooner, if medically indicated. Clinically significant laboratory abnormalities could have been a basis for exclusion from study entry. Non-eCRF data including, but not limited to, laboratory tests and results, were sent to the contract research organization (CRO) by data transfer from the central laboratory for assimilation into the database.

1.5.1.3.7 Electrocardiograms

ECG equipment was provided by the central reader. ECG data was recorded at the study center and included general findings, HR (beats/minute), QRS complex and PR and QTc intervals (milliseconds). Results were provided by the central reader to the investigators within 72 hours and any significant findings were reported within 24 hours. ECG abnormalities present at screening were recorded as medical history. Any changes from the ECG status at screening that were deemed to be clinically significant by the investigators were recorded as AEs. Any clinically significant abnormal ECG was discussed with the study medical monitor and if necessary was repeated within a 1-week period. Non-eCRF data including, but not limited to, ECG tests and results, were sent to the CRO by data transfer from the central reader for assimilation into the database. A resting 12-lead ECG was performed at all visits as indicated in Table 1. At Stage 1 baseline (Day 1), and Stage 2 baseline visit 4 (Day 43), 2 ECGs were performed; 1 prior to study medication dosing, and another 2-3 hours after dosing. Triplicate ECGs were performed at screening.

1.5.1.3.8 Columbia Suicide Severity Rating Scale (C-SSRS)

The C-SSRS (Appendix 10) is a low-burden measure of the spectrum of suicidal ideation and behavior that was developed by Columbia University researchers for the National Institute of Mental Health Treatment of Adolescent Suicide Attempters Study to assess severity and track suicidal events through any treatment. It is a clinical interview providing a summary of both ideation and behavior that can be administered during any evaluation or risk assessment to identify the level and type of suicidality present. The C-SSRS can also be used during treatment to monitor for clinical worsening. The C-SSRS rating was performed at all clinic visits.

1.5.1.3.9 Simpson Angus Scale for Extrapyramidal Symptoms (SAS)

The SAS (Appendix 9A) is composed of 10 items and is used to assess pseudoparkinsonism. Grade of severity of each item is rated using a 5-point scale. SAS scores can range from 0 to 40. Signs assessed include gait, arm-dropping, shoulder-shaking, elbow rigidity, wrist rigidity, leg pendulousness, head dropping, glabella tap, tremor, and salivation. The SAS evaluation was performed at baseline/Visit 1 (Day 1), Visit 4 (Day 43) and Visit 7/Early Termination visit (Day 85).

1.5.1.3.10 Barnes Akathisia Scale (BAS)

The BAS (Appendix 8) consists of items that assess the objective presence and frequency of akathisia, the level of an individual's subjective awareness and distress, and global severity. The BAS is scored as follows: Objective Akathisia, Subjective Awareness of Restlessness and Subjective Distress Related to Restlessness are rated on a 4-point scale from 0-3 and are summed yielding a total score ranging from 0 to 9. The Global Clinical Assessment of Akathisia uses a 5-point scale ranging from 0-4. The BAS evaluation was performed at baseline/Visit 1 (Day 1), Visit 4 (Day 43) and Visit 7/Early Termination visit (Day 85).

1.5.1.3.11 Abnormal Involuntary Movements Scale (AIMS)

The AIMS (Appendix 7A) is composed of 12 items and was used to assess dyskinesia. Items related to severity of orofacial, extremity, and trunk movements, global judgment about incapacitation, and patient awareness were rated using a 5-point scale (0=none to 4=severe). Two items related to dental status were scored using “yes” or “no” responses. The AIMS evaluation was performed at baseline/Visit 1 (Day 1), Visit 4 (Day 43) and Visit 7/Early Termination visit (Day 85).

1.5.2 Drug Concentration Measurements

All patients had plasma samples collected for analysis of d6-DM, d3-DX, d3-3-MM, and Q plasma concentrations at baseline/Visit 1 (Day 1, post-dose), Visit 4 (Day 43) and Visit 7/Early Termination visit (Day 85), 2 to 3 hours after the morning dose of study medication. Maximum concentration (Cₘₐₓ) and area under the curve (AUC) were estimated based on a PK model for d6-DM, its metabolite deuterated (d3)-dextrorphan (d3-DX), and Q for those patients taking d6-DM/Q. Drug concentrations and estimated PK parameters were summarized by visit, treatment group and metabolizer type.

Plasma samples were separated by centrifugation and then frozen at −20° C. until assayed at the analytical unit.

1.5.3 Cytochrome P450 2D6 and Genotype Assessments

A blood sample for CYP2D6 genotyping was collected at the baseline visit (Day 1) prior to study medication administration.

A sample of whole blood was also collected at any visit when blood was already being drawn, for exploratory biomarker analyses. The samples were processed and stored in a biobank in aliquots for up to 5 years (or until the aliquots were depleted).

1.5.4 Appropriateness of Measurements

The rating scales used to assess efficacy of the study medication are well-established instruments that are clinically validated and widely used in clinical studies of schizophrenia and other psychiatric and behavioral disorders. The safety assessments used in this study are standard in clinical research and are generally recognized as reliable, accurate, and relevant.

1.6 Data Quality Assurance

1.6.1 Study Administration and Conduct

The study was routinely monitored to ensure compliance with the study protocol and the overall quality of data collected.

For each patient enrolled in the study, an eCRF was completed and electronically signed by the investigators to certify that the data within each eCRF was completed and correct. This also applied to those patients who failed to complete the study. If a patient was withdrawn from the study because of a treatment-limiting AE, thorough efforts were to be made to document the outcome. The eCRFs were reviewed by the study monitor at the study site for completeness and adherence to protocol. Errors detected by subsequent in-house data review may necessitate clarification or correction of errors, and the changes were documented and approved by the investigators.

Any site personnel with the responsibility for data entry, query resolution, or eCRF approval completed training prior to accessing the eCRF. The electronic data capture vendor provided a username once training completion was confirmed, and the account was then approved. A change to the data once initially saved was tracked via audit trail, and the reason for change was mandatory. The audit trail also included information of who made the change and a date/time stamp.

Investigator meetings and site initiation visits (Initial Site Study Protocol and Procedures Training) took place to prepare investigators and standardize performance. Investigators and study staff were kept up to date and reminded of important study updates, such as protocol amendments, via Web-Ex trainings and quarterly newsletters, and field monitors reviewed the quarterly newsletters with study sites during interim monitoring visits. In-house clinical research associates (CRAs), overseen by a clinical study manager, called study sites on a regular basis to review important study updates, patient screening, and enrollment status and to answer any site questions. In-house CRAs were also available for any site and monitor support on an ad-hoc basis.

1.7 Statistical Methods Planned in the Protocol and Determination of Sample Size

Statistical analyses were performed using statistical analysis software (SAS®) version 9.3 or higher.

1.7.1 Endpoints

1.7.1.1 Efficacy Endpoints

The primary efficacy endpoint was the change from baseline in the NSA-16 total score.

Secondary endpoints were the change from baseline (or actual scores for the CGI-C and PGI-C) in the scores of the assessments listed below:
PANSS total score, PANSS subscales (positive, negative, general psychopathology, Marder negative factors, excitement component, and prosocial factors)
NSA-16 factor domains, global symptom/functioning score, individual items, and NSA-4
MCCB composite score
CGI-S, CGI-C, and PGI-C scores
CDSS total score
EEfRT score
Proportion of patients with a reduction of 20% or greater in the PANSS total score
Smoking cessation question

1.7.1.2 Safety Endpoints

Safety endpoints in this study included frequency and nature of AEs reported, changes over time in vital signs, weight, urine pregnancy tests, clinical laboratory assessments, resting 12-lead ECGs, C-SSRS, AIMS, BAS, and SAS.

1.7.2 Analysis Populations

1.7.2.1 Modified Intent-to-treat Population

The modified intent-to treat (mITT) population was the primary efficacy analysis population for SPCD analyses. Patients included in this population were determined separately for Stage 1 and Stage 2. Patients included in the Stage 2 analyses of the mITT population were a subset of those included in the Stage 1 analyses. The mITT population was defined as follows:
Stage 1: All patients randomized in Stage 1 who had at least 1 post-baseline NSA-16 total score assessment in Stage 1.
Stage 2: All patients who were re-randomized into Stage 2 (regardless of Stage 1 treatment group) and had at least 1 NSA-16 total score assessment in Stage 2 (after Visit 4 [Week 6]).

When implementing changes in Protocol Amendment 4, the IRT vendor who carried out the IVRS found that approximately 14 Stage 1 Placebo patients were re-randomized for Stage 2 at Week 3 instead of Week 6. Patients with randomization error (13 patients) were excluded from the mITT analysis population.

The Stage 2 mITT population subsets used in SPCD analyses were determined by Stage 1 placebo responder status:
Stage 1 placebo non-responders (this was the primary efficacy Stage 2 mITT subset for SPCD analyses)
Stage 1 placebo responders
Stage 1 placebo responders and non-responders.

1.7.2.2 Per-Protocol Population

The per-protocol (PP) population included those mITT patients who had no major protocol violations which may have substantially impacted efficacy assessments. The following criteria was used as a guide to exclude patients from the per protocol analysis population.
Violation of inclusion and exclusion criteria which may have substantially impacted efficacy assessment.
Study medication compliance <80%
Received incorrect treatment during the study. Specifically, active treatment group patient received placebo or placebo treatment group patient received active re-treatment.
Took disallowed medication(s) post baseline which may have substantially impacted efficacy assessment.
d6-DM or Q concentrations that were below the limit of quantification at Week 6 or 12 when receiving active treatment.

1.7.2.3 Intent-to-Treat Population

The intent-to-treat (ITT) population was used for sensitivity analysis of the SPCD methodology. It included all patients randomized in Stage 1 and all patients who were correctly re-randomized into Stage 2. The 13 patients with randomization error were excluded from this ITT population.

1.7.2.4 Safety Population

The safety population was used for all safety analyses. It included all patients who received at least one dose of study medication.

1.7.2.5 12-Week Parallel Group Population

The 12-week parallel group population comprised patients who were randomized into placebo/placebo or d6-DM/Q/d6-DM/Q (randomized to d6-DM/Q in Stage 1). It was intended to assess efficacy or safety under a 12-week study duration, as would be done if the study had been a parallel group design. Note that patients randomized to Placebo/d6-DM/Q, whether or not they dropped out in Stage 1, were not part of this population.

mITT 12-week Parallel Group Population: This population comprised patients in the 12-week parallel group population who had at least one post-baseline NSA-16 total score.

Safety 12-week Parallel Group Population: This population comprised patients in the 12-week parallel group population who received at least one dose of study medication.

Per-protocol 12-week Parallel Group Population: This population comprised patients in the mITT 12-week parallel group population who were also in the PP population defined in Section 1.7.2.1.

1.7.3 Efficacy Analysis

All statistical testing was 2-sided and performed at the 0.05 level. Quantitative displays were summarized using descriptive statistics (mean, standard deviation [SD], median, minimum, and maximum).

1.7.3.1 Primary Efficacy Analysis (SPCD, Mixed-Model Repeated Measures)

The primary efficacy endpoint (change from baseline in NSA-16 total score) was analyzed using a SPCD weighted test statistic with the treatment effects in each stage estimated by a likelihood-based mixed-model repeated measures (MMRM) analysis on the observed data (Chen et al. Contemp Clin Trials. 2011; 32(4):592-604). This analysis included patients in the mITT population (Stage 1 mITT population and the placebo non-responder Stage 2 mITT subset). The MMRM analysis included terms for treatment, visit, treatment-by-visit interaction, baseline NSA-16 value, and baseline-by-visit interaction. Unstructured covariance was used. Treatment effect and standard error were obtained directly from the model output.

Separate MMRMs were run to generate the least square (LS) mean difference between treatment groups at Week 6 for Stage 1 and Week 12 (Week 6 to 12) for Stage 2, to generate a combined weighted test statistic. Z_{MMRM}. A prespecified weight of w=0.6 was used for Stage 1, and the weight of 1−w=0.4 was used for Stage 2. The weighted test statistic was then used to generate a 2-sided p-value for the hypothesis test.

1.7.3.2 Sensitivity Analyses for the Primary Efficacy Endpoint

The following sensitivity analyses were performed on the primary endpoint in order to show the robustness of the data when analyzed using different statistical analyses, imputation methods, or patient populations:
SPCD with ordinary least squares (OLS) analysis of covariance (ANCOVA) using the mITT population and last observation carried forward (LOCF) for missing values.
Seemingly unrelated regression (SUR) method (Tamura and Huang, Clin Trials. 2007; 4(4):309-17) that accounted for the fact that random error from the 2 stages of the study may have been correlated for patients with data in both stages. This analysis was performed on the mITT population and LOCF was used for imputation of missing values.
SPCD with MMRM on the PP population.

1.7.3.3 Secondary Efficacy Endpoints Analyses

The secondary endpoints listed in Section 1.7.1.1 were analyzed using the methods described below. In addition, some efficacy analyses were performed on NSA-16 total scores (the primary efficacy measure).

1.7.3.3.1 Secondary Efficacy Endpoint Analyses by MMRM SPCD OLS ANCOVA SPCD

The change from baseline for all quantitative secondary endpoints which were measured at baseline, Week 6 and 12 (except the CGI-C and the PGI-C) were analyzed using the SPCD OLS ANCOVA method on the mITT population and LOCF within a study stage was used for missing values at Week 6 or Week 12. The ANCOVA model included treatment as a factor and baseline value as a covariate. In addition, the secondary endpoints derived from the NSA-16, PANSS and CDSS (which were assessed at baseline, Week 3, 6, 9 and 12) were analyzed using the MMRM SPCD method.

For the CGI-C and the PGI-C, an ANCOVA model with treatment as a factor and baseline NSA-16 total score as a covariate was used.

1.7.3.3.2 PANSS Response Analysis

The number and percent of patients in the mITT population who had a favorable treatment response, i.e., ≥20% reduction in the PANSS total score, at Week 6 and Week 12, were summarized by stage and treatment group. LOCF was used for patients' missing data. Overall Stage 1 and 2 treatment differences were tested via SPCD 1 degree of freedom score test assuming Stage 2 and 1 treatment effect ratio p=1 (Ivanova et al. Stat Med. 2011; 30(23):2793-2803). In addition, the treatment effect was tested at each visit by Chi-square test or Fisher's Exact.

For the binary response variable of treatment response, a generalized estimating equation (GEE) model analysis on the observed data was performed. The generalized estimating equation model included terms of treatment, visit, treatment-by-visit interaction, baseline value, and baseline-by-visit interaction. P-value, odds ratio (OR), and its 95% confidence interval (CI) were provided for each visit.

1.7.3.3.3 12-Week Parallel Group Analysis

To evaluate the treatment effect under 12 weeks of exposure to the same treatment, a repeated measures analysis of the NSA-16 total score using observed data from all scheduled visits was performed on the mITT 12-Week Parallel Group Population and PP 12-Week Parallel Group Population. This analysis compared treatment groups over time using a linear mixed effects model. The model included fixed effects for treatment, visit, treatment-by-visit interaction, baseline NSA-16 total score, and baseline-by-visit interaction. Unstructured covariance was used as first preference, as in the primary.

This analysis was performed on the NSA-16 total score in addition to all other secondary endpoints for the mITT 12-Week population. The model for CGI-C and PGI-C contained treatment, visit, and treatment-by-visit interaction.

1.7.3.3.4 Analyses Using the PP Population

In addition to the primary efficacy end point NSA-16 analysis on the PP population (sensitivity analysis), the following secondary efficacy end points were also analyzed using the PP population: PANSS total score, PANSS Negative Subscale, PANSS Marder Negative Factors, MCCB composite score and CGI-C. Both SPCD and 12-week parallel group comparison analyses were performed.

1.7.3.4 Supplementary Analyses

1.7.3.4.1 12-Week Parallel Group ANCOVA

For all efficacy variables, separate by-visit ANCOVA models was performed on the mITT 12-Week Parallel Group population. Missing values were imputed by LOCF (regardless of stage). The ANCOVA model included treatment as a factor, and baseline value as a covariate. For the CGI-C and the PGI-C, the model did not contain baseline value. For the CGI-C, the model for Week 12 assessed the difference relative to baseline.

1.7.3.4.2 CGI-C and PGI-C Proportional Odds Ratio

For the CGI-C and PGI-C, odds ratios were obtained through a proportional odds regression model at each visit and represented the odds of a favorable response with d6-DM/Q compared to placebo. Odds ratios >1 indicated an increased likelihood of a favorable response in patients taking d6-DM/Q. This analysis was performed the mITT population and on the 12-Week Parallel Group population.

1.7.3.4.3 Smoking Cessation Analysis

Descriptive summaries of the number and percentage of patients in each tobacco use category (never, current, and former) were presented. Among those who were former tobacco users, descriptive statistics of the rate of use (defined as the number of units per day) were calculated. Among those who were current users at baseline or begin smoking post-baseline, the rate and change from baseline of the rate at each visit were calculated and summarized descriptively. All analyses were summarized by treatment group and performed on the mITT (Stage 1) and mITT 12-Week Parallel Group populations.

1.7.3.4.4 SPCD Analysis by Subgroup

The primary endpoint was analyzed using the subgroups defined by each category below on the mITT population, using the OLS ANCOVA methodology.
Age group (<45, ≥45)
Gender (male, female)
Baseline MCCB composite score (<30, ≥30)
Baseline concomitant benzodiazepine/SSRI/SSNI medication use
Onset of schizophrenia (<20 years, ≥20 years) and residual schizophrenia (<4 years, ≥4 years)

1.7.3.4.5 Treatment Effect by Baseline Concomitant Medication

Analyses of the primary endpoint (NSA-16 total score) were performed for the subgroup of patients using concomitant psychotropic medications (antidepressants, antipsychotics) considered to be CYP2D6 major substrates (aripiprazole, risperidone, duloxetine, fluoxetine, fluvoxamine, mirtazapine, paroxetine, and venlafaxine) and the subgroup with concomitant psychotropic medications that were considered not to be CYP2D6 major substrates. The ANCOVA analyses (LOCF) were conducted using the mITT (Stage 1) and mITT 12-Week Parallel Group Population.

In addition, concomitant use of beta blockers that are CYP2D6 substrates was analyzed for TEAEs, such as cardiovascular-related AEs, falls, etc. The analysis was conducted for the subgroup of patients who were taking concomitant beta blockers that are considered to be major substrates of CYP2D6 and for patients taking beta blockers that are not major substrates of CYP2D6.

1.7.3.4.6 NSA-16 Band-Pass Filter Analysis

Band-pass filtering is a statistical methodology that filters out data from trial sites generating non-plausible high or low levels of placebo response, thus yielding a more accurate effect size and greater separation of active drug (when efficacious) from placebo (Targum et al. Eur Neuropsychopharmacol. 2014; 24(8):1188-1195). The NSA-16 total score change from baseline (on observed data) was analyzed using one band-pass filter (>0 or <−7). Mean NSA-16 change from baseline scores for each site were calculated and the sites that had scores exceeding the boundaries of the band-pass filter threshold were considered non-informative and were excluded from the analysis. After applying the band-filter, NSA-16 total score change from baseline were analyzed using SPCD methodology (mITT) and on the 12-week parallel group population.

1.7.4 Pharmacokinetics and Pharmacodynamics Analysis

Plasma concentrations of d6-DM, d3-DX, d3-3-MM and Q obtained from blood samples collected at baseline (Day 1), Visit 4 (Week 6) and Visit 7 (Week 12/ET) were summarized descriptively overall and by CYP2D6 metabolizer group. PK parameter (Cₘₐₓ and AUC) estimation was performed for d6-DM, d3-DX and Q. Predicted PK parameters were also summarized descriptively overall and by metabolizer group.

Correlations between the estimated PK parameters of d6-DM, d3-DX and Q and the change from baseline in the NSA-16 were provided.

Blood draws to assess concentrations of SGAs were performed at screening, Week 6, and Week 12, and results from the plasma concentrations were summarized descriptively.

1.7.5 Safety Analysis

Unless otherwise specified, safety analyses that include summaries of number and percent (e.g., AEs) were displayed using the following treatment groups:
Placebo/Placebo: patients receiving Placebo/Placebo during the study. Note that patients randomized to Placebo/d6-DM/Q but dropped out in Stage 1 were not included in this population. Instead, these were summarized under the ‘All Placebo’ treatment group.
d6-DM/Q/d6-DM/Q: patients receiving d6-DM/Q for the entire duration of the study.
Placebo/d6-DM/Q: patients who switched from placebo to d6-DM/Q. This group was further divided into data that occurred while on placebo and data that occurred while on d6-DM/Q.
All Placebo: This included data from the stages when patients received placebo.
All d6-DM/Q: This included data from the stages when patients received d6-DM/Q.

For quantitative summaries (e.g., ECGs, labs), the All Placebo and All d6-DM/Q groups were not included.

1.7.5.1 Adverse Events

AEs were coded using Medical Dictionary for Regulatory Activities (MedDRA) version 18.1. Tabular summaries of TEAEs, AEs leading to discontinuation, treatment-related AEs, and SAEs were summarized by system organ class (SOC) and preferred term (PT). Patients who took only d6-DM/Q or only placebo and had multiple AEs within the same SOC or PT were only counted once within a level of MedDRA. If patients switched from placebo to d6-DM/Q and had the same AE start in both study stages, it was counted under both placebo and d6-DM/Q.

1.7.5.2 Clinical Laboratory Assessments

Hematology, chemistry, and urinalysis assessments were summarized descriptively using change from baseline and percent change from baseline, by visit and by stage. Out-of-range values were assessed through shift tables. Each value was assessed as low, normal, or high based on the normal ranges provided by the central lab. Frequencies of each combination of shifts were provided by treatment group.

Shift tables were created by stage and also for the safety 12-week parallel group population (Placebo/Placebo and d6-DM/Q/d6-DM/Q). In both stages, patients taking d6-DM/Q were compared to those taking placebo. The Stage 1 shift table included all patients in the safety population, while the Stage 2 shift table included only re-randomized patients. Baseline in all shift tables was the last assessment prior to first dose in each stage.

The number and percent of patients meeting PCS criteria any time post-baseline were summarized by treatment group.

Over the course of the study, there may have been some lab tests performed that were not mentioned in the protocol. These tests were not to be summarized but were to be included in the listings and flagged as non-protocol tests.

1.7.5.3 Electrocardiogram

The quantitative parameters of HR, PR interval, QRS duration, QT interval (uncorrected), and QTcF were reported by a central reader. Change from baseline and percent change from baseline were calculated for each parameter and summarized by treatment group. In addition, since ECGs were recorded pre-dose and post-dose at baseline, Week 6, and Week 12, change from pre-dose to post-dose was summarized at these visits.

The number and percentage of patients meeting the PCS criteria were summarized by treatment group. Summaries were given for any time post-baseline and by visit. For QTcF, males and females were assessed separately. Patients were included in all categories for which they qualified.

ECG overall interpretations were summarized by the number and percentage that were normal or abnormal. Cardiologist interpretations (i.e., central ECG) were used for these summaries. All interpretations and corresponding details were listed by-patient.

1.7.5.4 Vital Signs

The parameters summarized from when patients were in supine/semi-recumbent positions included SBP. DBP, and HR. These measurements were recorded twice, so the mean of the 2 measurements was taken and used for all summaries mentioned below. Weight was also summarized. These parameters were summarized through change from baseline and percentage change from baseline in similar fashion as the ECG parameters.

Vital signs were also assessed through PCS criteria. Patients were counted if they met the established criteria at any time post-baseline. All vital sign parameters were included in by-patient listings.

1.7.5.5 Physical and Neurological Exams

Physical and neurological exams were scheduled for assessment at screening only and were presented in by-patient listings.

1.7.5.6 Columbia Suicide Severity Rating Scale (C-SSRS)

The scoring for the C-SSRS was analyzed using the following indicators:
Ideation severity: each of the 5 questions regarding type of ideation (yes/no)
Intensity of most severe ideation: a sum of the 5 intensity items
Suicidal behavior types: each of the 4 suicidal behavior types (yes/no)
Suicidal behavior: suicidal behavior question
Actual lethality: actual lethality question (0-5 scale) on most lethal attempt
Potential lethality: potential lethality question (0-2 scale) on most lethal attempt

The individual questions listed above that had yes/no responses were summarized by treatment group and visit. Intensity of most or severe ideation was summarized, descriptive by treatment group and visit. Items that contain an ordinal response were summarized through descriptive statistics, number, and percentage. All C-SSRS data, including individual text descriptions (included as part of some questions), were included in by-patient listings.

1.7.5.7 Simpson Angus Scale for Extrapyramidal Symptoms (SAS)

The individual items, as well as the total score of the SAS were summarized. Descriptive statistics were provided by treatment group and visit for males and females separately, as well as both sexes overall. For each item, a shift table of the number and percent of patients in each score (0 to 4) from baseline to Week 6 and baseline to Week 12 was summarized overall and for males and females separately.

1.7.5.8 Barnes Akathisia Scale (BAS)

The objective assessment, subjective awareness, subjective distress, and global clinical assessment, as well as the total score for the BAS were summarized. Descriptive statistics were provided by treatment group and visit for males and females separately, as well as both sexes overall. Shift tables of the number and percent of patients of the total score and the global clinical assessment were presented for baseline to Week 6 and baseline to Week 12, for males and females separately as well as both sexes overall.

1.7.5.9 Abnormal Involuntary Movements Scale (AIMS)

The scores on each subscale of the AIMS were summarized using descriptive statistics by treatment group and by visit for males and females separately, as well as both sexes overall. Shift tables of the number and percent of patients of the total score and the global clinical assessment were presented for baseline to Week 6 and baseline to Week 12, for males and females separately as well as both sexes overall.

1.7.6 Determination of Sample Size

Sample size calculations were performed assuming a bivariate normal distribution for the primary endpoint, based on published studies such as Kane et al. (Arch Gen Psychiatry. 1988; 45(9):789-796) and Buchanan et al. (Schizophr Bull. 2015; 41(4):900-908). Both Stage 1 and 2 treatment differences were assumed to be −2.5, and the standard deviation in drug and placebo groups was assumed to be equal to 5 (effect size of −0.5). It was further assumed that 82% of patients would complete Stage 1, 70% of these patients assigned to placebo would be placebo non-responders, and 91% of these would complete Stage 2. A sample size of 120 patients randomized in 1:2 ratio (active:placebo) in Stage 1 would have an approximate 80% power for this SPCD study with 2-sided type I error α=0.05. If the effect sizes in both stages were assumed to be −0.425, the power would be approximately 70%.

1.7.7 Statistical/Analytical Issues

1.7.7.1 Adjustments for Covariates

The planned and actual covariates and methods used in analyses did not differ in this study.

1.7.7.2 Handling of Dropouts or Missing Data

Missing data were handled differently depending on the parameter and analysis. Note that analyses done on ‘observed cases’ (such as MMRM analyses) did not follow any imputation rules below. See below for considerations:
Missing baseline values were not imputed in any situation.
For SPCD and by-stage and visit efficacy analyses (excluding observed cases analyses), missing data were imputed by LOCF within a study stage. No imputation was done for patients without data in Stage 2.
A LOCF approach was also used for efficacy analyses on the mITT 12-week parallel group population, meaning the last non-missing post-baseline observation was carried forward to each visit.

1.7.7.3 Interim Analyses and Data Monitoring

No data monitoring safety board was appointed for this study and no interim analyses were planned or carried out.

1.7.7.4 Multicenter Studies

The data collected in this study were analyzed as a whole.

1.7.7.5 Multiple Comparisons/Multiplicity

No adjustments were made for testing multiple secondary outcome measures. Since it was possible that some significant results could have occurred by chance alone, undue consideration was not given to isolated significant differences; rather, interpretations were made based on patterns of significant differences and their consistency with the primary endpoint analysis.

1.7.7.6 Use of an “Efficacy Subset” of Patients

A post-hoc analysis was performed excluding all patients who had more than one rater from both the placebo and d6-DM/Q treatment groups to assess the impact of the use of a single-rater vs. multiple raters assessing the same endpoints throughout the study. Results of this analysis are presented in Section 3.4.1.4.1.

1.7.7.7 Examination of Subgroups

Factors used for subgroup analysis of efficacy are described in Section 1.7.3.4.4. The categories included age, gender, baseline MCCB composite score, baseline concomitant benzodiazepine/SSRI/SSNI medication use, and onset of schizophrenia.

1.8 Changes in the Conduct of the Study or Planned Analyses

12-Week Parallel Group ANCOVA was specified for all efficacy endpoints in Section 1.7.3.4.1 but was only performed for NSA-16 related efficacy endpoints. This is because similar 12-Week Parallel Group MMRM was performed as specified in Section 1.7.3.3.3 and MMRM is the preferred method.

After database lock, one investigator (Site 117, Principal Investigator) communicated that there were some data entry errors identified at the site that were not corrected prior to database lock. These discrepancies were reviewed and it was concluded that the changes had no effect on the efficacy results or the overall integrity of the data, and there were no safety concerns, thus the database was not unlocked and relocked. A deviation report was filed in the trial master file per the SOP.

2 Study Patients

2.1 Disposition of Patients

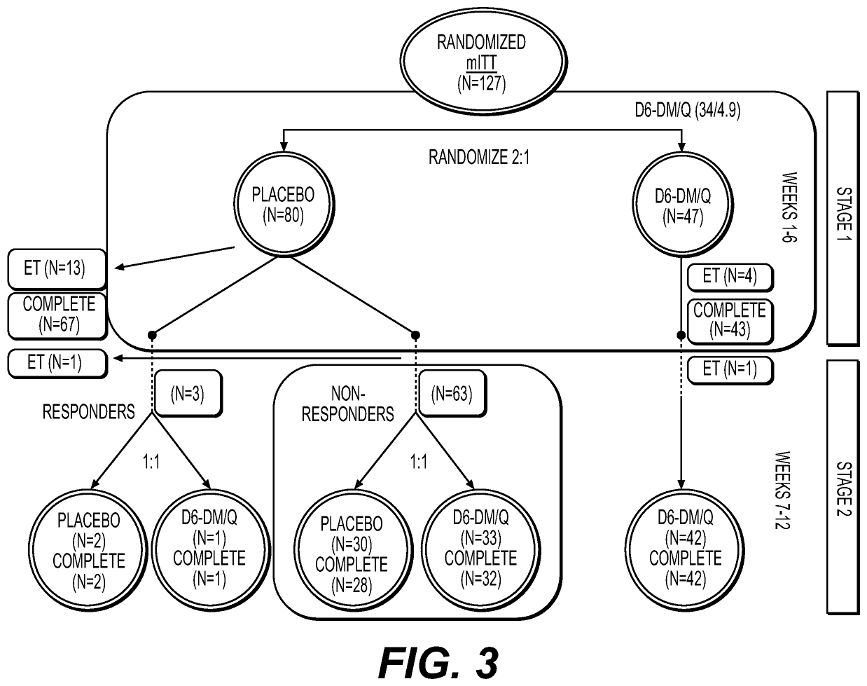

Overall patient disposition is summarized in Table 4 and patient disposition for the mITT population is depicted in FIG. 3. Of the 286 patients screened, 145 patients were enrolled and randomized in Stage 1. Of the 145 patients randomized, 48 patients were randomized to d6-DM/Q and 97 patients to placebo in Stage 1; one patient was randomized to placebo but did not receive study drug. For all randomized patients, a total of 123 patients completed Stage 1 (d6-DM/Q: 43 patients; placebo: 80 patients) and the remaining 21 patients discontinued during Stage 1. For the mITT population, of the 47 patients received d6-DM/Q in Stage 1, 42 patients entered Stage 2 and continued to receive d6-DM/Q, while 80 patients received placebo in Stage 1, 66 entered Stage 2 and were re-randomized (d6-DM/Q: 32 patients; placebo: 34 patients). At the end of Stage 1, 64 patients were non-responders (<20% reduction in PANSS total score) and 3 patients were responders (≥20% reduction in PANSS total score) in the placebo group, and 42 were non-responders and 5 were responders in the d6-DM/Q group.

Overall, for all randomized patients, 118 patients completed the study (d6-DM/Q in Stages 1 and 2: 42 patients; placebo in Stages 1 and 2: 37 patients; placebo in Stage 1 and d6-DM/Q in Stage 2: 39 patients). Of the 27 patients (18.6%) who discontinued the study, 10 were due to an AE, 8 were withdrawal by patient, 4 were lost to follow up, 3 were withdrawn due non-compliance with study drug, 1 was withdrawn due to physician decision, and 1 was withdrawn due to other reason(s) not listed. Reasons for discontinuation were similar across treatment groups.

TABLE 4
Overall Patient Disposition-All Patients

| | Placebo/ | d6-DM/Q/ | Placebo/ | All | All |
|---|---|---|---|---|---|
| | Placebo | d6-DM/Q | d6-DM/Q | d6-DM/Q | Patients |
| | N 49 | N = 48 | N = 48 | N = 96 | N = 286 |
| Patients screened, n (%) | — | — | — | — | 286 |
| Screen failures | — | — | — | — | 141 (49.3) |
| Inclusion/exclusion criteria unmet | — | — | — | — | 115 (40.2) |
| Lost to follow-up | — | — | — | — | 120 (42.0) |
| Withdrew consent | — | — | — | — | 128 (44.8) |
| Other | — | — | — | — | 5 (1.7) |
| Patients randomized¹ | 42 (85.7) | 48 (100.0) | 42 (87.5) | 90 (93.8) | 132 (91.0) |
| Randomized but did not receive | 1 (2.0) | 0 (0.0) | 0 (0.0) | 0 (0.0) | 1 (0.7) |
| study drug, n (%) | | | | | |
| Completed study, n (%) | 37 (75.5) | 42 (87.5) | 39 (81.3) | 81 (84.4) | 118 (81.4) |
| Patients who discontinued | 12 (24.5) | 6 (12.5) | 9 (18.8) | 15 (15.6) | 27 (18.6) |
| from study, n (%) | | | | | |
| Adverse event | 5 (10.2) | 2 (4.2) | 3 (6.3) | 5 (5.2) | 10 (6.9) |
| Lost to follow-up | 3 (6.1) | 0 (0.0) | 1 (2.1) | 1 (1.0) | 4 (2.8) |
| Physician decision | 1 (2.0) | 0 (0.0) | 0 (0.0) | 0 (0.0) | 1 (0.7) |
| Protocol deviation | 0 (0.0) | 0 (0.0) | 0 (0.0) | 0 (0.0) | 0 (0.0) |
| Withdrawal by patient | 1 (2.0) | 3 (6.3) | 4 (8.3) | 7 (7.3) | 8 (5.5) |
| Noncompliance with study drug | 1 (2.0) | 1 (2.1) | 1 (2.1) | 2 (2.1) | 3 (2.1) |
| Other | 1 (2.0) | 0 (0.0) | 0 (0.0) | 0 (0.0) | 1 (0.7) |

Denominators for screen failures and reasons for screen failures are the number of patients screened. Denominators for all other categories are the number of patients randomized (N = 145). — = not applicable.
¹A total of 13 patients with randomization error were excluded.

3 Efficacy Evaluation

3.1 Data Sets Analyzed

Analysis populations are summarized by treatment groups in Table 5. Of the 145 patients randomized at the beginning of the study, 132 were included in the ITT population. 144 were included in the safety population, and 110 were included in the PP population. Of the randomized patients, 127 patients met the criteria for mITT population and were included in the Stage 1 mITT population. A total of 108 patients were included in the Stage 2 ITT population, and Stage 2 mITT population. The disposition of patients included in the mITT population is provided in FIG. 3.

For all data sets analyzed in this study, the patient criteria for each population are described in Section 1.7.2.

TABLE 5
Summary of Analysis Populations and Stage 2 Subsets-All Randomized
Patients

| Population | Stage 1 | | | Stage 2 | | |
|---|---|---|---|---|---|---|
| Subset | Overall | d6-DM/Q | Placebo | Overall | d6-DM/Q | Placebo |
| mITT | 127 | 47 | 80 | 108 | 76 | 32 |
| Stage 2 mITT subsets | | | | | | |
| Placebo non-responders | — | — | — | 63 | 33 | 30 |
| Placebo responders | — | — | — | 3 | 1 | 2 |
| Placebo non-responders + | — | — | — | 66 | 34 | 32 |
| responders | | | | | | |
| PP | 110 | 37 | 73 | 94 | 65 | 29 |
| ITT | 132 | 48 | 84 | 108 | 76 | 32 |
| Safety | 144 | 48 | 96 | — | — | — |
| 12-week parallel group | | 48 | 42 | | | |
| mITT 12-week parallel group | 87 | 47 | 40 | — | — | — |
| Safety 12-week parallel group | 89 | 48 | 41 | | | |
| PP 12-week parallel group | 74 | 37 | 37 | — | — | — |

Analyses on the 12-week parallel group and safety populations do not consider study stage, so it is not necessary to define Stage 2 Ns.
ITT = intent-to-treat;
mITT = modified intent-to-treat;
PP = per-protocol;
— = not applicable.

3.2 Demographic and Other Baseline Characteristics

3.2.1 Demographics

Baseline and demographic characteristics of the Stage 1 mITT population are summarized in Table 6. The mean (SD) age at enrollment was 45.5 (11.19) years. There were 88 male patients (69.3%) and 39 female patients (30.7%) in the mITT population. The majority of the patients included in the mITT population were Black (69 patients [54.3%]) or White (48 patients, [37.8%]). Baseline and demographic characteristics were similar across treatment groups. The demographic and baseline characteristics of the mITT 12-week parallel group and safety populations were comparable to the mITT population found in Table 6 across treatment groups.

TABLE 6
Demographics and Baseline Characteristics-mITT Population

| | d6-DM/Q | Placebo | All Patients |
|---|---|---|---|
| Characteristics, n | (N = 47) | (N = 80) | (N = 127) |
| Gender, n (%) | | | |
| Female | 17 (36.2) | 22 (27.5) | 39 (30.7) |
| Male | 30 (63.8) | 58 (72.5) | 88 (69.3) |
| Race, n (%) | | | |
| Black or African American | 25 (53.2) | 44 (55.0) | 69 (54.3) |
| White | 18 (38.3) | 30 (37.5) | 48 (37.8) |
| Asian | 4 (8.5) | 4 (5.0) | 8 (6.3) |
| American Indian or Alaska Native | 0 (0.0) | 0 (0.0) | 0 (0.0) |
| Native Hawaiian or Other Pacific Islander | 0 (0.0) | 0 (0.0) | 0 (0.0) |
| Other | 0 (0.0) | 2 (2.5) | 2 (1.6) |
| Ethnicity, n (%) | | | |
| Hispanic or Latino | 4 (8.5) | 9 (11.3) | 13 (10.2) |
| Not Hispanic or Latino | 43 (91.5) | 71 (88.8) | 114 (89.8) |
| Age, years | | | |
| Mean (SD) | 46.5 (12.17) | 44.9 (10.60) | 45.5 (11.19) |
| Median | 51.0 | 47.0 | 48.0 |
| Min, Max | 18, 60 | 18, 60 | 18, 60 |
| Age group, n (%) | | | |
| <55 years | 31 (66.0) | 66 (82.5) | 97 (76.4) |
| ≥55 years | 16 (34.0) | 14 (17.5) | 30 (23.6) |
| Height, cm | | | |
| Mean (SD) | 173.2 (8.95) | 173.3 (8.99) | 173.2 (8.94) |
| Median | 173.4 | 174.5 | 174.0 |
| Min, Max | 156, 191 | 152, 191 | 152, 191 |
| Weight, kg | | | |
| Mean (SD) | 98.5 (24.21) | 96.7 (22.65) | 97.4 (23.16) |
| Median | 95.9 | 95.7 | 95.8 |
| Min, Max | 53,192 | 55, 182 | 53, 192 |
| BMI, kg/m² | | | |
| Mean (SD) | 32.9 (7.75) | 32.3 (7.30) | 32.5 (7.45) |
| Median | 31.6 | 31.8 | 31.8 |
| Min, Max | 17, 57 | 19, 56 | 17, 57 |

For each categorical parameter, the denominator for the percent was the number of patients who had that parameter assessed.
BMI = body mass index;
mITT = modified intent-to-treat;
SD = standard deviation.

3.2.2 Medical History

Because of the population being investigated in this study, the most commonly reported SOC and PT for medical history were psychiatric disorders (144 patients [100.0%]) and schizophrenia, residual type (144 patients [100.0%]), respectively. The next most frequently reported PTs in patient medical history was hypertension (35 patients [36.5%] placebo group and 12 patients [25.0%] in d6-DM/Q group).

3.2.3 Prior and Concomitant Medications

Prior medications are presented by anatomical therapeutic subgroup and preferred base name for the safety population in Table 67. Concomitant medications are presented by anatomical therapeutic subgroup and preferred base name for the safety population in Table 68. Tables 17 to 94 are included in Appendix 1.

Baseline concomitant use of SGAs was summarized for the safety population in Table 17. The most frequently used SGA at baseline by patients in the placebo and d6-DM/Q treatment group were aripiprazole (16.7% placebo, 31.3% d6-DM/Q), olanzapine (27.1% placebo, 25.0% d6-DM/Q), and risperidone (26.0% placebo, 20.8% d6-DM/Q).

3.2.4 Other Baseline Characteristics

Baseline values at Stage 1 for the efficacy measures in the mITT population are summarized and presented in Table 7. Baseline values were comparable between the 2 treatment groups.

Stage 2 baseline efficacy assessments for placebo non-responders and placebo responders in the mITT study population are presented in Table 19 and Table 20, respectively.

TABLE 7
Stage 1 Baseline Efficacy Assessments-mITT Population

| | Placebo | d6-DM/Q | All Patients |
|---|---|---|---|
| Measure, Mean (SD) | (N = 80) | (N = 47) | (N = 127) |
| NSA-16 Total Score | 60.4 (7.71) | 61.0 (7.53) | 60.6 (7.62) |
| NSA-16 Factor Domain: Communication | 12.6 (2.71) | 12.5 (254) | 12.6 (2.64) |
| NSA-16 Factor Domain: Emotion/Affect | 12.2 (1.81) | 12.6 (2.11) | 12.3 (1.93) |
| NSA-16 Factor Domain: Social Involvement | 12.2 (2.20) | 12.2 (2.02) | 12.2 (2.13) |
| NSA-16 Factor Domain: Motivation | 16.7 (2.33) | 16.5 (2.37) | 16.6 (2.33) |
| NSA-16 Factor Domain: Retardation | 6.7 (1.61) | 7.2 (1.54) | 6.9 (1.59) |
| NSA-4 Total Score | 17.3 (2.36) | 17.4 (2.44) | 17.3 (2.38) |
| NSA-16 Item 1: Prolonged Time to Respond | 3.1 (1.13) | 3.1 (1.22) | 3.1 (1.15) |
| NSA-16 Item 2: Restricted Speech Quantity | 3.8 (1.12) | 3.5 (1.10) | 3.7 (1.12) |
| NSA-16 Item 3: Impoverished Speech Content | 3.7 (0.91) | 3.6 (0.80) | 3.6 (0.87) |
| NSA-16 Item 4: Inarticulate Speech | 2.0 (1.07) | 2.3 (1.16) | 2.1 (1.11) |
| NSA-16 Item 5: Emotion: Reduced Range | 4.2 (0.81) | 4.4 (0.92) | 4.3 (0.85) |
| NSA-16 Item 6: Affect: Reduce Modulation Intensity | 4.2 (0.74) | 4.2 (0.89) | 4.2 (0.80) |
| NSA-16 Item 7: Affect: Reduced Display on Demand | 3.8 (0.93) | 4.0 (0.92) | 3.9 (0.94) |
| NSA-16 Item 8: Reduced Social Drive | 4.7 (0.75) | 4.9 (0.55) | 4.8 (0.68) |
| NSA-16 Item 9: Poor Rapport with Interviewer | 3.4 (1.02) | 3.3 (0.93) | 3.3 (0.99) |
| NSA-16 Item 10: Sexual Interest | 4.2 (1.52) | 4.0 (1.54) | 4.1 (1.52) |
| NSA-16 Item 11: Poor Grooming and Hygiene | 2.6 (1.20) | 2.5 (1.08) | 2.6 (1.15) |
| NSA-16 Item 12: Reduced Sense of Purpose | 4.7 (0.92) | 4.5 (1.02) | 4.6 (0.96) |
| NSA-16 Item 13: Reduced Interests | 4.5 (0.84) | 4.8 (0.84) | 4.6 (0.84) |
| NSA-16 Item 14: Reduced Daily Activity | 4.8 (0.57) | 4.7 (0.74) | 4.8 (0.64) |
| NSA-16 Item 15: Reduced Expressive Gestures | 3.9 (1.09) | 4.1 (1.13) | 4.0 (1.11) |
| NSA-16 Item 16: Slowed Movements | 2.8 (0.96) | 3.0 (0.78) | 2.9 (0.91) |
| NSA-16: Global Negative Symptoms Rating | 4.6 (0.61) | 4.6 (0.64) | 4.6 (0.62) |
| NSA-16: Global Level of Functioning | 4.6 (0.63) | 4.7 (0.73) | 4.6 (0.66) |
| PANSS Total Score | 68.7 (7.99) | 67.4 (8.26) | 68.2 (8.08) |
| PANSS Total Score Reduction ≥20% n, (%)¹ | | | |
| Yes | 3 (3.8) | 5 (10.6) | 8 (6.3) |
| No | 77 (96.3) | 42 (89.4) | 119 (93.7) |
| PANSS Negative Subscale | 25.2 (3.64) | 24.6 (3.51) | 25.0 (3.59) |
| PANSS Positive Subscale | 13.4 (2.81) | 13.6 (3.65) | 13.5 (3.13) |
| PANSS General Psychopathology Subscale | 30.1 (5.05) | 29.1 (4.66) | 29.7 (4.91) |
| PANSS Prosocial Factors | 18.4 (2.92) | 18.3 (3.24) | 18.4 (3.03) |
| PANSS Marder Negative Factors | 24.2 (3.81) | 24.1 (4.24) | 24.2 (3.96) |
| PANSS Excitement Component | 6.3 (2.10) | 6.2 (1.57) | 6.3 (1.92) |
| CGI-S | 3.9 (0.74) | 3.7 (0.74) | 3.9 (0.74) |
| COSS Total Score | 0.9 (1.31) | 1.1 (1.34) | 0.9 (1.32) |

COSS = Calgary Depression Scale for Schizophrenia;
CGI-S = Clinical Global Impression of Severity of Illness;
mITT = modified intent-to treat;
NSA-16 = 16-Item Negative Symptom Assessment;
PANSS = Positive and Negative Syndrome Scale;
SD = standard deviation
¹The data presented is the frequency (%) of patients who had (yes/no) a reduction of 20% from baseline at Week 6 in PANSS Total score. Source: Table 18

3.3 Measurements of Treatment Compliance

Study drug compliance is summarized and presented for the safety population in Table 8. The majority of patients in each treatment group took between 80% and 120% of their scheduled doses and were considered compliant.

TABLE 8
Treatment Compliance-Safety Population

| | d6-DM/Q/ | Placebo/ | Placebo/d6-DM/Q | |
|---|---|---|---|---|
| | d6-DM/Q | Placebo | (N = 40) | |
| | (N = 48) | (N = 56) | Placebo | d6-DM/Q |
| Compliance (%), n | 48 | 52 | 40 | 39 |
| Mean (SD) | 82.17 (13.628) | 75.37 (21.206) | 85.97 (11.013) | 85.97 (9.424) |
| Median (min, max) | 87.75 (12.5, 89.6) | 86.20 (6.3, 92.2) | 87.50 (43.8, 99.0) | 86.50 (64.6, 132.3) |
| Compliance Ratio, n (%) | 48 | 52 | 40 | 39 |
| <80% | 10 (20.8) | 17 (32.7) | 4 (10.0) | 6 (15.4) |
| 80-120% | 38 (79.2) | 35 (67.3) | 36 (90.0) | 32 (82.1) |
| >120% | 0 (0.0) | 0 (0.0) | 0 (0.0) | 1 (2.6) |

SD = standard deviation.

3.4 Efficacy Results and Tabulations of Individual Patient Data

3.4.1 Analysis of Efficacy

3.4.1.1 Primary Efficacy Endpoint (16-Item Negative Symptom Assessment [NSA-16] Total Score)

3.4.1.1.1 Primary Analysis

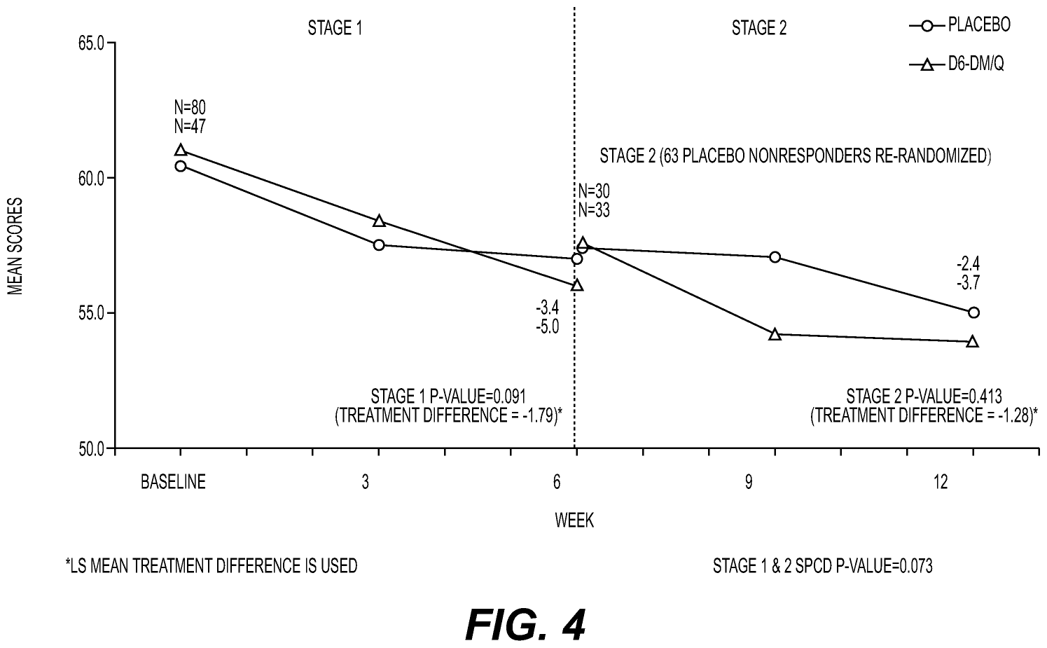

The primary efficacy analysis was the SPCD analysis of the change from baseline in the NSA-16 total score for d6-DM/Q versus placebo. A numerically higher improvement was observed with d6-DM/Q compared to placebo in the change from baseline in the NSA-16 total score with the SPCD analysis using the mITT population (SPCD weighted Z-statistic=−1.79, p=0.073, Table 9). In Stage 1 (which mimicked a parallel-group design), the mean (SD) change from baseline in NSA-16 total scores was −5.0 (5.64) with d6-DM/Q and −3.4 (5.54) with placebo, resulting in a LS mean treatment difference of −1.79 (95% CI −3.86, 0.29; p=0.091). In Stage 2 which includes only placebo non-responders randomized to d6-DM/Q or placebo, the mean (SD) change from baseline in NSA-16 total scores was −3.7 (6.41) with d6-DM/Q and −2.4 (5.88) with placebo resulting in a LS mean treatment difference of −1.28 (95% CI −4.39, 1.83; p=0.413; FIG. 4).

TABLE 9
NSA-16 Total Score: Change from Baseline
SPCD MMRM (Observed Data)-mITT Population

| Stage | Parameter/Result | d6-DM/Q | Placebo |
|---|---|---|---|
| Stage 1 | | | |
| | Baseline, n | 47 | 80 |
| | Mean (SD) | 61.0 (7.53) | 60.4 (7.71) |
| | Visit 4 (Week 6), n | 47 | 70 |
| | Mean change (SD) | −5.0 (5.64) | −3.4 (5.54) |
| | Treatment difference | | |
| | vs. placebo | | |
| (95% CI)¹ | | −1.79 (−3.86, 0.29) | |
| | p-value | 0.091 | |
| Stage 2 (Stage 1 | | | |
| placebo non-responders) | | | |
| | Baseline, n | 33 | 30 |
| | Mean² (SD) | 57.6 (9.09) | 57.6 (9.39) |
| | Visit 7 (Week 12), n | 32 | 29 |
| | Mean change (SD) | −3.7 (6.41) | −2.4 (5.88) |
| | Treatment difference | | |
| | vs. placebo | | |
| (95% CI)¹ | | −1.28 ( −4.39, 1.83) | |
| | p-value | 0.413 | |
| MMRM weighted z- | | −1.79, p = 0.073 | |
| statistic, overall p-value³ | | | |

Possible NSA-16 total score ranged from 16 to 96;
a higher score indicated a worse condition.
Cl = confidence interval;
NSA-16 = Negative Symptom Assessment Scale;
mITT = modified intent-to-treat;
MMRM = mixed model repeat measures;
SD = standard deviation;
SPCD = sequential parallel comparison design.
¹The MMRM had a fixed effect of treatment, visit, treatment-by-visit interaction, baseline. NSA-16, and baseline NSA-16-by-visit. Unstructured covariance matrix was used.
²Stage 2 baseline was the last non-missing assessment prior to Stage 2 re-randomization (re-randomization visit).
³An SPCD weighted z-statistic was used with a Stage 1 weight of 0.6 and a Stage 2 weight of 0.4. Treatment differences in each stage were estimated by the MMRM.

3.4.1.1.2 Sensitivity Analysis

Sensitivity analyses on the primary endpoint using different statistical analyses methods (SUR method [LOCF] and SPCD with OLS ANCOVA [LOCF]) and different analysis population (PP population) corroborated the findings of the primary analysis; moreover, statistically significant treatment differences between d6-DM/Q and placebo, in favor of d6-DM/Q, were observed with both the SUR method (p=0.048, Table 23) and SPCD OLS ANCOVA (p=0.042, Table 24) but not using the PP population (Table 25). A summary of the results is provided in Table 10.

TABLE 10
Summary of Primary and Sensitivity Analysis of the NSA-16 Total Score

| | Stage 1 | | | | | Stage 2 |
|---|---|---|---|---|---|---|
| | N (d6- | Change from Baseline, | | Treatment | | N (d6- |
| Analyses | DM/Q: | Mean (SD) | | Difference, | p- | DM/Q: |
| (NSA-16 Total Score) | Placebo) | d6-DM/Q | Placebo | LS Mean (Cl) | value | Placebo) |
| Primary Analysis | | | | | | |
| MMRM, mITT | 47:80 | −5.0 (5.64) | −3.4 (5.54) | −1.79 (−3.86. 0.29) | 0.091 | 33:30 |
| Sensitivity Analyses | | | | | | |
| SUR Analysis, LOCF | 47:80 | −5.0 (5.64) | −3.0 (5.78) | −2.05 (1.05)¹ | | 33:30 |
| OLS ANCOVA, LOCF | 47:80 | −5.0 (5.64) | −3.0 (5.78) | −2.05 (4.13, 0.04) | 0.054 | 33:30 |
| MMRM, Per-protocol | 37:73 | −4.8 (5.77) | −3.5 (5.76) | −1.57 (−3.94, 0.80) | 0.191 | 29:27 |
| | Stage 2 | | | | | |
| | Change from Baseline, | | Treatment | | SPCD Analysis | |
| | Mean (SD) | | Difference, | p- | Z- | P- |
| | d6-DM/Q | Placebo | LS Mean (CI) | value | Statistic | value |
| Primary Analysis | | | | | | |
| MMRM, mITT | −2.4 (5.88) | −3.7 (6.41) | −1.28 (−4.39, 1.83) | 0.413 | −1.79 | 0.073 |
| Sensitivity Analyses | | | | | | |
| SUR Analysis, LOCF | −3.6 (6.34) | −2.2 (5.89) | −1.26 (1.52)¹ | | −1.98 | 0.048¹ |
| OLS ANCOVA, LOCF | −3.6 (6.34) | −2.2 (5.89) | −1.40 (−4.46, 1.65) | 0.362 | −2.04 | 0.042 |
| MMRM, Per-protocol | −4.5 (5.96) | −3.0 (5.81) | −1.43 (−4.63, 1.76) | 0.372 | −1.58 | 0.114 |

ANCOVA = analysis of covariance;
CI = confidence interval;
LOCF = last observation carried forward;
LS = least square;
mITT = modified intent-to-treat;
MMRM = mixed mode repeated measure;
NSA-16 = 16-Item Negative Symptom Assessment;
OLS = ordinary least squares;
SD = standard deviation;
SPCD = sequential parallel comparison design;
SUR = seemingly unrelated regression.
Note:
SPCD weighted Z-statistic was calculated using Stage 1 weight = 0.6 and Stage 2 weight = 0.4.
¹The data displayed is: SUR estimated difference versus placebo (standard error) and p-value from SUR.
Source: Tables 23-25

3.4.1.1.3 12-Week Analyses

Analysis of the change in NSA-16 total scores for the patient cohort who were randomized and remained (or early withdraw) in the same treatment assignment in both stages, mimicking a traditional 12-week parallel comparison design instead of 2-stage SPCD, showed no difference between d6-DM/Q and placebo treatment. The mean (SD) change from baseline in NSA-16 total score was −6.6 (7.81) for the d6-DM/Q/d6-DM/Q group and −7.0 (7.71) for the placebo/placebo group of the mITT 12-week parallel group, population.

Similar results were observed when analyzed with ANCOVA (LOCF) using the mITT 12-week parallel group population or with MMRM (observed data) using the PP 12 week parallel group population.

3.4.1.1.4 Subgroup Analysis

The primary endpoint was analyzed using the subgroups defined by each category below on the mITT population, using the OLS ANCOVA SPCD methodology.
Age group (<45; ≥45)
Gender (male; female)
Baseline MCCB composite score (<30; ≥30)
Baseline concomitant benzodiazepine (Table 69), SNRI (Table 70), or SSRI medication use (Table 71)
Onset of schizophrenia (<20 years; ≥20 years) and residual schizophrenia (<4 years; ≥4 years)

No meaningful differential treatment effects between d6-DM/Q and placebo were observed for the subgroup analysis by age, gender, baseline MCCB composite score, baseline concomitant medication use, or onset of schizophrenia and residual schizophrenia.

3.4.1.1.5 Band-Pass Filter Analysis

The primary endpoint was analyzed using one band-pass filter which excluded from the analysis, sites with mean NSA-16 change from baseline scores for placebo that exceeded the boundary (>0 or <−7). Results of this analysis showed a statistically significant treatment difference in favor of d6-DM/Q with the SPCD ANCOVA analysis (SPCD weighted OLS Z-statistic=−2.25, p=0.025, Table 26).

Results for similar analysis performed for the mITT 12-week parallel group population are presented in Table 27.

3.4.1.2 Secondary Endpoints

3.4.1.2.1 Positive and Negative Syndrome Scale (PANSS)

Analysis of the PANSS included the total score of all the 30-items and various subscales derived from these 30 items which include: negative subscale (N1-N7), positive subscale (P1-P7), general psychopathology subscale (G1-G16), prosocial factors (G16, N2, N4, N7, P3, and P6), Marder negative factors (N1, N2, N3, N4, N6, G7, and G16), and excitement component (P4, P7, G4, G8, and G14). A summary of the results for the PANSS total score and subscales is included in Table 11.

TABLE 11
Summary of PANSS Results (SPCD, MMRM; mITT Population)

| | Stage 1 | | | | Stage 2 | | | | | |
|---|---|---|---|---|---|---|---|---|---|---|
| | d6-DM/Q N = 47, Placebo = 80 | | | | d6-DM/Q N = 33, Placebo N = 30 | | | | | |
| | Change from Baseline, | | Treatment | | Change from Baseline, | | Treatment | | SPCD Analysis | |
| | Mean (SD) | | Difference, LS | p- | Mean (SD) | | Difference, LS | p- | Z- | P- |
| PANSS | d6-DM/Q | Placebo | Mean (CI) | value | d6-DM/Q | Placebo | Mean (CI) | value | Statistic | value |
| Total score | −4.7 (6.98) | −2.5 (6.50) | −2.36 (−4.77, 0.06) | 0.055 | −4.0 (7.71) | −1.4 (7.64) | −2.53 (−6.51, 1.45) | 0.209 | −2.25 | 0.025 |
| Negative subscale | −2.2 (3.33) | −1.5 (3.81) | −0.86 (−2.17, 0.45) | 0.198 | −2.3 (3.12) | −1.0 (2.69) | −1.43 (−2.88, 0.03) | 0.054 | −2.20 | 0.027 |
| Marder negative factors | −2.1 (3.34) | −1.6 (3.48) | −0.63 (−1.89, 0.62) | 0,320 | −2.5 (4.27) | −0.8 (2.80) | −1.93 (−3.62, −0.24) | 0.026 | −2.26 | 0.024 |
| Prosocial factors | −2.0 (2.18) | −1.1 (2.53) | −0.89 (−1.75, −0.03) | 0.042 | −1.4 (2.35) | −0.7 (2.02) | −0.89 (−2.00, 0.22) | 0.115 | −2.60 | 0.009 |
| Positive subscale | −0.8 (2.63) | −0.3 (2.57) | −0.42 (−1.36, 0.52) | 0.376 | −0.3 (2.47) | −0.4 (1.99) | 0.28 (−0.90, 1.45) | 0.640 | −0.39 | 0.700 |
| General psychopathology | −1.7 (4.04) | −0.7 (3.21) | −1.14 (−2.40, 0.13) | 0.077 | −1.3 (5.10) | 0.0 (5.14) | −1.40 (−3.99, 1.19) | 0.284 | −1.93 | 0.054 |
| Excitement component | −0.4 (1.64) | −0.2 (1.57) | −0.34 (−0.83, 0.16) | 0.177 | −0.1 (1.77) | 0.0 (2.04) | 0.30 (−0.61, 1.21) | 0.512 | −0.35 | 0.723 |

CI = confidence interval;
LS = least squares;
mITT = modified intent-to-treat;
MMRM = mixed model repeated measure;
PANSS = Positive and Negative Syndrome Scale;
SD = standard deviation;
SPCD = sequential parallel comparison design.
Note:
SPCD weighted Z-statistic was calculated using Stage 1 weight = 0.6 and Stage 2 weight = 0.4. Treatment difference in each stage was estimated by the MMRM.
Source: Tables 28-34

3.4.1.2.1.1 PANSS Total Score

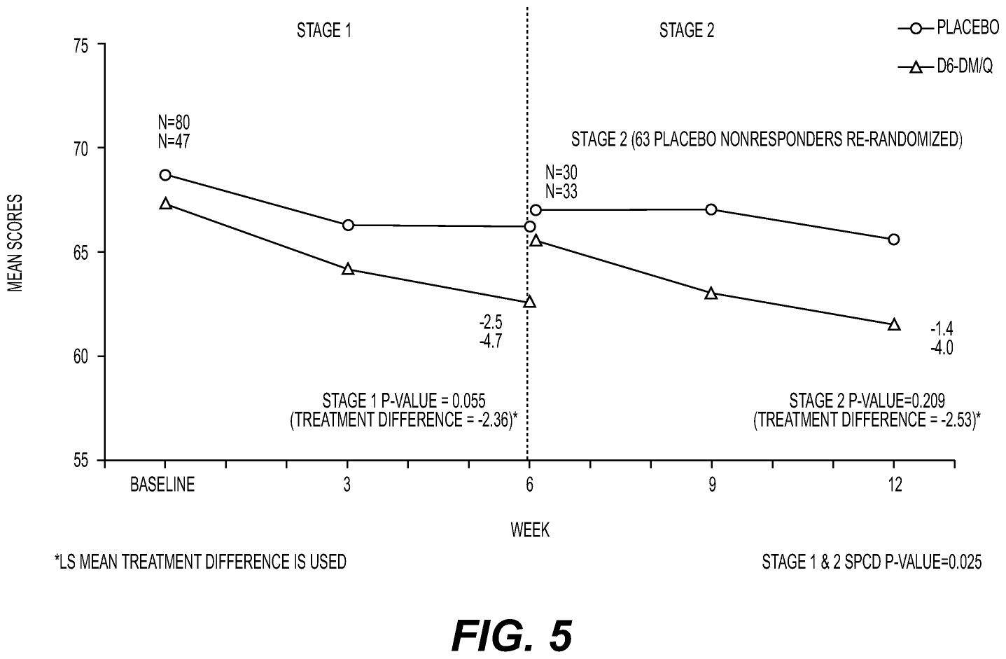

The PANSS total score ranges from 30 to 210 with higher scores indicating greater severity of symptoms. A statistically significant difference between d6-DM/Q and placebo, in favor of d6-DM/Q, was observed in the PANSS total score with the primary SPCD MMRM analysis (SPCD weighted Z-statistic=−2.25, p=0.025, Table 28). In Stage 1, the mean (SD) change from baseline in the PANSS total score was −4.7 (6.98) with d6-DM/Q and −2.5 (6.50) with placebo, resulting in a LS mean treatment difference of −2.36 (95% CI −4.77, 0.06; p=0.055). In Stage 2 which includes only placebo non-responders randomized to d6-DM/Q or placebo, the mean (SD) change from baseline in the PANSS total score was −4.0 (7.71) with d6-DM/Q and −1.4 (7.64) with placebo resulting in a LS mean treatment difference of −2.53 (95% CI −6.51, 1.45; p=0.209; FIG. 5). Similar results were observed with the OLS ANCOVA SPCD (p=0.024, Table 36) using the mITT population and with the SPCD MMRM analysis (p=0.022, Table 38) using the PP population.

Analysis of change from baseline in the PANSS total score are summarized in Table 76 for the mITT 12-week parallel group population and in Table 39 for the PP 12-week parallel group population.

3.4.1.2.1.2 PANSS Negative Subscale

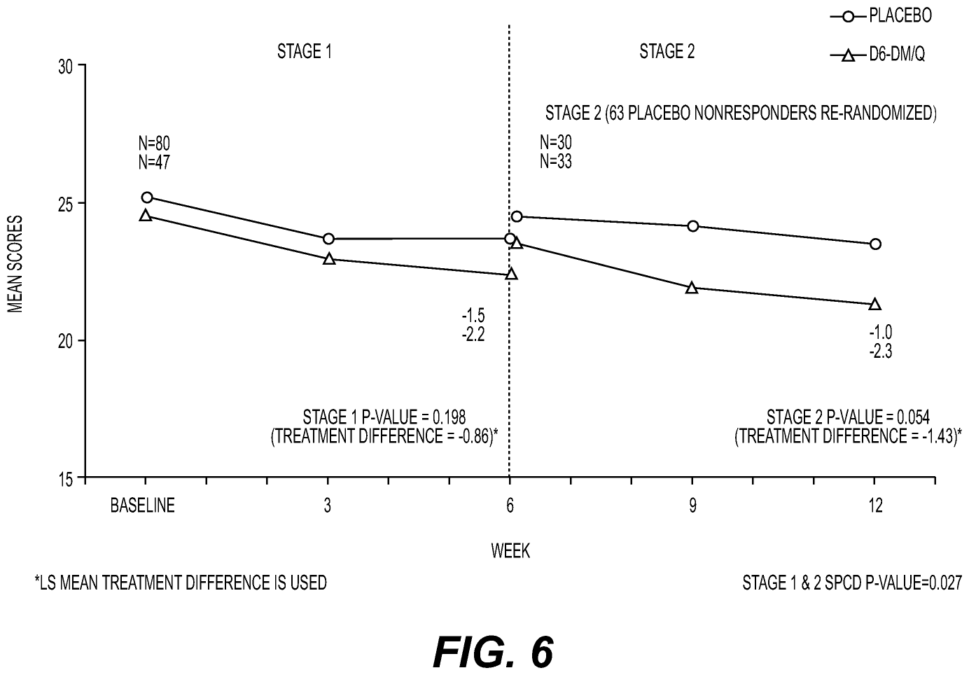

The negative subscale comprises 7 items of the PANSS and the score ranges from 7 to 49, with higher scores indicative of greater severity of the negative symptoms. A statistically significant difference between d6-DM/Q and placebo, in favor of d6-DM/Q, was observed in the PANSS negative subscale with the primary SPCD MMRM analysis (SPCD weighted Z-statistic=−2.20, p=0.027, Table 30) in the mITT population (FIG. 6) and in the PP population (p=0.019, Table 40). Similar results were observed with the ANCOVA SPCD analysis in the mITT population (p=0.019, Table 41).

Analysis of change from baseline in the PANSS negative subscale score are summarized in Table 42 for the mITT 12-week parallel group population and in Table 43 for the PP 12-week parallel group population.

3.4.1.2.1.3 PANSS Marder Negative Factors

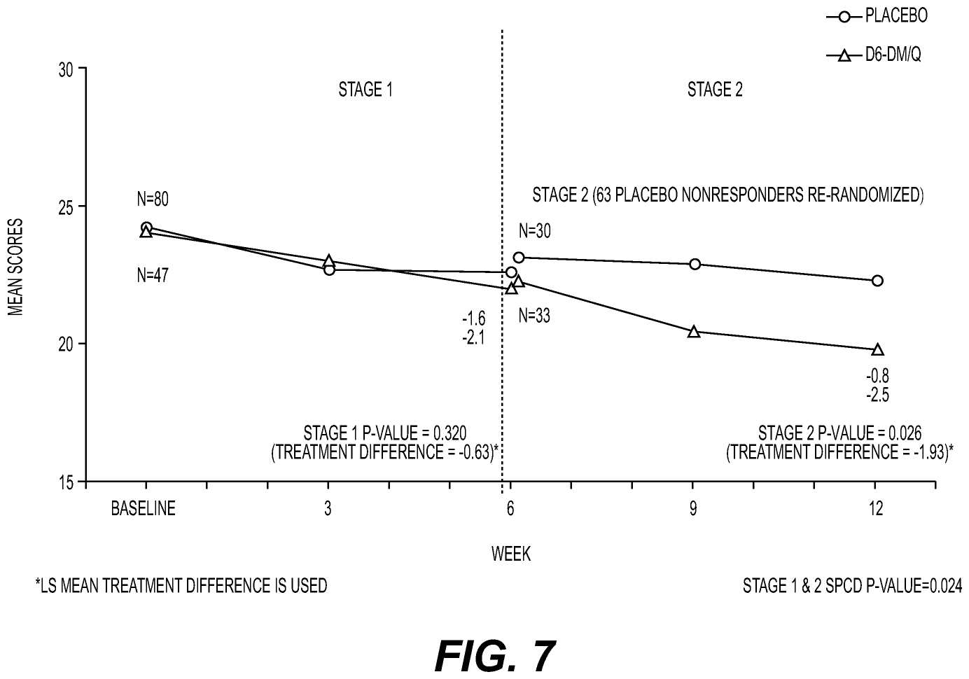

The PANSS Marder negative factors comprise 7 items of the PANSS and the score ranges from 7 to 49, with higher scores indicative of greater severity of the negative symptoms of schizophrenia. A statistically significant difference between d6-DM/Q and placebo, in favor of d6-DM/Q, was observed in the PANSS Marder negative factors with the primary SPCD MMRM analysis (SPCD weighted Z-statistic=−2.26, p=0.024, Table 32) in the mITT population (FIG. 7) and in the PP population (p=0.019, Table 44). Similar results were observed with the SPCD ANCOVA analysis in the mITT population (p=0.019, Table 45).

Analysis of change from baseline in the PANSS Marder negative subscale score are summarized in Table 46 for the mITT 12-week parallel group population and in Table 47 for the PP 12-week parallel group population.

3.4.1.2.1.4 PANSS Prosocial Factors

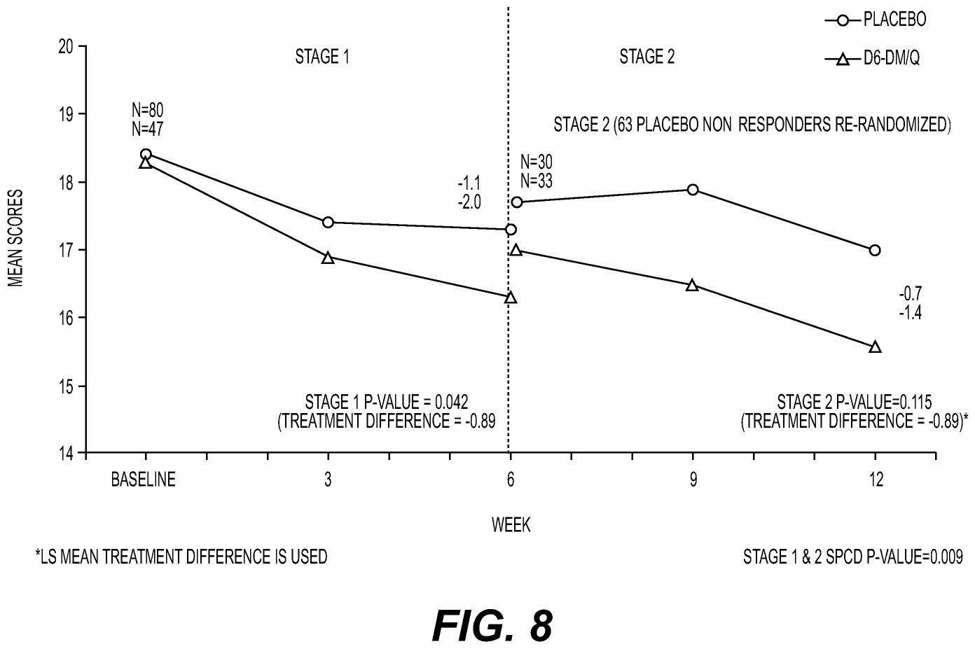

The PANSS prosocial factors comprises 6 items of the PANSS and the score ranges from 6 to 42, with higher scores indicative of greater severity of the specific negative symptoms. A statistically significant difference between d6-DM/Q and placebo, in favor of d6-DM/Q, was observed in the PANSS prosocial factors with the primary SPCD MMRM analysis (SPCD weighted Z-statistic=−2.60, p=0.009, Table 34) in the mITT population (FIG. 8). Similar results were observed with the SPCD ANCOVA analysis in the mITT population (p=0.007. Table 48).

Analysis of change from baseline in the PANSS prosocial factors score are summarized in Table 49 for the mITT 12-week parallel group population.

3.4.1.2.1.5 PANSS Positive Subscale

The positive subscale comprises 7 items of the PANSS and the score ranges from 7 to 49, with higher scores indicative of greater severity of the positive symptoms of schizophrenia. No significant difference in the PANSS positive subscale score was observed between the d6-DM/Q treated patients and the placebo treated patients by the SPCD MMRM analysis (Table 29), SPCD ANCOVA analysis (Table 50), or the 12-week analysis using the mITT 12-week parallel group population (Table 51).

3.4.1.2.1.6 PANSS General Psychopathology Subscale

The general psychopathology subscale comprises 16 items of the PANSS and the score ranges from 16 to 112, with higher scores indicative of greater severity of symptoms of schizophrenia. A numerically higher improvement was observed with d6-DM/Q compared to placebo in the change from baseline in the PANSS general psychopathology subscale score with the SPCD analysis using the mITT population (SPCD weighted Z-statistic=−1.93, p=0.054). In Stage 1, the mean (SD) change from baseline score was −1.7 (4.04) with d6-DM/Q and −0.7 (3.21) with placebo, resulting in a LS mean treatment difference of −1.14 (95% CI −2.40, 0.13; p=0.077). In Stage 2, the mean (SD) change from baseline was −1.3 (5.10) with d6-DM/Q and 0.0 (5.14) with placebo resulting in a LS mean treatment difference of −1.40 (95% CI −3.99, 1.19; p=0.284) (Table 31). Analysis using SPCD ANCOVA (Table 37) and using the mITT 12-week parallel group population (Table 35) showed a similar trend with statistically significant difference, in favor of d6-DM/Q seen in the 12-week analysis using the mITT 12-week parallel group population (p=0.028).

3.4.1.2.1.7 PANSS Excitement Component

The excitement component comprises 5 items of the PANSS and the score ranges from 5 to 35, with higher scores indicative of greater severity of symptoms. No significant difference in the PANSS excitement component was observed between the d6-DM/Q treated patients and the placebo treated patients by the SPCD MMRM analysis, SPCD ANCOVA analysis, or the 12-week analysis using the mITT 12-week parallel group population.

3.4.1.2.1.8 PANSS Responder Analysis

Treatment effect was evaluated by analyzing the proportion of patients with a 20% reduction from baseline in the PANSS total score with SPCD analysis using the mITT population and with GEE analysis using the mITT 12-week parallel group population. No statistically significant differences between d6-DM/Q and placebo were observed in these analyses.

Posthoc analysis were also performed using the same threshold (20% reduction from baseline) for the PANSS Marder Negative factors and the PANSS negative subscale (data not shown).

The proportion of patients with 20% reduction from baseline in PANSS Marder Negative factors was statistically significantly higher in the d6-DM/Q group compared to placebo in both stages (Stage 1: 21.3% vs 16.3%; Stage 2: 27.3% vs 3.3%; SPCD p=0.012).

The proportion of patients with 20% reduction from baseline in PANSS negative subscale was higher in the d6-DM/Q group compared to placebo in both stages (Stage 1: 23.4% vs 13.8%; Stage 2: 21.2% vs 10%; SPCD p=0.054).

3.4.1.2.2 NSA-16: Global Negative Symptoms, Global Level of Functioning,

5-Factor Domains, and NSA-4

3.4.1.2.2.1 Global Negative Symptoms Rating

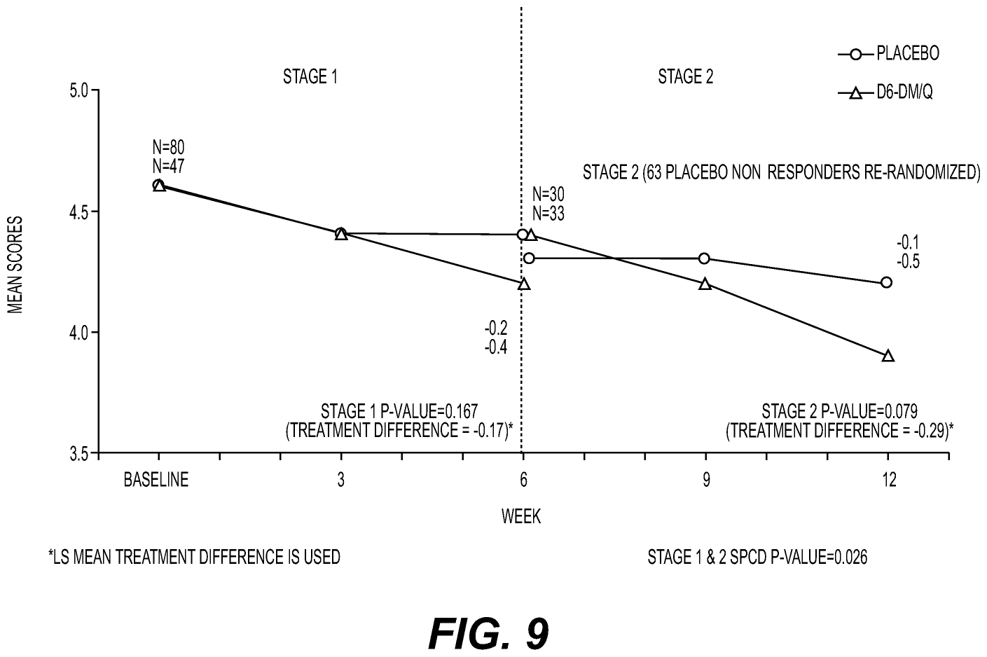

The global negative symptoms rating in the NSA-16 is a single score based on the overall impression of severity of negative symptoms on a 1 to 7 scale, where higher scores indicate greater severity. A statistically significant difference between d6-DM/Q and placebo, in favor of d6-DM/Q, was observed in the NSA-16 global negative symptoms rating with the primary SPCD MMRM analysis in the mITT population (SPCD weighted Z-statistic=−2.23, p=0.026, Table 52). In Stage 1, the mean (SD) change from baseline in the NSA-16 global negative symptoms rating was −0.4 (0.68) with d6-DM/Q and −0.2 (0.65) with placebo, resulting in a LS mean treatment difference of −0.17 (95% CI −0.40, 0.07; p=0.167). In Stage 2 which includes only placebo non-responders randomized to d6-DM/Q or placebo, the mean (SD) change from baseline in the NSA-16 global negative symptoms rating was −0.5 (0.76) with d6-DM/Q and −0.1 (0.52) with placebo resulting in a LS mean treatment difference of −0.29 (95% CI −0.61, 0.03; p=0.079; FIG. 9). Similar results were observed with the SPCD OLS ANCOVA (p=0.016, Table 53).

Analysis of change from baseline in the NSA-16 global negative symptoms rating using the mITT 12-week parallel group population are summarized in Table 54 (MMRM analysis) and Table 55 (ANCOVA).

3.4.1.2.2.2 Global Level of Functioning

The global level of functioning is a single score on a scale of 1 to 7 that provides the overall assessment of the patient's level of functioning, with higher scores indicative of severe impairment in functioning. No significant difference in the NSA-16 global level of functioning score was observed between the d6-DM/Q treated patients and the placebo treated patients by the SPCD MMRM analysis, SPCD ANCOVA analysis, or the 12-week analysis using the mITT 12-week parallel group population.

3.4.1.2.2.3 NSA-4 Total Score

The NSA-4 total score comprises items 2, 5, 8, and 13 of the NSA-16 which allow clinicians to rapidly determine the severity of negative symptoms of schizophrenia. These items focus on the following behaviors: restricted speech quantity (2), reduced range of emotion (5), reduced social drive (8) and reduced interest (13). No significant difference in the NSA-4 total score was observed between the d6-DM/Q treated patients and the placebo treated patients by the SPCD MMRM analysis, SPCD ANCOVA analysis with the mITT population, or the 12-week analysis using the mITT 12-week parallel group population.

3.4.1.2.2.4 NSA-16 Factor Domains

The items in the NSA-16 are grouped to describe negative symptoms using a 5-factor model, which includes the following domains: communication (items 1, 2, 3, and 4), emotion/affect (items 5, 6, and 7), social involvement (items 8, 9, and 10), motivation (items 11, 12, 13, and 14), and retardation (items 15 and 16). No significant differences between d6-DM/Q and placebo were observed in any of the 5-factor domains of the NSA-16 with the SPCD analysis using mITT population or the 12-week analysis using the mITT 12-week parallel group population.

Results by domain were determined as follows:
Communication: SPCD MMRM analysis, SPCD ANCOVA analysis with the mITT population; 12-week analysis using the mITT 12-week parallel group population
Emotion/affect: SPCD MMRM analysis, SPCD ANCOVA analysis with the mITT population; 12-week analysis using the mITT 12-week parallel group population
Social involvement: SPCD MMRM analysis, SPCD ANCOVA analysis with the mITT population; 12-week analysis using the mITT 12-week parallel group population
Motivation: SPCD MMRM analysis, SPCD ANCOVA analysis with the mITT population; 12-week analysis using the mITT 12-week parallel group population
Retardation: SPCD MMRM analysis, SPCD ANCOVA analysis with the mITT population; 12-week analysis using the mITT 12-week parallel group population

3.4.1.2.3 Patient Global Impression of Change (PGI-C)

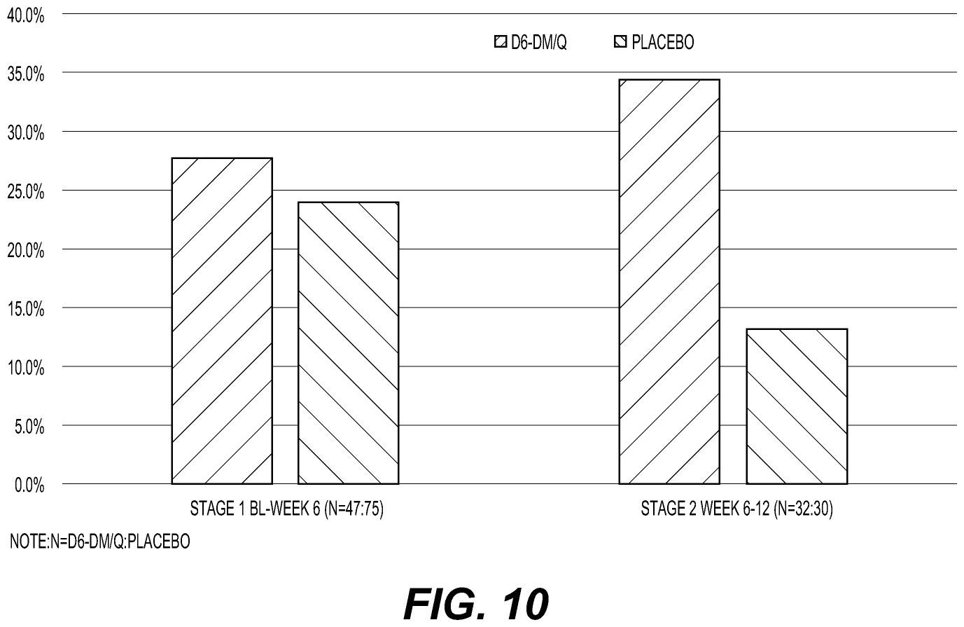

The PGI-C was used to evaluate patient's impression of treatment response relative to baseline at Week 6 (Stage 1) and relative to Visit 4 (end of Stage 1) at Week 12 (Stage 2), with 1=Very much improved to 7=Very much worse. A summary of the categorical responses by stage for the mITT population was determined. No statistically significant differences in PGI-C scores were observed between the d6-DM/Q and placebo group with SPCD ANCOVA analysis on observed data (SPCD p=0.170). In Stage 1, 27.7% of patients treated with d6-DM/Q vs. 24% on placebo rated their change in symptoms as “much improved” or “very much improved.” In Stage 2, 34.4% of patients treated with d6-DM/Q vs. 13.3% on placebo reported that their symptoms at Week 12 were “much improved” or “very much improved” (FIG. 10, SPCD p=0.170). Analysis of PGI-C using proportional odds regression by stage are summarized for the mITT population.

The 12-week analysis using the mITT 12-week parallel group population for the PGI-C scores is presented in Table 21 and for the proportional odds regression by stage in Table 22. The proportion of patients with ‘much improved’ or ‘very much improved’ rating at Week 12 relative to baseline was 33.3% (14/42) for the d6-DM/Q/d6-DM/Q group and 18.8% (6/32) for the placebo/placebo group (p=0.158 by proportional odds regression) in the mITT 12-week parallel group population.

3.4.1.2.4 Clinical Global Impression of Change (CGI-S)

The CGI-S score ranges from 1 to 7 with higher score indicative of greater severity of illness. The mean (SD) CGI-S scores at baseline were 3.7 (0.74) and 3.9 (0.74) for d6-DM/Q and placebo groups, respectively, in the mITT population. No significant difference in the CGI-S scores between d6-DM/Q and placebo was observed with the SPCD ANCOVA analysis or the 12-week analysis using the mITT 12-week parallel group population.

3.4.1.2.5 Clinical Global Impression of Change (CGI-C)

The CGI-C was used to evaluate the global impression of change in mental illness relative to baseline at Week 6 (Stage 1) and relative to Visit 4 (end of Stage 1) at Week 12 (Stage 2), with 1=Very much improved to 7=Very much worse. A summary of the categorical responses by stage was determined for the mITT population. No statistically significant differences were observed between the d6-DM/Q and placebo group with SPCD ANCOVA analysis on observed data or proportional odds regression by stage for the mITT population. In Stage 1, 48.9% (23/47) of patients in the d6-DM/Q group and 35.9% (28/78) of patients in the placebo group had ‘minimally improved’, ‘much improved’ or ‘very much improved’ rating on the CGI-C and in Stage 2, 43.7% (14/32) of patients in the d6-DM/Q group and 36.7% (11/30) of patients in the placebo had ‘much improved’ or ‘very much improved’ rating on the CGI-C(SPCD p=0.057).

Results of the analysis on the mITT 12-week parallel group population and PP 12-week parallel group population were similar, with no statistically significant differences between groups in CGI-C scores, however, statistically significant differences between groups were observed in the SPCD ANCOVA using the PP population (SPCD p=0.044.

3.4.1.2.6 Calgary Depression Scale for Schizophrenia (CDSS)

The CDSS score ranges from 0 to 27 with higher scores indicative of severe symptoms of depression. In this study, only patients with a score of <6 were to be included. Overall, the patients enrolled in the study had low scores on the CDSS at baseline; the mean (SD) score was 1.1 (1.34) for patients randomized to d6-DM/Q and 0.9 (1.31) for patients randomized to placebo in Stage 1. No significant difference in CDSS score was observed between the d6-DM/Q treated patients and the placebo treated patients by the SPCD MMRM analysis (Table 56), SPCD ANCOVA analysis (Table 57), or the 12-week analysis using the mITT 12-week parallel group population (Table 58). The mean (SD) change from baseline in CDSS score at Week 12 was −0.2 (1.27) for the d6-DM/Q/d6-DM/Q group and −0.4 (0.99) for the placebo/placebo group in the mITT 12-week parallel group population (Table 58).

3.4.1.2.7 MATRICS Consensus Cognitive Battery (MCCB)

The MCCB composite score ranges from 0 to 70 with higher scores indicative of less severe cognition symptoms. A numerically favorable improvement was observed with d6-DM/Q compared to placebo in the change from baseline in the MCCB composite score with the SPCD ANCOVA analysis using the mITT population (SPCD weighted OLS Z-statistic=1.78, p=0.074, Table 63). In Stage 1, the mean (SD) change from baseline in the MCCB composite score was 1.2 (5.11) with d6-DM/Q and 1.6 (4.55) with placebo, resulting in a LS mean treatment difference of −0.12 (95% CI −1.88, 1.64; p=0.893). In Stage 2, the mean (SD) change from baseline in the MCCB composite score was 1.6 (3.71) with d6-DM/Q and −1.6 (4.06) with placebo resulting in a LS mean treatment difference of 3.21 (95% CI 1.11, 5.30; p=0.003). Similar analyses using the PP population showed statistically significant difference in favor of d6-DM/Q (p=0.046, Table 64).

Results of the SPCD ANCOVA analysis of change from baseline in the MCCB composite score using the mITT 12-week parallel group population are presented in Table 65 and with the PP 12-week parallel group population in Table 66. No statistically significant differences between d6-DM/Q and placebo treatment were observed in these analyses.

3.4.1.2.8 Effort Expenditure for Reward Task (EEfRT)

EEfRT scores were analyzed for the following 8 variables:
Baseline Press Rate, subjects were first prompted to press the key for the hard task with their non-dominant pinky finger as fast as they can for 21 seconds. Value was coded as average presses per second.
Choice RT 1st 50, average reaction time (in milliseconds) for making a choice during the first 50 trials. Only trials in which subjects made a choice.
Completed, Proportion of completed tasks (Easy or Hard) during the first 50 trials.
12% Prob.-Prop. Hi Effort Opts—1st 50, Proportion of hard task choices made for the low (12%) probability condition during the first 50 trials of the task.
50% Prob.-Prop. Hi Effort Opts—1st 50, Proportion of hard task choices made for the medium (50%) probability condition during the first 50 trials of the task.
88% Prob.-Prop. Hi Effort Opts—1st 50, Proportion of hard task choices made for the high (88%) probability condition during the first 50 trials of the task.
All-Proportion High Effort Opts—1st 50, Overall proportion of hard task choices made for the first 50 trials of the task.
Diff-Proportion High Effort Opts—1st 50, Difference between proportion of hard task choices made for the high probability condition during the first 50 trials of the task.

No significant differences were observed between the d6-DM/Q treated patients and the placebo treated patients by the SPCD ANCOVA analysis, or the 12-week analysis using the mITT 12-week parallel group population.

3.4.1.2.9 Smoking Cessation

The change from baseline in number of cigarettes smoked per day for current smokers and for the mITT 12-week parallel group population were assessed, and there were no meaningful differences between the d6-DM/Q and placebo treated patients, in smoking cessation.

3.4.1.3 Exploratory Biomarkers

Whole blood samples were collected at any visit when blood was already being drawn for exploratory biomarker analyses. The results of the biomarker analysis are presented in a separate report when available.

3.4.1.4 Posthoc Analyses

3.4.1.4.1 Assessments Performed by a Single-rater Throughout the Study

Rater variance and drift from prescribed anchor points has long contributed to inconsistencies in reliable ratings despite training efforts. Approximately one third of patients in the study had more than one rater assessing the primary and secondary endpoints during the course of the study. A post-hoc analysis was performed excluding all patients who had more than one rater from both the placebo and d6-DM/Q treatment groups to assess impact of the use of a single-rater assessing the same endpoints throughout the study. These findings are summarized in Table 12.

TABLE 12
A Summary of NSA-16 and PANSS Score Results Among Patients with a Single-Rater Throughout
Study

| | Stage 1: Change from Baseline to Week 6 | | | Stage 2: Change from Week 6 to Week 12 | | | |
|---|---|---|---|---|---|---|---|
| | (d6-DM/Q N = 32; Placebo N = 55) | | | (d6-DM/Q N = 23; Placebo N = 20) | | | SPCD |
| | LS Mean Treatment | Standard | p- | LS Mean Treatment | Standard | p- | P- |
| Outcome Measure | Difference (95% CI) | Effect Size | value | Difference (95% Cl) | Effect Size | value | value |
| NSA-16 Total Score | −3.49 (−5.82 to −1.15) | −0.69 | 0.004 | −1.51 (−4.71 to 1.70) | −0.30 | 0.347 | 0.004 |
| NSA-16 Global | −0.30 (−0.59 to −0.02) | −0.45 | 0.039 | −0.30 (−0.70 to 0.10) | −0.48 | 0.138 | 0.010 |
| Negative Symptoms | | | | | | | |
| NSA-16 | −1.16 (−2.05 to −0.27) | −0.58 | 0.011 | −1.19 (−2.13 to −0.24) | −0.79 | 0.015 | <0.001 |
| Communication | | | | | | | |
| Domain | | | | | | | |
| PANSS Total | −3.57 (−6.58 to −0.56) | −0.52 | 0.021 | −3.82 (−9.13 to 1.49) | −0.50 | 0.153 | 0.008 |
| PANSS Negative | −1.36 (−3.05 to 0.34) | −0.36 | 0.115 | −1.90 (−3.54 to −0.26) | −0.75 | 0.024 | 0.009 |
| Subscale | | | | | | | |
| PANSS Marder | −1,44 (−3.07 to 0.19) | −0.40 | 0.083 | −2.15 (−4.19 to −0,12) | −0.63 | 0.038 | 0.007 |
| Factor | | | | | | | |
| PANSS General | −1.58 (−3.06 to −0.09) | −0.46 | 0.038 | −1.85 (−5.54 to 1.85) | −0.33 | 0.317 | 0.049 |
| Psychopathology | | | | | | | |
| PANSS Prosocial | −1.28 (−2.37 to −0.19) | −0.53 | 0.021 | −0.87 (−2.25 to 0.51) | −0.42 | 0.208 | 0.009 |
| Factors | | | | | | | |

MMRM = mixed-mode repeated measures;
NSA-16 = Negative Symptom Assessment scale;
PANSS = Positive and Negative Syndrome Scale;
SPCD = sequential parallel comparison design;
LS Mean = least square mean
Standard Effect Size was estimated by LS Mean Difference/Pooled Standard Deviation.
Note:
Negative value indicates improvement. Stage 1 and 2 treatment effects were analyzed by MMRM on observed data with fixed effect of treatment, visit, treatment-by-visit interaction, baseline value, and baseline-by-visit interaction. An unstructured covariance matrix was used.
SPCD weighted z-statistic was calculated using Stage 1 weight = 0.6 and Stage 2 weight = 0.4.

Despite the smaller patient cohorts, the positive treatment effects on negative symptoms were enhanced across many endpoints of the NSA-16 and the PANSS. Statistically significant treatment benefit was observed in the SPCD Combined Stage 1 and Stage 2 NSA-16 Total score (p=0.004), the NSA-16 Global Negative Symptoms score (p=0.010), and the NSA-16 Communication Domain (p<0.001).

Single-rater analysis of the SPCD Combined Stage 1 and Stage 2 Total score (p=0.008), Negative subscale (p=0.009), PANSS Marder Negative factors (p=0.007), General Psychopathology score (p=0.049), and Prosocial Factors score (p=0.009) also consistently demonstrated augmentation of positive treatment effects seen in the primary data analysis.

3.4.1.4.2 Assessments on Communication and Expression

Individuals with schizophrenia often have severe expressive deficits in both verbal and non-verbal communication. This impairment in communicative abilities causes severe functional deficits that results in impaired adaptive prosocial behaviors, social isolation and withdrawal. Post-hoc analysis was conducted for specific factors of the PANSS, as well as the NSA-16 and MCCB that measure communicative and expressive domains. The instruments used in the study capture both verbal and non-verbal communication (Axelrod et al. J Psychiatr Res. 1993; 27(3):253-8) used by the participant.

In patients treated with d6-DM/Q there is a strong trend toward improvement in the subdomains that focus on communication and interaction. It is believed that this overall pattern of change reflects an improvement in the patients' ability to interact and engage with others, impairment of which is one of the hallmarks of negative symptoms of schizophrenia and a key reason for long-term withdrawal from society of these patients. Support for improvement in these communicative domains was also evident in the Attention/Vigilance subdomain of the MCCB and in the NSA-16 Communication Factors Domain. These results are presented in Table 13.

TABLE 13
Communication and Expression Factor Analysis

| | Stage 1: Change from Baseline to Week 6 | | | Stage 2: Change from Week 6 to Week 12 | | | |
|---|---|---|---|---|---|---|---|
| | (d6-DM/Q N = 47; Placebo N = 80) | | | (d6-DM/Q N = 33; Placebo N = 30) | | | SPCD |
| | LS Mean Treatment | Standard | p- | LS Mean Treatment | Standard | p- | p- |
| Outcome Measure | Difference (95% CI) | Effect Size | value | Difference (95% CI) | Effect Size | value | value |
| NSA-16 Communication | −0.46 (−1.22, 0.29) | −0.22 | 0.228 | −0.64 (−1.52, 0.23) | −0.38 | 0.147 | 0.064 |
| Factor Domain | | | | | | | |
| PANSS | −0.89 (−1.75, −0.03) | −0.38 | 0.042* | −0.89 (−2.00, 0.22) | −0.41 | 0.115 | 0.009* |
| Prosocial factors | | | | | | | |
| PANSS | −0.42 (−1.36, 0.52) | −0.16 | 0.381 | −1.27 (−2.34, −0.19) | −0.58 | 0.022* | 0.034* |
| Expressive deficits domain | | | | | | | |
| PANSS N3 | −0.17 (−0.48, 0.15) | −0.18 | 0.299 | −0.20 (−0.63, 0.24) | −0.23 | 0.373 | 0.169 |
| Poor Rapport | | | | | | | |
| PANSS N4 | −0.15 (−0.46, 0.16) | −0.18 | 0.351 | −0.60 (−0.99, −0.21) | −0.73 | 0.003* | 0.007* |
| Passive! Apathetic Social | | | | | | | |
| Withdrawal | | | | | | | |
| PANSS N6 | 0.04 (−0.29, 0.37) | 0.04 | 0.813 | −0.35 (−0.71, 0.02) | −0.47 | 0.060 | 0.346 |
| Lack of Spontaneity | | | | | | | |
| PANSS G7 | 0.04 (−0.24, 0.32) | 0.05 | 0.777 | −0.40 (−0,80, −0.00) | −0.43 | 0.048* | 0.243 |
| Motor Retardation | | | | | | | |
| PANSS G11 | −0.10 (−0.30, 0.10) | −0.17 | 0.328 | −0.20 (−0.58, 0.19) | −0.25 | 0.313 | 0.159 |
| Poor Attention | | | | | | | |
| NACCB: Attention/Vigilance | 0.65 (−1.87, 3.18) | 0.09 | 0.611 | 3.35 (0.02, 6.68) | 0.53 | 0.049* | 0.088 |

CI = confidence interval;
MCCB = MATRICS Consensus Cognitive Battery;
MMRM = mixed-model repeated measures;
NSA-16 = Negative Symptom Assessment scale;
PANSS = Positive and Negative Syndrome Scale;
SPCD = sequential parallel comparison design;
LS Mean = least square mean
Standard Effect Size was estimated by LS Mean Difference/Pooled Standard Deviation.
Note:
Negative value indicates improvement (except MCCB). Stage 1 and 2 treatment effects were analyzed by MMRM on observed data with fixed effect of treatment, visit, treatment-by-visit interaction, baseline value, and baseline-by-visit interaction. An unstructured covariance matrix was used.
SPCD weighted z-statistic was calculated using Stage 1 weight = 0.6 and Stage 2 weight = 0.4.

3.4.2 Tabulation of Individual Response Data

By-patient listings of response data and other relevant study information were determined for the following:
Randomization scheme
Discontinued patients
Protocol deviations
Excluded patients from analysis
Demographic and baseline characteristics data
Concomitant medication
Compliance/drug concentration
By-patient efficacy response

3.4.3 Drug Dose, Drug Concentration, and Relationship to Response

3.4.3.1 Drug Dose

The dose for patients randomized or re-randomized to d6-DM/Q was based on a fixed-titration scheme of d6-DM/Q-24/4.9 QD for 1 week, d6-DM/Q-24/4.9 BID for 1 week, and d6-DM/Q-34/4.9 BID for 4 or 10 weeks. The duration of d6-DM/Q exposure in the safety population is presented in Table 14.

TABLE 14
Duration of Exposure-Safety Population

| | Placebo/ | d6-DM/Q/ | Placebo/d6-DM/Q | | |
|---|---|---|---|---|---|
| | Placebo | d6-DM/Q | (N = 40) | | All d6-DM/Q |
| | (N = 56) | (N = 48) | Placebo | d6-DM/Q | (N = 88) |
| Duration of exposure, days | | | | | |
| Mean (SD) | 61.41 (34.786) | 75.94 (24.059) | 43.18 (2.406) | 41.83 (4.113) | 60.43 (24.741) |
| Median (min, max) | 84.0 (1, 89) | 75.0 (1, 88) | 42.0 (40, 51) | 43.0 (20, 47) | 45.0 (1, 88) |
| Number of patient-days | 3439 | 3645 | 1727 | 1673 | 5318 |
| Number of patient-years | 9.42 | 9.98 | 4.73 | 4.58 | 14.56 |

Number of patient-years equals the sum of all patient exposure duration in days divided by 365.25.
SD = standard deviation.

3.4.3.2 Drug Concentrations and PK Parameter Estimates: d6-DM/Q

Plasma concentrations were measured for d6-DM, d3-DX, and Q at baseline (Day 1), Visit 4 (Week 6) and Visit 7 (Week 12) and summarized by metabolizer subgroup and all metabolizer types. Mean concentrations of d6-DM for all patients were 49.7 ng/mL (d6-DM/Q/d6-DM/Q group at Visit 4 [Week 6]), 53.2 ng/mL (d6-DM/Q/d6-DM/Q group at Visit 7 [Week 12]), and 54.0 ng/mL (placebo/d6-DM/Q group at Visit 7 [Week 12]). Mean concentrations of d3-DX for all patients were 101.2 ng/mL (d6-DM/Q/d6-DM/Q group at Visit 4 [Week 6]), 111.6 ng/mL (d6-DM/Q/d6-DM/Q group at Visit 7 [Week 12]), and 124.5 ng/mL (placebo/d6-DM/Q group at Visit 7 [Week 12]). Mean concentrations of d3-3-MM for all patients were 20.5 ng/mL (d6-DM/Q/d6-DM/Q group at Visit 4 [Week 6]), 19.4 ng/mL (d6-DM/Q/d6-DM/Q group at Visit 7 [Week 12]), and 23.4 ng/mL (placebo/d6-DM/Q group at Visit 7 [Week 12]). Mean values for d6-DM, d3-DX and d3-3-MM varied by metabolizer type. Mean concentrations of Q for all patients were 17.9 ng/mL (d6-DM/Q/d6-DM/Q group at Visit 4 [Week 6]), 20.1 ng/mL (d6-DM/Q/d6-DM/Q group at Visit 7 [Week 12]), and 21.2 ng/mL (placebo/d6-DM/Q group at Visit 7 [Week 12]).

Cₘₐₓ and AUC for d6-DM, d3-DX, and Q were estimated at Visit 4 (Week 6) and Visit 7 (Week 12) for all patients randomized to receive d6-DM/Q and are summarized for all metabolizer types and by metabolizer subgroups in Table 15.

3.4.3.3 d6-DM/Q PK Parameter Relationship to Response

The association between the NSA-16 total score change from baseline and the Cₘₐₓ and AUC for d6-DM, d3-DX, or Q, respectively, was summarized at Visit 4 (Week 6; Cₘₐₓ, Table 77, Table 80, and Table 83; AUC, Table 86, Table 89, and Table 92) and Visit 7 (Week 12; Cₘₐₓ, Table 78, Table 81, and Table 84; AUC, Table 87, Table 90, and Table 93) for the mITT population. The Pearson correlation coefficient values generally indicated poor correlation between the NSA-16 total score change from baseline and the Cₘₐₓ and AUC values for all 3 analytes. These analyses were repeated for the 12-week mITT population subset with similar results (Cₘₐₓ, Table 79, Table 82, and Table 85; AUC, Table 88, Table 91, and Table 94).

TABLE 15
Summary of PK Parameters Estimated from Measured Drug Concentrations
of Study Drug and Metabolites-Safety Population

| | Metabolizer Subgroup | | | | |
|---|---|---|---|---|---|
| | Poor | Intermediate | Extensive | Ultra-Rapid | All |
| Visit 4 (Week 6) (d6- | | | | | |
| DM/Q/d6-DM/Q) N = 48 | | | | | |
| d6-DM | | | | | |
| Cₘₐₓ (ng/mL), n | 1 | 22 | 19 | 1 | 43 |
| Mean (SD) | 28.7 (na) | 63.9 (44.15) | 38.2 (27.99) | 58.6 (na) | 51.6 (38.53) |
| Median (min, max) | 28.7 (29, 29) | 48.1 (17, 192) | 32.2 (8, 130) | 58.6 (59, 59) | 40.0 (8, 192) |
| AUC (h × ng/mL), n | 1 | 22 | 19 | 1 | 43 |
| Mean (SD) | 204.6 (na) | 573.2 (418.66) | 333.3 (255.24) | 468.0 (na) | 456.2 (362.05) |
| Median (min, max) | 204.6 (205, 205) | 401.7 (139, 1793) | 271.9 (73, 1183) | 468.0 (468, 468) | 355.4 (73, 1793) |
| d3-DX | | | | | |
| Cₘₐₓ (ng/mL), n | 1 | 22 | 19 | 1 | 43 |
| Mean (SD) | 87.2 (na) | 111.5 (38.83) | 144.6 (56.42) | 193.1 (na) | 127.4 (50.25) |
| Median (min, max) | 87.2 (87, 87) | 101.2 (56, 195) | 134.9 (72, 332) | 193.1 (193, 193) | 121.6 (56, 332) |
| AUC (h × ng/mL), n | 1 | 22 | 19 | 1 | 43 |
| Mean (SD) | 887.2 (na) | 1044.8 (340.96) | 1235.3 (389.01) | 1774.7 (na) | 1142.3 (378.08) |
| Median (min, max) | 887.2 (887, 887) | 991.6 (506, 1847) | 1119.6 (729, 2091) | 1774.7 (1775, 1775) | 1083.8 (506, 2091) |
| Q | | | | | |
| Cₘₐₓ (ng/mL), n | 1 | 22 | 19 | 1 | 43 |
| Mean (SD) | 9.3 (na) | 20.8 (9.22) | 19.0 (6.27) | 33.7 (na) | 20.0 (8.21) |
| Median (min, max) | 9.3 (9, 9) | 18.2 (10, 43) | 17.6 (10, 32) | 33.7 (34, 34) | 17.7 (9, 43) |
| AUC (h × ng/mL), n | 1 | 22 | 19 | 1 | 43 |
| Mean (SD) | 74.6 (na) | 164.4 (72.16) | 152.0 (48.60) | 261.2 (na) | 159.1 (63.81) |
| Median (min, max) | 74.6 (75, 75) | 139.9 (80, 325) | 143.2 (83, 258) | 261.2 (261, 261) | 142.1 (75, 325) |
| Visit 7 (Week 12) (d6- | | | | | |
| DM/Q/d6-DM/Q) N = 48 | | | | | |
| d6-DM | | | | | |
| Cₘₐₓ (ng/mL), n | 0 | 20 | 19 | 1 | 40 |
| Mean (SD) | na | 67.0 (46.98) | 38.7 (26.74) | 62.1 (na) | 53.4 (40.09) |
| Median (min, max) | na | 45.9 (16, 194) | 34.1 (8, 124) | 62.1 (62, 62) | 42.3 (8, 194) |
| AUC (h × ng/mL), n | 0 | 20 | 19 | 1 | 40 |
| Mean (SD) | na | 601.9 (442.90) | 336.7 (244.01) | 498.8 (na) | 473.4 (375.01) |
| Median (min, max) | na | 375.7 (132, 1810) | 288.9 (70, 1127) | 498.8 (499, 499) | 369.4 (70, 1810) |
| d3-DX | | | | | |
| Cₘₐₓ (ng/mL), n | 0 | 20 | 19 | 1 | 40 |
| Mean (SD) | na | 106.3 (33.76) | 144.9 (56.98) | 190.3 (na) | 126.8 (50.32) |
| Median (min, max) | na | 100.6 (57, 187) | 133.9 (76, 337) | 190.3 (190, 190) | 123.0 (57, 337) |
| AUC (h × ng/mL), n | 0 | 20 | 19 | 1 | 40 |
| Mean (SD) | na | 1015.0 (312.22) | 1243.6 (394.70) | 1783.3 (na) | 1142.8 (378.46) |
| Median (min, max) | na | 1000.2 (511, 1833) | 1123.4 (740, 2115) | 1783.3 (1783, 1783) | 1087.1 (511, 2115) |
| Q | | | | | |
| Cₘₐₓ (ng/mL), n | 0 | 20 | 19 | 1 | 40 |
| Mean (SD) | na | 20.9 (10.25) | 19.4 (6.39) | 35.3 (na) | 20.6 (8.73) |
| Median (min, max) | na | 17.7 (10, 45) | 19.4 (10, 33) | 35.3 (35, 35) | 18.8 (10, 45) |
| AUC (h × ng/mL), n | 0 | 20 | 19 | 1 | 40 |
| Mean (SD) | na | 166.3 (80.69) | 155.2 (50.01) | 274.8 (na) | 163.7 (68.42) |
| Median (min, max) | na | 141.5 (81, 347) | 149.0 (82, 262) | 274.8 (275, 275) | 146.5 (81, 347) |
| Visit 7 (Week 12) | | | | | |
| (placebold6-DM/Q) N = 40 | | | | | |
| d6-DM | | | | | |
| Cₘₐₓ (ng/mL), n | 1 | 13 | 19 | 0 | 33 |
| Mean (SD) | 47.9 (na) | 56.2 (29.94) | 47.2 (26.57) | Na | 50.8 (27.44) |
| Median (min, max) | 47.9 (48, 48) | 56.1 (15, 106) | 44.2 (7, 121) | Na | 47.7 (7, 121) |
| AUC (h × ng/mL), n | 1 | 13 | 19 | 0 | 33 |
| Mean (SD) | 427.4 (na) | 492.1 (271.83) | 404.3 (239.02) | Na | 439.6 (248.41) |
| Median (min, max) | 427.4 (427, 427) | 492.9 (124, 945) | 368.0 (53, 1081) | Na | 413.0 (53, 1081) |
| d3-DX | | | | | |
| Cₘₐₓ (ng/mL), n | 1 | 13 | 19 | 0 | 33 |
| Mean (SD) | 65.1 (na) | 108.0 (24.89) | 142.5 (56.08) | Na | 126.6 (49.10) |
| Median (min, max) | 65.1 (65, 65) | 105.2 (76, 168) | 130.9 (80, 318) | Na | 116.4 (65, 318) |
| AUC (h × ng/mL), n | 1 | 13 | 19 | 0 | 33 |
| Mean (SD) | 651.2 (na) | 1014.1 (202.39) | 1323.8 (623.07) | Na | 1181.4 (515.68) |
| Median (min, max) | 651.2 (651, 651) | 940.4 (745, 1367) | 1131.7 (750, 3622) | Na | 1085.8 (651, 3622) |
| Q | | | | | |
| C_{max,} n (ng/mL) | 1 | 13 | 19 | 0 | 33 |
| Mean (SD) | 23.1 (na) | 19.7 (4.28) | 21.7 (9.93) | Na | 21.0 (7.96) |
| Median (min, max) | 23.1 (23, 23) | 19.5 (11, 27) | 20.3 (10, 45) | Na | 20.3 (10, 45) |
| AUC, n (h × ng/mL) | 1 | 13 | 19 | 0 | 33 |
| Mean (SD) | 185.1 (na) | 153.8 (31.55) | 168.9 (74.91) | Na | 163.5 (60.00) |
| Median (min, max) | 185.1 (185, 185) | 155.5 (90, 208) | 157.9 (84, 336) | Na | 157.9 (84, 336) |

Patients who were not assigned a metabolizer group are excluded from this table. Visit 7 (Week 12) includes early termination visits.
AUC = area under the curve;
Cmax = maximum concentration;
d6-DM = deudextromethorphan hydrobromide;
d3-DX = deuterated (d3) dextrorphan;
Q = quinidine sulfate;
SD = standard deviation.

3.4.4 Drug Concentration: Second-Generation Antipsychotics (SGA)

Per the study entry criteria, patients were on at least 1 SGA at baseline. Plasma concentrations were analyzed for the following SGAs, aripiprazole, lurasidone, olanzapine, paliperidone, quetiapine, risperidone, and ziprasidone. Results are summarized by metabolizer subgroups for patients who were randomized to receive d6-DM/Q in Stage 1 and 2 (d6-DM/Q/d6-DM/Q), placebo in Stage 1 and 2 (d6-DM/Q/d6-DM/Q) or d6-DM/Q in Stage 2 only (placebo/d6-DM/Q) and. The number of patients taking each SGA at baseline were as follows: aripiprazole 32 patients, lurasidone 6 patients, olanzapine 36 patients, paliperidone 18 patients, quetiapine 23 patients, risperidone 31 patients, and ziprasidone 3 patients.

3.4.5 Drug-Drug and Drug-Disease Interactions

Analyses of the primary endpoint (NSA-16 total score) were performed for the subset of patients in the mITT population with baseline concomitant use of benzodiazepines (Table 69), SNRIs (Table 70), or SRRIs (Table 71) to determine whether the concomitant drugs being taken at baseline affected patient NSA-16 total scores. Similar analyses were performed for subgroup with concomitant psychotropic medications (antidepressants, antipsychotics) considered as CYP2D6 major substrates (Tables 72 and 74) and subgroup with concomitant psychotropic medications not considered as CYP2D6 major substrates (Tables 73 and 75). The number of patients in each subgroup were too few to make a meaningful interpretation of the analysis.

Analyses of the primary endpoint was also performed on the subgroup of patients with a composite score of <30 (Table 59) versus patients with ≥30 (Table 60) on the MCCB at baseline to assess the effect of cognition level on the patient NSA-16 total scores. Although there were no statistically significant differences between placebo and d6-DM/Q in either subgroup, numerically higher improvement in NSA-16 total scores were observed in the subgroup with MCCB score of <30 at baseline. The standard effect size was −0.477 (Stage 1) and −0.527 (Stage 2) for the subgroup with MCCB score of ≥30 at baseline and was −0.226 (Stage 1) and 0.059 (Stage 2) for the subgroup with MCCB score of ≤30 at baseline.

3.4.6 By-Patient Displays

By-patient display data were not reported in this study because group mean data represent the principal analyses.

3.4.7 Efficacy
A numerically higher improvement was observed with d6-DM/Q compared to placebo in the primary efficacy endpoint, change from baseline in the NSA-16 total score, with the SPCD analysis (SPCD weighted Z-statistic=−1.79, p=0.073). The LS mean treatment difference between d6-DM/Q and placebo was −1.79 (95% CI −3.86, 0.29) in Stage 1 and −1.28 (95% CI −4.39, 1.83) in Stage 2. Significant treatment effects were seen in the sensitivity analysis of the primary endpoint with both the SUR method (p=0.048) and SPCD OLS ANCOVA LOCF Data (p=0.042) method of analyses.
Statistically significant differences in favor of d6-DM/Q versus placebo were observed in the SPCD analysis of PANSS total score (p=0.025). PANSS negative subscale (p=0.027), PANSS Marder negative factors (p=0.024), and PANSS prosocial factors (p=0.009). No statistically significant differences were observed between the 2 groups in the positive subscale (p=0.700), excitement component, (p=0.723), and general psychopathology subscale (p=0.054) of the PANSS.
Statistically significant differences (p=0.026) in favor of d6-DM/Q versus placebo were also observed in the SPCD analysis of the NSA-16 global negative symptoms rating. The LS mean treatment difference between d6-DM/Q and placebo was −0.17 (95% CI −0.40, 0.07) in Stage 1 and −0.29 (95% CI −0.61, 0.03) in Stage 2.
In Stage 1, 27.7% of patients treated with d6-DM/Q vs. 24% on placebo rated their change in symptoms as “much improved” or “very much improved” on the PGI-C scale. In Stage 2, 34.4% of patients treated with d6-DM/Q vs. 13.3% on placebo reported that their symptoms at Week 12 were “much improved” or “very much improved” (SPCD p=0.170). In the 12-week parallel group mITT population, the proportion of patients with ‘much improved’ or ‘very much improved’ rating at Week 12 relative to baseline was 33.3% (14/42) for the d6-DM/Q/d6-DM/Q group and 18.8% (6/32) for the placebo/placebo group (p=0.158 by proportional odds regression).
No statistically significant differences were observed between the d6-DM/Q and placebo group with SPCD analysis of other secondary endpoints including CGI-S, CGI-C, CDSS total score, MCCB composite score, and NSA-4 total score, however numerically higher improvements in favor of d6-DM/Q were observed for the CGI-C score and the MCCB composite score.
Mean concentrations of d6-DM for all patients were 49.7 ng/mL (d6-DM/Q/d6-DM/Q group at Visit 4 [Week 6]), 53.2 ng/mL (d6-DM/Q/d6-DM/Q group at Visit 7 [Week 12]), and 54.0 ng/mL (placebo/d6-DM/Q group at Visit 7 [Week 12]). Concentrations of d6-DM, d3-DX and d3-3MM varied with metabolizer type. PK parameters were estimated for d6-DM, d3-DX and Q. In a PK/Pharmacodynamic (PD) analysis using these estimated PK parameters, Pearson correlation coefficient values generally indicated poor correlation with the NSA-16 total score change from baseline.

4 Safety Evaluation

4.1 Vital Signs, Physical Findings, and Other Observations Related to Safety

4.1.1 Vital Signs

Vital sign actual values, the change from baseline, and percent change from baseline were determined. No trends suggesting an impact of d6-DM/Q on measured vital sign values were observed.

A summary of potentially clinically significant (PCS) vital sign abnormalities was determined. There were no meaningful differences in the incidence of PCS vital sign abnormalities between treatment groups.

4.1.2 Physical and Neurological Exam

A full physical and neurological examination was scheduled for all patients at the screening visit only. Clinically significant abnormalities in physical examination were reported for 3 patients and recorded in the medical history of the patient.

4.1.3 Electrocardiogram

Summaries of ECG parameters and the change from baseline and percent change from baseline in these parameters were determined by treatment group. Additionally, changes in ECG parameters from pre-dose to post-dose at Day 1 and Week 6 were determined. Overall, the changes from baseline in ECG parameters were small with no trends suggesting an impact of d6-DM/Q on ECG results.

None of the patients had a QTcF change from baseline of ≥60 msec, and no patient had a PR interval of >200 msec in either the all placebo group or all d6-DM/Q group. Overall. 2 females (1 in the all d6-DM/Q and 1 in the all placebo group) met the PCS criteria for ECG abnormality in QTcF interval with postbaseline values in the >460 to ≤485 msec range and 3 males (1 in the all d6-DM/Q and 2 in the all placebo group) met the PCS criteria for ECG abnormality in QTcF interval, with postbaseline values in the >450 to ≤480 msec range.

Two patients reported AEs in the cardiac disorders SOC; 1 patient (120-003) in the all d6-DM/Q group reported tachycardia and 1 patient (119-004) in the all placebo group reported atrial fibrillation.

4.1.4 Other Safety Observations

4.1.4.1 Columbia Suicide Severity Rating Scale (C-SSRS)

Summaries of C-SSRS ideation severity by visit and C-SSRS suicidal behavior type by visit were determined. The intensity of most severe ideation by was determined. Additionally, actual and potential lethality on most lethal attempts by visit was determined. Overall, no clinically meaningful signals related to suicidal ideation, severity, and lethality were observed with either placebo or d6-DM/Q treatment.

4.1.4.2 Simpson Angus Scale for Extrapyramidal Symptoms (SAS)

A summary of the total scores and individual item scores on the SAS is provided by gender and for males and females combined in Table 61. In addition, shift tables of individual items on the SAS are presented for the safety population, and by the female and male subgroups, in Table 62.

Overall, there were no signals or trends indicating that treatment with d6-DM/Q played a role in worsening of extrapyramidal symptoms in patients with such symptoms at baseline.

4.1.4.3 Barnes Akathisia Scale (BAS)

A summary of the total scores and individual item scores on the BAS was determined by gender and for males and females combined. In addition, shift tables of the global clinical assessment and individual items on the BAS for the safety population were determined and by the female and male subgroups.

Overall, there were no signals or trends indicating that treatment with d6-DM/Q played a role in worsening of restlessness symptoms in patients with such symptoms at baseline.

4.1.4.4 Abnormal Involuntary Movements Scale (AIMS)

A summary of the total scores and subscale scores on the AIMS was determined by gender and for males and females combined. In addition, shift tables of individual items on the AIMS were determined for the safety population and by the female and male subgroups.

Overall, there were no signals or trends indicating that treatment with d6-DM/Q played a role in worsening of dyskinesia symptoms in patients with such symptoms at baseline. There were no AEs of dyskinesia reported in patients treated with d6-DM/Q.

One patient in the all placebo group reported dyskinesia as an AE in Stage 2. The AE was deemed to be mild and possibly related to the study drug, by the investigator.

4.2 Safety
No deaths were reported during this study.
Overall, 30 (34.1%) patients in the all d6-DM/Q treatment group and 39 (40.6%) patients in the all placebo treatment group reported ≥1 TEAE. The TEAEs were mild to moderate in severity.
The most frequently (>2 patient) reported TEAEs in either the all d6-DM/Q group or the all placebo group, respectively, were dry mouth (4.5% vs. 1.0%), diarrhea (2.3% vs. 3.1%), dizziness (3.4% vs. 3.1%), headache (2.3% vs. 5.2%), somnolence (1.1% vs. 3.1%) and nasopharyngitis (3.4% vs. 4.2%).
Treatment-related TEAEs were reported by 13.6% and 18.8% for the all d6-DM/Q and the all placebo treatment group, respectively. Treatment-related TEAEs reported in ≥2 patients in the all d6-DM/Q included dry mouth (2.3%) and headache (2.3%), and in the all placebo group, these included headache (3.1%), dizziness (2.1%), somnolence (3.1%), diarrhea (2.1%), and sedation (2.1%).
SAEs were reported by 2 patients in the all placebo group and none in the all d6-DM/Q group. The 2 SAEs were exacerbation of schizophrenia and urinary tract infection. Study drug was discontinued in the patient who experienced the SAE of exacerbation of schizophrenia upon their hospitalization and was not resumed. Study drug was interrupted for one day in the patient who experienced the SAE of urinary tract infection. This patient completed the study. Neither of the SAEs were considered related to the study drug.
Overall, 10 patients discontinued the study due to TEAEs; 2 patients in the all d6-DM/Q group and 8 patients in the all placebo group. TEAEs that led to study discontinuation in the all d6-DM/Q group were vision blurred and rash, and, in the all placebo group, were eye pain, rash, atrial fibrillation, dizziness, headache, schizophrenia, and erectile dysfunction.
None of the patients had a QTcF change from baseline of ≥60 msec, and no patient had a PR interval of >200 msec in either the all placebo group or all d6-DM/Q group.
Patients had low scores on the SAS (extrapyramidal symptoms), BAS (restlessness) and AIMS (dyskinesia) at baseline and there were no AEs events or trends indicating that treatment with d6-DM/Q played a role in worsening of such symptoms during the course of the study.

5 Results

In this study conducted in patients with negative symptoms of schizophrenia, consistent efficacy signals for d6-DM/Q were observed across multiple, well-validated, and reliable instruments that have historically been accepted for evaluating negative symptoms of schizophrenia. Although the primary endpoint, change in the NSA-16 Total Score did not reach statistical significance, there was a trend for improvement (p=0.073). Patients treated with d6-DM/Q experienced a significantly greater improvement from baseline compared to placebo in the combined (Stage 1 and Stage 2) SPCD mITT analyses for several efficacy rating scales specifically assessing negative symptoms. These included the NSA-16 Global Negative Symptoms score (p=0.026), the PANSS Marder Negative Factors (p=0.024), and PANSS Negative Factors score (p=0.027). There were significant improvements in favor of d6-DM/Q over placebo treatment in the PANSS Expressive Deficit Domain (p=0.034) and the PANSS Experiential Deficit Domain (p=0.022), the PANSS Prosocial Factors Score (p=0.009), and a trend towards positive effect on the PANSS General Psychopathology Subscale Score (p=0.054). Improvement in the PANSS total was driven by improvement in negative symptoms and in the General Psychopathology Subscale. Patients had low baseline PANSS positive scores and demonstrated little change during the study, hence the findings were not pseudo-specific.

The standardized effect size (absolute value) by stage ranged from 0.30 to 0.75 for NSA-16 Total score, PANSS Total score, PANSS Marder, and PANSS Negative (data on file), suggesting that even when one of the results was not statistically significant, meaningful treatment effects were observed when compared to other approved neuroscience compounds.

To determine the impact of rater variability on efficacy end points, a post-hoc analysis of patients who were consistently rated by the same rater throughout the study was performed for the NSA-16 and the PANSS. Results of this post-hoc analysis were highly significant and enhanced on several outcome measures including the NSA-16 Total Score (p=0.004), the NSA-16 Global Negative Symptoms score (p=0.010), and the PANSS Marder negative factors (p=0.007) and negative Factor Scores (p=0.009). This analysis included a total of 87 patients in the mITT population.

In a consensus statement, Schooler and colleagues (Schooler et al. Schizophr Res. 2015; 162(1-3):169-174) suggest that a meaningful therapeutic benefit on negative symptoms of schizophrenia would be “a significant improvement, greater than or equal to 20% of the total score, on a valid and reliable measure assessing negative symptoms”. In this study (Example 1), analyses of the PANSS Negative subscale and the PANSS Marder negative factors scores were performed using this threshold of a 20% or more improvement.

A significantly greater percentage of d6-DM/Q treated patients compared to placebo demonstrated a 20% or greater improvement from baseline on the PANSS Marder Negative Factors score (Stage 1: 21.3% vs 16.3%; Stage 2: 27.3% vs 3.3%; p=0.012). The proportion of patients with 20% reduction from baseline in PANSS Negative subscale was also higher in the d6-DM/Q group compared to placebo in both stages (Stage 1: 23.4% vs 13.8%; Stage 2: 21.2% vs 10%; SPCD p=0.054).

Using a patient-reported outcome measure, the PGI-C, the proportion of patients with ‘much improved’ or ‘very much improved’ rating on the PGI-C at the end of Stage 1 was 27.7% for patients treated with d6-DM/Q and 24% for patients treated with placebo and in Stage 2 (Week 12 relative to baseline). In Stage 2, there was more than a 2-fold higher percentage of patients given d6-DM/Q than placebo (34.4% vs 13.3%) that rated their symptoms as “very much” or “much” improved from baseline to end of study. In the 12-week parallel group mITT population, the proportion of patients with ‘much improved’ or ‘very much improved’ rating at Week 12 relative to baseline was 33.3% (14/42) for the d6-DM/Q/d6-DM/Q group and 18.8% (6/32) for the placebo/placebo group (p=0.158 by proportional odds regression). In this population of stable patients who were in a residual state of their illness and on a stable dose of an atypical antipsychotics (˜3 months), the PGI-C could be used in a reliable manner in support of clinically meaningful information.

The clinician's view of the patient's global severity of negative symptoms was assessed through the NSA-16 Global negative symptoms severity score, while the overall level of illness was assessed with the CGI-S and CGI-C. A statistically significant benefit was observed on the NSA-16 Global negative symptoms severity score (SPCD; p=0.026). Although no statistically significant differences between d6-DM/Q and placebo were observed with the CGI-C and CGI-S measures, the results indicated a trend for benefit of d6-DM/Q over placebo. The different anchor points and type of illness assessed in the NSA-16 Global Negative symptoms score vs. the CGI-C/S may explain the stronger, statistically significant results seen with the NSA-16 Global Negative symptoms score. The NSA-16 Global is very specific for negative symptoms in comparison to a normal healthy young adult, whereas the CGI-C/S use the patient themselves or other patients with schizophrenia as the anchor point and are specific to overall illness and not to negative symptoms. Modified versions of the CGI-C/S which assess the severity and change in target symptomatology may provide more meaningful information and could be considered in future studies.

There was also a trend towards improvement in cognition as assessed by the MCCB Composite Score and the Attention/Vigilance domain of the MCCB with significant benefits that were seen in Stage 2 of the study. Ability to demonstrate improvements in cognition has been another difficult hurdle for the field and here it appears there may be a potential towards a cognitive benefit that merits exploration in additional studies.

In this study (Example 1), there was a signal of efficacy demonstrated on aspects of verbal and non-verbal communication in specific factors of the PANSS in patients treated with d6-DM/Q vs placebo. These were also evident in the Attention/Vigilance subdomain of the MCCB and in the NSA-16 Communication Factors Domain.

Patients had low baseline symptoms of depression (evaluated with the Calgary Depression Scale for Schizophrenia) and extrapyramidal symptoms and did not experience significant changes in these symptoms throughout the study, supporting the specificity of the study findings for negative symptoms. In general, d6-DM/Q was safe and well-tolerated in this study (Example 1) with a safety profile consistent with other clinical trials of the same compound. The most frequently (>2 patients) reported TEAEs in either the all d6-DM/Q group or the all placebo group, respectively, were dry mouth (4.5% vs. 1.0%), diarrhea (2.3% vs. 3.1%), dizziness (3.4% vs. 3.1%), headache (2.3% vs. 5.2%), somnolence (1.1% vs. 3.1%) and nasopharyngitis (3.4% vs. 4.2%). No deaths were reported in the study and no serious AEs occurred in the all d6-DM/Q group.

Taken together, the results from this study (Example 1) demonstrate a consistent, positive signal of efficacy across a wide range of reliable and validated measures indicating that adjunctive treatment with d6-DM/Q could provide a safe, effective, and clinically meaningful therapeutic option for stable schizophrenia patients exhibiting negative symptoms. The safety profile of d6-DM/Q was favorable and supportive of further development for this indication.

### Example 2

Exemplary Parameters for a Multicenter, Randomized, Double-Blind, Placebo-Controlled, Parallel-Arm Studies to Assess the Efficacy, Safety, and Tolerability of d6-DM/Q (Deudextromethorphan Hydrobromide [d6-DM]/Quinidine Sulfate [Q]) for the Treatment of Negative Symptoms of Schizophrenia

In the study from Example 1, patients treated with d6-DM/Q at a dose of 34/4.9 mg twice daily (BID) demonstrated improvements in negative symptoms of schizophrenia compared to patients on placebo. d6-DM/Q was safe and well-tolerated in that study.

To evaluate the efficacy, safety, and tolerability of a higher dose of d6-DM/Q (42.63/4.9 mg), patients randomized to d6-DM/Q are titrated from an initial dose of d6-DM/Q of d6-DM 28 mg/Q 4.9 mg (d6-DM/Q-28/4.9) once to twice daily during the first week of the randomized treatment period; patients then receive d6-DM 42.63 mg/Q 4.9 mg (d6-DM/Q-42.63/4.9) administered orally BID for the remainder of the randomized treatment period.

The d6-DM/Q-42.63/4.9 BID dose is associated with d6-DM exposures within the range shown to be generally well tolerated in Phase 1 and Phase 2 studies of d6-DM/Q, and within the range where receptor binding is sufficient to test the effectiveness hypothesis. This higher dose of d6-DM/Q 42.63/4.9 mg BID is studied across multiple indications, without new safety signals emerging across the d6-DM/Q clinical development programs to date.

The 12-week treatment duration ensures exposure to the targeted optimal dose of d6-DM/Q for an adequate period of time to observe a treatment response and to assess duration of response. The consensus group comprising the National Institute of Mental Health (NIMH), FDA, academic, and industry representatives, have identified a minimum 12-week duration of treatment as appropriate for designing studies of negative symptoms of schizophrenia (Laughren and Levin, Schizophr Bull. 2006; 32(2):220-222).

1 Investigational Plan

1.1 Overall Study Design

This is a possible design for multicenter, randomized, double-blind, placebo-controlled study to assess the efficacy, safety, and tolerability of d6-DM/Q in patients with negative symptoms of schizophrenia. The study includes up to a 4-week Screening period, a 12-week double-blind treatment period, and a 30-day follow-up period. Up to 370 patients are enrolled

Screening Period (Day −28 to Day −1)

The Screening period is 28 days (4 weeks) and begins when consent has been obtained. An extension of up to 14 additional days (42-day maximum total) can be requested from the Medical Monitor, if needed, to meet eligibility requirements.

Double-Blind Treatment Period

Patients are randomized in a 1:1 ratio to receive either d6-DM/Q or matching placebo capsules during. Patients randomized to d6-DM/Q begin a one-week titration period in which they receive a dose of d6-DM 28 mg/Q 4.9 mg (d6-DM/Q-28/4.9) every morning (qam) and a dose of placebo every evening (qpm) for the first 3 days, followed by d6-DM/Q-28/4.9 BID for the next 4 days. Following this one-week titration period, patients receive d6-DM 42.63 mg/Q 4.9 mg (d6-DM/Q-42.63/4.9) administered orally BID for the remaining 11 weeks of the double-blind treatment period.

Follow-Up Period

The 30-day follow-up period begins following the last visit (Visit 5 [Week 12] or Early Termination [ET]). The patient is contacted by phone at Week 16 to collect adverse event (AE) and concomitant medication information for the 30 days following the last dose of study medication.

Assessments and Visits:

Patients attend clinic visits at Screening, Visit 1 (Day 1 (Baseline)), and at Weeks 3, 6, 9, and 12, or at ET. A phone call occurs at Week 1, Week 4, Week 7, Week 10, and 30 days after the Week 12 or ET Visit.

Study procedures are performed at each visit as outlined in the Schedule of Evaluations and Visits (Table 16). The primary efficacy measure is the PANSS Marder negative factors score. Secondary efficacy measures include: NSA-16 Global Negative Symptom Score, Patient Global Impression of Severity (PGI-S), and Patient Global Impression of Change (PGI-C). Other outcome measures include: PANSS positive subscale and Calgary Depression Scale for Schizophrenia (CDSS). The NSA-16 is conducted at all visits where the NSA-16 Global is assessed, however the NSA-16 is not an efficacy measure in the study.

Pharmacokinetic (PK) measurements of plasma concentrations of d6-DM. Q, and certain metabolites are measured from samples collected at Visits 2, 4, and 6 (or ET).

The safety and tolerability of d6-DM/Q is assessed by reported AEs, physical examination, vital signs, clinical laboratory measures, 12-lead electrocardiograms (ECG), and the following scales: Columbia Suicide Severity Rating Scale (C-SSRS), Abnormal Involuntary Movements Scale (AIMS), Barnes Akathisia Scale (BAS), and the Simpson Angus Scale for Extrapyramidal Symptoms (SAS).

1.2 Number of Patients

Up to 370 patients are enrolled.

1.3 Treatment Assignment

Treatment (12-week, double-blind, treatment period; d6-DM/Q or placebo) is randomly assigned (1:1 ratio) based on a randomization scheme provided by the Sponsor.

1.4 Study Assessments and Procedures

A description of the study assessments and procedures is provided in Section 8.

TABLE 16
Schedule of Evaluation and Visits

| | Visit: | | | Study Treatment Period | | | | | | | | |
|---|---|---|---|---|---|---|---|---|---|---|---|---|
| | Study Day: | | Baseline | | | | | | | | Visit 5¹/ | Post Exit |
| | End of | Screening | Visit 1¹ | Phone² | Visit 2¹ | Phone² | Visit 3¹ | Phone² | Visit 4¹ | Phone² | ET | Phone² |
| | Study | Day −28 to −1 | Day 1 | Day 8 | Day 22 | Day 29 | Day 43 | Day 50 | Day 64 | Day 71 | Day 85 | Day 115 |
| Procedure | Week: | Week −4 to −1 | Week 0 | Week 1 | Week 3 | Week 4 | Week 6 | Week 7 | Week 9 | Week 10 | Week 12 | Week 16 |

| | | | | | | | | | | | |
|---|---|---|---|---|---|---|---|---|---|---|---|
| Informed Consent Form | X | | | | | | | | | | |
| signed | | | | | | | | | | | |
| CTSdatabase Entry | X | | | | | | | | | X | |
| Medical History/Psychiatric | X | | | | | | | | | | |
| History | | | | | | | | | | | |
| M.I.N.I. Exam | X | | | | | | | | | | |
| Review Inclusion and | X | X | | | | | | | | | |
| Exclusion Criteria | | | | | | | | | | | |
| Document Patient's | X | | | | | | | | | | |
| Informant Information | | | | | | | | | | | |
| NSA-16 and NSA Global | | X | | X | | X | | X | | X | |
| PANSS | X | X | | X | | X | | X | | X | |
| PGI-S | | X | | X | | X | | X | | X | |
| PGI-C | | X | | X | | X | | X | | X | |
| Enrollment (Visit 1)/ | | X | | | | | | | | | |
| Randomization (Visit 2) | | | | | | | | | | | |
| AiCure (Patient Training) | | | | | | | | | | | |
| Physical and Neurological | X | | | | | | | | | X | |
| Examinations | | | | | | | | | | | |
| Resting 12-lead ECG³ | X | X | | X | | X | | X | | X | |
| Chemistry, Hematology, and | X | X | | | | X | | | | X | |
| Urinalysis⁴ | | | | | | | | | | | |
| Pregnancy Test⁵ | X | X | | X | | X | | X | | X | |
| Urine Drug Screen | X | | | | | | | | | | |
| Hepatitis B and C; HIV | X | | | | | | | | | | |
| antibody | | | | | | | | | | | |
| Lab-CYP2D6⁶ | | X | | | | | | | | | |
| Pharrnacokinetic sampling⁷ | | X | | | | X | | | | X | |
| Plasma Antipsychotic | X | X | | | | X | | | | X | |
| Levels | | | | | | | | | | | |
| Review of Adverse Events | | X | X | X | X | X | X | X | X | X | X |
| Review Concomitant | X | X | X | X | X | X | X | X | X | X | X |
| Medications | | | | | | | | | | | |
| Record Vital Signs/Weight⁸ | X | X | | X | | X | | X | | X | |
| CDSS | X | X | | | | X | | | | X | |
| Extrapyramidal Symptoms | X⁹ | X | | | | X | | | | X | |
| Assessment Scales (AIMS, | | | | | | | | | | | |
| BAS, SAS) | | | | | | | | | | | |
| C-SSRS | X | X | | X | | X | | X | | X | |
| Dispense Study Drug | | X | | X | | X | | X | | | |
| Dose in Clinic (first dose of | | X | | X | | X | | X | | X | |
| the day) | | | | | | | | | | | |
| Review Returned Unused | | X | X¹⁰ | X | X¹⁰ | X | X¹⁰ | X | X¹⁰ | X | |
| Study Drug | | | | | | | | | | | |

¹Visit 1 has a ±3 day window; Visits 2-5 have a ±6 day window.
²Telephone calls have a +3 day window.
³Triplicate ECGs at Screening only. Electrocardiogram is performed pre-dose and 1-2 hours (±15 minutes) post-dose at Visit 1 and Visit 2; ECGs at all other visits are performed pre-dose only (Visits 3, 4, and 5).
⁴Fasting glucose and lipids are measured at Screening, Visits 1, 3, and Visit 5/ET visit for patients who withdraw prior to study completion. HbA1c is measured at Screening and Visit 5/ET. Thyroid function tests are measured at Screening only.
⁵Urinary (beta-hCG) test is performed for all females of childbearing potential (serum beta-hCG at Screening only).
⁶Blood samples for CYP2D6 genotyping are taken at Visit 1.
⁷Plasma concentrations of d6-DM, its metabolites (d3-DX and d3-3-MM), and Q are measured from samples collected post-dose on Day 1 (Week 0), pre- and post-dose on Day 43 (Week 6), and pre-dose on Day 85 (Week 12).
⁸BP and HR measurements are performed (in duplicate) at all clinic visits. Orthostatic BP and HR is measured at the Screening visit only; all other BP and HR measurements are taken while the patient is sitting/semi-recumbent. Respiratory rate and body temperature are measured at Screening, Day 1 (Visit 1), and Day 85 (Visit 5)/ET. Weight and height are measured at Screening, Day 1 (Visit 1), and Day 85 (Visit 5).
⁹Only the SAS is measured at Screening (AIMS and BAS not measured at Screening).
¹⁰Patient is asked if they have taken their medications as directed during telephone calls at Days 8, 29, 50, and 71.

2 Selection and Withdrawal of Patients

Eligible patients are adult outpatients, between 18 and 60 years of age, who are diagnosed with schizophrenia, show evidence of negative symptoms of schizophrenia, are clinically stable, and meet all the inclusion criteria and none of the exclusion criteria noted in the following sections.

The initial diagnostic assessment is performed by the Investigator to assess if the patients meet the diagnostic criteria for schizophrenia according to the Diagnostic and Statistical Manual of Mental Disorders, 5^{th} edition (DSM-V), using the Mini International Neuropsychiatric Interview (M.I.N.I.) Version 6.0.0 or 7.0.2 (Appendix 11A or 11B, respectively).

2.1 Patient Inclusion Criteria
1. Males and females 18 to 60 years of age, inclusive, at time of informed consent.
2. Patients who meet DSM-V diagnostic criteria for schizophrenia confirmed by the M.I.N.I Version 7.0.2, with onset at least 1 year before Screening and have been clinically stable for at least 6 months (no psychiatric hospital admissions or acute exacerbations). Patients who have been hospitalized within the last 6 months before Screening for social reasons are allowed upon consultation with the Medical Monitor, but currently hospitalized patients are excluded.
3. Patients are required to have been in a stable living situation for at least 30 days before Screening.
4. Patients should be treated with a second-generation atypical antipsychotic drug (SGA) other than clozapine in any approved dosage form for at least 3 months prior to Screening and be on a stable dose for at least 30 days prior to Screening; patients must remain clinically stable (in the opinion of the Principal Investigator) through Visit 1 and may not be treated with more than one SGA with the exception of low dose quetiapine (up to 50 mg at night) for insomnia.
5. Negative symptoms that have been present for at least 6 months, in the judgment of the Investigator.
6. Patients must have a score of:
   a. ≤4 on PANSS items P1: delusions; P3: hallucinations; P6: suspiciousness/persecution; and P7: hostility; and
   b. ≥4 (moderate) on any 2, or ≥5 on any 1, of the following items of the PANSS: N1: blunted affect; N2: emotional withdrawal; N4: passive/apathetic social withdrawal; or N6: lack of spontaneity/flow of conversation.
7. Women of childbearing potential who are sexually active must use an effective method of birth control during the course of the trial, and for at least 30 days after the last dose of study drug. To minimize the risk of failure of one method of birth control, two of the following precautions must be used: vasectomy, tubal ligation, vaginal diaphragm, intrauterine device, birth control pills, birth control depot injection, birth control implant, or condom with spermicide or sponge with spermicide. Periodic abstinence (e.g., calendar, ovulation, symptothermal, post-ovulation methods), declaration of abstinence for the duration of exposure to study drug, or withdrawal are not acceptable methods of contraception. Women who are sterile (i.e., had an oophorectomy and/or hysterectomy), postmenopausal (12 consecutive months with no menses without an alternative medical cause), or practice true abstinence (when this method is in line with the preferred and usual lifestyle of the patient) are exempt from this requirement.
8. Concomitant use of antidepressants such as selective serotonin reuptake inhibitors (SSRIs; e.g., fluoxetine, sertraline, citalopram) and serotonin-norepinephrine reuptake inhibitors (SNRIs; e.g., venlafaxine, desvenlafaxine, duloxetine, vortoxetine, vilazodone) are allowed as long as the dose has been stable for 3 months (90 days) prior to Screening, remains stable throughout the study, and the dose used is within the guidance from the U.S. label for that drug. Paroxetine, a cytochrome P450 (CYP) 2D6 substrate, is allowed provided the dose does not exceed 10 mg/day.
9. Concomitant use of hypnotics at bedtime (e.g., zolpidem at a dose of 5 to 10 mg or equivalent) for the nighttime treatment of insomnia is allowed, provided the dose has been stable for at least 1 month (30 days) prior to Visit 1 and remains stable throughout the study.
10. Patients on lorazepam up to a total dose of 2 mg per day for treatment of insomnia, anxiety, restlessness, or agitation. Patients should be on a stable dose for at least 1 month (30 days) prior to Visit 1 and the dose should remain stable for the duration of the study.
11. Willing to sign and receive a copy of the patient informed consent form (ICF) after the nature and risks of study participation have been fully explained.
12. Sufficient comprehension and cooperation to enable compliance with all procedures and assessments.
13. Patients must have a reliable informant (e.g., case manager, social worker, family member). In the opinion of the Investigator, the informant should spend an adequate amount of time with the patient to be able to address behaviors, activities and symptoms. The Investigator (or designee) should address this relationship in their initial assessment of the patient (or during the Screening period).

2.2 Patient Exclusion Criteria

Patients are not enrolled in the study if they meet any of the following criteria:
1. Patients with current major depressive disorder (MDD) and/or a CDSS score ≥6 at the Screening Visit.
2. Patients with a diagnosis of schizoaffective disorder or bipolar disorder.
3. Patients with a history of clozapine use within 3 months (90 days) prior to Visit 1. Clozapine is not allowed during the study.
4. Patients with pseudo-parkinsonism secondary to their ongoing antipsychotic medication based on Investigator judgment.
5. Patients who score a >3 on the sum of the first eight items of the Simpson-Angus Scale (SAS) during Screening.
6. Patients treated with any typical antipsychotic (e.g. haloperidol, thorazine, etc.) within 3 months (90 days) prior to Visit 1 or during the study duration.
7. Patients using anticholinergic medications within 1 month (30 days) prior to Visit 1 for treatment of AEs associated with antipsychotic medications.
8. Patients on levodopa.
9. Patients with undetectable levels of SGA at Screening. Patients with subtherapeutic levels of SGA at Screening are reviewed by the Medical Monitor for eligibility.
10. Patients on any benzodiazepine other than lorazepam as described in Inclusion Criteria number 10. Benzodiazepines other than lorazepam are not allowed in the study.
11. Patients with any of the following cardiovascular history or findings at Screening or Visit 1:
   a. History or evidence of complete heart block, ventricular tachycardia, presence of clinically significant premature ventricular contractions (PVCs) as evaluated by a central reader, QTc prolongation or torsades de pointes.
   b. QTc using the Fridericia's formula (QTcF) at Screening >450 msec for males and >470 msec for females based on central review at the Screening Visit, unless due to ventricular pacing.
   c. Any family history of congenital QT interval prolongation syndrome.
   d. History or presence of clinically significant syncope, orthostatic hypotension or postural tachycardia.
12. Patients with current clinically significant neurological, hepatic, renal, metabolic, hematological, immunological, cardiovascular, pulmonary, or gastrointestinal disorders, such as any history of myocardial infarction, congestive heart failure, HIV seropositive status/acquired immunodeficiency syndrome, or chronic hepatitis B or C. Medical conditions that are minor or well-controlled may be considered acceptable if the condition does not expose the patient to an undue risk of a significant adverse event or interfere with assessments of safety or efficacy during the course of the trial. The Medical Monitor should be contacted in any instance where the Investigator is uncertain regarding the stability of a patient's medical condition(s) and the potential impact of the condition(s) on trial participation.
13. Patients with known hypersensitivity to DM, d6-DM, Q, opiate drugs (codeine, etc.), or any other ingredient of the study medication.
14. Patients who have received DM co-administered with Q within 3 months (90 days) prior to Visit 1.
15. Patients who have ever received d6-DM co-administered with Q or were enrolled in the study from Example 1.
16. Patients with myasthenia gravis (a contraindication for Q).
17. Patients treated with monoamine oxidase inhibitors (MAOIs) within 2 weeks prior to Visit 1. MAOIs are prohibited throughout the study until 2 weeks post study drug discontinuation.
18. Patients who have been taking prohibited concomitant medications within 2 weeks or 5 half-lives, whichever is longer, prior to Visit 1.
19. Patients who use recreational or medicinal marijuana as evidenced by a positive urine drug screen for cannabis at Screening.
20. Patients who answer “Yes” on the C-SSRS Suicidal Ideation Item 4 (active suicidal ideation with some intent to act, without specific plan) and whose most recent episode meeting criteria for this C-SSRS Item 4 occurred within the last 6 months, OR
   Patients who answer “Yes” on the C-SSRS Suicidal Ideation Item 5 (active suicidal ideation with specific plan and intent) and whose most recent episode meeting criteria for this C-SSRS Item 5 occurred within the last 6 months OR
   Patients who answer “Yes” on any of the 5 C-SSRS Suicidal Behavior Items (actual attempt, interrupted attempt, aborted attempt, preparatory acts, or behavior) and whose most recent episode meeting criteria for any of these 5 C-SSRS Suicidal Behavior Items occurred within the last 2 years, OR
   Patients who, in the opinion of the Investigator, present a serious risk of suicide or homicide.
21. Patients with clinically significant laboratory abnormalities (hematology, chemistry, and urinalysis) or with safety values of potential clinical concern or with aspartate aminotransferase (AST) or alanine aminotransferase (ALT) >2×upper limit of normal (ULN) at the Screening Visit.
22. Unwilling or unable, in the opinion of the Investigator, to comply with study instructions.
23. Patients with a history of substance and/or alcohol abuse within 6 months prior to Screening. All tobacco and nicotine products are allowed.
24. Patients who test positive for hepatitis B surface antigen, hepatitis C antibody, or HIV antibody.
25. Patients who have received electroconvulsive treatment (ECT), repetitive transcranial magnetic stimulation (rTMS) or deep brain stimulation (DBS) in the past year prior to Screening.
26. Women who are lactating, pregnant, or plan to become pregnant.
27. Patients who are found to be a “Virtually Certain” match in the Clinical Trial Subject Database (CTSdatabase) with a patient who has participated in another interventional drug or device study within 30 days prior to Visit 1.

2.3 Optional Patient Inclusion and Exclusion Criteria

In inclusion criterion 1 above, the 6 month period with no hospitalizations can be reduced to a 4 month period.

Inclusion criterion 6(a) above can be replaced with the following inclusion criterion: patients must have a score of s 4 on PANSS items P1: delusions; P3: hallucinations; and P7: hostility.

The following additional inclusion criterion can be applied: patients must have a PANSS Marder negative factors score of a 20 at Screening and Visit 1 AND <4 points absolute difference between the 2 visits. PANSS Marder negative factors: N1: blunted affect; N2: emotional withdrawal; N3: poor rapport; N4: passive/apathetic social withdrawal; N6: lack of spontaneity/flow of conversation; G7: motor retardation; G16: active social avoidance.

Inclusion criteria 9 and 10 above can be replaced with the following inclusion criterion: concomitant use of hypnotics at bedtime (e.g., eszopiclone, solpidem, zaleplon, trazodone [up to 100 mg/day]) for the nighttime treatment of insomnia is allowed, provided the dose has been stable for at least 1 month (30 days) prior to baseline and remains stable throughout the study. Patients on lorazepam for anxiety, restlessness, or agitation prior to study entry should remain on the same treatment regimen for the duration of the study. All other benzodiazepines are disallowed, except for lorazepam use for short term or prn treatment of insomnia and behavioral disturbances. The duration of dosing should not exceed 3 days in a 7-day period.

Exclusion criterion 12 above can be replaced with the following exclusion criterion: patients with concurrent clinically significant or unstable systemic diseases that could confound the interpretation of safety results of the study (e.g., malignancy [except skin basal-cell carcinoma or untreated prostate cancer], poorly controlled diabetes, poorly controlled hypertension, unstable pulmonary, renal or hepatic disease, unstable ischemic cardiac disease, dilated cardiomyopathy, or unstable valvular heart disease), cognitive and other neurodegenerative disorders. Some cases might be evaluated individually with the Investigator and medical monitor.

The following additional exclusion criterion can be applied: patients who are currently participating in, or who had participated in other interventional (drug or device) clinical study within 30 days prior to baseline.

2.4 Patient Informant

It is recognized that due to their illness, patients with negative symptoms often have difficulty engaging in relationships with others. However, for the accuracy of data, it is critical that an informant be familiar enough with the patient in order to be able to report on their behaviors, activities and symptoms, as well as any changes in these. The informant should spend a sufficient amount of time with the patient each week to be able to accurately report on these items. While there are no specific requirements, Investigators are asked to document the relationship between the informant and the patient (i.e., family member, social worker, or case manager) and to specify the length of the relationship. This documentation is reviewed by the Medical Monitor and the informant relationship is taken into account in the eligibility process for the study.

2.5 Patient Withdrawal Criteria

Patients and caregivers are advised verbally and in the written ICF that they have the right to withdraw from the study at any time without prejudice or loss of benefits to which they are otherwise entitled. The Investigator or Sponsor may discontinue a patient from the study in the event of an intercurrent illness, adverse event, other reasons concerning the health or well-being of the patient, decline in patient's comprehension or cognitive function that affects their ability to continue in the study, or in the case of lack of cooperation, non-compliance, protocol violation, or other administrative reasons. If a patient does not return for a scheduled visit, every effort should be made to contact the patient/caregiver. Regardless of the circumstance, every effort should be made to document patient outcome. The Investigator should inquire about the reason for withdrawal, request the patient/caregiver return all unused study drug, and follow-up with the patient/caregiver regarding any unresolved adverse events.

In addition, patients who present with a QTc interval (QTcF) >500 msec (unless due to ventricular pacing) or a QTcF interval change from the pre-dose Baseline ECG of >60 msec at any time after randomization, are withdrawn from the study. The QTcF values are assessed for clinical significance and recorded.

3 Treatment of Patients

3.1 Description of Study Drug

d6-DM/Q is provided as an immediate release, blue, opaque, printed hard gelatin capsule (size 3) containing the active ingredient d6-DM and Q. The inactive ingredients are croscarmellose sodium, microcrystalline cellulose, colloidal silicone dioxide, and magnesium stearate. Each capsule of study drug contains one of the following:
d6-DM/Q-28/4.9: 28 mg of d6-DM and 4.9 mg of Q
d6-DM/Q-42.63/4.9: 42.63 mg of d6-DM and 4.9 mg of Q
Matching Placebo: identical in appearance to d6-DM/Q capsules; containing inactive ingredients only

Drug supplies are provided to the study sites in blinded (double-blind), individual, prelabeled blister cards.

Study drug is prepared, packaged, and labeled in accordance with Good Manufacturing Practice (GMP) guidelines, International Council on Harmonisation (ICH), Good Clinical Practice (GCP) guidelines, and applicable laws and regulations.

3.2 Administration of Study Drug

The administration of study drug is as follows.

Double-Blind, Randomized, Treatment Period:

Patients are randomized in a 1:1 ratio, stratified by placebo-responder status and center, to receive either d6-DM/Q or matching placebo capsules administered orally.

Patients randomized to d6-DM/Q have the following fixed-titration schedule: d6-DM/Q-28/4.9 qam for the first 3 days, d6-DM/Q-28/4.9 BID for the following 4 days, and then d6-DM/Q-42.63/4.9 BID beginning on the eighth day through the remaining 11 weeks.

Administration of study drug during the double-blind treatment period is as follows:
d6-DM/Q Group:
Day 1-Day 3: d6-DM/Q-28/4.9 capsule every morning; a dose of matching placebo every evening
Day 4-Day 7: d6-DM/Q-28/4.9 capsule, BID
Day 8-Day 85: d6-DM/Q-42.63/4.9 capsule, BID

Placebo Group:
Day 22-Day 106: Matching d6-DM/Q placebo capsule, BID

Packaging of the study drug is described in Section 4.2.

3.3 Concomitant Medications

Patients may not take any of the prohibited medications listed in Section 3.3.2 during the study or 2 weeks or 5 half-lives (whichever is longer) prior to the start of dosing on Day 1. At each visit, patients are queried as to whether they had taken any concomitant medications and, if so, the Investigator records the medications taken and the reasons for their use.

d6-DM/Q contains quinidine sulfate (Q), which is a P-glycoprotein inhibitor. Concomitant administration of Q at higher doses used for cardiac indications, with digoxin, a P-glycoprotein substrate, results in plasma or serum digoxin concentrations that may be as much as doubled; digoxin concentrations should be closely monitored in patients taking digoxin concomitantly and the dose reduced, as necessary.

In cases of prodrugs whose actions are mediated by cytochrome P450 isoenzyme 2D6 (CYP2D6-produced metabolites) for example, codeine and hydrocodone, whose analgesic and antitussive effects appear to be mediated by morphine and hydromorphone, respectively, it may not be possible to achieve the desired clinical benefits in the presence of d6-DM/Q due to Q-mediated inhibition of CYP2D6. An alternative treatment should be considered.

3.3.1 Allowed Concomitant Medications, Use of Benzodiazepines and Hypnotics

Allowed concomitant medications should be evaluated by the Investigator and discussed with the Medical Monitor as necessary to determine if there is any concern for use during the study.

Concomitant use of hypnotics at bedtime (e.g., zolpidem at a dose of 5 to 10 mg or equivalent) for the nighttime treatment of insomnia is allowed, provided the dose has been stable for at least 1 month prior to Visit 1 and remains stable throughout the study. Patients on lorazepam up to a total dose of 2 mg per day for treatment of insomnia, anxiety, restlessness, or agitation should be on a stable dose for 1 month (30 days) prior to Visit 1 and remain on the same treatment regimen for the duration of the study.

All other benzodiazepines are disallowed except for lorazepam use for short term or “as needed” treatment of anxiety, insomnia, and/or behavioral disturbances. The dose should not exceed 2 mg per day for 3 days in a 7-day period and no more than 45 total days of such therapy is allowed during the study.

Benzodiazepines and non-benzodiazepine sleep aids must not be administered within 8 hours prior to any scheduled efficacy or safety scale assessments, including extrapyramidal symptoms (EPS) scales. Investigators are encouraged to delay scale administration until a full 8 hours have elapsed since the last benzodiazepine or sleep aid dose, if at all possible, including at Screening and Baseline assessments. However, if delaying administration of efficacy and safety scales is not feasible, the scales should still be administered and the use of benzodiazepine or sleep aid documented, including a notation of the drug name, dose, and time of administration on the electronic case report forms (eCRF).

Concomitant use of antidepressants, such as SSRIs (e.g., fluoxetine, sertraline, citalopram) and SNRIs (e.g., venlafaxine, desvenlafaxine, duloxetine, vortoxetine, vilazodone), are allowed as long as the dose has been stable for 3 months (90 days) prior to Screening, remains stable throughout the study, and the dose used is within the guidance from the U.S. label for that drug. Paroxetine, a CYP2D6 substrate, is allowed provided the dose does not exceed 10 mg/day.

3.3.2 Prohibited Medications

Prohibited concomitant medications include medications that could potentially alter plasma levels of Q and/or d6-DM, or medications related to Q. A list of examples of prohibited medications is provided in Table 3 above.

Patients may not take any of the prohibited medications listed in Table 3 during the study or within 2 weeks or 5 half-lives, whichever is longer, of study drug dosing (Day 1). Monoamine oxidase inhibitors (MAOIs) are prohibited throughout the study. Patients taking an MAOI should allow at least 14 days before taking the first dose of study drug and at least 14 days after the last dose of study drug before starting an MAOI.

Use of levodopa is not allowed in the study. Recreational and/or medicinal marijuana use is not allowed during the study.

3.4 Treatment Compliance

Each patient is instructed to return any unused study drug and empty drug blister cards during each study visit during treatment. Site staff conduct a capsule count and record the compliance on the Drug Accountability log as well as enter the information in the eCRF.

Study drug compliance is assessed after a capsule count and patient interview; compliance is defined as ingesting at least 80% of the scheduled dose of study drug (compliance range: 80 to 120%).

An additional exploratory tool (AiCure) is used to help ensure patient compliance; however, the primary method of measuring compliance is a capsule count conducted by the site staff.

3.5 Randomization and Blinding

Upon entry into the study (after the ICF is signed at Screening), each patient is assigned a 9-digit patient number. The first 6 digits consist of the country code—center number; the last 3 digits are assigned sequentially by the interactive web response system (IWRS) starting with 001. This 9-digit number is the main identifier for each patient.

Patients are randomized to receive either d6-DM/Q capsules or matching placebo capsules (double-blind manner) in a 1:1 ratio. The randomization scheme is devised by Sponsor or designee and managed within an IWRS. Each patient has a 50% chance of receiving d6-DM/Q.

4 Study Drug Materials and Management

4.1 Study Drug

d6-DM/Q is provided as an immediate release, blue, opaque, printed hard gelatin capsule (size 3) containing the active ingredients d6-DM (28 mg or 42.63 mg) and Q (4.9 mg). The composition of the d6-DM/Q and matching placebo capsules is described in Section 3.1.

4.2 Study Drug Packaging and Labeling

Packaging

Each study drug kit is an individually labeled blister card pre-packaged for 3 weeks of treatment plus an additional week of supply. Inside the blister card there are four panels, each consisting of 2 rows of blister strips, one row for the morning dose (as indicated AM) and one row for the evening dose (as indicated PM) for 1 week of supply.

4.3 Study Drug Storage

Clinical supplies must be stored in compliance with label requirements in a secure place and kept at room temperature; 25° C. (77° F.) with excursions permitted to 15° C. to 30° C. (59° F. to 86° F.).

4.4 Study Drug Administration

Each patient receives study drug according to their medicine identification number (randomization number) assigned by the IWRS randomization scheme. Designated staff at each study site dispense the study drug blister cards. Study drug is self-administered, except on the applicable study visit days when patients take their morning dose of study drug in the clinic in the presence of study site personnel, regardless of the time of day.

Each patient is instructed to ingest 1 capsule of study drug orally with water BID, approximately every 12 (±4) hours (morning and evening) (2 capsules daily). For each patient, the time each dose of study drug is ingested should remain consistent throughout double-blind treatment. Study drug doses are recorded in the AiCure medication adherence monitoring platform.

All study drug is supplied and administered in a double-blind manner.

5 Assessment of Efficacy

Efficacy assessments include the PANSS Marder negative factors score as the primary assessment and NSA Global Negative Symptom Score, Patient Global Impression-Severity (PGI-S), and Patient Global Impression-Change (PGI-C) as secondary endpoints. Other outcome measures include the PANSS positive subscale and the Calgary Depression Scale for Schizophrenia (CDSS). These scales are described in the following sections.

5.1 Positive and Negative Syndrome Scale (PANSS) Marder Negative Factors Score

The PANSS (Appendix 2) is a validated clinical scale that has been extensively used as a reliable and valid measure of the negative and positive symptoms of schizophrenia (Daniel, Schizophr Res. 2013; 150(2-3):343-5). The scale comprises 30 disparate items that collectively assess the positive and negative syndromes in schizophrenia, including their relationship to one another and to global psychopathology. Each is scored for “1” (absent) to “7” (extremely severe).

The established psychometric properties of the PANSS allow for the assessment of positive, negative, and general psychopathology as part of a categorical or dimensional perspective of schizophrenia (Kay et al. Schizophr Bull. 1987; 13(2):261-276; Kumari et al. J Addict Res Ther. 2017; 8(3)). Thus, different combinations of items are generally analyzed as factor structures to score specific aspects of the negative syndrome of schizophrenia.

The concept of a five-factor solution for the PANSS has been successfully used in clinical trials, and identifies five factors or dimensions of schizophrenia: 1) negative symptoms; 2) positive symptoms; 3) disorganized thought; 4) uncontrolled hostility/excitement; and 5) anxiety/depression (Lindenmayer et al. Psychopathology. 1995; 28(1):22-31; Marder et al. J Clin Psychiatry. 1997; 58(12):538-546). Marder investigated the five-factor solution in two controlled trials and found that risperidone produced significantly greater improvements than haloperidol on all five dimensions, with a particularly potent effect of risperidone vs. haloperidol on Factor 1 (negative symptoms). Factor 1, i.e., the PANSS Marder negative factors has several aspects of improved content validity versus the negative subscale, meeting more consistently with the domains identified in the 2006 NIMH-MATRICS Consensus Statement (Kirkpatrick et al. Schizophr Bull. 2006; 32(2):214-219).

PANNS Marder Negative Factors Score

The PANSS Marder negative factors score is a reliable and validated measure of the negative symptoms of schizophrenia, and is comprised of the following 7 items of the 30-item PANSS:

Marder Negative Factors:

1. N1: Blunted affect

2. N2: Emotional withdrawal

3. N3: Poor rapport

4. N4: Passive/apathetic social withdrawal

5. N6: Lack of spontaneity and flow of conversation

6. G7: Motor retardation

7. G16: Active social avoidance

Each of the PANSS Marder negative factors correlates with one of the five main domains of negative symptoms (Kirkpatrick et al. Schizophr Bull. 2006; 32(2):214-219). PANSS item N1: blunted affect, correlates with Blunted affect; N6: lack of spontaneity and conversation flow, correlates with Alogia; and N4: passive/apathetic social withdrawal; G16: active social avoidance; and N3: poor rapport, are factors that correlate with Asociality. PANSS item N2: emotional withdrawal, correlates with Anhedonia; and G7: motor retardation, correlates with both Anhedonia and Avolition (Daniel, Schizophr Res. 2013; 150(2-3):343-5).

Two of the items in the PANSS Marder negative factors score (N4 and G16) are based solely on information obtained from an informant. Patients need to identify a reliable informant (e.g., case manager, social worker, family member) who spends sufficient time with them to be able to provide information to PANSS raters.

The PANSS positive subscale (P1-P7) is also assessed for changes in psychotic symptoms, and includes P1: delusions; P2: conceptual disorganization; P3: hallucinatory behavior; P4: excitement; P5: grandiosity; P6: suspicious/persecution; and P7: hostility.

The PANSS evaluation is performed at Screening (Day −28 to −1), Visit 1 (Day 1), Visit 2 (Day 22), Visit 3 (Day 43), Visit 4 (Day 64), and Visit 5/ET Visit (Day 85).

5.2 Negative Symptom Assessment-16 (NSA-16) Global Negative Symptom Score

The NSA-16 (Appendix 3A or 3B) is considered a valid and reliable measure of the presence, severity, and range of negative symptoms associated with schizophrenia; it has high inter-rater and test-retest-reliability across languages and cultures (Daniel, Schizophr Res. 2013; 150(2-3):343-345; Axelrod et al. J Psychiatr Res. 1993; 27(3):253-258). The NSA-16 uses a 5-factor model to describe negative symptoms: 1) Communication, 2) Emotion/affect, 3) Social involvement, 4) Motivation, and 5) Retardation. These factors, assessed through a structured interview, are comprehensive and well-defined to help standardize assessment. As a truncated version of the 25-item NSA, the NSA-16 still captures the multidimensionality of negative symptoms but can be completed in approximately 15 to 20 minutes. The “normal” reference population against which the subject is to be compared is a healthy young person in their twenties.

NSA Global negative symptom score rates the overall severity of negative symptoms when defined as the absence or reduction of behaviors normally present in a healthy young person. Ratings should not depend on any specific item or items from the NSA or any other similar instrument. Instead, it should measure the raters gestalt of the interview and is assessed following completion of the NSA-16 interview (Alphs et al. Int J Methods Psychiatr Res. 2011; 20(2):e31-37).

The NSA-16 and NSA Global Negative Symptom Score evaluations are performed at Visit 1 (Day 1), Visit 2 (Day 22), Visit 3 (Day 43), Visit 4 (Day 64), and Visit 5/ET Visit (Day 85).

5.3 Patient Global Impression-Severity (PGI-S)

The PGI-S (Appendix 4) is a 7-point (1-7), patient-rated scale used to assess the severity of the patient's schizophrenia as follows: 1) normal, not at all ill; 2) borderline ill; 3) mildly ill; 4) moderately ill; 5) markedly ill; 6) severely ill; 7) extremely ill.

The PGI-S evaluation is performed at Visit 1 (Day 1), Visit 2 (Day 22), Visit 3 (Day 43), Visit 4 (Day 64), and Visit 5/ET (Day 85), and is focused on the negative symptoms of schizophrenia.

5.4 Patient Global Impression-Change (PGI-C)

The PGI-C (Appendix 5A or 5B) is a 7-point (1-7), patient-rated scale used to assess treatment response with respect to the patient's schizophrenia as follows: very much improved, much improved, minimally improved, no change, minimally worse, much worse, or very much worse.

The PGI-C evaluation is performed at Visit 2 (Day 22), Visit 3 (Day 43), Visit 4 (Day 64), and Visit 5/ET Visit (Day 856), and is focused on the negative symptoms of schizophrenia.

5.5 Calgary Depression Scale for Schizophrenia (CDSS)

The CDSS (Appendix 6) is a 9-item scale derived from the Hamilton Depression Scale (Ham-D) that is designed to assess depression specifically in patients with schizophrenia (Addington et al. Schizophr Res. 1996; 19(2-3):205-212). Unlike the Ham-D, the CDSS does not contain depressive symptoms that overlap with negative symptoms of schizophrenia, such as anhedonia and social withdrawal. The CDSS has shown excellent psychometric properties. Each item on the scale is scored as 0, Absent; 1, Mild; 2, Moderate; or 3, Severe. The CDSS score is obtained by adding each of the item scores. A score above 6 has an 82% specificity and 85% sensitivity for predicting the presence of a major depressive episode.

The CDSS evaluation is performed at Screening (Day −28 to −1), Visit 1 (Day 1), Visit 3 (Day 43), and Visit 5/ET Visit (Day 85).

6 Assessment of Pharmacokinetics

Plasma concentrations of d6-DM, its metabolites (d3-DX and d3-3-MM), and Q are measured from samples collected post-dose on Day 1 (Week 0), pre- and post-dose on Day 43 (Week 6), and pre-dose on Day 85 (Week 12). These samples are collected per instructions provided by the sponsor.

The date and time of each sample collection and the date and time of the last dose of study drug prior to the sample collection are recorded on the eCRF.

Blood samples are separated by centrifugation and then frozen at −20° C. until assayed at the analytical unit. Procedures for the collection, storage, and shipping of samples for analysis are provided to the study sites by the sponsor at the time of study initiation.

Plasma concentrations of d6-DM, d3-DX, d3-3-MM, and Q are summarized descriptively and may be used in future population PK analyses.

7 Assessment of Safety

7.1 Adverse Events

An AE is any untoward medical occurrence or unintended change (including physical, psychiatric [e.g., depression], or behavioral) from the time the ICF is signed, including intercurrent illness, which occurs during the course of a clinical trial after treatment has started, whether considered related to treatment or not. An AE can therefore be any unfavorable and unintended sign (including an abnormal laboratory finding, for example), symptom, or disease temporally associated with the use of a medicinal product, whether or not considered related to the medicinal product. Changes associated with normal growth and development that do not vary in frequency or magnitude from that ordinarily anticipated clinically are not AEs (e.g., onset of menstruation occurring at a physiologically appropriate time).

Clinical AEs should be described by diagnosis and not by symptoms when possible (e.g., cold, seasonal allergies, instead of “runny nose”).

An overdose is a deliberate or inadvertent administration of a treatment at a dose higher than specified in the protocol and higher than known therapeutic doses. It must be reported irrespective of outcome, even if toxic effects were not observed.

AEs are graded on a 3-point scale and reported in detail as indicated on the eCRF:
Mild: easily tolerated, causing minimal discomfort and not interfering with normal everyday activities
Moderate: sufficiently discomforting to interfere with normal everyday activities
Severe: incapacitating and/or preventing normal everyday activities

The relationship of each AE to study medication should be determined by the Investigator using the following explanations:
Not related: the event is clearly related to other factors such as the subject's clinical state, therapeutic interventions, or concomitant medications administered to the subject
Unlikely related: the event is most likely produced by other factors such as the subject's clinical state, therapeutic interventions, or concomitant medications administered to the subject; and does not follow a known response pattern to the study medication
Possibly related: the event follows a reasonable temporal sequence from the time of drug administration; and/or follows a known response pattern to the study medication; but could have been produced by other factors such as the subject's clinical state, therapeutic interventions, or concomitant medications administered to the subject
Related: the event follows a reasonable temporal sequence from the time of drug administration; and follows a known response pattern to the study medication; and cannot be reasonably explained by other factors such as the subject's clinical state, therapeutic interventions, or concomitant medications administered to the subject

7.2 Serious Adverse Events

A serious adverse event (SAE) is any AE occurring at any dose that results in any of the following outcomes:
Death
Life-threatening experience (one that places the subject, in the view of the initial reporter, at immediate risk of death from the AE as it occurred, i.e., it does not include an AE that, had it occurred in a more severe form, might have caused death)
Persistent or significant disability/incapacity (disability is a substantial disruption of a person's ability to conduct normal life functions)
Inpatient hospitalization or prolongation of hospitalization
Congenital anomaly/birth defect

Important medical events that may not result in death, or be life-threatening, or require hospitalization may be considered an SAE when, based upon appropriate medical judgment, they may jeopardize the subject or require medical or surgical intervention to prevent one of the listed outcomes.

Pregnancy is not considered to be an AE or an SAE, unless a complication occurs that meets the requirements for an AE or SAE, but must be reported on a pregnancy report form. Females who are pregnant or likely to become pregnant are excluded from this study. In the event a patient becomes pregnant during the study, study medication must be discontinued, a pregnancy report form must be completed to capture potential drug exposure during pregnancy, and the pregnancy must be reported within 24 hours of notice. Any pregnant patient must be followed until the outcome of her pregnancy is known (i.e., normal delivery, abnormal delivery, spontaneous/voluntary/therapeutic abortion). The pregnancy (i.e., mother and fetus) must be followed-up through delivery with regard to outcome.

A pregnancy report form must also be completed in the event that a female partner of a male patient becomes pregnant within 30 days after the last dose of study drug or study completion, whichever is greater.

The term “severe” is a measure of intensity; thus, a severe AE is not necessarily serious. For example, nausea of several hours duration may be rated as severe, but may not be clinically serious.

7.3 Reporting Adverse Events

Patients are queried regarding AEs at all visits post Screening. The Investigator assesses and records all reported AEs. Any AE newly reported after a patient has received the last dose of study drug and up until the End-of-Study visit is followed until resolution (patient's health has returned to his/her Baseline status or all variables have returned to normal) or until stabilization of the event has occurred (the Investigator does not expect any further improvement or worsening of the event).

A death occurring during the study, or which comes to the attention of the Investigator within 30 days after stopping study drug, whether considered treatment-related or not, must be reported to the Sponsor.

For all SAEs, including an abnormal laboratory test value assessed as clinically significant, the Investigator should consult with the sponsor's Medical Monitor or designated representative as needed and report any SAE by fax/email form as detailed below no later than 24 hours after becoming aware of the event. Subsequently, the SAE must be assessed for the following details: seriousness of event, start date, stop date, intensity, frequency, relationship to study drug, action taken regarding test drug, treatment required, and outcome to date.

7.4 Physical and Neurological Examinations

Physical and neurological examinations are performed at Screening (Day −28 to Day −1), and at the final clinic visit (Visit 5 [Week 12]). The physical examination includes assessments of head, eyes, ears, nose, throat, lymph nodes, skin, extremities, respiratory, gastrointestinal, musculoskeletal, cardiovascular, and nervous systems. The neurological examination includes assessments of mental status, cranial nerves, motor system, reflexes, coordination, gait and station, and sensory system. The physical and neurological examinations should be performed by the same person each time, whenever possible.

Physical and neurological examination abnormalities determined by the Investigator to be clinically significant at Screening should be recorded as medical history. Any clinically significant changes in physical and neurological examination findings from the Screening examination should be recorded as AEs.

7.5 Vital Sign Measurements

Blood pressure (BP) and heart rate (HR) measurements are performed (in duplicate) at all clinic visits. Orthostatic BP and HR is measured at the Screening visit only, and is described below; all other BP and HR measurements are taken while the patient is sitting/semi-recumbent.

Orthostatic BP and HR at Screening Visit: supine BP and HR is measured after the patient has rested for at least 5 minutes in the supine position; BP and HR measurements are taken twice in the same position and recorded. Following the measurement of supine BP and HR, the patient stands still for up to 3 minutes and a single measurement of standing BP and HR is recorded (within 3 minutes of standing).

Respiratory rate (breaths/minute) and body temperature (° F.) are assessed at Screening, Day 1 (prior to dosing), and at the final clinic visit (Visit 5 [Week 12]).

Weight and height are recorded at Screening, Day 1 (Visit 1), and Day 85 (Visit 5).

7.6 Electrocardiogram (ECG)

A resting 12-lead ECG is performed at Screening and each clinic visit (Visits 1, 2, 3, 4, 5, and 6); the Screening ECG is performed in triplicate (1 minute apart). ECGs are performed pre-dose and 1-2 hours (±15 minutes) post-dose at Visit 1 and Visit 2; ECGs at all other visits are performed pre-dose only (Visits 3, 4, and 5).

The ECG equipment is provided to the study center by the central reader. The ECG assessment is recorded at the study center and includes general findings, including heart rate (beats/minute), QRS complex and PR and QTc intervals. Results are provided by the central reader to the Investigators within 72 hours. Any ECG abnormality present at Screening is recorded as medical history. Any changes from the ECG status at Screening that are deemed to be clinically significant by the Investigator should be captured as AEs.

Any clinically significant abnormal ECG should be discussed with the Medical Monitor and, if necessary, the ECG should be repeated within approximately 1 week. In addition, any patient who presents with a QTcF interval >500 msec (unless due to ventricular pacing) or a QTcF interval increase from the pre-dose Baseline ECG (Day 22/Week 3) >60 msec at any time after randomization, is withdrawn from the study.

7.7 Safety Scales

The AIMS, BAS, SAS, and C-SSRS scales are assessed for purposes of safety, and are briefly described in the following sections.

7.7.1 Abnormal Involuntary Movement Scale (AIMS)

The AIMS (Appendix 7A or 7B) is composed of 12 items and used to assess dyskinesia. Items related to severity of orofacial, extremity, and trunk movements, global judgment about incapacitation, and patient awareness are rated using a 5-point scale (0=none to 4=severe). Two items related to dental status are scored using “yes” or “no” responses.

The AIMS evaluation is performed at Visit 1 (Day 1), Visit 3 (Day 43), and Visit 5/ET Visit (Day 85).

7.7.2 Barnes Akathisia Scale (BAS)

The BAS (Appendix 8) consists of items that assess the objective presence and frequency of akathisia, the level of an individual's subjective awareness and distress, and global severity.

The BAS is scored as follows:
Objective Akathisia, Subjective Awareness of Restlessness and Subjective Distress Related to Restlessness are rated on a 4-point scale from 0-3 and are summed yielding a total score ranging from 0 to 9. The Global Clinical Assessment of Akathisia uses a 5-point scale ranging from 0-4.

The BAS evaluation is performed at Visit 1 (Day 1), Visit 3 (Day 43), and Visit 5/ET Visit (Day 85).

7.7.3 Simpson Angus Scale for Extrapyramidal Symptoms (SAS)

The SAS (Appendix 9A or 9B) is composed of 10 items and is used to assess pseudoparkinsonism. Grade of severity of each item is rated using a 5-point scale. SAS scores can range from 0 to 40. Signs assessed include gait, arm-dropping, shoulder-shaking, elbow rigidity, wrist rigidity, leg pendulousness, head rotation, glabella tap, tremor, and salivation.

The SAS evaluation is performed at Screening (Day −28 to −1), Visit 1 (Day 1), Visit 3 (Day 43), and Visit 5/ET Visit (Day 85).

7.7.4 Columbia Suicide Severity Rating Scale (C-SSRS)

The C-SSRS (Appendix 10) is a clinical interview providing a summary of both ideation and behavior that can be administered during any evaluation or risk assessment to identify the level and type of suicidality present. The C-SSRS can also be used during treatment to monitor for clinical worsening.

Suicidality is monitored during the study using the “Baseline/Screening” and “Since Last Visit” versions of the C-SSRS scale. The “Baseline/Screening” version, which assesses the lifetime experience of the patient with suicide events and suicidal ideation and the occurrence of suicide events or ideation within a specified time period prior to entry into the study, is completed for all patients at Screening to determine eligibility. Any patient with active suicidal ideation within the last 6 months, suicidal behaviors within the last 2 years, or who in the clinical judgement of the Investigator presents a serious risk of suicide should be excluded from the study (see Section 2.2, Exclusion Criteria). The “Since Last Visit” C-SSRS form is also completed at visits after Screening.

The C-SSRS evaluation is performed at Screening (Day −28 to −1), Visit 1 (Day 1), Visit 2 (Day 22), Visit 3 (Day 43), Visit 4 (Day 64), and Visit 5/ET Visit (Day 85).

8 Schedule of Evaluations and Procedures

A schedule of evaluations and procedures is provided in Table 16.

Description of Study Procedures

At each study visit, a member of the study staff is required to enter information into the IWRS database regarding patient data and pre-defined study assessment results. Further instructions are provided in the IRT Site Manual.

The pre- and post-dose procedures listed below are generally listed in the order in which they should be performed, within a reasonable amount of flexibility. The NSA-16, NSA-16 Global, PANSS, PGI-S, and PGI-C assessments are all done pre-dose and should be completed as early as possible during the study procedures for the applicable visits. Any pre-dose ECGs and pre-dose blood draws should only be conducted after the above scales have been completed at each visit. The CTSdatabase check for dual enrollment at Screening should be performed as one of the first post-consent procedures, so that sites are aware of potential dual enrollment before more complex tasks are performed.

Screening Visit (Days −28 to −1)

The following procedures are performed at Screening within 28 days prior to Day 1.
1. The Investigator or delegated site staff provide the patients with informed consent documents and explain the rationale for the study, providing ample time for patients to ask questions. Patients must provide written informed consent before any study-related procedures are performed.
2. Authorization form completed and patient information is entered into the CTSdatabase.
3. A qualified and certified rater administers the PANSS.
4. Psychiatric history is recorded and M.I.N.I. exam is conducted.
5. An adequately trained and certified rater administers the CDSS to assess for symptoms of depression.
6. The Investigator (or qualified designee) who is adequately trained completes the “Baseline/Screening” C-SSRS form to exclude patients with a significant risk of suicidal behavior.
7. Medical history, including patient demographics, and any concomitant medications use (including over-the-counter [OTC] medications, vitamins, and supplements) are reviewed and recorded.
8. Inclusion and exclusion criteria (eligibility form) are reviewed.
9. Detailed information on the patient's relationship with their informant is collected and documented.
10. Vital signs are measured in duplicate and recorded (orthostatic BP and HR at Screening only [See Section 7.5], respiratory rate, and body temperature); weight and height are also recorded.
11. Physical and neurological examinations are conducted.
12. An adequately trained and experienced clinician administers the SAS to assess for EPS.
13. Resting 12-lead ECGs are performed in triplicate (1 minute apart).
14. Blood and urine specimens are collected for clinical laboratory assessments (chemistry, including fasting glucose, lipids, TSH, and HbA1c; hematology; and urinalysis).
15. Blood specimen is collected for assessment of plasma antipsychotic levels.
16. A serum pregnancy test is performed on all females of childbearing potential.
17. A blood specimen is collected for hepatitis B and C (HBsAg and HCAb) and HIV antibody screening.
18. Urine specimen is collected for drug screening.
19. Review assessments to verify that patients are eligible to continue study participation.

Following Screening procedures for assessment of inclusion and exclusion criteria, a protocol eligibility form is submitted to the Medical Monitor for approval. Patients deemed eligible by the Investigator and the Medical Monitor return for Visit 1 (Day 1). Patients having ECG findings or laboratory test results outside of the reference normal range that the Investigator considers as clinically significant, and that may place the patient at a higher risk for study participation, are discontinued (not randomized).

Visit 1 (Baseline, Randomization; Day 1/Week 0±3-Day Window)

Patients are randomized (1:1 ratio) to receive either d6-DM/Q or matching placebo capsules for 12 weeks during the double-blind treatment period. The first dose of study drug for the double-blind treatment period is administered in the clinic on Day 1.

Randomization occurs after all Visit 2 pre-dose procedures have been completed; this visit should occur within a 3-day window. The following procedures are performed at Visit 1 (Day 1±3-day window):

Pre-Dose:
1. A qualified and certified rater administers the NSA-16, NSA-16 Global, PANSS, PGI-S, and PGI-C.
2. An adequately trained and certified rater administers the CDSS to assess for symptoms of depression.
3. The Investigator (or qualified designee) who is adequately trained completes the “Since Last Visit” C-SSRS form.
4. Vital signs are measured and recorded (sitting/semi-recumbent BP and HR [in duplicate]).
5. Resting 12-lead ECG is performed (pre-dose, after the above scales have been administered).
6. Patient is queried regarding AEs and concomitant medication use (including OTC medications, vitamins, and supplements).
7. Blood and/or urine specimens are collected for safety laboratory assessments (chemistry [including fasting glucose and lipids], hematology, urinalysis)-all blood draws should be done after the above scales have been administered.
8. Blood specimen is collected for assessment of plasma antipsychotic levels.
9. Urine pregnancy test is performed on all females of childbearing potential.
10. An adequately trained and experienced clinician administers the AIMS, SAS, and BAS to assess for EPS.
11. A review of all unused study drug is performed by site staff. Sufficient study drug is dispensed for BID dosing through the following visit.

Patients are randomized once it is determined that they satisfy all of the inclusion and none of the exclusion criteria (on the basis of the Screening and Visit 1 assessments described above), and are assigned a study drug kit number via IWRS.

Study Drug Dosing:

The first dose of study medication is administered from the AM strip of the study drug blister card at the clinic regardless of the time of day.

Post-Dose:
1. Blood specimen is collected for PK assessment of plasma d6-DM, Q, and d6-DM metabolites (between 1 and 3 hours post-dose).
2. Resting 12-lead ECG is performed between 1 and 2 hours post-dose (±15 minutes).

Patient Instructions

Patients are instructed to take the study drug BID (1 capsule from the blister pack labeled AM in the morning and 1 capsule from the blister pack labeled PM in the evening; approximately every 12 hours) until the next visit. Patients are also instructed to bring any unused study drug at each study visit.

Patients are reminded to use AiCure to monitor study drug compliance outside of the clinic.

The Investigator and/or study coordinator gives patients detailed instructions regarding study procedures. They are also instructed to consult with the study site prior to taking any non-study medications. These requirements are reviewed in person, and written instructions may also be provided to the patient in addition to the informed consent per Investigator discretion.

The Investigator queries patients at the end of each visit to be certain they understand what is required of them.

12-Week, Double-Blind Treatment Period

8.1.3.1 Safety Telephone Contact (Day 8/Week 1±3-Day Window)

Patients receive a telephone call to assess AEs and query regarding concomitant medications at Week 1 (Day 8±3 days). Patients are also asked if they have taken their study drug as directed.

Visit 2 (Day 22/Week 3±6-Day Window)

The following procedures are performed on the morning of Day 22 (±6-day window):

Pre-Dose:
1. A qualified and certified rater administers the NSA-16, NSA-16 Global, PANSS, PGI-S, and PGI-C.
2. Resting 12-lead ECG is performed (pre-dose, after the above scales have been administered).

Study Drug Dosing:

Study drug is administered from the AM strip of the newly dispensed blister pack at the clinic after the completion of the above procedures.

Post-Dose:
1. Patient is queried regarding AEs and concomitant medication use (including OTC medications, vitamins, and supplements).
2. The Investigator (or qualified designee) who is adequately trained completes the “Since Last Visit” C-SSRS form.
3. Vital signs are measured in duplicate and recorded (sitting/semi-recumbent BP and HR).
4. Urine pregnancy test is performed on all females of childbearing potential.
5. A review of all unused study drug is performed by site staff. Sufficient study drug is dispensed for BID dosing through the following visit.
6. Resting 12-lead ECG is performed 1 to 2 hours post-dose (±15 minutes).

Patient Instructions

Patients are instructed to take the study drug BID (1 capsule from the blister pack labeled AM in the morning and 1 capsule from the blister pack labeled PM in the evening; approximately every 12 hours) until the next visit. Patients are also instructed to bring any unused study drug at each study visit.

Patients are reminded to use AiCure to monitor study drug compliance outside of the clinic.

The Investigator and/or study coordinator gives patients detailed instructions regarding study procedures. They are also instructed to consult with the study site prior to taking any non-study medications. These requirements are reviewed in person, and written instructions may also be provided to the patient in addition to the informed consent per Investigator discretion.

The Investigator queries patients at the end of each visit to be certain they understand what is required of them.

Safety Telephone Contact (Day 29/Week 4±3-Day Window)

Patients receive a telephone call to assess AEs and query regarding concomitant medications at Week 4 (Day 29±3 days). Patients are also asked if they have taken their study drug as directed.

Visit 3 (Day 43/Week 6±6-Day Window)

The following procedures are performed on the morning of Day 64 (±6-day window):

Pre-Dose:
1. A qualified and certified rater administers the NSA-16, NSA-16 Global, PANSS, PGI-S, and PGI-C.
2. Resting 12-lead ECG is performed (pre-dose, after the above scales have been administered).
3. Blood specimen is collected for PK assessment of plasma d6-DM, Q, and metabolites (pre-dose, after the above scales have been administered).

Study Drug Dosing:

Study drug is administered from the AM strip of the newly dispensed blister pack at the clinic, regardless of the time of day.

Post-Dose:
1. Patient is queried regarding AEs and concomitant medication use (including OTC medications, vitamins, and supplements).
2. An adequately trained and experienced clinician administers the AIMS, SAS, and BAS to assess for EPS.
3. An adequately trained and certified rater administers the CDSS to assess for symptoms of depression.
4. The Investigator (or qualified designee) who is adequately trained completes the “Since Last Visit” C-SSRS form.
5. Urine pregnancy test is performed on all females of childbearing potential.
6. Blood and/or urine specimens are collected for safety laboratory assessments (chemistry [including fasting glucose and lipids], hematology, urinalysis).
7. Blood specimen is collected for assessment of plasma antipsychotic levels.
8. Blood specimen is collected for PK assessment of plasma d6-DM, Q, and metabolites (between 1 and 3 hours post-dose).
9. A review of all unused study drug is performed by site staff. Sufficient study drug is dispensed for BID dosing through the following visit.

Patient Instructions

Patients are instructed to take the study drug BID (1 capsule from the blister pack labeled AM in the morning and 1 capsule from the blister pack labeled PM in the evening; approximately every 12 hours) until the next visit. Patients are also instructed to bring any unused study drug at each study visit.

Patients are reminded to use AiCure to monitor study drug compliance outside of the clinic.

The Investigator and/or study coordinator gives patients detailed instructions regarding study procedures. They are also instructed to consult with the study site prior to taking any non-study medications. These requirements are reviewed in person, and written instructions may also be provided to the patient in addition to the informed consent per Investigator discretion.

The Investigator queries patients at the end of each visit to be certain they understand what is required of them.

Safety Telephone Contact (Day 50/Week 7±3-Day Window)

Patients receive a telephone call to assess AEs and query regarding concomitant medications at Week 7 (Day 50±3 days). Patients are also asked if they are taking their study drug as directed.

Visit 4 (Day 64/Week 9±6-Day Window)

The following procedures are performed on the morning of Day 85 (±6-day window).

Pre-Dose:
1. A qualified and certified rater administers the NSA-16, NSA-16 Global, PANSS, PGI-S, and PGI-C.
2. Resting 12-lead ECG is performed (pre-dose, after the above scales have been administered).

Study Drug Dosing:

Study drug is administered from the AM strip of the newly dispensed blister pack at the clinic, regardless of the time of day.

Post-Dose:
1. Vital signs are measured in duplicate and recorded (sitting/semi-recumbent BP and HR).
2. Patient is queried regarding AEs and concomitant medication use (including OTC medications, vitamins, and supplements).
3. The Investigator (or qualified designee) who is adequately trained completes the “Since Last Visit” C-SSRS form.
4. Urine pregnancy test is performed on all females of childbearing potential.
5. A review of all unused study drug is performed by site staff. Sufficient study drug is dispensed for BID dosing through the following visit.

Patient Instructions

Patients are instructed to take the study drug BID (1 capsule from the blister pack labeled AM in the morning and 1 capsule from the blister pack labeled PM in the evening; approximately every 12 hours) until the final visit (Visit 6; Day 106). Patients are also instructed to bring any unused study drug at each study visit.

Patients are reminded to use AiCure to monitor study drug compliance outside of the clinic.

The Investigator and/or study coordinator gives patients detailed instructions regarding study procedures. They are also instructed to consult with the study site prior to taking any non-study medications. These requirements are reviewed in person, and written instructions may also be provided to the patient in addition to the informed consent per Investigator discretion.

The Investigator queries patients at the end of each visit to be certain they understand what is required of them.

Safety Telephone Contact (Day 71/Week 10±3-Day Window)

Patients receive a telephone call to assess AEs and query regarding concomitant medications at Week 10 (Day 71±3 days). Patients are also asked if they have taken their study drug as directed.

Visit 5 (Day 85/Week 12±6-Day Window)/Early-Termination Visit

The following procedures are performed on Day 85 (±6-day window) or for the ET Visit for those patients who withdraw prior to Week 12:

Pre-Dose:
A qualified and certified rater administers the NSA-16, NSA-16 Global, PANSS, PGI-S, and PGI-C.
Resting 12-lead ECG is performed (pre-dose, after the above scales have been administered).
Blood specimen is collected for PK assessment of plasma d6-DM, Q. and metabolites (pre-dose, after the above scales have been administered).

Study Drug Dosing:

Last dose of study drug is administered from the AM strip of the study drug blister card brought in by the patient, regardless of the time of day, for those patients who complete double-blind treatment (not those who prematurely discontinue).

Post-Dose:
Vital signs are measured in duplicate and recorded (sitting/semi-recumbent BP and HR, respiratory rate, and body temperature); weight and height are also recorded.
Physical and neurological examinations are conducted.
Patient is queried regarding AEs and concomitant medication use (including OTC medications, vitamins, and supplements).
An adequately trained and experienced clinician administers the AIMS, SAS, and BAS to assess for EPS.
An adequately trained and certified rater administers the CDSS to assess for symptoms of depression.
The Investigator (or qualified designee) who is adequately trained completes the “Since Last Visit” C-SSRS form.
Blood and/or urine specimens are collected for safety laboratory assessments (chemistry [including fasting glucose, lipids, and HbA1c], hematology, urinalysis).
Blood specimen is collected for assessment of plasma antipsychotic levels.
Urine pregnancy test is performed on all females of childbearing potential.
Patients return all unused study medication; a review of all unused study drug is performed by site staff.

At the last patient contact, site staff access CTSdatabase, enter the patient Study ID and the nature of the last contact (i.e., completer or ET).

Procedures for Early Termination:

All patients undergo a complete evaluation at Visit 5/ET. In addition, Visit 5/ET evaluations are completed for any patient withdrawn at any time after randomization into the trial; evaluations should be completed within 48 hours of the last dose of study drug, whenever possible. Attempts should be made to complete all evaluations, particularly efficacy assessments, for the Visit 5/ET visit prior to the administration of any new psychotropic medications. However, if the patient receives a new rescue medication for worsening schizophrenia symptoms prior to the V5/ET procedures, no efficacy assessments should be performed.

For patients who discontinue treatment, study drug is not administered in the clinic on that day and, therefore, there is no specific time frame for the 12-lead ECG and the blood/urine specimen collection (clinical laboratory tests or pharmacokinetics).

Safety Follow-Up Telephone Contact (Week 16/Day 115±3-Day Window)

Patients are contacted by phone at Week 16 (Day 115±3 days) and asked if they have experienced any AEs or changes to their medications since their final visit. Patients who experience AEs may need to return to the clinic for an unscheduled visit for safety assessments.

Any AE previously reported and not yet resolved at the time of this phone contact, is followed-up until resolution (patient's health has returned to his/her Baseline status or all variables have returned to normal) or until stabilization of the event has occurred (the Investigator does not expect any further improvement or worsening of the event).

Any newly reported AE after receiving and up to 30 days after the last dose of study drug is followed up until resolution (patient's health has returned to his/her Baseline status or all variables have returned to normal) or until stabilization of the event has occurred (the Investigator does not expect any further improvement or worsening of the event).

9 Statistics

An overview of the planned statistical methods for this study are provided below. The full statistical methods are provided in the statistical analysis plan (SAP) prior to the unblinding of the study.

9.1 Analysis Populations

Analysis populations for this study are defined as follows:

Safety Population: all patients who are randomized and take at least one dose of study drug. All safety analyses are based on the safety population.

Modified Intent-to-Treat (mITT): all patients in the Safety Population with both Baseline and at least one post Baseline PANSS measurement. All efficacy analyses are based on mITT population.

9.2 Demographics and Baseline Characteristics

Demographics and Baseline characteristics are summarized by treatment group using descriptive statistics.

9.3 Efficacy Analysis

9.3.1 Study Endpoints

9.3.1.1 Primary Efficacy Endpoint

The primary efficacy endpoint is the change from Baseline to Visit 5 (Week 12) in the PANSS Marder negative factors score.

9.3.1.2 Secondary Efficacy Endpoints

Secondary efficacy endpoints include change from Baseline to Visit 5 (Week 12) for the following outcomes measures (PGI-C raw score measures change):
NSA-16 Global Negative Symptom Score
PGI-S
PGI-C

9.3.1.3 Other Outcome Measures

Other outcome measures include change from Baseline to Visit 5 (Week 12) for the following measures:
PANSS positive subscale
Calgary Depression Scale for Schizophrenia (CDSS)

9.3.2 Primary Efficacy Analysis

The primary efficacy endpoint is the change in PANSS Marder negative factors score from Baseline to Week 12. The primary efficacy endpoint is analyzed using the likelihood-based linear mixed effects model repeated measures (MMRM) on observed data. The model includes fixed effects for treatment, trial center, visit, treatment-by-visit interaction, and Baseline-by-visit interaction. An unstructured covariance model is used. Small trial centers are pooled according to region and practice for the analysis before database unblinding.

The purpose of this study is to assess the expected drug effect in a future population that results from patients initiating d6-DM/Q vs Placebo. The estimand of primary interest is defined as follows:
Population: mITT
Variable: change in PANSS Marder negative factors score from Baseline to Week 12.
Intercurrent event(s): The “treatment policy” strategy is followed, whereby the value for the variable of interest is used regardless of adherence to randomized treatment and/or initiation of prohibited medications.
Population-level summary: The between-treatment difference in the mean changes from Baseline to Week 12 in the PANSS Marder negative factors score.

All data collected during the study are used in the statistical analysis. For the primary efficacy analysis, the treatment effect is estimated using the MMRM method described above. Under the missing at random (MAR) assumption, MMRM provides an unbiased estimate of treatment effect for the treatment period. Analyses with missing values imputed by multiple imputation (MI) under missing not at random (MNAR), and other methods are performed as sensitivity analyses.

9.3.3 Secondary Efficacy and Other Outcome Measures Analysis

Secondary efficacy endpoints and Other outcome measures are analyzed in a similar manner as the primary efficacy analysis when appropriate. Further details and analysis methods are described in the SAP.

9.4 Pharmacokinetic Analysis

Plasma concentrations of d6-DM, its metabolites d3-DX and d3-3-MM, and Q are summarized descriptively.

9.5 Safety Analysis

Safety analyses are based on safety population defined as all patients who are randomized and take at least one dose of study drug. It consists of data summaries for biological parameters and AEs. Safety analyses are tabulated by treatment.

9.5.1 Adverse Events

AEs are coded using the Medical Dictionary for Regulatory Activities (MedDRA). The percentages of patients experiencing 1 or more AEs are summarized by treatment, system organ class (SOC), deaths, nonfatal SAEs, AEs, AEs resulting in study discontinuation, and treatment-emergent AEs (TEAEs).

9.5.2 Vital Signs and Electrocardiograms

Summary statistics of absolute values and percentage change from Baseline (Visit 1; Day 1/Week 0) for BP (diastolic and systolic), heart rate, respiratory rate, weight, and ECG parameters are provided. All values outside a predefined normal range are highlighted in the individual patient data listings.

9.5.3 Clinical Laboratory Measures

Laboratory parameters are summarized via descriptive statistics and via shifts in results with respect to normal ranges between Visit 1 (Week 0) and end-of-treatment as increased, decreased, or no change.

9.5.4 Safety Scales

The C-SSRS, SAS, BAS, and AIMS are summarized via descriptive statistics and via shift tables of the number and percent of patients in each score from Baseline (Visit 1; Day 1/Week 0) to post-Baseline visits and end of treatment.

### APPENDIX 1

TABLE 17
Baseline Second-Generation Antipsychotic (SGA) Use Safely Population (N = 144)

| | Stage 1 | Stage 1 |
|---|---|---|
| Anatomical Therapeutic Subgroup (ATC Level 2), n (%) | Placebo | d6-DM/Q |
| Preferred Term, n (%) | (N = 96) | ( N = 48) |
| Number of Patients taking Baseline SGAs | 96 (100.0) | 48 (100.0) |
| PSYCHOLEPT1CS | 96 (100.0) | 48 (100.0) |
| Aripiprazole | 16 (16.7) | 15 (31.3) |
| Lithium | 1 (1.0) | 0 (0.0) |
| Lurasidone | 2 (2.1) | 1 (2.1) |
| Lurasidone Hydrochloride | 2 (2.1) | 3 (6.3) |
| Olanzapine | 26 (27.1) | 12 (25.0) |
| Paliperidone | 3 (3.1) | 3 (6.3) |
| Paliperidone Palmitate | 11 (11.5) | 2 (4.2) |
| Quetiapine | 2 (2.1) | 2 (4.2) |
| Quetiapine Fumarate | 14 (14.6) | 7 (14.6) |
| Risperidone | 25 (26.0) | 10 (20.8) |
| Ziprasidone | 2 (2.1) | 1 (2.1) |

Note:
Baseline concomitant medications are defined as medications with a start date on or before the first dose date of randomized study medication, and an end date on or after the first dose date (or the medication is ongoing) of randomized study medication. Medications are coded using WHO Drug Dictionary Version September 2015 Treatment allocation based on Stage I randomization.

TABLE 18
Stage 1 Baseline Efficacy Assessments
Stage 1 mITT Population (N = 127)

| Assessment | Stage 1 Placebo | Stage 1 d6-DM/Q | All Patients |
|---|---|---|---|
| Statistics | (N = 80) | (N = 47) | (N = 127) |
| NSA-16 Total Score | | | |
| N | 80 | 47 | 127 |
| Mean (SD) | 60.4 (7.71) | 61.0 (7.53) | 60.6 (7.62) |
| Median | 60.0 | 60.0 | 60.0 |
| Min, Max | 43, 85 | 45, 79 | 43, 85 |
| NSA-16 Factor Domain: Communication | | | |
| N | 80 | 47 | 127 |
| Mean (SD) | 12.6 (2.71) | 12.5 (2.54) | 12.6 (2.64) |
| Median | 13.0 | 12.0 | 13.0 |
| Min, Max | 6, 21 | 8, 21 | 6, 21 |
| NSA-16 Factor Domain: Ernotion7, Affect | | | |
| N | 80 | 47 | 127 |
| Mean (SD) | 12.2 (1.81) | 12.6 (2.1.1) | 12.3 (1.93) |
| Median | 12.0 | 13.0 | 12.0 |
| Min, Max | 8, 17 | 8, 17 | 8, 17 |
| NSA-16 Factor Domain: Social involvement | | | |
| N | 80 | 47 | 127 |
| Mean (SD) | 12.2 (2.20) | 12.2 (2.02) | 12.2 (2.13) |
| Median | 12.0 | 12.0 | 12.0 |
| Min, Max | 8, 17 | 9, 16 | 8, 17 |
| NSA-16 Total Score | | | |
| N | 80 | 47 | 127 |
| Mean (SD) | 60.4 (7.71) | 61.0 (7.53) | 60.6 (7.62) |
| Median | 60.0 | 60.0 | 60.0 |
| Min, Max | 43, 85 | 45, 79 | 43, 85 |
| NSA-16 Factor Domain: Communication | | | |
| N | 80 | 47 | 127 |
| Mean (SD) | 12.6 (2.71) | 1.2.5 (2.54) | 12.6 (2.64) |
| Median | 13.0 | 12.0 | 13.0 |
| Min, Max | 6, 21 | 8, 21 | 6, 21 |
| NSA-16 Factor Domain: Ernotion7, Affect | | | |
| N | 80 | 47 | 127 |
| Mean (SD) | 12.2 (1.81) | 12.6 (2.11) | 12.3 (1.93) |
| Median | 12.0 | 13.0 | 12.0 |
| Min, Max | 8, 17 | 8, 17 | 8, 17 |
| NSA-16 Factor Domain: Social involvement | | | |
| N | 80 | 47 | 127 |
| Mean (SD) | 12.2 (2.20) | 12.2 (2.02) | 12.2 (2.13) |
| Median | 12.0 | 12.0 | 12.0 |
| Min, Max | 8, 17 | 9, 16 | 8, 17 |
| NSA-16 Factor Domain: Motivation | | | |
| N | 80 | 47 | 127 |
| Mean (SD) | 16.7 (2.33) | 16.5 (2.37) | 16.6 (2.33) |
| Median | 17.0 | 17.0 | 17.0 |
| Min, Max | 9, 22 | 11, 22 | 9, 22 |
| NSA-16 Factor Domain: Retardation | | | |
| N | 80 | 47 | 127 |
| Mean (SD) | 6.7 (1.61) | 7.2 (1.54) | 6.9 (1.59) |
| Median | 7.0 | 7.0 | 7.0 |
| Min, Max | 2, 10 | 4, 10 | 2, 10 |
| NSA-4 Total Score | | | |
| N | 80 | 47 | 127 |
| Mean (SD) | 17.3 (7.36) | 17.4 (2.44) | 17.3 (2.38) |
| Median | 17.0 | 17.0 | 17.0 |
| Min, Max | 12, 24 | 13, 22 | 17, 24 |
| NSA-16 Item 1: Prolonged Time to Respond | | | |
| N | 80 | 47 | 127 |
| Mean (SD) | 3.1 (1.13) | 3.1 (1.22) | 3.1 (1.15) |
| Median | 3.0 | 3.0 | 3.0 |
| Min, Max | 1, 6 | 1, 6 | 1, 6 |
| NSA-16 item 2: Restricted Speech Quantity | | | |
| N | 80 | 47 | 127 |
| Mean (SD) | 3.8 (1.12) | 3.5 (1.10) | 3.7 (1.12) |
| Median | 4.0 | 3.0 | 4.0 |
| Min, Max | 1, 6 | 1, 5 | 1, 6 |
| NSA-16 Item 3: Empoverished Speech Content | | | |
| N | 80 | 47 | 127 |
| Mean (SD) | 3.7 (0.91) | 3.6 (0.80) | 3.6 (0.87) |
| Median | 4.0 | 4.0 | 4.0 |
| Min, Max | 1, 5 | 1, 5 | 1, 5 |
| NSA-16 item 4: Inarticulate Speech | | | |
| N | 80 | 47 | 127 |
| Mean (SD) | 2.0 (1.07) | 2.3 (1.16) | 2.1 (1.11) |
| Median | 2.0 | 2.0 | 2.0 |
| Min, Max | 1, 5 | 1, 5 | 1, 5 |
| NSA-16 Item 5: Emotion:Reduced Range | | | |
| N | 80 | 47 | 127 |
| Mean (SD) | 4.2 (0.81) | 4.4 (0.92) | 4.3 (0.85) |
| Median | 4.0 | 4.0 | 4.0 |
| Min, Max | 3, 6 | 3, 6 | 3, 6 |
| NSA-16 Item 6: Affect:Reduce Modulation Intensity | | | |
| N | 80 | 47 | 127 |
| Mean (SD) | 4.2 (0.74) | 4.2 (0.89) | 4.2 (0.80) |
| Median | 4.0 | 4.0 | 4.0 |
| Min, Max | 3, 6 | 3, 6 | 3, 6 |
| NSA-16 Item 7: Affect:Reduced Display on Demand | | | |
| N | 80 | 47 | 127 |
| Mean (SD) | 3.8 (0.93) | 4.0 (0.92) | 3.9 (0.94) |
| Median | 4.0 | 4.0 | 4.0 |
| Min, Max | 1, 5 | 2, 6 | 1, 6 |
| NSA-16 item 8: Reduced Social Drive | | | |
| N | 80 | 47 | 127 |
| Mean (SD) | 4.7 (0.75) | 4.9 (0.55) | 4.8 (0.68) |
| Median | 5.0 | 5.0 | 5.0 |
| Min, Max | 1, 6 | 3, 6 | 1, 6 |
| NSA-16 Item 9: Poor Rapport With Interviewer | | | |
| N | 80 | 47 | 127 |
| Mean (SD) | 3.4 (1.02) | 3.3 (0.93) | 3.3 (0.99) |
| Median | 3.0 | 3.0 | 3.0 |
| Min, Max | 1, 5 | 1, 5 | 1, 5 |
| NSA-16 Item 10: Sexual Interest | | | |
| N | 80 | 47 | 127 |
| Mean (SD) | 4.2 (1.52) | 4.0 (1.54) | 4.1 (1.52) |
| Median | 4.0 | 4.0 | 4.0 |
| Min, Max | 1, 6 | 1, 6 | 1, 6 |
| NSA-16 Item 11: Poor Grooming and Hygiene | | | |
| N | 80 | 47 | 127 |
| Mean (SD) | 2.6 (1.20) | 2.5 (1.08) | 2.6 (1.15) |
| Median | 2.0 | 2.0 | 2.0 |
| Min, Max | 1, 5 | 1, 5 | 1, 5 |
| NSA-16 Item 12: Reduced Sense of Purpose | | | |
| N | 80 | 47 | 127 |
| Mean (SD) | 4.7 (0.92) | 4.5 (1.02) | 4.6 (0.96) |
| Median | 5.0 | 5.0 | 5.0 |
| Min, Max | 2, 6 | 2, 6 | 2, 6 |
| NSA-16 Item 13: Reduced Interests | | | |
| N | 80 | 47 | 127 |
| Mean (SD) | 4.5 (0.84) | 4.8 (0.84) | 4.6 (0.84) |
| Median | 5.0 | 5.0 | 5.0 |
| Min, Max | 2, 6 | 2, 6 | 2, 6 |
| NSA-16 Item 14: Reduced Daily Activity | | | |
| N | 80 | 47 | 127 |
| Mean (SD) | 4.8 (0.57) | 4.7 (0.74) | 4.8 (0.64) |
| Median | 5.0 | 5.0 | 5.0 |
| Min, Max | 3, 6 | 2, 6 | 2, 6 |
| NSA-16 Item 15: Reduced Expressive Gestures | | | |
| N | 80 | 47 | 127 |
| Mean (SD) | 3.9 (1.09) | 4.1 (1.13) | 4.0 (1.11) |
| Median | 4.0 | 4.0 | 4.0 |
| Min, Max | 1, 6 | 2, 6 | 1, 6 |
| NSA-16 item 16: Slowed Movements | | | |
| N | 80 | 47 | 127 |
| Mean (SD) | 2.8 (0.96) | 3.0 (0.78) | 2.9 (0.91) |
| Median | 3.0 | 3.0 | 3.0 |
| Min, Max | 1, 5 | 2, 5 | 1, 5 |
| NSA-16: Global Negative Symptoms Rating | | | |
| N | 80 | 47 | 127 |
| Mean (SD) | 4.6 (0.61) | 4.6 (0.64) | 4.6 (0.62) |
| Median | 5.0 | 5.0 | 5.0 |
| Min, Max | 3, 6 | 3, 6 | 3, 6 |
| NSA-16: Global Level of Functioning | | | |
| N | 80 | 46 | 126 |
| Mean (SD) | 4.6 (0.63) | 4.7 (0.73) | 4.6 (0.66) |
| Median | 5.0 | 5.0 | 5.0 |
| Mm, Max | 3, 6 | 3, 6 | 3, 6 |
| PANSS Total Score | | | |
| N | 80 | 47 | 127 |
| Mean (SD) | 68.7 (7.99) | 67.4 (8.26) | 68.2 (8.08) |
| Median | 69.0 | 68.0 | 69.0 |
| Min, Max | 51, 93 | 47, 83 | 47, 93 |
| PANSS Total Score Reduction >= 20% | 80 | 47 | 127 |
| Yes | 3 (3.51 | 5 (10.6) | 8 (6.3) |
| No | 77 (96.3) | 42 (89.4) | 119 (93.7) |
| PANSS Negative Subscale | | | |
| N | 80 | 47 | 127 |
| Mean (SD) | 25.2 (3.64) | 24.6 (3.51) | 25.0 (3.59) |
| Median | 26.0 | 24.0 | 25.0 |
| Min, Max | 18, 36 | 19, 35 | 18, 36 |
| PANSS Positive Subscale | | | |
| N | 80 | 47 | 127 |
| Mean (SD) | 13.4 (2.81) | 13.6 (3.65) | 13.5 (3.13) |
| Median | 13.0 | 13.0 | 13.0 |
| Min, Max | 7, 22 | 7, 23 | 7, 23 |
| PANSS General Psychopathology Subscale | | | |
| N | 80 | 47 | 127 |
| Mean (SD) | 30.1 (5.05) | 29.1 (4.66) | 29.7 (4.91) |
| Median | 29.0 | 30.0 | 30.0 |
| Min, Max | 18, 45 | 18, 38 | 18, 45 |
| PANSS Prosocial Factors | | | |
| N | 80 | 47 | 127 |
| Mean (SD) | 18.4 (2.92) | 18.3 (3.24) | 18.4 (3.03) |
| Median | 19.0 | 18.0 | 19.0 |
| Min, Max | 11, 28 | 10, 28 | 10, 28 |
| PANSS Marder Negative Factors | | | |
| N | 80 | 47 | 127 |
| Mean (SD) | 24.2 (3.81) | 24.1 (4.24) | 24.2 (3.96) |
| Median | 24.0 | 23.0 | 24.0 |
| Min, Max | 17, 35 | 17 ,37 | 17, 37 |
| PANSS Excitement Component | | | |
| N | 80 | 47 | 127 |
| Mean (SD) | 6.3 (2.10) | 6.2 (1.57) | 6.3 (1.92) |
| Median | 6.0 | 6.0 | 6.0 |
| MM, Max | 5, 17 | 5, 12 | 5, 17 |
| CGI-S | | | |
| N | 80 | 47 | 127 |
| Mean (SD) | 3.9 (0.74) | 3.7 (0.74) | 3.9 (0.74) |
| Median | 4.0 | 4.0 | 4.0 |
| Min, Max | 2, 6 | 1, 5 | 1, 6 |
| CGI-S Group | 78 | 46 | 124 |
| Percent Change = <65% | 78 (100.0) | 46 (100.0) | 124 (100.0) |
| Percent Change >65% | 0 (0.0) | 0 (0.0) | 0 (0.0) |
| CDSS Total Score | | | |
| N | 80 | 47 | 127 |
| Mean (SD) | 0.9 (1.31) | 1.1 (1.34) | 0.9 (1.32) |
| Median | 0.0 | 1.0 | 0.0 |
| Min, Max | 0, 5 | 0, 5 | 0, 5 |
| MCCB Composite Score | | | |
| N | 80 | 47 | 127 |
| Mean (SD) | 28.8 (13.09) | 32.5 (11.37) | 30.2 (12.56) |
| Median | 28.5 | 35.0 | 30.0 |
| Min, Max | 4, 56 | 9, 53 | 4, 56 |
| MCCB Speed of Processing | | | |
| N | 80 | 47 | 127 |
| Mean (SD) | 35.0 (11.37) | 35.3 (10.88) | 35.1 (11.15) |
| Median | 35.5 | 37.0 | 36.0 |
| Min, Max | 12, 58 | 12, 61 | 12, 61 |
| MCCB Attention/Vigilance | | | |
| N | 80 | 47 | 127 |
| Mean (SD) | 37.4 (13.28) | 40.6 (13.18) | 38.6 (13.28) |
| Median | 39.0 | 38.0 | 39.0 |
| Min, Max | 11, 70 | 15, 69 | 11, 70 |
| MCCB Working Memory | | | |
| N | 80 | 47 | 127 |
| Mean (SD) | 35.7 (12.05) | 40.6 (11.94) | 37.5 (12.19) |
| Median | 37.0 | 41.0 | 38.0 |
| Min, Max | 4, 60 | 17, 69 | 4, 69 |
| MCCB Verbal Learning | | | |
| N | 80 | 47 | 127 |
| Mean (SD) | 36.1 (8.14) | 38.9 (8.84) | 37.1 (8.48) |
| Median | 35.0 | 38.0 | 36.0 |
| Min, Max | 21, 65 | 25, 73 | 21, 73 |
| MCCB Visual Learning | | | |
| N | 80 | 47 | 127 |
| Mean (SD) | 34.5 (11.18) | 37.1 (12.90) | 35.5 (11.87) |
| Median | 32.0 | 35.0 | 34.0 |
| Min, Max | 13, 62 | 17, 67 | 13,67 |
| MCCB Reasoning and Problem Solving | | | |
| N | 80 | 47 | 127 |
| Mean (SD) | 43.9 (9.61) | 43.5 (9.25) | 43.8 (9.44) |
| Median | 43.0 | 42.0 | 42.0 |
| Min, Max | 24, 63 | 28, 65 | 24, 65 |
| MCCB Social Cognition | | | |
| N | 80 | 47 | 127 |
| Mean (SD) | 36.5 (13.29) | 39.6 (11.04) | 37.6 (12.55) |
| Median | 36.0 | 40.0 | 38.0 |
| Min, Max | 11, 68 | 17, 64 | 11, 68 |

TABLE 19
Stage 2 Baseline Efficacy Assessments-Stage 1 Placebo Non-Responders
Stage 2 mITT Population (N = 108)

| | | | All Stage 1 Placebo |
|---|---|---|---|
| Assessment | Placebo/Placebo | Placebo/d6-DM/Q | Non-Responder |
| Statistics | (N = 30) | (N = 33) | (N = 63) |
| NSA-16 Total Score | | | |
| N | 30 | 33 | 63 |
| Mean (SD) | 57.6 (9.39) | 57.6 (9.09) | 57.6 (9.16) |
| Median | 57.0 | 57.0 | 57.0 |
| Min, Max | 43, 86 | 43, 76 | 43, 86 |
| NSA-16 Factor Domain: Communication | | | |
| N | 30 | 33 | 63 |
| Mean (SD) | 12.0 (2.98) | 11.8 (3.12) | 11.9 (3.03) |
| Median | 12.0 | 12.0 | 12.0 |
| Min, Max | 7, 21 | 4, 19 | 4, 21 |
| NSA-16 Factor Domain: Emotion/Affect | | | |
| N | 30 | 33 | 63 |
| Mean (SD) | 11.7 (1.84) | 11.7 (2.09) | 11.7 (1.96) |
| Median | 11.0 | 11.0 | 11.0 |
| Min, Max | 8, 16 | 8, 16 | 8, 16 |
| NSA-16 Factor Domain: Social Involvement | | | |
| N | 30 | 33 | 63 |
| Mean (SD) | 12.1 (2.12) | 12.0 (2.56) | 12.0 (2.34) |
| Median | 12.0 | 13.0 | 12.0 |
| Min, Max | 8, 17 | 5, 16 | 5, 17 |
| NSA-16 Factor Domain: Motivation | | | |
| N | 30 | 33 | 63 |
| Mean (SD) | 15.8 (2.61) | 15.8 (2.39) | 15.8 (2.48) |
| Median | 16.0 | 16.0 | 16.0 |
| Min, Max | 11, 23 | 9, 19 | 9, 23 |
| NSA-16 Factor Domain: Retardation | | | |
| N | 30 | 33 | 63 |
| Mean (SD) | 6.0 (2.00) | 6.3 (2.05) | 6.2 (2.02) |
| Median | 6.0 | 6.0 | 6.0 |
| Min, Max | 2, 9 | 3, 11 | 2, 11 |
| NSA-4 Total Score | | | |
| N | 30 | 33 | 63 |
| Mean (SD) | 16.5 (2.64) | 16.4 (2.84) | 16.4 (2.72) |
| Median | 16.0 | 17.0 | 16.0 |
| Min, Max | 12, 24 | 8, 21 | 8, 24 |
| NSA-16 item 1: Prolonged Time to Respond | | | |
| N | 30 | 33 | 63 |
| Mean (SD) | 3.1 (1.21) | 2.5 (1.12) | 2.8 (1.20) |
| Median | 3.0 | 2.0 | 3.0 |
| Min, Max | 1, 6 | 1, 6 | 1, 6 |
| NSA-16 Item 2: Restricted Speech Quantity | | | |
| N | 30 | 33 | 63 |
| Mean (SD) | 3.4 (1.28) | 3.8 (1.25) | 3.6 (1.27) |
| Median | 3.0 | 4.0 | 3.0 |
| Min, Max | 1, 6 | 1, 6 | 1, 6 |
| NSA-16 item 3: Impoverished Speech Content | | | |
| N | 30 | 33 | 63 |
| Mean (SD) | 3.7 (0.80) | 3.4 (0.90) | 3.5 (0.86) |
| Median | 4.0 | 3.0 | 4.0 |
| Min, Max | 2, 5 | 1, 5 | 1, 5 |
| NSA-16 Item 4: Inarticulate Speech | | | |
| N | 30 | 33 | 63 |
| Mean (SD) | 1.9 (1.01) | 2.2 (1.18) | 2.0 (1.10) |
| Median | 2.0 | 2.0 | 2.0 |
| Min, Max | 1, 4 | 1, 4 | 1, 4 |
| NSA-16 item 5: Emotion:Reduced Range | | | |
| N | 30 | 33 | 63 |
| Mean (SD) | 4.1 (0.74) | 4.1 (0.89) | 4.1 (0.82) |
| Median | 4.0 | 4.0 | 4.0 |
| Min, Max | 3, 6 | 2, 6 | 2, 6 |
| NSA-16 Item 6: Affect: Reduce Modulation Intensity | | | |
| N | 30 | 33 | 63 |
| Mean (SD) | 4.1 (0.69) | 4.0 (0.90) | 4.0 (0.60) |
| Median | 4.0 | 4.0 | 4.0 |
| Min, Max | 3, 5 | 2, 6 | 2, 6 |
| NSA-16 item 7: Affect:Reduced Display on Demand | | | |
| N | 30 | 33 | 63 |
| Mean (SD) | 3.5 (0.94) | 3.5 (1.15) | 3.5 (1.04) |
| Median | 4.0 | 4.0 | 4.0 |
| Min, Max | 2, 5 | 1, 6 | 1, 6 |
| NSA-16 Item 8: Reduced Social Drive | | | |
| N | 30 | 33 | 63 |
| Mean (SD) | 4.6 (0.67) | 4.2 (1.20) | 4.4 (0.99) |
| Median | 5.0 | 5.0 | 5.0 |
| Min, Max | 3, 6 | 1, 5 | 1, 6 |
| NSA-16 item 9: Poor Rapport With Interviewer | | | |
| N | 30 | 33 | 63 |
| Mean (SD) | 3.3 (1.09) | 3.5 (1.25) | 3.4 (1.17) |
| Median | 3.5 | 4.0 | 4.0 |
| Min, Max | 1, 5 | 1, 5 | 1 ,5 |
| NSA-16 Item 10: Sexual Interest | | | |
| N | 30 | 33 | 63 |
| Mean (SD) | 4.1 (128) | 4.3 (1.67) | 4.2 (1.49) |
| Median | 4.0 | 4.0 | 4.0 |
| Min, Max | 2, 6 | 1, 6 | 1, 6 |
| NSA-16 item 11: Poor Grooming and Hygiene | | | |
| N | 30 | 33 | 63 |
| Mean (SD) | 2.2 (1.34) | 2.3 (1.08) | 2.3 (1.20) |
| Median | 2.0 | 2.0 | 2.0 |
| Min, Max | 1, 5 | 1, 4 | 1, 5 |
| NSA-16 Item 12: Reduced Sense of Purpose | | | |
| N | 30 | 33 | 63 |
| Mean (SD) | 4.4 (1.10) | 4.5 (0.97) | 4.5 (1.03) |
| Median | 5.0 | 5.0 | 5.0 |
| Min, Max | 2, 6 | 3, 6 | 2, 6 |
| NSA-16 item 13: Reduced-Interests | | | |
| N | 30 | 33 | 63 |
| Mean (SD) | 4.4 (0.81) | 4.3 (1.01) | 4.3 (0.92) |
| Median | 4.0 | 5.0 | 5.0 |
| Min, Max | 3, 6 | 1, 5 | 1, 6 |
| NSA-16 Item 14: Reduced Daily Activity | | | |
| N | 30 | 33 | 63 |
| Mean (SD) | 4.8 (0.46) | 4.6 (0.78) | 4.7 (0.65) |
| Median | 5.0 | 5.0 | 5.0 |
| Min, Max | 4, 6 | 2, 6 | 2, 6 |
| NSA-16 item 15: Reduced Expressive Gestures | | | |
| N | 30 | 33 | 63 |
| Mean (SD) | 3.5 (1.25) | 3.8 (1.48) | 3.7 (1.38) |
| Median | 4.0 | 4.0 | 4.0 |
| Min, Max | 1, 5 | 1, 6 | 1, 6 |
| NSA-16 Item 16: Slowed Movements | | | |
| N | 30 | 33 | 63 |
| Mean (SD) | 2.5 (1.07) | 2.5 (1.18) | 2.5 (1.12) |
| Median | 3.0 | 2.0 | 3.0 |
| Min, Max | 1, 4 | 1, 5 | 1, 5 |
| NSA-16: Global Negative Symptoms Rating | | | |
| N | 30 | 33 | 63 |
| Mean (SD) | 4.3 (0.71) | 4.4 (0.75) | 4.4 (0.73) |
| Median | 4.0 | 4.0 | 4.0 |
| Min, Max | 3 ,6 | 3, 6 | 3, 6 |
| NSA-16: Global Level of Functioning | | | |
| N | 29 | 33 | 62 |
| Mean (SD) | 4.5 (0.74) | 4.4 (0.70) | 4.4 (0.72) |
| Median | 4.0 | 4.0 | 4.0 |
| Min, Max | 3, 6 | 3, 6 | 3, 6 |
| PANSS Total Score | | | |
| N | 30 | 33 | 63 |
| Mean (SD) | 67.1 (9.00) | 65.6 (8.07) | 66.3 (8.49) |
| Median | 66.5 | 64.0 | 66.0 |
| Min, Max | 51, 87 | 53, 80 | 51, 87 |
| PANSS Total Score Reduction >= 20% | 30 | 33 | 63 |
| Yes | 0 (0.0) | 0 (0.0) | 0 (0.0) |
| No | 30 (100.0) | 33 (100.0) | 63 (100.0) |
| PANSS Negative Subscale | | | |
| N | 30 | 33 | 63 |
| Mean (SD) | 24.4 (4.55) | 23.6 (4.53) | 24.0 (4.52) |
| Median | 25.0 | 23.0 | 24.0 |
| Min, Max | 16, 35 | 13, 32 | 13, 35 |
| PANSS Positive Subscale | | | |
| N | 30 | 33 | 63 |
| Mean (SD) | 13.3 (3.64) | 13.3 (3.39) | 13.2 (3.48) |
| Median | 13.0 | 13.0 | 13.0 |
| Min, Max | 8, 23 | 7, 22 | 7, 23 |
| PANSS General Psychopathology Subscale | | | |
| N | 30 | 33 | 63 |
| Mean (SD) | 29.7 (5.40) | 28.7 (4.84) | 29.2 (5.10) |
| Median | 29.0 | 28.0 | 29.0 |
| Min, Max | 20, 43 | 20, 39 | 20, 43 |
| PANSS Prosocial Factors | | | |
| N | 30 | 33 | 63 |
| Mean (SD) | 17.7 (3.27) | 17.0 (3.30) | 17.3 (3.28) |
| Median | 18.0 | 17.0 | 18.0 |
| Min, Max | 12, 26 | 11, 24 | 11, 26 |
| PANSS Marder Negative Factors | | | |
| N | 30 | 33 | 63 |
| Mean (SD) | 23.0 (4.62) | 22.3 (5.45) | 22.6 (5.05) |
| Median | 24.0 | 21.0 | 22.0 |
| Min, Max | 13, 34 | 8, 38 | 8, 38 |
| PANSS Excitement Component | | | |
| N | 30 | 33 | 63 |
| Mean (SD) | 5.8 (127) | 6.7 (2.59) | 6.3 (2.10) |
| Median | 5.5 | 6.0 | 6.0 |
| Min, Max | 5, 11 | 5, 14 | 5, 14 |
| CGI-S | | | |
| N | 30 | 31 | 61 |
| Mean (SD) | 3.8 (0.79) | 3.7 (0.69) | 3.8 (0.74) |
| Median | 4.0 | 4.0 | 4.0 |
| Min, Max | 3, 6 | 2, 5 | 2, 6 |
| CGI-S Group | 30 | 31 | 61 |
| Percent Change = <65% | 30 (100.0) | 31 (100.0) | 61 (100.0) |
| Percent Change >65% | 0 (0.0) | 0 (0.0) | 0 (0.0) |
| CDSS Total Score | | | |
| N | 30 | 33 | 63 |
| Mean (SD) | 1.0 (1.56) | 0.8 (1.54) | 0.9 (1.54) |
| Median | 0.0 | 0.0 | 0.0 |
| Min, Max | 0, 6 | 0, 7 | 0, 7 |
| MCCB Composite Score | | | |
| N | 27 | 30 | 57 |
| Mean (SD) | 31.7 (14.99) | 28.9 (10.69) | 30.2 (12.87) |
| Median | 32.0 | 31.0 | 32.0 |
| Min, Max | 7, 56 | 8, 50 | 7, 56 |
| MCCB Speed of Processing | | | |
| N | 30 | 32 | 62 |
| Mean (SD) | 36.7 (12.76) | 35.2 (11.68) | 35.9 (12.14) |
| Median | 34.5 | 35.5 | 35.5 |
| Min, Max | 7, 58 | 13, 58 | 7, 58 |
| MCCB Attention/Vigilance | | | |
| N | 29 | 31 | 60 |
| Mean (SD) | 40.4 (13.00) | 36.2 (12.98) | 38.3 (13.05) |
| Median | 40.0 | 36.0 | 36.5 |
| Min, Max | 17, 68 | 14, 67 | 14, 68 |
| MCCB Working Memory | | | |
| N | 30 | 32 | 62 |
| Mean (SD) | 38.8 (12.21) | 36.0 (9.96) | 37.4 (11.11) |
| Median | 40.0 | 39.0 | 39.5 |
| Min, Max | 15, 60 | 15, 53 | 15, 60 |
| MCCB Verbal Learning | | | |
| N | 30 | 32 | 62 |
| Mean (SD) | 36.3 (7.35) | 34.8 (6.77) | 35.6 (7.04) |
| Median | 36.0 | 33.0 | 34.0 |
| Min, Max | 22, 55 | 25, 56 | 22, 56 |
| MCCB Visual Learning | | | |
| N | 29 | 31 | 60 |
| Mean (SD) | 37 (13.46) | 39.9 (9.69) | 38.7 (11.64) |
| Median | 36.0 | 40.0 | 36.5 |
| Min, Max | 16, 61 | 21, 57 | 16, 61 |
| MCCB Reasoning and Problem Solving | | | |
| N | 30 | 32 | 62 |
| Mean (SD) | 43.4 (9.76) | 42.8 (8.45) | 43.1 (9.04) |
| Median | 41.0 | 42.0 | 41.0 |
| Min, Max | 26, 68 | 29, 57 | 26, 66 |
| MCCB Social Cognition | | | |
| N | 29 | 32 | 61 |
| Mean (SD) | 38.0 (14.80) | 34.5 (12.91) | 36.2 (13.84) |
| Median | 36.0 | 33.5 | 36.0 |
| Min, Max | 14, 73 | 10, 62 | 10, 73 |

TABLE 20
Stage 2 Baseline Efficacy Assessments-Stage 1 Placebo Responders
Stage 2 mITT Population (N = 108)

| | | | | | All Stage 1 Placebo | |
|---|---|---|---|---|---|---|
| Assessment | Placebo/Placebo | | Placebo/d6-DM/Q | | Responder | |
| Statistics | (N = 2) | | (N = 1) | | (N = 3) | |
| NSA-16 Total Score | | | | | | |
| N | 2 | | 1 | | 3 | |
| Mean (SD) | 48.0 | (4.24) | 46.0 | (na) | 47.3 | (3.21) |
| Median | 48.0 | | 46.0 | | 46.0 | |
| Min, Max | 45, 51 | | 46, 46 | | 45, 51 | |
| NSA-16 Factor Domain: Communication | | | | | | |
| N | 2 | | 1 | | 3 | |
| Mean (SD) | 10.0 | (0.00) | 9.0 | (na) | 9.7 | (0.58) |
| Median | 10.0 | | 9.0 | | 10.0 | |
| Min, Max | 10, 10 | | 9, 9 | | 9, 10 | |
| NSA-16 Factor Domain: Emotion/Affect | | | | | | |
| N | 2 | | 1 | | 3 | |
| Mean (SD) | 9.5 | (0.71) | 9.0 | (na) | 9.3 | (0.58) |
| Median | 9.5 | | 9.0 | | 9.0 | |
| Min, Max | 9, 10 | | 9, 9 | | 9, 10 | |
| NSA-16 Factor Domain: Social Involvement | | | | | | |
| N | 2 | | 1 | | 3 | |
| Mean (SD) | 7.5 | (2.12) | 13.0 | (na) | 9.3 | (3.51) |
| Median | 7.5 | | 13.0 | | 9.0 | |
| Min, Max | 6, 9 | | 13, 13 | | 6, 13 | |
| NSA-16 Factor Domain: Motivation | | | | | | |
| N | 2 | | 1 | | 3 | |
| Mean (SD) | 15.5 | (0.71) | 12.0 | (na) | 14.3 | (2.08) |
| Median | 15.5 | | 12.0 | | 15.0 | |
| Min, Max | 15, 16 | | 12, 12 | | 12, 16 | |
| NSA-I6 Factor Domain: Retardation | | | | | | |
| N | 2 | | 1 | | 3 | |
| Mean (SD) | 5.5 | (0.71) | 3.0 | (na) | 4.7 | (1.53) |
| Median | 5.5 | | 3.0 | | 5.0 | |
| Min, Max | 5, 6 | | 3, 3 | | 3, 6 | |
| NSA-4 Total Score | | | | | | |
| N | 2 | | 1 | | 3 | |
| Mean (SD) | 15.0 | (1.41) | 13.0 | (na) | 14.3 | (1.53) |
| Median | 15.0 | | 13.0 | | 14.0 | |
| Min, Max | 14, 16 | | 13, 13 | | 13, 16 | |
| NSA-16 item 1: Prolonged Time to Respond | | | | | | |
| N | 2 | | 1 | | 3 | |
| Mean (SD) | 2.0 | (0.00) | 2.0 | (na) | 2.0 | (0.00) |
| Median | 2.0 | | 2.0 | | 2.0 | |
| Min, Max | 2, 2 | | 2, 2 | | 2, 2 | |
| NSA-16 Item 2: Restricted Speech Quantity | | | | | | |
| N | 2 | | 1 | | 3 | |
| Mean (SD) | 3.0 | (0, 00) | 3.0 | (na) | 3.0 | (0.00) |
| Median | 3.0 | | 3.0 | | 3.0 | |
| Min, Max | 3, 3 | | 3, 3 | | 3, 3 | |
| NSA-16 item 3: Impoverished Speech Content | | | | | | |
| N | 2 | | 1 | | 3 | |
| Mean (SD) | 3.0 | (0.00) | 3.0 | (na) | 3.0 | (0.00) |
| Median | 3.0 | | 3.0 | | 3.0 | |
| Min, Max | 3, 3 | | 3, 3 | | 3, 3 | |
| NSA-16 Item 4: Inarticulate Speech | | | | | | |
| N | 2 | | 1 | | 3 | |
| Mean (SD) | 2.0 | (0.00) | 1.0 | (na) | 1.7 | (0.58) |
| Median | 2.0 | | 1.0 | | 2.0 | |
| Min, Max | 2, 2 | | 1, 1 | | 1, 2 | |
| NSA-16 item 5: Emotion:Reduced Range | | | | | | |
| N | 2 | | 1 | | 3 | |
| Mean (SD) | 3.5 | (0.71) | 3.0 | (na) | 3.3 | (0.58) |
| Median | 3.5 | | 3.0 | | 3.0 | |
| Min, Max | 3, 4 | | 3, 3 | | 3, 4 | |
| NSA-16 Item 6: Affect: Reduce Modulation Intensity | | | | | | |
| N | 2 | | 1 | | 3 | |
| Mean (SD) | 3.5 | (0.71) | 3.0 | (na) | 3.3 | (0.58) |
| Median | 3.5 | | 3.0 | | 3.0 | |
| Min, Max | 3, 4 | | 3, 3 | | 3, 4 | |
| NSA-16 item 7: Affect: Reduced Display on Demand | | | | | | |
| N | 2 | | 1 | | 3 | |
| Mean (SD) | 2.5 | (0.71) | 3.0 | (na) | 27 | (0.58) |
| Median | 2.5 | | 3.0 | | 3.0 | |
| Min, Max | 2, 3 | | 3, 3 | | 2, 3 | |
| NSA-16 Item 8: Reduced Social Drive | | | | | | |
| N | 2 | | 1 | | 3 | |
| Mean (SD) | 4.0 | (0.00) | 4.0 | (na) | 4.0 | (0.00) |
| Median | 4.0 | | 4.0 | | 4.0 | |
| Min, Max | 4, 4 | | 4, 4 | | 4, 4 | |
| NSA-16 item 9: Poor Rapport With Interviewer | | | | | | |
| N | 2 | | 1 | | 3 | |
| Mean (SD) | 2.0 | (1.41) | 3.0 | (na) | 2.3 | (1.15) |
| Median | 9.0 | | 3.0 | | 3.0 | |
| Min, Max | 1, 3 | | 3, 3 | | 1, 3 | |
| NSA-16 Item 10: Sexual Interest | | | | | | |
| N | 2 | | 1 | | 3 | |
| Mean (SD) | 1.5 | (0.71) | 6.0 | (na) | 3.0 | (2.65) |
| Median | 1.5 | | 6.0 | | 2.0 | |
| Min, Max | 1, 2 | | 6, 6 | | 1, 6 | |
| NSA-16 item 11: Poor Grooming and Hygiene | | | | | | |
| N | 2 | | 1 | | 3 | |
| Mean (SD) | 1.5 | (0.71) | 10 | (na) | 1.3 | (0.58) |
| Median | 1.5 | | 1.0 | | 1.0 | |
| Mm. Max | 1, 2 | | 1, 1 | | 1, 2 | |
| NSA-16 Item 12: Reduced Sense of Purpose | | | | | | |
| N | 2 | | 1 | | 3 | |
| Mean (SD) | 5.0 | (0.00) | 4.0 | (na) | 4.7 | (0.58) |
| Median | 5.0 | | 4.0 | | 5.0 | |
| Min, Max | 5, 5 | | 4, 4 | | 4, 5 | |
| NSA-16 item 13: Reduced Interests | | | | | | |
| N | 2 | | 1 | | 3 | |
| Mean (SD) | 4.5 | (0.71) | 3.0 | (na) | 4.0 | (1.00) |
| Median | 4.5 | | 3.0 | | 4.0 | |
| Min, Max | 4, 5 | | 3, 3 | | 3, 5 | |
| NSA-16 Item 14: Reduced Daily Activity | | | | | | |
| N | 2 | | 1 | | 3 | |
| Mean (SD) | 4.5 | (0.71) | 4.0 | (na) | 4.3 | (0.58) |
| Median | 4.5 | | 4.0 | | 4.0 | |
| Min, Max | 4, 5 | | 4, 4 | | 4, 5 | |
| NSA-16 item 15: Reduced Expressive Gestures | | | | | | |
| N | 2 | | 1 | | 3 | |
| Mean (SD) | 3.5 | (0.71) | 2.0 | (na) | 3.0 | (1.00) |
| Median | 3.5 | | 2.0 | | 3.0 | |
| Min, Max | 3, 4 | | 2, 2 | | 2, 4 | |
| NSA-16 Item 16: Slowed Movements | | | | | | |
| N | 2 | | 1 | | 3 | |
| Mean (SD) | 2.0 | (0.00) | 1.0 | (na) | 1.7 | (0.58) |
| Median | 2.0 | | 1.0 | | 2.0 | |
| Min, Max | 2, 2 | | 1, 1 | | 1, 2 | |
| NSA-16: Global Negative Symptoms Rating | | | | | | |
| N | 2 | | 1 | | 3 | |
| Mean (SD) | 3.5 | (0.71) | 3.0 | (na) | 3.3 | (0.58) |
| Median | 3.5 | | 3.0 | | 3.0 | |
| Min, Max | 3, 4 | | 3, 3 | | 3, 4 | |
| NSA 16: Global Level of Functioning | | | | | | |
| N | 2 | | 1 | | 3 | |
| Mean (SD) | 3.5 | (0.71) | 3.0 | (na) | 3.3 | (0.58) |
| Median | 3.5 | | 3.0 | | 3.0 | |
| Min, Max | 3, 4 | | 3, 3 | | 3, 4 | |
| PANSS Total Score | | | | | | |
| N | 2 | | 1 | | 3 | |
| Mean (SD) | 51.5 | (0.71) | 51.0 | (na) | 51.3 | (0.58) |
| Median | 51.5 | | 51.0 | | 51.0 | |
| Min, Max | 51, 52 | | 51, 51 | | 51, 52 | |
| PANSS Total Score Reduction >= 20% | 2 | | 1 | | 3 | |
| Yes | 2 | (100.0) | 1 | (100.0) | 3 | (100.0) |
| No | 0 | (0.0) | 0 | (0.0) | 0 | (0.0) |
| PANSS Negative Subscale | | | | | | |
| N | 2 | | 1 | | 3 | |
| Mean (SD) | 18.5 | (2.12) | 20.0 | (na) | 19.0 | (1.73) |
| Median | 18.5 | | 20.0 | | 20.0 | |
| Min, Max | 17, 20 | | 20, 20 | | 17, 20 | |
| PANSS Positive Subscale | | | | | | |
| N | 2 | | 1 | | 3 | |
| Mean (SD) | 10.0 | (0.00) | 7.0 | (na) | 9.0 | (1.73) |
| Median | 10.0 | | 7.0 | | 10.0 | |
| Min, Max | 10, 10 | | 7, 7 | | 7, 10 | |
| PANSS General Psychopathology Subscale | | | | | | |
| N | 2 | | 1 | | 3 | |
| Mean (SD) | 23.0 | (1.41) | 24.0 | (na) | 23.3 | (1.5) |
| Median | 23.0 | | 24.0 | | 24.0 | |
| Min, Max | 22, 24 | | 24, 24 | | 22, 24 | |
| PANSS Prosocial Factors | | | | | | |
| N | 2 | | 1 | | 3 | |
| Mean (SD) | 135 | (0.71) | 12.0 | (na) | 13.0 | (1.00) |
| Median | 13.5 | | 12.0 | | 13.0 | |
| Min, Max | 13, 14 | | 12, 12 | | 12, 14 | |
| PANSS Marder Negative Factors | | | | | | |
| N | 2 | | 1 | | 3 | |
| Mean (SD) | 19.0 | (2.83) | 19.0 | (na) | 19.0 | (2.00) |
| Median | 19.0 | | 19.0 | | 19.0 | |
| Min, Max | 17, 21 | | 19, 19 | | 17, 21 | |
| PANSS Excitement Component | | | | | | |
| N | 2 | | 1 | | 3 | |
| Mean (SD) | 6.0 | (1.41) | 5.0 | (na) | 5.7 | (1.15) |
| Median | 6.0 | | 5.0 | | 5.0 | |
| Min, Max | 5, 7 | | 5, 5 | | 5, 7 | |
| CGI-S | | | | | | |
| N | 2 | | 1 | | 3 | |
| Mean (SD) | 3.0 | (0.00) | 3.0 | (na) | 3.0 | (0.00) |
| Median | 3.0 | | 3.0 | | 3.0 | |
| Min, Max | 3, 3 | | 3, 3 | | 3, 3 | |
| CGI-S Group | 2 | | 1 | | 3 | |
| Percent Change =< 65% | 2 | (100.0) | 1 | (100.0) | 3 | (100.0) |
| Percent Change > 65% | 0 | (0.0) | 0 | (0.0) | 0 | (0, 0) |
| CDSS Total Score | | | | | | |
| N | 2 | | 1 | | 3 | |
| Mean (SD) | 0.5 | (0.71) | 0.0 | (na) | 0.3 | (0.58) |
| Median | 0.5 | | 0.0 | | 0.0 | |
| Min, Max | 0, 1 | | 0, 0 | | 0, 1 | |
| MCCB Composite Score | | | | | | |
| N | 2 | | 1 | | 3 | |
| Mean (SD) | 35.0 | (11.31) | 41.0 | (na) | 37.0 | (8.72) |
| Median | 35.0 | | 41.0 | | 41.0 | |
| Min, Max | 27, 43 | | 41, 41 | | 27, 43 | |
| MCCB Speed of Processing | | | | | | |
| N | 2 | | 1 | | 3 | |
| Mean (SD) | 32, 0 | (4.24) | 47.0 | (na) | 37.0 | (9.17) |
| Median | 32.0 | | 47.0 | | 35.0 | |
| Min, Max | 29, 35 | | 47, 47 | | 29, 47 | |
| MCCB Attention/Vigilance | | | | | | |
| N | 2 | | 1 | | 3 | |
| Mean (SD) | 45.5 | (13.44) | 39.0 | (na) | 43.3 | (10.21) |
| Median | 45.5 | | 39.0 | | 39.0 | |
| Min, Max | 36, 55 | | 39, 39 | | 36, 55 | |
| MCCB Working Memory | | | | | | |
| N | 2 | | 1 | | 3 | |
| Mean (SD) | 45.5 | (6.36) | 50.0 | (na) | 47.0 | (5.20) |
| Median | 45.5 | | 50.0 | | 50.0 | |
| Min, Max | 41, 50 | | 50, 50 | | 41, 50 | |
| MCCB Verbal Learning | | | | | | |
| N | 2 | | 1 | | 3 | |
| Mean (SD) | 35.5 | (3.54) | 46.0 | (na) | 39.0 | (6.56) |
| Median | 35.5 | | 46.0 | | 38.0 | |
| Min, Max | 33, 38 | | 46, 46 | | 33, 46 | |
| MCCB Visual Learning | | | | | | |
| N | 2 | | 1 | | 3 | |
| Mean (SD) | 38.5 | (0.71) | 50.0 | (na) | 42.3 | (6.66) |
| Median | 38.5 | | 50.0 | | 39.0 | |
| Min, Max | 38, 39 | | 50, 50 | | 38, 50 | |
| MCCB Reasoning and Problem Solving | | | | | | |
| N | 2 | | 1 | | 3 | |
| Mean (SD) | 46.0 | (5.66) | 46.0 | (na) | 46.0 | (4.00) |
| Median | 46.0 | | 46.0 | | 46.0 | |
| Min, Max | 42, 50 | | 46, 46 | | 42, 50 | |
| MCCB Social Cognition | | | | | | |
| N | 2 | | 1 | | 3 | |
| Mean (SD) | 44.5 | (17.68) | 35.0 | (na) | 41.3 | (13.65) |

| | | | |
|---|---|---|---|
| Median | 44.5 | 35.0 | 35.0 |
| Min, Max | 32, 57 | 35, 35 | 32, 57 |

TABLE 21
PGIC Score: Change from Baseline Parallel Group MMRM Analysis, Observed Data
mITT 12-Week Parallel Group (N = 87)

| Visit/Statistics | Placebo/Placebo | | d6-DM/Q/d6-DM/Q | |
|---|---|---|---|---|
| Week 6 Change from Baseline: N, Mean (SD) | 37, 3.2 | (0.89) | 47, 3.1 | (0.94) |
| Week 12 Change from Baseline: N. Mean (SD) | 32, 3.2 | (0.88) | 42, 2.9 | (0.95) |

| | | |
|---|---|---|
| Week 12 Treatment Difference vs. Placebo: p-value, LS Mean Difference (95% CI) | 0.106, −0.35 | (−0.77, 0.08) |

Note:
PGIC Score ranges from 1 to 7, with higher scores indicating greater clinical severity of symptoms. Patients within each treatment group received the same treatment throughout their participation in the study. Repeated measures model includes fixed effect for treatment, visit, treatment-by-visit interaction, baseline value, and baseline value-by-visit interaction, An unstructured covariance matrix was used.

TABLE 22
PGIC Score: Proportional Odds Regression by Stage, Observed Data
mITT 12-Week Parallel Group Population (N = 87)

| Visit | Result/Statistics | Placebo/Placebo | | d6-DM/Q/d6-DM/Q | |
|---|---|---|---|---|---|
| Stage 1, Week 6 (Relative to Baseline) | N | 37 | | 47 | |
| Stage 1, Week 6 (Relative to Baseline) | 1 = Very much improved, n (%) | 2 | (5.4) | 2 | (4.3) |
| Stage 1, Week 6 (Relative to Baseline) | 2= Much improved, n (%) | 5 | (13.5) | 11 | (23.4) |
| Stage 1, Week 6 (Relative to Baseline) | 3 = Minimally improved, n (%) | 13 | (35.1) | 15 | (31.9) |
| Stage 1, Week 6 (Relative to Baseline) | 4 = No change, n (%) | 17 | (45.9) | 18 | (38.3) |
| Stage 1, Week 6 (Relative to Baseline) | 5 = Minimally worse, n (%) | 0 | (0.0) | 1 | (2.1) |
| Stage 1, Week 6 (Relative to Baseline) | 6 = Much worse, n (%) | 0 | (0.0) | 0 | (0.0) |
| Stage 1, Week 6 (Relative to Baseline) | 7 = Very much worse, n (%) | 0 | (0.0) | 0 | (0.0) |
| Stage 1, Week 6 (Relative to Baseline) | Odds ratio (d6-DM/Q to Placebo) (95% CI), p-value | | | 1.29 (0.58, 2.86), 0.530 | |
| Stage 2, Week 12 (Relative to Stage 1 Baseline) | N | 32 | | 42 | |
| Stage 2, Week 12 (Relative to Stage 1 Baseline) | 1 = Very much improved, n (%) | 2 | (6.3) | 4 | (9.5) |
| Stage 2, Week 12 (Relative to Stage 1 Baseline) | 2 = Much improved, n (%) | 4 | (12.5) | 10 | (23.8) |
| Stage 2, Week 12 (Relative to Stage 1 Baseline) | 3 = Minimally improved, n (%) | 13 | (40.6) | 16 | (38.1) |
| Stage 2, Week 12 (Relative to Stage 1 Baseline) | 4 = No change, n (%) | 13 | (40.6) | 12 | (28.6) |
| Stage 2, Week 12 (Relative to Stage 1 Baseline) | 5 = Minimally worse, n (%) | 0 | (0.0) | 0 | (0.0) |
| Stage 2, Week 12 (Relative to Stage 1 Baseline) | 6 = Much worse, n (%) | 0 | (0.0) | 0 | (0.0) |
| Stage 2, Week 12 (Relative to Stage 1 Baseline) | 7 = Very much worse, n (%) | 0 | (0.0) | 0 | (0.0) |

| | | |
|---|---|---|
| Stage 2, Week 12 (Relative to Stage 1 Baseline) | Odds ratio (d6-DM/Q to Placebo) (95% CI), p-value | 1.85 (0.79, 4.36), 0.158 |

p-values are calculated using proportional odds regression analysis.

TABLE 23
NSA-16 Total Score: Change from Baseline SPCD SUR, LOCF Data
mITT Population (N = 127)

| | Visit | | | | |
|---|---|---|---|---|---|
| Stage | Statistics | Placebo | | d6-DM/Q | |
| Stage 1 | N | 80 | | 47 | |
| Stage 1 | Baseline: Mean (SD) | 60.4 | (7.71) | 61.0 | (7.53) |
| Stage 1 | Week 6: Mean (SD) | 57.4 | (9.21) | 55.9 | (8.75) |
| Stage 1 | Change from Baseline Mean (SD) | −3.0 | (5.78) | −5.0 | (5.64) |
| Stage 1 | Standard Effect Size | | | −0.365 | |
| Stage 1 | SUR Estimated Difference versus Placebo (SE) | | | −2.05 | (1.05) |
| Stage 2 (Stage 1 Placebo Non-responders) | N | 30 | | 33 | |
| Stage 2 (Stage 1 Placebo Non-responders) | Baseline: Mean (SD) | 57.6 | (9.39) | 57.6 | (9.09) |
| Stage 2 (Stage 1 Placebo Non-responders) | Week 6: Mean (SD) | 55.4 | (11.28) | 54.0 | (8.63) |
| Stage 2 (Stage 1 Placebo Non-responders) | Change from Baseline Mean (SD) | −2.2 | (5.89) | −3.6 | (6.34) |

| Stage 2 (Stage 1 Placebo Non-responders) | Standard Effect Size | −0.230 | |
|---|---|---|---|
| Stage 2 (Stage 1 Placebo Non-responders) | SUR Estimated Difference versus Placebo (SE) | −1.26 | (1.52) |

| | |
|---|---|
| SUR z−statistic, overall p-value | −1.98, 0.0481 |

Note:
NSA-16 Total Score ranges from 16 to 96, with higher scores indicating greater clinical severity of symptoms. Standard Effect Size is defined as (mean change in d6-DM/Q − mean change in Placebo)/Change from Baseline Pooled SD. Stage 2 Baseline is the last non-missing assessment prior to re-randomization into Stage 2 (re-randomization visit), SPCD SUR z-statistic was calculated using Stage 1 weight = 0.6 and Stage 2 weight = 0.4.

TABLE 24
NSA-16 Total Score: Change from Baseline SPCD ANCOVA, LOCF Data
mITT Population (N = 127)

| | Visit | | | | |
|---|---|---|---|---|---|
| Stage | Statistics | Placebo | | d6-DM/Q | |
| Stage 1 | Baseline: N, Mean (SD) | 80, 60.4 | (7.71) | 47, 61.0 | (7,53) |
| | Week 6: N, Mean (SD) | 80, 57.4 | (9.21) | 47, 55.9 | (6.75) |
| | Change from Baseline: N, Mean (SD) | 80, −3.0 | (5.78) | 47, −5.0 | (5.64) |
| | Standard Effect Size | | | −0.365 | |
| | % Change from Baseline: N, Mean (SD) | 80, −4.8 | (9.47) | 47, −8.2 | (9.36) |
| | Treatment Difference vs. Placebo: LS Mean Difference, 95% CI [1] | | | −2,05 | (−4.13, 0.04) |
| | p−value [1] | | | 0.054 | |
| Stage 2 (Stage 1 Placebo Non-Baseline [2]; N, Mean (SD) | | 30, 57.6 | (9.39) | 33, 57.6 | (9.09) |
| responders) | | | | | |
| | Week 12: N, Mean (SD) | 30, 55.4 | (11.28) | 33, 54.0 | (8.63) |
| | Change from Baseline [2]: N, Mean (SD) | 30, −2.2 | (5,89) | 33, −3.6 | (6.34) |
| | Standard Effect Size | | | −0.230 | |
| | % Change from Baseline [2]: N, Mean (SD) | 30, −4.0 | (981) | 33, −5.6 | (10.70) |
| | Treatment Difference vs. Placebo: LS Mean Difference, 95% CI [1] | | | −1.40 | (−4.46, 1.65) |

| | |
|---|---|
| p−value [1] | 0.362 |
| SPCD Weighted OLS z-statistic, overall p-value [3] | −2.04, 0.042 |

Note:
NSA-16 Total Score ranges from 16 to 96, with higher scores indicating greater clinical severity of symptoms. Standard Effect Size is defined as (mean change in d6-DM/Q − mean change in Placebo)/Change from Baseline Pooled SD.
[1] Change from Baseline was analyzed at each stage by ANCOVA with treatment as fixed effect and baseline value as covariate. Missing values were imputed by LOCF within each stage.
[2] Stage 2 Baseline is the last non-missing assessment prior to re-randomization into Stage 2 (re-randomization visit).
[3] SPCD Weighted OLS z-statistic was calculated using Stage 1 weight = 0.6 and Stage 2 weight = 0.4.

TABLE 25
NSA-16 Total Score: Change from Baseline SPCD MMRM, Observed Data
Per Protocol Population (N = 110)

| | Visit | | | | |
|---|---|---|---|---|---|
| Stage | Statistics | Placebo | | d6-DM/Q | |
| Stage 1 | Baseline: N, Mean (SD) | 73, 60.9 | (7.57) | 37, 60.4 | (7.83) |
| | Week 3 Change from Baseline: N, Mean (SD) | 72, −3.0 | (6.53) | 37, −1.8 | (5.60) |
| | Treatment Difference vs. Placebo: LS Mean Difference, 95% CI [1] | | | 1.19 | (−1.29, 3.67) |
| | p-value [1] | | | 0.342 | |
| | Week 6 Change from Baseline: N, Mean (SD) | 63, −3.5 | (5.76) | 37, −4,8 | (5.77) |
| | Treatment Difference vs, Placebo: LS Mean Difference, 95% CI [1] | | | −1.57 | (−3.94, 0.80) |
| | p-value [1] | | | 0.191 | |
| Stage 2 (Stage 1 Placebo Non-Baseline [2]: N, Mean (SD) | | 27, 57.4 | (9.85) | 29, 59.0 | (8.67) |
| responders) | | | | | |
| | Week 9 Change from Baseline [2]: N, Mean (SD) | 27, −0.4 | (5.42) | 29, −4.2 | (5.98) |
| | Treatment Difference vs. Placebo: LS Mean Difference, 95% CI [1] | | | −3.65 | (−6.73, −0.56) |
| | p-value [1] | | | 0.021 | |
| | Week 12 Change from Baseline [2]: N, Mean (SD) | 26, −3,0 | (5.81) | 29, −4.5 | (5.96) |

| | | |
|---|---|---|
| Treatment Difference vs. Placebo: LS Mean Difference, 95% CI [1] | −1.43 | (−4.63, 1.76) |

| | |
|---|---|
| p-value [1] | 0.372 |
| SPCD Week 6 and 12 MMRM Weighted z-statistic, overall p-value [3] | −1.58, 0.114 |

Note:
NSA-16 Total Score ranges from 16 to 96, with higher scores indicating greater clinical severity of symptoms.
[1] MMRM with fixed effect of treatment, visit, treatment-by-visit interaction, baseline NSA-16, and baseline NSA-16-by-visit interaction. An unstructured covariance matrix was used.
[2] Stage 2 Baseline is the last non-missing assessment prior to re-randomization into Stage 2 (re-randomization visit).
[3] SPCD Weighted a-statistic was calculated using Stage 1 weight = 0.6 and Stage 2 weight = 0.4. Treatment differences in each stage were estimated by the MMRM.

TABLE 26
NSA-16 Total Score: Change from Baseline SPCD ANCOVA, LOCF Data
mITT Population (N = 127)
Subgroup: Band-Pass Filter #1

| | Visit | | | | |
|---|---|---|---|---|---|
| Stage | Statistics | Placebo | | d6-DM/Q | |
| Stage 1 | Baseline: N Mean (SD) | 14, 60.9 | (7.59) | 19, 63.9 | (7.61) |
| | Week 6: N, Mean (SD) | 14, 61.8 | (10.64) | 19, 59.5 | (7.36) |
| | Change from Baseline: N, Mean (SD) | 14, 0.9 | (6.87) | 19, −4.5 | (5.12) |
| | Standard Effect Size | | | −0.900 | |
| | % Change from Baseline: N, Mean (SD) | 14, 1.3 | (11.61) | 19, −6.7 | (8.28) |
| | Treatment Difference vs. Placebo: LS Mean Difference, 95% CI [1] | | | −4.97 | (−9.33, −0.60) |
| | p-value [1] | | | 0.027 | |
| Stage 2 (Stage 1 Placebo Non-Baseline [2]: N, Mean (SD) | | 1, 72.0 | ( ) | 11, 59.8 | (10.94) |
| responders) | | | | | |
| | Week 12: N, Mean (SD) | 1, 74.0 | ( ) | 11, 55.0 | (9.55) |
| | Change from Baseline [2]: N, Mean (SD) | 1, 2.0 | ( ) | 11, −4.8 | (7.83) |
| | Standard Effect Size | | | | |
| | % Change from Baseline [2]: N, Mean (SD) | 1, 2.8 | ( ) | 11, −7.2 | (11.64) |
| | Treatment Difference vs. Placebo: LS Mean Difference, 95% CI [1] | | | −11.39 | (−28.92, 6.14) |

| | |
|---|---|
| p-value [1] | 0.176 |
| SPCD Weighted OLS z-statistic, overall p-value [3] | −2.25, 0.025 |

Note:
Band-pass filter: Any site with mean placebo NSA-16 total score changes from baseline in Stage 1 (Baseline to Week 6) more than 0 or less than −7 is excluded. Standard Effect Size is defined as (mean change in d6-DM/Q-mean change in Placebo)/Change from Baseline Pooled SD.
[1] Change from Baseline was analyzed at each stage by ANCOVA with treatment as fixed effect and baseline value as covariate. Missing values were imputed by LOCF within each stage.
[2] Stage 2 Baseline is the last non-missing assessment prior to re-randomization into Stage 2 (re-randomization visit).
[3] SPCD Weighted OLS z-statistic was calculated using Stage 1 weight = 0.6 and Stage 2 weight = 0.4,
Note:
Band-pass filter: Any site with mean placebo NSA-16 total score changes from baseline in Stage 1 (Baseline to Week 6) more than 0 or less than −7 is excluded.

TABLE 27
NSA-16 Total Score: Change from Baseline Parallel Group MMRM Analysis, Observed Data
mITT 12-Week Parallel Group Population (N = 87)
Subgroup: Band-Pass Filter #1

| Visit/Statistics | Placebo/Placebo | | d6-DM/Q/d6-DM/Q | |
|---|---|---|---|---|
| Baseline: N, Mean (SD) | 2, 70.5 | (2,12) | 19, 63.9 | (7.61) |
| Week 3 Change from Baseline: N, Mean (SD) | 2, −4.5 | (3.54) | 18, −2.1 | (5.98) |
| Week 6 Change from Baseline: N, Mean (SD) | 1, 0.0 | (NA) | 19, −4.5 | (5.12) |
| Week 9 Change from Baseline: N, Mean (SD) | 1, 7.0 | (NA) | 18, −7.2 | (8.43) |
| Week 12 Change from Baseline: N, Mean (SD) | 1, 2.0 | (NA) | 18, −3.3 | (7.85) |

| | | |
|---|---|---|
| Week 12 Treatment Difference vs. Placebo: p-value, LS Mean Difference (95% CI) | 0.329, −8.38 | (−26.02, 9.27) |

Note:
NSA-16 Total Score ranges from 16 to 96, with higher scores indicating greater clinical severity of symptoms.
Note:
Band-pass filter: Any site with mean placebo NSA-16 total score changes from baseline in Stage 1 (Baseline to Week 6) more than 0 or less than −7 is excluded. Patients within each treatment group received the same treatment throughout their participation in the study. Repeated measures model includes fixed effect for treatment, visit, treatment-by-visit interaction, baseline value, and baseline value-by-visit interaction, An unstructured covariance matrix was used.

TABLE 28
PANSS Total Score: Change from Baseline SPCD MMRM, Observed Data
mITT Population (N = 127)

| | Visit | | | | |
|---|---|---|---|---|---|
| Stage | Statistics | Placebo | | d6-DM/Q | |
| Stage 1 | Baseline: N, Mean (SD) | 80, 68.7 | (7.99) | 47, 67.4 | (8,26) |
| | Week 3 Change from Baseline: N, Mean (SD) | 79, −2.4 | (6.97) | 45, −3.2 | (6.84) |
| | Treatment Difference vs. Placebo: LS Mean Difference 95% CI [1] | | | −1.08 | (−3.55, 1.39) |
| | p-value [1] | | | 0.389 | |
| | Week 6 Change from Baseline: N, Mean (SD) | 70, −2.5 | (6.50) | 47, −4.7 | (6.98) |
| | Treatment Difference vs. Placebo: LS Mean Difference, 95% CI [1] | | | −2.36 | (−4.77, 0.06) |
| | p-value [1] | | | 0.055 | |
| Stage 2 (Stage 1 Placebo Non-Baseline [2]: N, Mean (SD) | | 30, 67.1 | (9.00) | 33, 65.6 | (8.07) |
| responders) | | | | | |
| | Week 9 Change from Baseline [2]: N, Mean (SD) | 30, 0.2 | (5.60) | 33, −2.5 | (8.26) |
| | Treatment Difference vs. Placebo: LS Mean Difference, 95% CI [1] | | | −3.04 | (−6.54, 0.45) |
| | p-value [1] | | | 0.087 | |
| | Week 12 Change from Baseline [2]: N, Mean (SD) | 29, −1,4 | (7.64) | 32, −4.0 | (7.71) |

| | | |
|---|---|---|
| Treatment Difference vs. Placebo: LS Mean Difference, 95% CI [1] | −2.53 | (−6.51, 1.45) |

| | |
|---|---|
| p-value [1] | 0.209 |
| SPCD Week 6 and 12 MMRM Weighted z-statistic, overall p-value [3] | −2.25, 0.025 |

Note:
PANSS Total Score ranges from 30 to 210, with higher scores indicating greater clinical severity of symptoms.
[1] MMRM with fixed effect of treatment, visit, treatment-by-visit interaction, baseline PANSS Total Score and baseline PANSS Total Score-by-visit interaction. An unstructured covariance matrix was used.
[2] Stage 2 Baseline is the last non-missing assessment prior to re-randomization into Stage 2 (re-randomization visit).
[3] SPCD Weighted z-statistic was calculated using Stage 1 weight = 0.6 and Stage 2 weight = 0.4. Treatment differences in each stage were estimated by the MMRM.

TABLE 29
PANSS Positive Subscale: Change from Baseline SPCD MMRM, Observed Data
mITT Population (N = 127)

| | Visit | | | | |
|---|---|---|---|---|---|
| Stage | Statistics | Placebo | | d6-DM/Q | |
| Stage 1 | Baseline: N, Mean (SD) | 80, 13.4 | (2.81) | 47, 13.6 | (3.65) |
| | Week 3 Change from Baseline: N, Mean (SD) | 79, −0.3 | (2.53) | 45, −0.6 | (1.83) |
| | Treatment Difference vs. Placebo: LS Mean Difference, 95% CI [1] | | | −0.25 | (−1.08, 0.58) |
| | p-value [1] | | | 0.553 | |
| | Week 6 Change from Baseline: N, Mean (SD) | 79, −0.3 | (2.57) | 47, −0.8 | (2.63) |
| | Treatment Difference vs, Placebo: LS Mean Difference, 95% CI [1] | | | −0.42 | (−1.36, 0.52) |
| | p-value [1] | | | 0.376 | |
| Stage 2 (Stage 1 Placebo Non-Baseline [2]: N, Mean (SD) | | 30, 13.1 | (3,64) | 33, 13.3 | (3.39) |
| responders) | | | | | |
| | Week 9 Change from Baseline [2]: N, Mean (SD) | 30, 0.3 | (1.80) | 33, 0.2 | (2.97) |
| | Treatment Difference vs. Placebo: LS Mean Difference, 95% CI [1] | | | −0.10 | (−1.30, 1.10) |
| | p-value [1] | | | 0.864 | |
| | Week 12 Change from Baseline [2]: N, Mean (SD) | 29, −0.4 | (1.99) | 32, −0.3 | (2.47) |

| | | |
|---|---|---|
| Treatment Difference vs. Placebo: LS Mean Difference, 95% CI [1] | 0.28 | (−0.90, 1.45) |

| | |
|---|---|
| p-value [1] | 0.640 |
| SPCD Week 6 and 12 MMRM Weighted z-statistic, overall p-value [3] | −0.39, 0.700 |

Note:
PANSS Positive Subscale ranges from 7 to 49, with higher scores indicating greater clinical severity of symptoms.
[1] MMRM with fixed effect of treatment, visit, treatment-by-visit interaction, baseline PANSS Positive Subscale and baseline PANSS Positive Subscale-by-visit interaction. An unstructured covariance matrix was used,
[2] Stage 2 Baseline is the last non-missing assessment prior to re-randomization into Stage 2 (re-randomization visit).
[3] SPCD Weighted z-statistic was calculated using Stage 1 weight = 0.6 and Stage 2 weight = 0.4. Treatment differences in each stage were estimated by the MMRM,

TABLE 30
PANSS Negative Subscale: Change from Baseline SPCD MMRM, Observed Data
mITT Population (N = 127)

| | Visit | | | | |
|---|---|---|---|---|---|
| Stage | Statistics | Placebo | | d6-DM/Q | |
| Stage 1 | Baseline: N, Mean (SD) | 80, 25.2 | (3.64) | 47, 24.6 | (3.51) |
| | Week 3 Change from Baseline: N, Mean (SD) | 79, −1.5 | (3.52) | 45, −1.6 | (2.78) |
| | Treatment Difference vs. Placebo: LS Mean Difference, 95% CI [1] | | | −0.13 | (−1.31, 1.06) |
| | p-value [1] | | | 0.830 | |
| | Week 6 Change from Baseline: N, Mean (SD) | 70, −1.5 | (3.81) | 47, −2.2 | (3.33) |
| | Treatment Difference vs, Placebo: LS Mean Difference, 95% CI [1] | | | −0.86 | (−2.17, 0.45) |
| | p-value [1] | | | 0.198 | |
| Stage 2 (Stage 1 Placebo Non- Baseline [2]: N, Mean (SD) | | 30, 24.4 | (4.55) | 33, 23.6 | (4.53) |
| responders) | | | | | |
| | Week 9 Change from Baseline [2]: N, Mean (SD) | 30, −0.4 | (2.30) | 33, −1.7 | (3.61) |
| | Treatment Difference vs. Placebo: LS Mean Difference, 95% CI [1] | | | −1.42 | (−2.94, 0.09) |
| | p-value [1] | | | 0.066 | |
| | Week 12 Change from Baseline [2]: N, Mean (SD) | 29, −1.0 | (2.69) | 32, −2.3 | (3.12) |
| | Treatment Difference vs. Placebo: LS Mean Difference, 95% CI [1] | | | −1.43 | (−2.88, 0.03) |

| | |
|---|---|
| p-value [1] | 0.054 |
| SPCD Week 6 and 12 MMRM Weighted z-statistic, overall p-value [3] | −2.20, 0.027 |

Note:
PANSS Negative Subscale ranges from 7 to 49, with higher scores indicating greater clinical severity of symptoms.
[1] MMRM with fixed effect of treatment, visit, treatment-by-visit interaction, baseline PANSS Negative Subscale and baseline PANSS Negative Subscale-by-visit interaction. An unstructured covariance matrix was used.
[2] Stage 2 Baseline is the last non-missing assessment prior to re-randomization into Stage 2 (re-randomization visit).
[3] SPCD Weighted z-statistic was calculated using Stage 1 weight = 0.6 and Stage 2 weight = 0.4. Treatment differences in each stage were estimated by the MMRM,

TABLE 31
PANSS General Psychopathology Subscale: Change from Baseline SPCD ;WARM, Observed Data
mITT Population (N = 127)

| | Visit | | | | |
|---|---|---|---|---|---|
| Stage | Statistics | Placebo | | d6-DM/Q | |
| Stage 1 | Baseline: N, Mean (SD) | 80, 30.1 | (5.05) | 47, 29.1 | (4.66) |
| | Week 3 Change from Baseline: N, Mean (SD) | 79, −0.6 | (3.85) | 45, −1.0 | (4.35) |
| | Treatment Difference vs. Placebo: LS Mean Difference, 95% CI [1] | | | −0.81 | (−2.19, 0.57) |
| | p-value [1] | | | 0.250 | |
| | Week 6 Change from Baseline: N, Mean (SD) | 70, −0.7 | (3.21) | 47, −1.7 | (4.04) |
| | Treatment Difference vs. Placebo: LS Mean Difference, 95% CI [1] | | | −1.14 | (−2.40, 0.13) |
| | p-value [1] | | | 0.077 | |
| Stage 2 (Stage 1 Placebo Non- Baseline [2]: N, Mean (SD) | | 30, 29.7 | (5.40) | 33, 28.7 | (4.84) |
| responders) | | | | | |
| | Week 9 Change from Baseline [2]: N, Mean (SD) | 30, 0.3 | (3.57) | 33, −0.9 | (4.44) |
| | Treatment Difference vs. Placebo: LS Mean Difference, 95% CI [1] | | | −1.49 | (−3.45, 0.46) |
| | p-value [1] | | | 0.132 | |
| | Week 12 Change from Baseline [2]: N, Mean (SD) | 29, 0.0 | (5.14) | 32, −1.3 | (5.10) |

| | | |
|---|---|---|
| Treatment Difference vs. Placebo: LS Mean Difference, 95% CI [1] | −1.40 | (−3.99, 1.19) |

| | |
|---|---|
| p-value [1] | 0.284 |
| SPCD Week 6 and 12 MMRM Weighted z-statistic, overall p-value [3] | −1.93, 0.054 |

Note:
PANSS General Psychopathology Subscale ranges from 16 to 112, with higher scores indicating greater clinical severity of symptoms.
[1] MMRM with fixed effect of treatment, visit, treatment-by-visit interaction, baseline PANSS General Psychopathology Subscale and baseline PANSS General Psychopathology Subscale-by-visit interaction. An unstructured covariance matrix was used.
[2] Stage 2 Baseline is the last non-missing assessment prior to re-randomization into Stage 2 (re-randomization visit).
[3] SPCD Weighted a-statistic was calculated using Stage 1 weight = 0.6 and Stage 2 weight = 0.4. Treatment differences in each stage were estimated by the MMRM.

TABLE 32
PANSS Mader Negative Factors: Change from Baseline SPCD MMRM, Observed Data
mITT Population (N = 127)

| | Visit | | | | |
|---|---|---|---|---|---|
| Stage | Statistics | Placebo | | d6-DM/Q | |
| Stage 1 | Baseline: N, Mean (SD) | 80, 24.2 | (3.81) | 47, 24.1 | (4,24) |
| | Week 3 Change from Baseline: N, Mean (SD) | 79, −1.5 | (3.28) | 45, −1.1 | (3.29) |
| | Treatment Difference vs. Placebo: LS Mean Difference, 95% CI [1] | | | 0.30 | (−0.87, 1.46) |
| | p-value [1] | | | 0.616 | |
| | Week 6 Change from Baseline: N, Mean (SD) | 70, −1.6 | (3.48) | 47, −2,1 | (3.34) |
| | Treatment Difference vs. Placebo; LS Mean Difference, 95% CI [1] | | | −0.63 | (−1.89, 0.62) |
| | p-value [1] | | | 0.320 | |
| Stage 2 (Stage 1 Placebo Non- Baseline [2]: N, Mean (SD) | | 30, 23.0 | (4.62) | 33 22.3 | (5.45) |
| responders) | | | | | |
| | Week 9 Change from Baseline [2]: N, Mean (SD) | 30, −0.2 | (2.55) | 33, −1.8 | (4.33) |
| | Treatment Difference vs. Placebo: LS Mean Difference, 95% CI [1] | | | −1.81 | (−3.51, −0.11) |
| | p-value [1] | | | 0.038 | |
| | Week 12 Change from Baseline [2]: N, Mean (SD) | 29, −0.8 | (2.80) | 32, −2.5 | (4.27) |

| | | |
|---|---|---|
| Treatment Difference vs. Placebo: LS Mean Difference, 95% CI [1] | −1.93 | (−3.62, −0,24) |

| | |
|---|---|
| p-value [1] | 0.026 |
| SPCD Week 6 and 12 MMRM Weighted z-statistic, overall p-value [3] | −2.26, 0.024 |

Note:
PANSS Marder Negative Factors ranges from 7 to 49, with higher scores indicating greater clinical severity of symptoms.
[1] MMRM with fixed effect of treatment, visit, treatment-by-visit interaction, baseline PANSS Marder Negative Factors and baseline PANSS Marder Negative Factors-by-visit interaction. An unstructured covariance matrix was used.
[2] Stage 2 Baseline is the last non-missing assessment prior to re-randomization into Stage 2 (re-randomization visit).
[3] SPCD Weighted z-statistic was calculated using Stage 1 weight = 0.6 and Stage 2 weight = 0.4. Treatment differences in each stage were estimated by the MMRM.

TABLE 33
PANSS Excitement Component: Change from Baseline SPCD MMRM, Observed Data
mITT Population (N = 127)

| Stage | Visit Statistics | Placebo | d6-DM/Q |
|---|---|---|---|
| Stage 1 | Baseline: N, Mean (SD) | 80, 6.3 (2.10) | 47, 6.2 (1.57) |
| | Week 3 Change from Baseline: N, Mean (SD) | 79, −0.1 (1.51) | 45, −0.4 (1.63) |
| | Treatment Difference vs. Placebo: LS Mean Difference, 95% CI [1] | | −0.39 (−0.88, 0.10) |
| | p-value [1] | | 0.114 |
| | Week 6 Change from Baseline: N, Mean (SD) | 70, −0.2 (1.57) | 47, −0.4 (1.64) |
| | Treatment Difference vs, Placebo: LS Mean Difference, 95% CI [1] | | −0.34 (−0.83, 0.16) |
| | p-value [1] | | 0.177 |
| Stage 2 | Baseline [2]: N, Mean (SD) | 30, 5.8 (1.27) | 33, 6.7 (2.59) |
| (Stage 1 | Week 9 Change from Baseline [2]: N, Mean (SD) | 30, 0.1 (1.52) | 33, −0.4 (1.60) |
| Placebo | Treatment Difference vs. Placebo: LS Mean Difference, 95% CI [1] | | −0.11 (−0.78, 0.56) |
| Non- | p-value [1] | | 0.739 |
| responders) | Week 12 Change from Baseline [2]: N, Mean (SD) | 29, 0.0 (2.04) | 32, −0.1 (1.77) |
| | Treatment Difference vs. Placebo: LS Mean Difference, 95% CI [1] | | 0.30 (−0.61, 1.21) |
| | p-value [1] | | 0.512 |
| | SPCD Week 6 and 12 MMRM Weighted z-statistic, overall p-value [3] | | −0.35, 0.723 |

Note:
PANSS Excitement Component ranges from 5 to 35, with higher scores indicating greater clinical severity of symptoms.
[1] MMRM with fixed effect of treatment, visit, treatment-by-visit interaction, baseline PANSS Excitement Component arid baseline PANSS Excitement Component-by-visit interaction. An unstructured covariance matrix was used.
[2] Stage 2 Baseline is the last non-missing assessment prior to re-randomization into Stage 2 (re-randomization visit).
[3] SPCD Weighted z-statistic was calculated using Stage 1 weight = 0.6 and Stage 2 weight = 0.4. Treatment differences in each stage were estimated by the MMRM,

TABLE 34
PANSS Prosocial Factors: Change from Baseline SPCD MMRM, Observed Data
rnITT Population (N = 127)

| Stage | Visit Statistics | Placebo | d6-DM/Q |
|---|---|---|---|
| Stage 1 | Baseline: N, Mean (SD) | 80, 18.4 (2.92) | 47, 18.3 (3.24) |
| | Week 3 Change from Baseline: N, Mean (SD) | 79, −1.0 (2.31) | 45, −1.4 (2.26) |
| | Treatment Difference vs. Placebo: LS Mean Difference, 95% CI [1] | | −0.44 (−1.27, 0.38) |
| | p-value [1] | | 0.288 |
| | Week 6 Change from Baseline: N, Mean (SD) | 70, −1.1 (2.53) | 47, −2.0 (2.18) |
| | Treatment Difference vs, Placebo: LS Mean Difference, 95% CI [1] | | −0.89 (−1.75, −0.03) |
| | p-value [1] | | 0.042 |
| Stage 2 | Baseline [2]: N, Mean (SD) | 30, 17.7 (3.27) | 33, 17.0 (3.30) |
| (Stage 1 | Week 9 Change from Baseline [2]: N, Mean (SD) | 30, 0.2 (1.90) | 33, −0.5 (3.05) |
| Placebo | Treatment Difference vs. Placebo: LS Mean Difference, 95% CI [1] | | −0.80 (−2.07, 0.46) |
| Non- | p-value [1] | | 0.209 |
| responders) | Week 12 Change from Baseline [2]: N, Mean (SD) | 29, −0.7 (2.02) | 32, −1.4 (2.35) |
| | Treatment Difference vs. Placebo: LS Mean Difference, 95% CI [1] | | −0.89 (−2.00, 0.22) |
| | p-value, [1] | | 0.115 |
| | SPCD Week 6 and 12 MMRM Weighted z-statistic, overall p-value [3] | | −2.60, 0.009 |

Note:
PANSS Prosocial Factors ranges from 6 to 42, with higher scores indicating greater clinical severity of symptoms.
[1] MMRM with fixed effect of treatment, visit, treatment-by-visit interaction, baseline PANSS Prosocial Factors and baseline PANSS Prosocial Factors-by-visit interaction An unstructured covariance matrix was used,
[2] Stage 2 Baseline is the last non-missing assessment prior to re-randomization into Stage 2 (re-randomization visit).
[3] SPCD Weighted z-statistic was calculated using Stage 1 weight = 0.6 and Stage 2 weight = 0.4. Treatment differences in each stage were estimated by the MMRM,

TABLE 35
PANSS General Psychopathology Subscale: Change from Baseline Parallel Group MMRM Analysis, Observed Data
mITT 12-Week Parallel Group (N = 87)

| Visit/Statistics | Placebo/Placebo | d6-DM/Q/d6-DM/Q. |
|---|---|---|
| Baseline: N, Mean (SD) | 40, 30.5 (5.03) | 47, 29.1 (4.66) |
| Week 3 Change from Baseline: N, Mean (SD) | 40, −0.9 (3.90) | 45, −1.0 (4.35) |
| Week 6 Change from Baseline: N, Mean (SD) | 35, −0.8 (3.46) | 47, −1.7 (4.04) |
| Week 9 Change from Baseline: N, Mean (SD) | 32, −0.6 (4.48) | 42, −2.5 (4.56) |
| Week 12 Change from Baseline: N, Mean (SD) | 31, −0.6 (5.97) | 42, −2.8 (4.51) |
| Week 12 Treatment Difference vs. Placebo: p-value, LS Mean Difference (95% CI) | 0.028, −2.53 | (−4.77, −0.28) |

Note:
PANSS General Psychopathology Subscale Score ranges from 16 to 112, with higher scores indicating greater clinical severity of symptoms.
Patients within each treatment group received the same treatment throughout their participation in the study.
Repeated measures model includes fixed effect for treatment, visit, treatment-by-visit interaction, baseline value, and baseline value-by-visit interaction. An unstructured covariance matrix was used.

TABLE 36
PANSS Total Score: Change from Baseline SPCD ANCOVA, LOCF Data mITT Population (N = 127)

| Stage | Visit Statistics | Placebo | d6-DM/Q |
|---|---|---|---|
| Stage 1 | Baseline: N, Mean (SD) | 80, 68.7 (7.99) | 47, 67.4 (8.26) |
| | Week 6: N, Mean (SD) | 80, 66.1 (9.11) | 47, 62.7 (9.30) |
| | Change from Baseline: N, Mean (SD) | 80, −2.6 (6,36) | 47, −4.7 (6.98) |
| | Standard Effect Size | | −0.331 |
| | % Change from Baseline: N, Mean (SD) | 80, −3.6 (9.24) | 47, −6.8 (10.68) |
| | Treatment Difference vs. Placebo: LS Mean Difference, 95% CI [1] | | −2.42 (−4.77, −0.07) |
| | p-value [1] | | 0.043 |
| Stage 2 | Baseline [2]: N, Mean (SD) | 30, 67.1 (9.00) | 33, 65.6 (8.07) |
| (Stage 1 | Week 12: N, Mean (SD) | 30, 65.9 (10.84) | 33, 62.3 (9.75) |
| Placebo | Change from Baseline [2]: N, Mean (SD) | 30, −1.2 (7.60) | 33, 3.3 (8.67) |
| Non- | Standard Effect Size | | −0.253 |
| responders) | % Change from Baseline [2]: N, Mean (SD) | 30, −1.6 (10.96) | 33, −4.6 (12.86) |
| | Treatment Difference vs. Placebo: LS Mean Difference, 95% CI [1] | | −2.43 (−6.49, 1.62) |
| | p-value [1] | | 0.235 |
| | SPCD Weighted OLS z-statistic, overall p-value [3] | | −2.25, 0.024 |

Note:
PANSS Total Score ranges from 30 to 210, with higher scores indicating greater clinical severity of symptoms.
Standard Effect Size is defined as (mean change in d6-DMIQ — mean change in Placebo) / Change front Baseline Pooled SD.
[1] Change from Baseline was analyzed at each stage by ANCOVA with treatment as fixed effect and baseline value as covariate. Missing values were imputed by LOCF within each stage.
[2] Stage 2 Baseline is the last non-missing assessment prior to re-randomization into Stage 2 (re-randomization visit).
[3] SPCD Weighted OLS z-statistic was calculated using Stage 1 weight = 0.6 and Stage 2 weight = 0.4.

TABLE 37
PANSS General Psychopathology Subscale: Change from Baseline SPCD ANCOVA, LOCF Data mITT Population (N = 127)

| Stage | Visit Statistics | Placebo | d6-DM/Q |
|---|---|---|---|
| Stage 1 | Baseline: N, Mean (SD) | 80, 30.1 (5.05) | 47, 29.1 (4.66) |
| | Week 6: N, Mean (SD) | 80, 29.2 (5.16) | 47, 27.4 (4.89) |
| | Change from Baseline: N, Mean (SD) | 80, −0.9 (3.22) | 47, −1.7 (4.04) |
| | Standard Effect Size | | −0.252 |
| | % Change from Baseline: N, Mean (SD) | 80, −2.4 (10.88) | 47, −5.2 (13.95) |
| | Treatment Difference vs. Placebo: LS Mean Difference, 95% CI [1] | | −1.11 (−2.34, 0.13) |
| | p-value [1] | | 0.078 |
| Stage 2 | Baseline [2]: N, Mean (SD) | 30, 29.7 (5.40) | 33, 28.7 (4.84) |
| (Stage 1 | Week 12: N, Mean (SD) | 30, 29.8 (5.96) | 33, 27.8 (6.32) |
| Placebo | Change from Baseline [2]: N, Mean (SD) | 30, 0.1 (5.07) | 33, −0.9 (5.53) |
| Non- | Standard Effect Size | | −0.196 |
| responders) | % Change from Baseline [2]: N, Mean (SD) | 30, 1.4 (18.50) | 33, −2.6 (18.42) |
| | Treatment Difference vs. Placebo: LS Mean Difference, 95% CI [1] | | −1.35 (−3.93, 1.24) |
| | p-value [1] | | 0.302 |
| | SPCD Weighted OLS z-statistic, overall p-value [3] | | −1.88, 0.060 |

Note:
PANSS General Psychopathology Subscale ranges from 16 to 112, with higher scores indicating greater clinical severity of symptoms.
Standard Effect Size is defined as (mean change in d6-DMIQ — mean change in Placebo) / Change from Baseline Pooled SD.
[1] Change from Baseline was analyzed at each stage by ANCOVA with treatment as fixed effect and baseline value as covariate. Missing values were imputed by LOCF within each stage.
[2] Stage 2 Baseline is the last non-missing assessment prior to re-randomization into Stage 2 (re-randomization visit).
[3] SPCD Weighted OLS z-statistic was calculated using Stage 1 weight = 0.6 and Stage 2 weight = 0.4.

TABLE 38
PANSS Total Score: Change from Baseline SPCD MMRM, Observed Data Per Protocol Population (N = 110)

| Stage | Visit Statistics | Placebo | d6-DM/Q |
|---|---|---|---|
| Stage 1 | Baseline: N, Mean (SD) | 73, 68.8 (7.88) | 37, 68.1 (8.72) |
| | Week 3 Change from Baseline: N, Mean (SD) | 72, −2.7 (6.98) | 37, −2.7 (6.33) |
| | Treatment Difference vs. Placebo: LS Mean Difference, 95% CI [1] | | −0.17 (−2.82, 2.48) |
| | p-value [1] | | 0.898 |
| | Week 6 Change from Baseline: N, Mean (SD) | 63, −2.9 (5.99) | 37, −4.9 (6.68) |
| | Treatment Difference vs. Placebo: LS Mean Difference, 95% CI [1] | | −2.06 (−4.57, 0.45) |
| | p-value [I] | | 0.106 |
| Stage 2 | Baseline [2]: N, Mean (SD) | 27, 66.4 (8.85) | 29, 65.8 (8.03) |
| (Stage 1 | Week 9 Change from Baseline [2]: N, Mean (SD) | 27, −0.6 (5.23) | 29, 4.0 (6.38) |
| Placebo | Treatment Difference vs. Placebo: LS Mean Difference, 95% CI [1] | | −3.61 (−6.55, −0.67) |
| Non- | p-value [1] | | 0.017 |
| responders) | Week 12 Change from Baseline [2]: N, Mean (SD) | 26, −2.3 (7.35) | 29, −4.9 (7.05) |
| | Treatment Difference vs. Placebo: LS Mean Difference, 95% CI [1] | | −2.92 (−6.57, 0.73) |
| | p-value [1] | | 0.115 |
| | SPCD Week 6 and 12 MMRM Weighted z-statistic, overall p-value [3] | | −2.29, 0.022 |

Note:
PANSS Total Score ranges from 30 to 210, with higher scores indicating greater clinical severity of symptoms.
[1] MMRM with fixed effect of treatment, visit, treatment-by-visit interaction, baseline PANSS Total Score and baseline PANSS Total Score-by-visit interaction. An unstructured covariance matrix was used.
[2] Stage 2 Baseline is the last non-missing assessment prior to re-randomization into Stage 2 (re-randomization visit).
[3] SPCD Weighted z-statistic was calculated using Stage 1 weight = 0.6 and Stage 2 weight = 0.4. Treatment differences in each stage were estimated by the MMRM.

TABLE 39
PANSS Total Score: Change from Baseline Parallel Group MMRM Analysis, Observed Data Per Protocol 12-Week Parallel Group (N = 74)

| Visit/Statistics | Placebo/Placebo | d6-DM/Q/d6-DM/Q |
|---|---|---|
| Baseline: N. Mean (SD) | 37, 69.4 (8.50) | 37, 68.1 (8.72) |
| Week 3 Change from Baseline: N, Mean (SD) | 37, −2.8 (7.50) | 37, −2.7(6.33) |
| Week 6 Change from Baseline: N, Mean (SD) | 32, −3.8 (5.37) | 37, −4.9 (6.68) |
| Week 9 Change from Baseline: N, Mean (SD) | 29, −4.6 (6.73) | 35, −6.7 (6.94) |
| Week 12 Change from Baseline: N, Mean (SD) | 28, −5.9 (7.88) | 35, −6.9 (8.17) |
| Week 12 Treatment Difference vs. Placebo: p-value, LS Mean Difference. (95% CI) | | 0.614, −0.98 (−4.86, 2.89) |

Note:
PANSS Total Score ranges from 30 to 210, with higher scores indicating greater clinical severity of symptoms.
Patients within each treatment group received the same treatment throughout their participation in the study.
Repeated measures model includes fixed effect for treatment, visit, treatment-by-visit interaction, baseline value, and baseline value-by-visit interaction. An unstructured covariance matrix was used.

TABLE 40
PANSS Negative Subscale: Change from Baseline SPCD MMRM, Observed Data Per Protocol Population (N = 110)

| Stage | Visit Statistics | Placebo | d6-DM/Q |
|---|---|---|---|
| Stage 1 | Baseline: N, Mean (SD) | 73, 25.5 (3.68) | 37, 24.9 (3.63) |
| | Week 3 Change from Baseline: N, Mean (SD) | 72, −1.6 (3.60) | 37, −1.3 (2.64) |
| | Treatment Difference vs. Placebo: LS Mean Difference, 95% CI [1] | | 0.28 (−1.03, 1.59) |
| | p-value [1] | | 0.675 |
| | Week 6 Change from Baseline: N, Mean (SD) | 63, −1.7 (3.60) | 37, −2.2 (3.11) |
| | Treatment Difference vs. Placebo: LS Mean Difference, 95% CI [1] | | −0.75 (−2.12, 0.62) |
| | p-value [1] | | 0.280 |
| Stage 2 | Baseline [2]: N, Mean (SD) | 27, 24.2 (4.74) | 29, 24.1 (4.09) |
| (Stage 1 | Week 9 Change from Baseline [2]: N, Mean (SD) | 27, −0.5 (2.31) | 29, −2.2 (2.80) |
| Placebo | Treatment Difference vs. Placebo: LS Mean Difference, 95% CI [1] | | −1.77 (−3.15, −0.38) |
| Non- | p-value [1] | | 0.014 |
| responders) | Week 12 Change from Baseline [2]: N, Mean (SD) | 26, −1.1 (2.78) | 29, −2.7 (2.83) |
| | Treatment Difference vs. Placebo: LS Mean Difference, 95% CI [1] | | −1.65 (−3.14, −0.15) |
| | p-value [1] | | 0.031 |
| | SPCD Week 6 and 12 MMRM Weighted z-statistic, overall p-value [3] | | −2.17, 0.030 |

Note:
PANSS Negative Subscale ranges from 7 to 49, with higher scores indicating greater clinical severity of symptoms.
[1] MMRM with fixed effect of treatment, visit, treatment-by-visit interaction, baseline PANSS Negative Subscale and baseline PANSS Negative Subscale-by-visit interaction, An unstructured covariance matrix was used,
[2] Stage 2 Baseline is the last non-missing assessment prior to re-randomization into Stage 2 (re-randomization visit).
[3] SPCD Weighted z-statistic was calculated using Stage 1 weight = 0.6 and Stage 2 weight = 0.4. Treatment differences in each stage were estimated by the MMRM.

TABLE 41
PANSS Negative Subscale: Change from Baseline SPCD ANCOVA, LOCF Data mITT Population (N = 127)

| Stage | Visit Statistics | Placebo | d6-DM/Q |
|---|---|---|---|
| Stage 1 | Baseline: N, Mean (SD) | 80, 25.2 (3.64) | 47, 24.6 (3.51) |
| | Week 6: N, Mean (SD) | 80, 23.8 (4.30) | 47, 22.4 (4.64) |
| | Change from Baseline: N, Mean (SD) | 80, −1.4 (3.65) | 47, −2.2 (3.33) |
| | Standard Effect Size | | −0.217 |
| | % Change from Baseline: N, Mean (SD) | 80, −5.1 (14.33) | 47, −8.9 (13.81) |
| | Treatment Difference vs. Placebo: LS Mean Difference, 95% CI [l] | | −0.90 (−2.16, 0.36) |
| | p-value [1] | | 0.159 |
| Stage 2 | Baseline [2]: N, Mean (SD) | 30, 24.4 (4.55) | 33, 23.6 (4.53) |
| (Stage 1 | Week 12: N, Mean (SD) | 30, 23.4 (4.94) | 33, 21.3 (4.49) |
| Placebo | Change from Baseline [2]: N, Mean (SD) | 30, −1.0 (2.65) | 33, −2.3 (3.08) |
| Non- | Standard Effect Size | | −0.452 |
| responders) | % Change from Baseline [2]: N, Mean (SD) | 30, −3.9 (10.14) | 33, −8.9 (14.10) |
| | Treatment Difference vs. Placebo: LS Mean Difference, 95% CI [1] | | −1.43 (−2.86, −0.01) |
| | p-value [1] | | 0.049 |
| | SPCD Weighted OLS z-statistic, overall p-value [3] | | −2.34, 0.019 |

Note:
PANSS Negative Subscale ranges from 7 to 49, with higher scores indicating greater clinical severity of symptoms.
Standard Effect Size is defined as (mean change in d6-DMIQ mean change in Placebo) / Change front Baseline Pooled SD.
[1] Change from Baseline was analyzed at each stage by ANCOVA with treatment as fixed effect and baseline value as covariate. Missing values were imputed by LOCF within each stage.
[2] Stage 2 Baseline is the last non-missing assessment prior to re-randomization into Stage 2 (re-randomization visit).
[3] SPCD Weighted OLS z-statistic was calculated using Stage 1 weight = 0,6 and Stage 2 weight = 0.4.

TABLE 42
PANSS Negative Subscale: Change from Baseline Parallel Group MMRM Analysis, Observed Data mITT 12-Week Parallel Group (N = 87)

| Visit/Statistics | Placebo/Placebo | d6-DM/Q/d6-DM/Q |
|---|---|---|
| Baseline: N, Mean (SD) | 40, 25.9 (3.93) | 47, 24.6 (3.51) |
| Week 3 Change from Baseline.: N, Mean (SD) | 40, −1.7 (3.61) | 45, −1.6 (2.78) |
| Week 6 Change from Baseline: N, Mean (SD) | 35, −2.0 (3.20) | 47, −2.2 (3.33) |
| Week 9 Change from Baseline: N, Mean (SD) | 32, −2.6 (4.16) | 42, −3.6 (.3.63) |
| Week 12 Change from Baseline: N, Mean (SD) | 31, −3.1 (4.35) | 42, −3.5 (.3.74) |
| Week 12 Treatment Difference vs. Placebo: p-value, LS Mean Difference (95% CI) | | 0.566, −0.52 (−2.31, 1.27) |

Note:
PANSS Negative Subscale Score ranges from 7 to 49, with higher scores indicating greater clinical severity of symptoms.
Patients within each treatment group received the same treatment throughout their participation in the study.
Repeated measures model includes fixed effect for treatment, visit, treatment-by-visit interaction, baseline value, and baseline value-by-visit interaction. An unstructured covariance matrix was used.

TABLE 43
PANSS Negative Subscale: Change from Baseline Parallel Group MMRM Analysis, Observed Data
Per Protocol 12-Week Parallel Group (N = 74)

| Visit/Statistics | Placebo/Placebo | d6-DM/Q/d6-DM/Q |
|---|---|---|
| Baseline: N, Mean (SD) | 37, 26.1 (4.03) | 37, 24.9 (3.63) |
| Week 3 Change from Baseline: N, Mean (SD) | 37, −1.8 (3.67) | 37, −1.3 (2.64) |
| Week 6 Change from Baseline: N, Mean (SD) | 32, −2.3 (3.13) | 37, −2.2 (3.11) |
| Week 9 Change from Baseline: N, Mean (SD) | 29, −3.0 (4.04) | 35, −3.2 (3.62) |
| Week 12 Change from Baseline: N, Mean (SD) | 28, −3.6 (4.25) | 35, −3.2 (3.65) |
| Week 12 Treatment Difference vs. Placebo: p-value, LS Mean Difference (95% CI) | | 0.872, 0.16 ( −1.76, 2.07) |

Note:
PANSS Negative Subscale Score ranges from 7 to 49, with higher scores indicating greater clinical severity of symptoms.
Patients within each treatment group received the same treatment throughout their participation in the study.
Repeated measures model includes fixed effect for treatment, visit, treatment-by-visit interaction, baseline value, and baseline value-by-visit interaction, An unstructured covariance matrix was used.

TABLE 44
PANSS Marder Negative Factors: Change from Baseline SPCD MMRM, Observed Data Per Protocol Population (N = 110)

| Stage | Visit Statistics | Placebo | d6-DM/Q |
|---|---|---|---|
| Stage 1 | Baseline: N, Mean (SD) | 73, 24.5 (3.85) | 37, 24.2 (4.27) |
| | Week 3 Change from Baseline: N, Mean (SD) | 72, −1.6 (3.29) | 37, −0.9 (3.47) |
| | Treatment Difference vs. Placebo: LS Mean Difference, 95% CI [1] | | 0.59 (−0.71, 1.89) |
| | p-value [1] | | 0.371 |
| | Week 6 Change from Baseline: N, Mean (SD) | 63, −1.6 (3.09) | 37, −2.1(3.31) |
| | Treatment Difference vs. Placebo: LS Mean Difference, 95% CI [1] | | −0.51 (−1.79, 0.77) |
| | p-value [1] | | 0.433 |
| Stage 2 | Baseline [2]: N, Mean (SD) | 27, 22.8 (4.82) | 29, 22.9 (5.02) |
| (Stage 1 | Week 9 Change from Baseline [2]: N, Mean (SD) | 27, −0.2 (2.54) | 29, −2.4 (3.47) |
| Placebo | Treatment Difference vs. Placebo: LS Mean Difference, 95% CI [1] | | −2.21 (−3.83, −0.60) |
| Non- | p-value [1] | | 0.008 |
| responders) | Week 12 Change from Baseline [2]: N, Mean (SD) | 26, −0.7 (2.92) | 29, −3.0 (3.90) |
| | Treatment Difference vs. Placebo: LS Mean Difference, 95% CI [1] | | −2.31 (−4.08, −0.53) |
| | p-value [1] | | 0.012 |
| | SPCD Week 6 and 12 MMRM Weighted z-statistic, overall p-value [3] | | −2.34, 0.019 |

Note:
PANSS Marder Negative Factors ranges from 7 to 49, with higher scores indicating greater clinical severity of symptoms.
[1] MMRM with fixed effect of treatment, visit, treatment-by-visit interaction, baseline PANSS Marder Negative Factors and baseline PANSS Marder Negative Factors-by-visit interaction. An unstructured covariance matrix was used.
[2] Stage 2 Baseline is the last non-missing assessment prior to re-randomization into Stage 2 (re-randomization visit).
[3] SPCD Weighted z-statistic was calculated using Stage 1 weight = 0.6 and Stage 2 weight = 0.4. Treatment differences in each stage were estimated by the MMRM.

TABLE 45
PANSS Marder Negative Factors: Change from Baseline SPCD ANCOVA, LOCF Data mITT Population (N = 127)

| Stage | Visit Statistics | Placebo | d6-DM/Q |
|---|---|---|---|
| Stage 1 | Baseline: N, Mean (SD) | 80, 24.2 (3.81) | 47, 24.1 (4.24) |
| | Week 6: N, Mean (SD) | 80, 22.7 (4.77) | 47, 21.9 (5.07) |
| | Change from Baseline: N. Mean (SD) | 80, −1.5 (3.33) | 47, −2.1(3.34) |
| | Standard Effect Size | | −0.187 |
| | % Change from Baseline: N, Mean (SD) | 80, −6.1 (14.26) | 47, −8.8 (15.30) |
| | Treatment Difference vs. Placebo: LS Mean Difference, 95% CI [1] | | −0.64 (−1.85, 0.57) |
| | p-value [1] | | 0.296 |
| Stage 2 | Baseline [2]: N, Mean (SD) | 30, 23.0 (4.62) | 33, 22.3 (5.45) |
| (Stage 1 | Week 12: N, Mean (SD) | 30, 22.2 (4.67) | 33, 19.8 (4.96) |
| Placebo | Change from Baseline [2]: N. Mean (SD) | 30, −0.8 (2.75) | 33, −2.5 (4.20) |
| Non- | Standard Effect Size | | −0.479 |
| responders) | % Change from Baseline [2]; N, Mean (SD) | 30, −2.6(11.93) | 33, −8.4 (24.53) |
| | Treatment Difference vs. Placebo: LS Mean Difference, 95% CI [1] | | −1.93 (−3.59, 0.28) |
| | p-value [1] | | 0.023 |
| | SPCD Weighted OLS z-statistic, overall p-value [3] | | −2.34, 0.019 |

Note:
PANSS Marder Negative Factors ranges from 7 to 49, with higher scores indicating greater clinical severity of symptoms.
Standard Effect Size is defined as (mean change in d6-DM/Q mean change in Placebo) / Change from Baseline Pooled SD.
[1] Change from Baseline was analyzed at each stage by ANCOVA with treatment as fixed effect and baseline value as covariate. Missing values were imputed by LOCF within each stage.
[2] Stage 2 Baseline is the last non-missing assessment prior to re-randomization into Stage 2 (re-randomization visit).
[3] SPCD Weighted OLS z-statistic was calculated using Stage 1 weight = 0.6 and Stage 2 weight = 0.4.

TABLE 46
PANSS Marder Negative Factors: Change from Baseline Parallel Group MMRM Analysis, Observed Data mITT 12-Week Parallel Group (N = 87)

| Visit/Statistics | Placebo/Placebo | d6-DM/Q/d6-DM/Q |
|---|---|---|
| Baseline: N, Mean (SD) | 40, 24.9 (3.93) | 47, 24.1 (4.24) |
| Week 3 Change from Baseline: N, Mean (SD) | 40, −1.7 (3.34) | 45, −1.1(3.29) |
| Week 6 Change from Baseline: N, Mean (SD) | 35, 2.0 (2.53) | 47, −2.1 (3.34) |
| Week 9 Change from Baseline: N, Mean (SD) | 32, −2.4 (3.65) | 42, −3.4 (3.66) |
| Week 12 Change from Baseline: N, Mean (SD) | 31, −3.0 (3.72) | 42, −3.4 (4.29) |
| Week 12 Treatment Difference vs. Placebo: p-value, LS Mean Difference (95% CI) | | 0.520, −0.59 (−2.39, 1.22) |

Note:
PANSS Marder Negative Factors Score ranges from 7 to 49, with higher scores indicating greater clinical severity of symptoms.
Patients within each treatment group received the same treatment throughout their participation in the study.
Repeated measures model includes fixed effect for treatment, visit, treatment-by-visit interaction, baseline value, and baseline value-by-visit interaction. An unstructured covariance matrix was used.

TABLE 47
PANSS Marcler Negative Factors: Change from Baseline Parallel Group MMRM Analysis, Observed Data
Per Protocol 1.2-Week Parallel Group (N = 74)

| Visit/Statistics | Placebo/Placebo | d6-DM/Q/d6-DM/Q |
|---|---|---|
| Baseline: N, Mean (SD) | 37, 25.0 (4.08) | 37, 24.2 (4.27) |
| Week 3 Change from Baseline: N, Mean (SD) | 37, −1.8(3.44) | 37, −0.9 (3.47) |
| Week 6 Change from Baseline: N, Mean (SD) | 32, −2.2 (2.50) | 37, −2.1 (3.31) |
| Week 9 Change from Baseline: N. Mean (SD) | 29, −2.7 (3.63) | 35, −3.1 (3.75) |
| Week 12 Change from Baseline: N, Mean (SD) | 28, −3.2 (3.85) | 35, −3.1 (4.45) |
| Week 12 Treatment Difference vs. Placebo: p-value, LS Mean Difference (95% CI) | | 0.846, −0.20 (−2.19, 1.80) |

Note:
PANSS Marder Negative Factors Score ranges from 7 to 49, with higher scores indicating greater clinical severity of symptoms.
Patients within each treatment group received the same treatment throughout their participation in the study.
Repeated measures model includes fixed effect for treatment, visit, treatment-by-visit interaction, baseline value, and baseline value-by-visit interaction. An unstructured covariance matrix was used.

TABLE 48
PANSS Prosocial Factors: Change from Baseline SPCD ANCOVA, LOCF Data
mITT Population (N = 127)

| | Visit | | | | |
|---|---|---|---|---|---|
| Stage | Statistics | Placebo | | d6-DM/Q | |
| Stage 1 | Baseline: N, Mean (SD) | 80, 18.4 | (2.92) | 47, 18.3 | (3.24) |
| | Week 6: N, Mean (SD) | 80, 17.4 | (3.30) | 47, 16.3 | (3.55) |
| | Change from Baseline: N, Mean (SD) | 80, −1.0 | (2.44) | 47, −2.0 | (2.18) |
| | Standard Effect Size | | | −0.392 | |
| | % Change from Baseline: | 80, −5.1 | (14.18) | 47, −10.7 | (12.27) |
| | N, Mean (SD) | | | | |
| | Treatment Difference vs. Placebo: | | | −0.94 | (−1.78, −0.11) |
| | LS Mean Difference, 95% CI [1] | | | | |
| | p-value [1] | | | 0.027 | |
| Stage 2 | Baseline [2]: N, Mean (SD) | 30, 17.7 | (3.27) | 33, 17.0 | (3.30) |
| (Stage 1 | Week 12: N, Mean (SD) | 30, 17.2 | (3.81) | 33, 15.8 | (3.00) |
| Placebo | Change from Baseline [2]: | 30, −0.5 | (2.16) | 33, −1.2 | (2.57) |
| Non- | N, Mean (SD) | | | | |
| re- | Standard Effect Size | | | −0.311 | |
| sponders) | % Change from Baseline [2]: | 30, −2.8 | (11.81) | 33, −5.9 | (16.78) |
| | N, Mean (SD) | | | | |
| | Treatment Difference vs. Placebo: | | | −0.91 | (−2.07, 0.25) |
| | LS Mean Difference, 95% CI [1] | | | | |
| | p-value [1] | | | 0.124 | |
| | SPC Weighted OLS z-statistic, | | | −2.70, | 0.007 |
| | overall p-value [3] | | | | |

Note:
PANSS Prosocial Factors ranges from 6 to 42, with higher scores indicating greater clinical severity of symptoms.
Standard Effect Size is defined as (mean change in d6-DM/Q-mean change in Placebo)/Change from Baseline Pooled SD.
[1] Change from Baseline was analyzed at each stage by ANCOVA with treatment as fixed effect and baseline value as covariate. Missing values were imputed by LOCF within each stage.
[2] Stage 2 Baseline is the last non-missing assessment prior to re-randomization into Stage 2 (re-randomization visit).
[3] SPCD Weighted OLS z-statistic was calculated using Stage 1 weight = 0.6 and Stage 2 weight = 0.4.

TABLE 49
PANSS Prosocial Factors: Change from Baseline
Parallel Group MMRM Analysis, Observed
Data mITT 12-Week Parallel Group (N = 87)

| | Placebo/ | | d6-DM/Q/ | |
|---|---|---|---|---|
| Visit/Statistics | Placebo | | d6-DM/Q | |
| Baseline: N, Mean (SD) | 40, 18.7 | (2.66) | 47, 18.3 | (3.24) |
| Week 3 Change from Baseline: | 40, −0.9 | (2.03) | 45, −1.4 | (2.26) |
| N, Mean (SD) | | | | |
| Week 6 Change from Baseline: | 35, −1.3 | (2.09) | 47, −2.0 | (2.18) |
| N, Mean (SD) | | | | |
| Week 9 Change from Baseline: | 32, −1.2 | (2.88) | 42, −2.4 | (2.58) |
| N, Mean (SD) | | | | |
| Week 12 Change from Baseline: | 31, −2.0 | (3.16) | 42, −2.5 | (2.66) |
| N, Mean (SD) | | | | |

| | |
|---|---|
| Week 12 Treatment Difference | 0.405, −0.54 |
| vs. Placebo: p-value, LS Mean | (−1.84, 0.75) |
| Difference (95% CI) | |

Note:
PANSS Prosocial Factors Score ranges from 6 to 42, with higher scores indicating greater clinical severity of symptoms.
Patients within each treatment group received the same treatment throughout their participation in the study.
Repeated measures model includes fixed effect for treatment, visit, treatment-by-visit interaction, baseline value, and baseline value-by-visit interaction. An unstructured covariance matrix was used.

TABLE 50
PANSS Positive Subscale: Change from Baseline SPCD ANCOVA, LOCF Data
mITT Population (N = 127)

| | Visit | | | | |
|---|---|---|---|---|---|
| Stage | Statistics | Placebo | | d6-DM/Q) | |
| Stage 1 | Baseline: N, Mean (SD) | 80, 13.4 | (2.81) | 47, 13.6 | (3.65) |
| | Week 6: N, Mean (SD) | 80, 13.1 | (3.67) | 47, 12.8 | (3.43) |
| | Change from Baseline: N, Mean (SD) | 80, −0.3 | (2.57) | 47, −0.8 | (2.63) |
| | Standard Effect Size | | | −0.201 | |
| | % Change from Baseline: N, Mean (SD) | 80, −1.7 | (19.66) | 47, −3.9 | (18.73) |
| | Treatment Difference vs. Placebo: | | | −0.47 | (−1.40, 0.45) |
| | LS Mean Difference, 95% CI [1] | | | | |
| | p-value [1] | | | 0.313 | |
| Stage 2 | Baseline [2]: N, Mean (SD) | 30, 13.1 | (3.64) | 33, 13.3 | (3,39) |
| (Stage 1 | Week 1.2: N, Mean | 30, 12.8 | (3.88) | 33, 13.2 | (3.63) |
| Placebo | Change from Baseline [2]: N, Mean (SD) | 30, −0.3 | (2.06) | 33, −0.1 | (2.93) |
| Non- | Standard Effect Size | | | 0.107 | |
| re- | % Change from Baseline [2]; | 30, −2.1 | (14.27) | 33, 1.3 | (23.34) |
| sponders) | N, Mean (SD) | | | | |
| | Treatment Difference vs. Placebo: | | | 0.31 | (−0.94, 1.56) |
| | LS Mean Difference, 95% CI [1] | | | | |
| | p-value [1] | | | 0.620 | |
| | SPCD Weighted OLS z-statistic, | | | −0.42, | 0.672 |
| | overall p-value [3] | | | | |

Note:
PANSS Positive Subscale ranges from 7 to 49, with higher scores indicating greater clinical severity of symptoms.
Standard Effect Size is defined as (mean change in d6-DM/Q-mean change in Placebo)/Change from Baseline Pooled SD.
[1] Change from Baseline was analyzed at each stage by ANCOVA with treatment as fixed effect and baseline value as covariate. Missing values were imputed by LOCF within each stage.
[2] Stage 2 Baseline is the last non-missing assessment prior to re-randomization into Stage 2 (re-randomization visit).
[3] SPCD Weighted OLS z-statistic was calculated using Stage 1 weight = 0.6 and Stage 2 weight = 0.4.

TABLE 51
PANSS Positive Subscale: Change from Baseline
Parallel Group MMRM Analysis, Observed
Data mITT 12-Week Parallel Group (N = 87)

| | Placebo/ | | d6-DM/Q/ | |
|---|---|---|---|---|
| Visit/Statistics | Placebo | | d6-DM/Q | |
| Baseline: N, Mean (SD) | 40, 12.9 | (2.42) | 47, 13.6 | (3.65) |
| Week 3 Change from Baseline: | 40, −0.2 | (2.75) | 45, −0.6 | (1.83) |
| N, Mean (SD) | | | | |
| Week 6 Change from Baseline: | 35, −0.2 | (3.06) | 47, −0.8 | (2.63) |
| N, Mean (SD) | | | | |
| Week 9 Change from Baseline: | 32, 0.2 | (2.87) | 42, −1.2 | (2.66) |
| N, Mean (SD) | | | | |
| Week 12 Change from Baseline: | 31, −0.6 | (3.04) | 42, −1.1 | (2.69) |
| N, Mean (SD) | | | | |

| | |
|---|---|
| Week 12 Treatment Difference | 0.791, −0.17 |
| vs. Placebo: p-value, LS | (−1.42, 1.09) |
| Mean Difference (95% CI) | |

Note:
PANSS Positive Subscale Score ranges from 7 to 49, with higher scores indicating greater clinical severity of symptoms.
Patients within each treatment group received the same treatment throughout their participation in the study.
Repeated measures model includes fixed effect for treatment, visit, treatment-by-visit interaction, baseline value, and baseline value-by-visit interaction. An unstructured covariance matrix was used.

TABLE 52
NSA-16 Global Negative Symptoms Rating: Change from Baseline SPCD MMRM,
Observed Data mITT Population (N =127)

| | Visit | | | | |
|---|---|---|---|---|---|
| Stage | Statistics | Placebo | | d6-DM/Q | |
| Stage 1 | Baseline: N, Mean (SD) | 80, 4.6 | (0.61) | 47, 4,6 | (0.64) |
| | Week 3 Change from Baseline: | 79, −0.2 | (0.65) | 45, −0.2 | (0.52) |
| | N, Mean (SD) | | | | |
| | Treatment Difference vs. Placebo: | | | 0.02 | (−0.19, 0.24) |
| | LS Mean Difference, 95% CI [1] | | | | |
| | p-value [1] | | | 0.840 | |
| | Week 6 Change from Baseline: | 70, −0.2 | (0.65) | 47, −0,4 | (0.68) |
| | N, Mean (SD) | | | | |
| | Treatment Difference vs. Placebo: | | | −0.17 | (−0.40, 0.07) |
| | LS Mean Difference, 95% CI [1] | | | | |
| | p-value [1] | | | 0.167 | |
| Stage 2 | Baseline [2]: N, Mean (SD) | 30, 4.3 | (0.71) | 33, 4.4 | (0.75) |
| (Stage 1 | Week 9 Change from Baseline [2]: | 30, −0.0 | (0,49) | 33, −0.2 | (0.55) |
| Placebo | N, Mean (SD) | | | | |
| Non- | Treatment Difference vs. Placebo: | | | −0.17 | (−0.43, 0.09) |
| re- | LS Mean Difference, 95% CI [1] | | | | |
| sponders) | p-value [1] | | | 0.206 | |
| | Week 12 Change from Baseline [2]: | 29, −0.1 | (0.52) | 32, −0.5 | (0.76) |
| | N, Mean (SD) | | | | |
| | Treatment Difference vs. Placebo: | | | −0.29 | (−0.61, 0.03) |
| | LS Mean Difference, 95% CI [1] | | | | |
| | p-value [1] | | | 0.079 | |
| | SPCD Week 6 and 12 MMRM | | | −2.23, | 0.026 |
| | Weighted z-statistic, | | | | |
| | overall p-value [3] | | | | |

Note:
NSA-16 Global Negative Symptoms Rating ranges from 1 to 7, with higher scores indicating greater clinical severity of symptoms.
[1] MMRM with fixed effect of treatment, visit, treatment-by-visit interaction, baseline NSA-16 Global Negative Symptoms Rating, and baseline NSA-16 Global Negative Symptoms Rating-by-visit interaction. An unstructured covariance matrix was used.
[2] Stage 2 Baseline is the last non-missing assessment prior to re-randomization into Stage 2 (re-randomization visit).
[3] SPCD Weighted z-statistic was calculated using Stage 1 weight = 0.6 and Stage 2 weight = 0.4. Treatment differences in each stage were estimated by the MMRM.

TABLE 53
NSA-16 Global Negative Symptoms Rating: Change from Baseline
SPCD ANCOVA, LOCF Data mITT Population (N = 127)

| | Visit | | | | |
|---|---|---|---|---|---|
| Stage | Statistics | Placebo | | d6-DM/Q | |
| | Stage 1 Baseline: N, Mean (SD) | 80, 4.6 | (0.61) | 47, 4.6 | (0.64) |
| | Week 6: N, Mean (SD) | 80, 4.4 | (0.73) | 47, 4.2 | (0.81) |
| | Change from Baseline: | 80, −0.2 | (0.64) | 47, −0.4 | (0.68) |
| | N, Mean (SD) | | | | |
| | Standard Effect Size | | | −0.310 | |
| | % Change from Baseline: | 80, −3.7 | (14.75) | 47, −8.4 | (14.46) |
| | N, Mean (SD) | | | | |
| | Treatment Difference vs. Placebo: | | | −0.18 | (−0.41, 0.05) |
| | LS Mean Difference, 95% CI [1] | | | | |
| | p-value [1] | | | 0.127 | |
| Stage 2 | Baseline [2]: N, Mean (SD) | 30, 4.3 | (0.71) | 33, 4.4 | (0.75) |
| (Stage 1 | Week 12: N, Mean (SD) | 30, 4.2 | (0.89) | 33, 4.0 | (0.73) |
| Placebo | Change from Baseline [2]: | 30, −0.1 | (0.51) | 33, −0.5 | (0.75) |
| Non- | N, Mean (SD) | | | | |
| re- | Standard Effect Size | | | −0.495 | |
| sponders) | % Change from Baseline [2]: | 30, −3.1 | (12.81) | 33, −9.2 | (14.28) |
| | N, Mean (SD) | | | | |
| | Treatment Difference vs. Placebo: | | | −0.30 | (−0.61, 0.02) |
| | LS Mean Difference, 95% CI [1] | | | | |
| | p-value [1] | | | 0.064 | |
| | SPCD Weighted OLS z-statistic, | | | −2.42, | 0.016 |
| | overall p-value [3] | | | | |

Note:
NSA-16 Global Negative Symptoms Rating ranges from 1 to 7, with higher scores indicating greater clinical severity of symptoms.
Standard Effect Size is defined as (mean change in d6-DM/Q-mean change in Placebo)/Change from Baseline Pooled SD.
[1] Change from Baseline was analyzed at each stage by ANCOVA with treatment as fixed effect and baseline value as covariate. Missing values were imputed by LOCF within each stage.
[2] Stage 2 Baseline is the last non-missing assessment prior to re-randomization into Stage 2 (re-randomization visit).
[3] SPCD Weighted OLS z-statistic was calculated using Stage 1 weight = 0.6 and Stage 2 weight = 0.4.

TABLE 54
NSA-16 Global Negative Symptoms Rating: Change
from Baseline Parallel Group MMRM Analysis,
Observed Data mITT 12-Week Parallel Group (N = 87)

| | Placebo/ | | d6-DM/Q/ | |
|---|---|---|---|---|
| Visit/Statistics | Placebo | | d6-DM/Q | |
| Baseline: N, Mean (SD) | 40, 4.6 | (0.59) | 47, 4.6 | (0.64) |
| Week 3 Change from Baseline: | 40, −0.2 | (0.64) | 45, −0.2 | (0.52) |
| N, Mean (SD) | | | | |
| Week 6 Change from Baseline: | 35, −0.3 | (0.52) | 47, −0.4 | (0.68) |
| N, Mean (SD) | | | | |
| Week 9 Change from Baseline: | 32, −0.3 | (0.60) | 42, −0.5 | (0.59) |
| N, Mean (SD) | | | | |
| Week 12 Change from Baseline: | 31, −0.5 | (0.68) | 42, −0.6 | (0.62) |
| N, Mean (SD) | | | | |

| | |
|---|---|
| Week 12 Treatment Difference | 0.250, −0.17 |
| vs. Placebo: p-value, | (−0.47, 0.12) |
| LS Mean Difference (95% CI) | |

Note:
NSA-16 Global Negative Symptoms Rating Score ranges from 1 to 7, with higher scores indicating greater clinical severity of symptoms.
Patients within each treatment group received the same treatment throughout their participation in the study.
Repeated measures model includes fixed effect tor treatment, visit, treatment-by-visit interaction, baseline value, and baseline value-by-visit interaction. An unstructured covariance matrix was used.

TABLE 55
NSA-16 Global Negative Symptoms Rating: Change from Baseline Parallel Group
ANCOVA by Visit, LOCF Data mITT 12-Week Parallel Group Population (N = 87)

| | | Placebo/ | | d6-DM/Q/ | |
|---|---|---|---|---|---|
| Visit | Result/Statistics | Placebo | | d6-DM/Q | |
| Baseline | N, Mean (SD) | 40, 4.6 | (0.59) | 47, 4,6 | (0.64) |
| Week 3 | N, Mean (SD) | 40, 4.4 | (0.81) | 45, 4,5 | (0.66) |
| Week 3 | Change from Baseline: N, Mean (SD) | 40, −0.2 | (0.64) | 45, −0.2 | (0.52) |
| Week 3 | Standard Effect Size | | | 0.034 | |
| Week 3 | Treatment Difference versus Placebo: | | | 0.810, 0.03 | |
| | p-value, LS Mean Difference (95% CI) | | | (−0.22, 0.27) | |
| Week 6 | N, Mean (SD) | 40, 4.3 | (0.69) | 47, 4.2 | (0.81) |
| Week 6 | Change from Baseline: N, Mean (SD) | 40, −0.3 | (0.49) | 47, −0.4 | (0.68) |
| Week 6 | Standard Effect Size | | | −0.256 | |
| Week 6 | Treatment Difference versus Placebo: | | | 0.272, −0.14 | |
| | p-value, LS Mean Difference (95% CI) | | | (−0.39, 0.11) | |
| Week 9 | N, Mean (SD) | 40, 4.3 | (0.79) | 47, 4.1 | (0.76) |
| Week 9 | Change from Baseline: N, Mean (SD) | 40, −0.3 | (0.55) | 47, −0.5 | (0.58) |
| Week 9 | Standard Effect Size | | | −0.450 | |
| Week 9 | Treatment Difference versus Placebo: | | | 0.046, −0.25 | |
| | p-value, LS Mean Difference (95% CI) | | | (−0.49, −0.00) | |
| Week 12 | N, Mean (SD) | 40, 4.2 | (0.85) | 47, 4.0 | (0.91) |
| Week 12 | Change from Baseline: N, Mean (SD) | 40, −0.4 | (0.63) | 47, −0.6 | (0.61) |

| | | |
|---|---|---|
| Week 12 | Standard Effect Size | −0.356 |
| Week 12 | Treatment Difference versus Placebo: | 0.103, −0.22 |
| | p-value, LS Mean Difference (95% CI) | (−0.49, 0.05) |

Note:
NSA-16 Global Negative Symptoms Rating Score ranges from 1 to 7, with higher scores indicating greater clinical severity of symptoms.
Standard Effect Size is defined as (mean change in d6-DM/Q-mean change in Placebo)/Change from Baseline Pooled SD.
Missing values were imputed by LOCF and visit windows were used to classify unscheduled or ET visits.
LS mean difference and p-values were from ANCOVA with treatment as fixed effect and Stage 1 Baseline value as a covariate.
Patients within each treatment group received the same treatment throughout their participation in the study.

TABLE 56
CDSS Score: Change from Baseline SPCD MMRM, Observed Data
mITT Population (N = 127)

| | Visit | | | | |
|---|---|---|---|---|---|
| Stage | Statistics | Placebo | | d6-DM/Q | |
| | Stage 1 Baseline: N, Mean (SD) | 80, 0.9 | (1.31) | 47, 1.1 | (1.34) |
| | Week 3 Change from Baseline: | 79, 0.2 | (1.11) | 45, 0.0 | (1.24) |
| | N, Mean (SD) | | | | |
| | Treatment Difference vs. Placebo: | | | −0.15 | (−0.56, 0.26) |
| | LS Mean Difference, 95% CI [1] | | | | |
| | p-value [1] | | | 0.474 | |
| | Week 6 Change from Baseline: | 70, −0.1 | (1.02) | 47, −0.2 | (1.34) |
| | N, Mean (SD) | | | | |
| | Treatment Difference vs. Placebo: | | | −0.07 | (−0.48, 0.35) |
| | LS Mean Difference, 95% CI [1] | | | | |
| | p-value [1] | | | 0.747 | |
| Stage 2 | Baseline [2]: N, Mean (SD) | 30, 1.0 | (1.56) | 33, 0.8 | (1.54) |
| (Stage 1 | Week 9 Change from Baseline [2]: | 30, 0.1 | (1.32) | 33, 0.1 | (0.91) |
| | N, Mean (SD) | | | | |
| Placebo | Treatment Difference vs. Placebo: | | | −0.04 | (−0.57, 0.48) |
| Non- | LS Mean Difference, 95% CI [1] | | | | |
| responders) | p-value [1] | | | 0.870 | |
| | Week 12 Change from Baseline [2]: | 29, −0.4 | (1.24) | 32, −0.0 | (1.66) |
| | N, Mean (SD) | | | | |
| | Treatment Difference vs. Placebo: | | | 0.34 | (−0.29, 0.96) |
| | LS Mean Difference, 95% CI [1] | | | | |
| | p-value [1] | | | 0.285 | |
| | SPCD Week 6 and 12 MMRM Weighted | | | 0.53, | 0.595 |
| | z-statistic, overall p-value [3] | | | | |

Note:
CDSS Score ranges from 0 to 27, with higher scores indicating greater clinical severity of symptoms.
[1 MMRM with fixed effect of treatment, visit, treatment-by-visit interaction, baseline CDSS Score and baseline CDSS Score-by-visit interaction. An unstructured covariance matrix was used.
[2] Stage 2 Baseline is the last non-missing assessment prior to re-randomization into Stage 2 (re-randomization visit).
[3] SPCD Weighted a-statistic was calculated using Stage 1 weight = 0.6 and Stage 2 weight = 0.4. Treatment differences in each stage were estimated by the MMRM.

TABLE 57
CDSS Score: Change from Baseline SPCD ANCOVA, LOCF Data
mITT Population (N = 127)

| | Visit | | | | |
|---|---|---|---|---|---|
| Stage | Statistics | Placebo | | d6-DM/Q | |
| | Stage 1 Baseline: N, Mean (SD) | 80, 0.9 | (1.31) | 47, 1.1 | (1.34) |
| | Week 6: N, Mean (SD) | 80, 0.8 | (1.52) | 47, 0.9 | (1.49) |
| | Change from Baseline: N, Mean (SD) | 80, −0.0 | (1.03) | 47, −0.2 | (1.34) |
| | Standard Effect Size | | | −0.126 | |
| | % Change from Baseline: N, Mean (SD) | 34, −28.6 | (68.86) | 25,−19.3 | (127.81) |
| | Treatment Difference vs. Placebo: | | | −0,10 | (−0.50, 0.31) |
| | LS Mean Difference, 95% CI [1] | | | | |
| | p-value [1] | | | 0.640 | |
| Stage 2 | Baseline [2]: N, Mean (SD) | 30, 1.0 | (1.56) | 33, 0.8 | (1.54) |
| (Stage 1 | Week 12: N, Mean (SD) | 30, 0.6 | (1.13) | 33,0.9 | (1.69) |
| Placebo | Change from Baseline [2]: N, Mean (SD) | 30, −0.3 | (1.24) | 33, 0.0 | (1.67) |
| Non- | Standard Effect Size | | | 0.246 | |
| responders) | % Change from Baseline [2]: N, Mean (SD) | 13, −41.0 | (65.48) | 12, −68.1 | (50.48) |
| | Treatment Difference vs. Placebo: | | | 0.30 | (−0.33, 0.94) |
| | LS Mean Difference, 95% CI [1] | | | | |
| | p-value [1] | | | 0.343 | |
| | SPCD Weighted OLS z-statistic, | | | 0.36, | 0.722 |
| | overall p-value [3] | | | | |

Note:
CDSS Score ranges from 0 to 27, with higher scores indicating greater clinical severity of symptoms.
Standard Effect Size is defined as (mean change in d6-DM/Q-mean change in Placebo)/Change from Baseline Pooled SD.
[1] Change from Baseline was analyzed at each stage by ANCOVA with treatment as fixed effect and baseline value as covariate. Missing values were imputed by LOCF within each stage.
[2] Stage 2 Baseline is the last non-missing assessment prior to re-randomization into Stage 2 (re-randomization visit).
[3] SPCD Weighted OLS z-statistic was calculated using Stage 1 weight = 0.6 and Stage 2 weight = 0.4.

TABLE 58
CDSS Score: Change from Baseline Parallel Group MMRM Analysis,
Observed Data mITT 12-Week Parallel Group (N =87)

| | Placebo/ | | d6-DMQ/ | |
|---|---|---|---|---|
| Visit/Statistics | Placebo | | d6-DM/Q | |
| Baseline: N, Mean (SD) | 40, 0.9 | (1.44) | 47, 1.1 | (1.34) |
| Week 3 Change from Baseline: | 40, 0.2 | (1.29) | 45, 0.0 | (1.24) |
| N, Mean (SD) | | | | |
| Week 6 Change from Baseline: | 35, −0.1 | (1.26) | 47, −0.2 | (1.34) |
| N, Mean (SD) | | | | |
| Week 9 Change from Baseline: | 32, 0.0 | (0.88) | 42, 0,0 | (1.68) |
| N, Mean (SD) | | | | |
| Week 12 Change from Baseline: | 31, −0.4 | (0.99) | 42, −0.2 | (1.27) |
| N, Mean (SD) | | | | |

| | |
|---|---|
| Week 12 Treatment Difference | 0.368, 0.19 |
| vs. Placebo: p-value, LS Mean | (−0.23, 0.61) |
| Difference (95% CI) | |

Note:
CDSS Score ranges from 0 to 27, with higher scores indicating greater clinical severity of symptoms.
Patients within each treatment group received the same treatment throughout their participation in the study.
Repeated measures model includes fixed effect for treatment, visit, treatment-by-visit interaction, baseline value, and baseline value-by-visit interaction. An unstructured covariance matrix was used.

TABLE 59
NSA-16 Total Score: Change from Baseline SPCD ANCOVA, LOCF Data
mITT Population (N = 127)
Subgroup: Baseline N1CCB Composite <30

| | Visit | | | | |
|---|---|---|---|---|---|
| Stage | Statistics | Placebo | | d6-DM/Q | |
| Stage 1 | Baseline: N, Mean (SD) | 42, 61.8 | (8.48) | 19, 60.8 | (7.38) |
| | Week 6: N, Mean (SD) | 42, 58.3 | (9.62) | 19, 56.1 | (8.50) |
| | Change from Baseline: N, Mean (SD) | 42, −3.5 | (5.28) | 19, −4.7 | (5.46) |
| | Standard Effect Size | | | −0.226 | |
| | % Change from Baseline: N, Mean (SD) | 42, −5.6 | (8.65) | 19, −7.7 | (9.53) |
| | Treatment Difference vs. Placebo: | | | −1.28 | (−4.24, 1.69) |
| | LS Mean Difference, 95% CI [1] | | | | |
| | p-value [1] | | | 0.393 | |
| Stage 2 | Baseline [2]: N, Mean (SD) | 15, 60.4 | (11.37) | 20, 56.3 | (7.75) |
| (Stage 1 | Week 12: N, Mean (SD) | 15, 58.4 | (13.25) | 20, 54.6 | (8.71) |
| Placebo | Change from Baseline [2]: N, Mean (SD) | 15, −2.0 | (4.84) | 20, −1.8 | (3.68) |
| Non- | Standard Effect Size | | | 0.059 | |
| re- | % Change from Baseline [2]: | 15, −3.8 | (8.37) | 20, −3.2 | (6.64) |
| sponders) | N, Mean (SD) | | | | |
| | Treatment Difference vs. Placebo: | | | 0.50 | (−2.52, 3.52) |
| | LS Mean Difference, 95% CI [1] | | | | |
| | p-value [1] | | | 0.737 | |
| | SPCD Weighted OLS | | | −0.53, | 0.598 |
| | z-statistic, overall p-value [3] | | | | |

Note:
NSA-16 Total Score ranges from 16 to 96, with higher scores indicating greater clinical severity of symptoms.
Standard Effect Size is defined as (mean change in d6-DM/Q-mean change in Placebo)/Change from Baseline Pooled SD.
[1] Change from Baseline was analyzed at each stage by ANCOVA with treatment as fixed effect and baseline value as covariate. Missing values were imputed by LOCF within each stage.
[2] Stage 2 Baseline is the last non-missing assessment prior to re-randomization into Stage 2 (re-randomization visit).
[3] SPCD Weighted OLS z-statistic was calculated using Stage 1 weight = 0.6 and Stage 2 weight = 0.4.

TABLE 60
NSA-16 Total Score: Change from Baseline SPCD ANCOVA, LOCF Data
mITT Population (N = 127)
Subgroup: Baseline MCCB Composite ≥30

| | Visit | | | | |
|---|---|---|---|---|---|
| Stage | Statistics | Placebo | | d6-DM/Q | |
| Stage 1 | Baseline: N, Mean (SD) | 38, 58.8 | (6.49) | 28,61.1 | (7.76) |
| | Week 6: N, Mean (SD) | 38, 56.4 | (8.75) | 28, 55.8 | (9.07) |
| | Change from Baseline: | 38, −2.4 | (6.30) | 28, −5.3 | (5.85) |
| | N, Mean (SD) | | | | |
| | Standard Effect Size | | | −0.477 | |
| | % Change from Baseline: | 38, −4.0 | (10.35) | 28, −8.6 | (9.40) |
| | N, Mean (SD) | | | | |
| | Treatment Difference vs. Placebo: | | | −2.73 | (−5.82, 0.37) |
| | LS Mean Difference, 95% CI [1] | | | | |
| | p-value [1] | | | 0.083 | |
| Stage 2 | Baseline 2: N, Mean (SD) | 15, 54.7 | (6.02) | 13, 59.6 | (10.85) |
| (Stage 1 | Week 12: N, Mean (SD) | 15, 52.4 | (8.28) | 13, 53.2 | (8.80) |
| Placebo | Change from Baseline [2]: | 15, −2.3 | (6.96) | 13, −6.4 | (8.47) |
| Non- | N, Mean (SD) | | | | |
| re- | Standard Effect Size | | | −0.527 | |
| sponders) | % Change from Baseline [2]: | 15, −4.1 | (11.36) | 13, −9.5 | (14.46) |
| | N, Mean (SD) | | | | |
| | Treatment Difference vs. Placebo: | | | −2.06 | (−7.74, 3.62) |
| | LS Mean Difference, 95% CI [1] | | | | |
| | p-value [1] | | | 0.463 | |
| | SPCD Weighted OLS z-statistic, | | | −1.71, | 0.088 |
| | overall p-value [3] | | | | |

Note:
NSA-1.6 Total Score ranges from 16 to 96, with higher scores indicating greater clinical severity of symptoms.
Standard Effect Size is defined as (mean change in d6-DM/Q-mean change in Placebo)/Change from Baseline Pooled SD.
[1] Change from Baseline was analyzed at each stage by ANCOVA with treatment as fixed effect and baseline value as covariate. Missing values were imputed by LOCF within each stage.
[2] Stage 2 Baseline is the last non-missing assessment prior to re-randomization into Stage 2 (re-randomization visit).
[3] SPCD Weighted OLS z-statistic was calculated using Stage 1 weight = 0.6 and Stage 2 weight = 0.4.

TABLE 61
Summary of Simpson-Angus Scale (SAS) by Gender and Visit
Safety Population (N = 144)

| | Males and Females | | | Males Only | | | Females Only | | |
|---|---|---|---|---|---|---|---|---|---|
| Treatment | | | Median | | | Median | | | Median |
| Visit | N | Mean (SD) | (Min, Max) | N | Mean (SD) | (Min, Max) | N | Mean (SD) | (Min, Max) |

Item: Total Score

| | | | | | | | | | |
|---|---|---|---|---|---|---|---|---|---|
| Placebo/Placebo (N = 56) | | | | | | | | | |
| Baseline [1] | 56 | 0.04 (0.130) | 0.00 (0.0, 0.8) | 36 | 0.05 (0.159) | 0.00 (0.0, 0.8) | 20 | 0.01 (0.031) | 0.00 (0.0, 0.1) |
| Week 6 | 51 | 0.03 (0.132) | 0.00 (0.0, 0.9) | 33 | 0.05 (0.162) | 0.00 (0.0, 0.9) | 18 | 0.00 (0.000) | 0.00 (0.0, 0.0) |
| Week 12 | 39 | 0.03 (0.132) | 0.00 (0.0, 0.8) | 26 | 0.04 (0.160) | 0.00 (0.0, 0.8) | 13 | 0.00 (0.000) | 0.00 (0.0, 0.0) |
| d6-DM/Q/d6-DM/Q (N = 48) | | | | | | | | | |
| Baseline [1] | 48 | 0.05 (0.092) | 0.00 (0.0, 0.4) | 30 | 0.05 (0.107) | 0.00 (0.0, 0.4) | 18 | 0.04 (0.062) | 0.00 (0.0, 0.2) |
| Week 6 | 47 | 0.05 (0.093) | 0.00 (0.0, 0.5) | 30 | 0.04 (0.073) | 0.00 (0.0, 0.2) | 17 | 0.05 (0.123) | 0.00 (0.0, 0.5) |
| Week 12 | 42 | 0.04 (0.062) | 0.00 (0.0, 0.2) | 28 | 0.03 (0.061) | 0.00 (0.0, 0.2) | 14 | 0.04 (0.065) | 0.00 (0.0, 0.2) |
| Placebo/d6-DM/Q (N = 40) | | | | | | | | | |
| Placebo | | | | | | | | | |
| Baseline [1] | 40 | 0.08 (0.137) | 0.00 (0.0, 0.5) | 33 | 0.09 (0.147) | 0.00 (0.0, 0.5) | 7 | 0.01 (0.038) | 0.00 (0.0, 0.1) |
| Week 6 | 40 | 0.07 (0.120) | 0.00 (0.0, 0.4) | 33 | 0.07 (0.115) | 0.00 (0.0, 0.4) | 7 | 0.07 (0.150) | 0.00 (0.0, 0.4) |
| d6-DM/Q | | | | | | | | | |
| Week 6 (BL) [2] | 39 | 0.07 (0.120) | 0.00 (0.0, 0.4) | 32 | 0.07 (0.115) | 0.00 (0.0, 0.4) | 7 | 0.07 (0.150) | 0.00 (0.0, 0.4) |
| Week 12 | 39 | 0.08 (0.188) | 0.00 (0.0, 1.0) | 32 | 0.09 (0.205) | 0.00 (0.0, 1.0) | 7 | 0.01 (0.038) | 0.00 (0.0, 0.1) |

Item: Total Gait

| | | | | | | | | | |
|---|---|---|---|---|---|---|---|---|---|
| Placebo/Placebo (N = 56) | | | | | | | | | |
| Baseline [1] | 56 | 0.1 (0.43) | 0.0 (0, 2) | 36 | 0.2 (0.51) | 0.0 (0, 2) | 20 | 0.1 (0.22) | 0.0 (0, 1) |
| Week 6 | 51 | 0.1 (0.36) | 0.0 (0, 2) | 33 | 0.2 (0.44) | 0.0 (0, 2) | 18 | 0.0 (0.00) | 0.0 (0, 0) |
| Week 12 | 39 | 0.1 (0.35) | 0.0 (0, 2) | 26 | 0.1 (0.43) | 0.0 (0, 2) | 13 | 0.0 (0.00) | 0.0 (0, 0) |
| d6-DM/Q/d6-DM/Q (N = 48) | | | | | | | | | |
| Baseline [1] | 48 | 0.2 (0.39) | 0.0 (0, 1) | 30 | 0.2 (0.38) | 0.0 (0, 1) | 18 | 0.2 (0.43) | 0.0 (0, 1) |
| Week 6 | 47 | 0.2 (0.43) | 0.0 (0, 1) | 30 | 0.2 (0.41) | 0.0 (0, 1) | 17 | 0.3 (0.47) | 0.0 (0, 1) |
| Week 12 | 42 | 0.2 (0.40) | 0.0 (0, 1) | 28 | 0.1 (0.36) | 0.0 (0, 1) | 14 | 0.3 (0.47) | 0.0 (0, 1) |
| Placebo/d6-DM/Q (N = 40) | | | | | | | | | |
| Placebo | | | | | | | | | |
| Baseline [1] | 40 | 0.3 (0.51) | 0.0 (0, 2) | 33 | 0.3 (0.53) | 0.0 (0, 2) | 7 | 0.1 (0.38) | 0.0 (0, 1) |
| Week 6 | 40 | 0.3 (0.49) | 0.0 (0, 2) | 33 | 0.3 (0.52) | 0.0 (0, 2) | 7 | 0.1 (0.38) | 0.0 (0, 1) |
| d6-DM/Q | | | | | | | | | |
| Week 6 (BL) [2] | 39 | 0.3 (0.50) | 0.0 (0, 2) | 32 | 0.3 (0.52) | 0.0 (0, 2) | 7 | 0.1 (0.38) | 0.0 (0, 1) |
| Week 12 | 39 | 0.2 (0.43) | 0.0 (0, 1) | 32 | 0.3 (0.44) | 0.0 (0, 1) | 7 | 0.1 (0.38) | 0.0 (0, 1) |

Item: Arm Dropping

| | | | | | | | | | |
|---|---|---|---|---|---|---|---|---|---|
| Placebo/Placebo (N = 56) | | | | | | | | | |
| Baseline [1] | 56 | 0.0 (0.13) | 0.0 (0, 1) | 36 | 0.0 (0.17) | 0.0 (0, 1) | 20 | 0.0 (0.00) | 0.0 (0, 0) |
| Week 6 | 51 | 0.0 (0.14) | 0.0 (0, 1) | 33 | 0.0 (0.17) | 0.0 (0, 1) | 18 | 0.0 (0.00) | 0.0 (0, 0) |
| Week 12 | 39 | 0.0 (0.16) | 0.0 (0, 1) | 26 | 0.0 (0.20) | 0.0 (0, 1) | 13 | 0.0 (0.00) | 0.0 (0, 0) |
| d6-DM/Q/d6-DM/Q (N = 48) | | | | | | | | | |
| Baseline [1] | 48 | 0.0 (0.14) | 0.0 (0, 1) | 30 | 0.0 (0.00) | 0.0 (0, 0) | 18 | 0.1 (0.24) | 0.0 (0, 1) |
| Week 6 | 47 | 0.0 (0.00) | 0.0 (0, 0) | 30 | 0.0 (0.00) | 0.0 (0, 0) | 17 | 0.0 (0.00) | 0.0 (0, 0) |
| Week 12 | 42 | 0.0 (0.00) | 0.0 (0, 0) | 28 | 0.0 (0.00) | 0.0 (0, 0) | 14 | 0.0 (0.00) | 0.0 (0, 0) |
| Placebo/d6-DM/Q (N = 40) | | | | | | | | | |
| Placebo | | | | | | | | | |
| Baseline [1] | 40 | 0.1 (0.22) | 0.0 (0, 1) | 33 | 0.1 (0.24) | 0.0 (0, 1) | 7 | 0.0 (0.00) | 0.0 (0, 0) |
| Week 6 | 40 | 0.0 (0.16) | 0.0 (0, 1) | 33 | 0.0 (0.17) | 0.0 (0, 1) | 7 | 0.0 (0.00) | 0.0 (0, 0) |
| d6-DM/Q | | | | | | | | | |
| Week 6 (BL) [2] | 39 | 0.0 (0.16) | 0.0 (0, 1) | 32 | 0.0 (0.18) | 0.0 (0, 1) | 7 | 0.0 (0.00) | 0.0 (0, 0) |
| Week 12 | 39 | 0.1 (0.22) | 0.0 (0, 1) | 32 | 0.1 (0.25) | 0.0 (0, 1) | 7 | 0.0 (0.00) | 0.0 (0, 0) |

Item: Shoulder Shaking

| | | | | | | | | | |
|---|---|---|---|---|---|---|---|---|---|
| Placebo/Placebo (N = 56) | | | | | | | | | |
| Baseline [1] | 56 | 0.0 (0.27) | 0.0 (0, 2) | 36 | 0.1 (0.33) | 0.0 (0, 2) | 20 | 0.0 (0.00) | 0.0 (0, 0) |
| Week 6 | 51 | 0.0 (0.20) | 0.0 (0, 1) | 33 | 0.1 (0.24) | 0.0 (0, 1) | 18 | 0.0 (0.00) | 0.0 (0, 0) |
| Week 12 | 39 | 0.1 (0.32) | 0.0 (0, 2) | 26 | 0.1 (0.39) | 0.0 (0, 2) | 13 | 0.0 (0.00) | 0.0 (0, 0) |
| d6-DM/Q/d6-DM/Q (N = 48) | | | | | | | | | |
| Baseline [1] | 48 | 0.0 (0.20) | 0.0 (0, 1) | 30 | 0.1 (0.25) | 0.0 (0, 1) | 18 | 0.0 (0.00) | 0.0 (0, 0) |
| Week 6 | 47 | 0.0 (0.20) | 0.0 (0, 1) | 30 | 0.0 (0.18) | 0.0 (0, 1) | 17 | 0.1 (0.24) | 0.0 (0, 1) |
| Week 12 | 42 | 0.0 (0.15) | 0.0 (0, 1) | 28 | 0.0 (0.19) | 0.0 (0, 1) | 14 | 0.0 (0.00) | 0.0 (0, 0) |
| Placebo/d6-DM/Q (N = 40) | | | | | | | | | |
| Placebo | | | | | | | | | |
| Baseline [1] | 40 | 0.1 (0.22) | 0.0 (0, 1) | 33 | 0.1 (0.24) | 0.0 (0, 1) | 7 | 0.0 (0.00) | 0.0 (0, 0) |
| Week 6 | 40 | 0.1 (0.27) | 0.0 (0, 1) | 33 | 0.1 (0.29) | 0.0 (0, 1) | 7 | 0.0 (0.00) | 0.0 (0, 0) |
| d6-DM/Q | | | | | | | | | |
| Week 6 (BL) [2] | 39 | 0.1 (0.27) | 0.0 (0, 1) | 32 | 0.1 (0.30) | 0.0 (0, 1) | 7 | 0.0 (0.00) | 0.0 (0, 0) |
| Week 12 | 39 | 0.1 (0.22) | 0.0 (0, 1) | 32 | 0.1 (0.25) | 0.0 (0, 1) | 7 | 0.0 (0.00) | 0.0 (0, 0) |

Item: Elbow Rigidity

| | | | | | | | | | |
|---|---|---|---|---|---|---|---|---|---|
| Placebo/Placebo (N = 56) | | | | | | | | | |
| Baseline [1] | 56 | 0.0 (0.13) | 0.0 (0, 1) | 36 | 0.0 (0.17) | 0.0 (0, 1) | 20 | 0.0 (0.00) | 0.0 (0, 0) |
| Week 6 | 51 | 0.0 (0.28) | 0.0 (0, 2) | 33 | 0.1 (0.35) | 0.0 (0, 2) | 18 | 0.0 (0.00) | 0.0 (0, 0) |
| Week 12 | 39 | 0.0 (0.16) | 0.0 (0, 1) | 26 | 0.0 (0.20) | 0.0 (0, 1) | 13 | 0.0 (0.00) | 0.0 (0, 0) |
| d6-DM/Q/d6-DM/Q (N = 48) | | | | | | | | | |
| Baseline [1] | 48 | 0.0 (0.14) | 0.0 (0, 1) | 30 | 0.0 (0.18) | 0.0 (0, 1) | [8 | 0.0 (0.00) | 0.0 (0, 0) |
| Week 6 | 47 | 0.0 (0.15) | 0.0 (0, 1) | 30 | 0.0 (0.18) | 0.0 (0, 1) | 17 | 0.0 (0.00) | 0.0 (0, 0) |
| Week 12 | 42 | 0.0 (0.00) | 0.0 (0, 0) | 28 | 0.0 (0.00) | 0.0 (0, 1) | 14 | 0.0 (0.00) | 0.0 (0, 0) |
| Placebo/d6-DM/Q (N = 40) | | | | | | | | | |
| Placebo | | | | | | | | | |
| Baseline [1] | 40 | 0.0 (0.16) | 0.0 (0, 1) | 33 | 0.0 (0.17) | 0.0 (0, 1) | 7 | 0.0 (0.00) | 0.0 (0, 0) |
| Week 6 | 40 | 0.0 (0.16) | 0.0 (0, 1) | 33 | 0.0 (0.17) | 0.0 (0, 1) | 7 | 0.0 (0.00) | 0.0 (0, 0) |
| d6-DM/Q | | | | | | | | | |
| Week 6 (BL) [2] | 39 | 0.0 (0.16) | 0.0 (0, 1) | 32 | 0.0 (0.18) | 0.0 (0, 1) | 7 | 0.0 (0.00) | 0.0 (0, 0) |
| Week 12 | 39 | 0.1 (0.27) | 0.0 (0, 1) | 32 | 0.1 (0.30) | 0.0 (0, 1) | 7 | 0.0 (0.00) | 0.0 (0, 0) |

Item: Wrist Rigid/Fixation of Position

| | | | | | | | | | |
|---|---|---|---|---|---|---|---|---|---|
| Placebo/Placebo (N = 56) | | | | | | | | | |
| Baseline [1] | 56 | 0.0 (0.27) | 0.0 (0, 2) | 36 | 0.1 (0.33) | 0.0 (0, 2) | 20 | 0.0 (0.00) | 0.0 (0, 0) |
| Week 6 | 51 | 0.0 (0.28) | 0.0 (0, 2) | 33 | 0.1 (0.35) | 0.0 (0, 2) | 18 | 0.0 (0.00) | 0.0 (0, 0) |
| Week 12 | 39 | 0.0 (0.00) | 0.0 (0, 0) | 26 | 0.0 (0.00) | 0.0 (0, 0) | 13 | 0.0 (0.00) | 0.0 (0, 0) |
| d6-DM/Q/d6-DM/Q (N = 48) | | | | | | | | | |
| Baseline [1] | 48 | 0.0 (0.14) | 0.0 (0, 1) | 30 | 0.0 (0.18) | 0.0 (0, 1) | 18 | 0.0 (0.00) | 0.0 (0, 0) |
| Week 6 | 47 | 0.0 (0.20) | 0.0 (0, 1) | 30 | 0.0 (0.18) | 0.0 (0, 1) | 17 | 0.1 (0.24) | 0.0 (0, 1) |
| Week 12 | 42 | 0.0 (0.00) | 0.0 (0, 0) | 28 | 0.0 (0.00) | 0.0 (0, 0) | 14 | 0.0 (0.00) | 0.0 (0, 0) |
| Placebo/d6-DM/Q (N = 40) | | | | | | | | | |
| Placebo | | | | | | | | | |
| Baseline [1] | 40 | 0.0 (0.00) | 0.0 (0, 0) | 33 | 0.0 (0.00) | 0.0 (0, 0) | 7 | 0.0 (0.00) | 0.0 (0, 0) |
| Week 6 | 40 | 0.0 (0.00) | 0.0 (0, 0) | 33 | 0.0 (0.00) | 0.0 (0, 0) | 7 | 0.0 (0.00) | 0.0 (0, 0) |
| d6-DM/Q | | | | | | | | | |
| Week 6 (BL) [2] | 39 | 0.0 (0.00) | 0.0 (0, 0) | 32 | 0.0 (0.00) | 0.0 (0, 0) | 7 | 0.0 (0.00) | 0.0 (0, 0) |
| Week 12 | 39 | 0.1 (0.27) | 0.0 (0, 1) | 32 | 0.1 (0.30) | 0.0 (0, 1) | 7 | 0.0 (0.00) | 0.0 (0, 0) |

Item: Leg Pendulousness

| | | | | | | | | | |
|---|---|---|---|---|---|---|---|---|---|
| Placebo/Placebo (N = 56) | | | | | | | | | |
| Baseline [1] | 56 | 0.0 (0.27) | 0.0 (0, 2) | 36 | 0.1 (0.33) | 0.0 (0, 2) | 20 | 0.0 (0.00) | 0.0 (0, 0) |
| Week 6 | 51 | 0.0 (0.00) | 0.0 (0, 0) | 33 | 0.0 (0.00) | 0.0 (0, 0) | 18 | 0.0 (0.00) | 0.0 (0, 0) |
| Week 12 | 39 | 0.0 (0.00) | 0.0 (0, 0) | 26 | 0.0 (0.00) | 0.0 (0, 0) | 13 | 0.0 (0.00) | 0.0 (0, 0) |
| d6-DM/Q/d6-DM/Q (N = 48) | | | | | | | | | |
| Baseline [1] | 48 | 0.0 (0.14) | 0.0 (0, 1) | 30 | 0.0 (0.18) | 0.0 (0, 1) | 18 | 0.0 (0.00) | 0.0 (0, 0) |
| Week 6 | 47 | 0.0 (0.15) | 0.0 (0, 1) | 30 | 0.0 (0.18) | 0.0 (0, 1) | 17 | 0.0 (0.00) | 0.0 (0, 0) |
| Week 12 | 42 | 0.1 (0.34) | 0.0 (0, 2) | 28 | 0.0 (0.19) | 0.0 (0, 1) | 14 | 0.1 (0.53) | 0.0 (0, 2) |
| Placebo/d6-DM/Q (N = 40) | | | | | | | | | |
| Placebo | | | | | | | | | |
| Baseline [1] | 40 | 0.1 (0.35) | 0.0 (0, 2) | 33 | 0.1 (0.38) | 0.0 (0, 2) | 7 | 0.0 (0.00) | 0.0 (0, 0) |
| Week 6 | 40 | 0.1 (0.22) | 0.0 (0, 1) | 33 | 0.1 (0.24) | 0.0 (0, 1) | 7 | 0.0 (0.00) | 0.0 (0, 0) |
| d6-DM/Q | | | | | | | | | |
| Week 6 (BL) [2] | 39 | 0.1 (0.22) | 0.0 (0, 1) | 32 | 0.1 (0.25) | 0.0 (0, 1) | 7 | 0.0 (0.00) | 0.0 (0, 0) |
| Week 12 | 39 | 0.1 (0.35) | 0.0 (0, 2) | 32 | 0.1 (0.39) | 0.0 (0, 2) | 7 | 0.0 (0.00) | 0.0 (0, 0) |

Item: Head Rotation

| | | | | | | | | | |
|---|---|---|---|---|---|---|---|---|---|
| Placebo/Placebo (N = 56) | | | | | | | | | |
| Baseline [1] | 56 | 0.0 (0.27) | 0.0 (0, 2) | 36 | 0.1 (0.33) | 0.0 (0, 2) | 20 | 0.0 (0.00) | 0.0 (0, 0) |
| Week 6 | 51 | 0.0 (0.14) | 0.0 (0, 1) | 33 | 0.0 (0.17) | 0.0 (0, 1) | 18 | 0.0 (0.00) | 0.0 (0, 0) |
| Week 12 | 39 | 0.1 (0.48) | 0.0 (0, 3) | 26 | 0.1 (0.59) | 0.0 (0, 3) | 13 | 0.0 (0.00) | 0.0 (0, 0) |
| d6-DM/Q/d6-DM/Q (N = 48) | | | | | | | | | |
| Baseline [1] | 48 | 0.0 (0.14) | 0.0 (0, 1) | 30 | 0.0 (0.00) | 0.0 (0, 0) | 18 | 0.1 (0.24) | 0.0 (0, 1) |
| Week 6 | 47 | 0.0 (0.15) | 0.0 (0, 1) | 30 | 0.0 (0.00) | 0.0 (0, 0) | 17 | 0.1 (0.24) | 0.0 (0, 1) |
| Week 12 | 42 | 0.0 (0.00) | 0.0 (0, 0) | 28 | 0.0 (0.00) | 0.0 (0, 0) | 14 | 0.0 (0.00) | 0.0 (0, 0) |
| Placebo/d6-DM/Q (N = 40) | | | | | | | | | |
| Placebo | | | | | | | | | |
| Baseline [1] | 40 | 0.1 (0.33) | 0.0 (0, 1) | 33 | 0.2 (0.36) | 0.0 (0, 1) | 7 | 0.0 (0.00) | 0.0 (0, 0) |
| Week 6 | 40 | 0.1 (0.22) | 0.0 (0, 1) | 33 | 0.1 (0.24) | 0.0 (0, 1) | 7 | 0.0 (0.00) | 0.0 (0, 0) |
| d6-DM/Q | | | | | | | | | |
| Week 6 (BL) [2] | 39 | 0.0 (0.16) | 0.0 (0, 1) | 32 | 0.0 (0.18) | 0.0 (0, 1) | 7 | 0.0 (0.00) | 0.0 (0, 0) |
| Week 12 | 39 | 0.1 (0.27) | 0.0 (0, 1) | 32 | 0.1 (0.30) | 0.0 (0, 1) | 7 | 0.0 (0.00) | 0.0 (0, 0) |

Item: Glabella Tap

| | | | | | | | | | |
|---|---|---|---|---|---|---|---|---|---|
| Placebo/Placebo (N = 56) | | | | | | | | | |
| Baseline [1] | 56 | 0.0 (0.13) | 0.0 (0, 1) | 36 | 0.0 (0.17) | 0.0 (0, 1) | 20 | 0.0 (0.00) | 0.0 (0, 0) |
| Week 6 | 51 | 0.0 (0.14) | 0.0 (0, 1) | 33 | 0.0 (0.17) | 0.0 (0, 1) | 18 | 0.0 (0.00) | 0.0 (0, 0) |
| Week 12 | 39 | 0.0 (0.00) | 0.0 (0, 0) | 26 | 0.0 (0.00) | 0.0 (0, 0) | 13 | 0.0 (0.00) | 0.0 (0, 0) |
| d6-DM/Q/d6-DM/Q (N = 48) | | | | | | | | | |
| Baseline [1] | 48 | 0.0 (0.20) | 0.0 (0, 1) | 30 | 0.0 (0.18) | 0.0 (0, 1) | 18 | 0.1 (0.24) | 0.0 (0, 1) |
| Week 6 | 47 | 0.0 (0.15) | 0.0 (0, 1) | 30 | 0.0 (0.00) | 0.0 (0, 0) | 17 | 0.1 (0.24) | 0.0 (0, 1) |
| Week 12 | 42 | 0.0 (0.22) | 0.0 (0, 1) | 28 | 0.1 (0.26) | 0.0 (0, 1) | 14 | 0.0 (0.00) | 0.0 (0, 0) |
| Placebo/d6-DM/Q (N = 40) | | | | | | | | | |
| Placebo | | | | | | | | | |
| Baseline [1] | 40 | 0.1 (0.22) | 0.0 (0, 1) | 33 | 0.1 (0.24) | 0.0 (0, 1) | 7 | 0.0 (0.00) | 0.0 (0, 0) |
| Week 6 | 40 | 0.1 (0.65) | 0.0 (0, 4) | 33 | 0.0 (0.17) | 0.0 (0, 1) | 7 | 0.6 (1.51) | 0.0 (0, 4) |
| d6-DM/Q | | | | | | | | | |
| Week 6 (BL) [2] | 39 | 0.1 (0.64) | 0.0 (0, 4) | 32 | 0.0 (0.00) | 0.0 (0, 0) | 7 | 0.6 (1.51) | 0.0 (0, 4) |
| Week 12 | 39 | 0.0 (0.16) | 0.0 (0, 1) | 32 | 0.0 (0.18) | 0.0 (0, 1) | 7 | 0.0 (0.00) | 0.0 (0, 0) |

Item: Tremor

| | | | | | | | | | |
|---|---|---|---|---|---|---|---|---|---|
| Placebo/Placebo (N = 56) | | | | | | | | | |
| Baseline ft) | 56 | 0.0 (0.13) | 0.0 (0, 1) | 36 | 0.0 (0.17) | 0.0 (0, 1) | 20 | 0.0 (0.00) | 0.0 (0, 0) |
| Week 6 | 51 | 0.0 (0.14) | 0.0 (0, 1) | 33 | 0.0 (0.17) | 0.0 (0, 1) | 18 | 0.0 (0.00) | 0.0 (0, 0) |
| Week 12 | 39 | 0.0 (0.16) | 0.0 (0, 1) | 26 | 0.0 (0.20) | 0.0 (0, 1) | 13 | 0.0 (0.00) | 0.0 (0, 0) |
| d6-DM/Q/d6-DM/Q (N = 48) | | | | | | | | | |
| Baseline [1] | 48 | 0.1 (0.33) | 0.0 (0, 1) | 30 | 0.2 (0.38) | 0.0 (0, 1) | 18 | 0.1 (0.24) | 0.0 (0, 1) |
| Week 6 | 47 | 0.1 (0.25) | 0.0 (0, 1) | 30 | 0.1 (0.31) | 0.0 (0, 1) | 17 | 0.0 (0.00) | 0.0 (0, 0) |
| Week 12 | 42 | 0.0 (0.15) | 0.0 (0, 1) | 28 | 0.0 (0.19) | 0.0 (0, 1) | 14 | 0.0 (0.00) | 0.0 (0, 0) |
| Placebo/d6-DM/Q (N = 40) | | | | | | | | | |
| Placebo | | | | | | | | | |
| Baseline [1] | 40 | 0.0 (0.16) | 0.0 (0, 1) | 33 | 0.0 (0.17) | 0.0 (0, 1) | 7 | 0.0 (0.00) | 0.0 (0, 0) |
| Week 6 | 40 | 0.1 (0.30) | 0.0 (0, 1) | 33 | 0.1 (0.33) | 0.0 (0, 1) | 7 | 0.0 (0.00) | 0.0 (0, 0) |
| d6-DM/Q | | | | | | | | | |
| Week 6 (BL) [2] | 39 | 0.1 (0.31) | 0.0 (0, 1) | 32 | 0.1 (0.34) | 0.0 (0, 1) | 7 | 0.0 (0.00) | 0.0 (0, 0) |
| Week 12 | 39 | 0.1 (0.22) | 0.0 (0, 1) | 32 | 0.1 (0.25) | 0.0 (0, 1) | 7 | 0.0 (0.00) | 0.0 (0, 0) |

Item: Salivation

| | | | | | | | | | |
|---|---|---|---|---|---|---|---|---|---|
| Placebo/Placebo (N = 56) | | | | | | | | | |
| Baseline [1] | 56 | 0.0 (0.13) | 0.0 (0, 1) | 36 | 0.0 (0.00) | 0.0 (0, 0) | 20 | 0.1 (0.22) | 0.0 (0, 1) |
| Week 6 | 51 | 0.0 (0.00) | 0.0 (0, 0) | 33 | 0.0 (0.00) | 0.0 (0, 0) | 18 | 0.0 (0.00) | 0.0 (0, 0) |
| Week [2] | 39 | 0.0 (0.00) | 0.0 (0, 0) | 26 | 0.0 (0.00) | 0.0 (0, 0) | 13 | 0.0 (0.00) | 0.0 (0, 0) |
| d6-DM/Q/d6-DM/Q (N = 48) | | | | | | | | | |
| Baseline [1] | 48 | 0.0 (0.00) | 0.0 (0, 0) | 30 | 0.0 (0.00) | 0.0 (0, 0) | 18 | 0.0 (0.00) | 0.0 (0, 0) |
| Week 6 | 47 | 0.0 (0.00) | 0.0 (0, 0) | 30 | 0.0 (0.00) | 0.0 (0, 0) | 17 | 0.0 (0.00) | 0.0 (0, 0) |
| Week 12 | 42 | 0.0 (0.00) | 0.0 (0, 0) | 28 | 0.0 (0.00) | 0.0 (0, 0) | 14 | 0.0 (0.00) | 0.0 (0, 0) |
| Placebo/d6-DM/Q (N = 40) | | | | | | | | | |
| Placebo | | | | | | | | | |
| Baseline [1] | 40 | 0.1 (0.44) | 0.0 (0, 2) | 33 | 0.1 (0.48) | 0.0 (0, 2) | 7 | 0.0 (0.00) | 0.0 (0, 0) |
| Week 6 | 40 | 0.0 (0.16) | 0.0 (0, 1) | 33 | 0.0 (0.17) | 0.0 (0, 1) | 7 | 0.0 (0.00) | 0.0 (0, 0) |
| d6-DM/Q | | | | | | | | | |
| Week 6 (BL) [2] | 39 | 0.0 (0.16) | 0.0 (0, 1) | 32 | 0.0 (0.18) | 0.0 (0, 1) | 7 | 0.0 (0.00) | 0.0 (0, 0) |
| Week 12 | 39 | 0.1 (0.27) | 0.0 (0, 1) | 32 | 0.1 (0.30) | 0.0 (0, 1) | 7 | 0.0 (0.00) | 0.0 (0, 0) |

Note:
SAS total score ranges from 0 to 40, with higher scores indicating greater severity of extrapyramidal symptoms. Individual item scores range from 0 to 4. Visit windows are used to classify unscheduled or early termination visits.
[1] Baseline for Placebo/Placebo and d6-DM/Q/d6-DM/Q is defined as the last non-missing assessment prior to randomization.
[2] Baseline for Stage 2 is the Week 6 assessment for the Placebo/d6-DM/Q group.

TABLE 62
Shift Table of SAS from Baseline to End of Stage
Safety Population (N = 144)

| | | End of | Baseline | | | | | |
|---|---|---|---|---|---|---|---|---|
| Stage | Treatment | Stage | 0 | 1 | 2 | 3 | 4 | Total |

Item: Gait

| | | | | | | | | |
|---|---|---|---|---|---|---|---|---|
| Stage 1 | Stage 1 Placebo (N = 96) | 0 | 75 (82.4) | 2 (2.2) | 1 (1.1) | 0 (0.0) | 0 (0.0) | 78 (85.7) |
| Stage 1 | Stage 1 Placebo (N = 96) | 1 | 2 (2.2) | 8 (8.8) | 1 (1.1) | 0 (0.0) | 0 (0.0) | 11 (2.1) |
| Stage 1 | Stage 1 Placebo (N = 96) | 2 | 0 (0.0) | 1 (1.1) | 1 (1.1) | 0 (0.0) | 0 (0.0) | 2 (2.2) |
| Stage 1 | Stage 1 Placebo (N = 96) | 3 | 0 (0.0) | 0 (0.0) | 0 (0.0) | 0 (0.0) | 0 (0.0) | 0 (0.0) |
| Stage 1 | Stage 1 Placebo (N = 96) | 4 | 0 (0.0) | 0 (0.0) | 0 (0.0) | 0 (0.0) | 0 (0.0) | 0 (0.0) |
| | Stage 1 d6-DM/Q (N = 48) | 0 | 35 (74.5) | 1 (2.1) | 0 (0.0) | 0 (0.0) | 0 (0.0) | 36 (76.6) |
| | Stage 1 d6-DM/Q (N = 48) | 1 | 3 (6.4) | 8 (17.0) | 0 (0.0) | 0 (0.0) | 0 (0.0) | 11 (23.4) |
| | Stage 1 d6-DM/Q (N = 48) | 2 | 0 (0.0) | 0 (0.0) | 0 (0.0) | 0 (0.0) | 0 (0.0) | 0 (0.0) |
| | Stage 1 d6-DM/Q (N = 48) | 3 | 0 (0.0) | 0 (0.0) | 0 (0.0) | 0 (0.0) | 0 (0.0) | 0 (0.0) |
| | Stage 1 d6-DM/Q (N = 48) | 4 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 0 | 37 (94.9) | 0 (0.0) | 0 (0.0) | 0 (0.0) | 0 (0.0) | 37 (94.9) |
| Stage 2 | Stage 1 Placebo Re-randomized to Placebo (N = 40) | 1 | 0 (0.0) | 1 (2.6) | 0 (0.0) | 0 (0.0) | 0 (0.0) | 1 (2.6) |
| Stage 2 | Stage 1 Placebo Re-randomized to Placebo (N = 40) | 2 | 0 (0.0) | 0 (0.0) | 1 (2.6) | 0 (0.0) | 0 (0.0) | 1 (2.6) |
| Stage 2 | Stage 1 Placebo Re-randomized to Placebo (N = 40) | 3 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 4 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 0 | 28 (71.8) | 1 (2.6) | 1 (2.6) | 0 (0.0) | 0 (0.0) | 30 (76.9) |
| | Stage 1 Placebo Re-randomized to d6-DM/Q (N = 40) | 1 | 2 (5.1) | 7 (17.9) | 0 (0.0) | 0 (0.0) | 0 (0.0) | 9 (23.1) |
| | Stage 1 Placebo Re-randomized to d6-DM/Q (N = 40) | 2 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 3 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 4 | 0 (0.0) | 0 (0.0) | 0 (0.0) | 0 (0.0) | 0 (0.0) | 0 (0.0) |

Item: Arm Dropping

| | | | | | | | | |
|---|---|---|---|---|---|---|---|---|
| Stage 1 | Stage 1 Placebo (N = 96) | 0 | 87 (95.6) | 2 (2.2) | 0 (0.0) | 0 (0.0) | 0 (0.0) | 89 (97.8) |
| Stage 1 | Stage 1 Placebo (N = 96) | 1 | 1 (1.1) | 1 (1.1) | 0 (0.0) | 0 (0.0) | 0 (0.0) | 2 (2.2) |
| Stage 1 | Stage 1 Placebo (N = 96) | 2 | 0 (0.0) | 0 (0.0) | 0 (0.0) | 0 (0.0) | 0 (0.0) | 0 (0.0) |
| Stage 1 | Stage 1 Placebo (N = 96) | 3 | 0 (0.0) | 0 (0.0) | 0 (0.0) | 0 (0.0) | 0 (0.0) | 0 (0.0) |
| Stage 1 | Stage 1 Placebo (N = 96) | 4 | 0 (0.0) | 0 (0.0) | 0 (0.0) | 0 (0.0) | 0 (0.0) | 0 (0.0) |
| | Stage 1 d6-DM/Q (N = 48) | 0 | 46 (97.9) | 1 (2.1) | 0 (0.0) | 0 (0.0) | 0 (0.0) | 47 (100.0) |
| | Stage 1 d6-DM/Q (N = 48) | 1 | 0 (0.0) | 0 (0.0) | 0 (0.0) | 0 (0.0) | 0 (0.0) | 0 (0.0) |
| | Stage 1 d6-DM/Q (N = 48) | 2 | 0 (0.0) | 0 (0.0) | 0 (0.0) | 0 (0.0) | 0 (0.0) | 0 (0.0) |
| | Stage 1 d6-DM/Q (N = 48) | 3 | 0 (0.0) | 0 (0.0) | 0 (0.0) | 0 (0.0) | 0 (0.0) | 0 (0.0) |
| | Stage 1 d6-DM/Q (N = 48) | 4 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 0 | 38 (97.4) | 0 (0.0) | 0 (0.0) | 0 (0.0) | 0 (0.0) | 38 (97.4) |
| Stage 2 | Stage 1 Placebo Re-randomized to Placebo (N = 40) | 1 | 0 (0.0) | 1 (2.6) | 0 (0.0) | 0 (0.0) | 0 (0.0) | 1 (2.6) |
| Stage 2 | Stage 1 Placebo Re-randomized to Placebo (N = 40) | 2 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 3 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 4 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 0 | 36 (92.3) | 1 (2.6) | 0 (0.0) | 0 (0.0) | 0 (0.0) | 37 (94.9) |
| | Stage 1 Placebo Re-randomized to d6-DM/Q (N = 40) | 1 | 2 (5.1) | 0 (0.0) | 0 (0.0) | 0 (0.0) | 0 (0.0) | 2 (5.1) |
| | Stage 1 Placebo Re-randomized to d6-DM/Q (N = 40) | 2 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 3 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 4 | 0 (0.0) | 0 (0.0) | 0 (0.0) | 0 (0.0) | 0 (0.0) | 0 (0.0) |

Item: Shoulder Shaking

| | | | | | | | | |
|---|---|---|---|---|---|---|---|---|
| Stage 1 | Stage 1 Placebo (N = 96) | 0 | 85 (93.4) | 1 (1.1) | 0 (0.0) | 0 (0.0) | 0 (0.0) | 86 (94.5) |
| Stage 1 | Stage 1 Placebo (N = 96) | 1 | 3 (3.3) | 1 (1.1) | 1 (1.1) | 0 (0.0) | 0 (0.0) | 5 (5.5) |
| Stage 1 | Stage 1 Placebo (N = 96) | 2 | 0 (0.0) | 0 (0.0) | 0 (0.0) | 0 (0.0) | 0 (0.0) | 0 (0.0) |
| Stage 1 | Stage 1 Placebo (N = 96) | 3 | 0 (0.0) | 0 (0.0) | 0 (0.0) | 0 (0.0) | 0 (0.0) | 0 (0.0) |
| Stage 1 | Stage 1 Placebo (N = 96) | 4 | 0 (0.0) | 0 (0.0) | 0 (0.0) | 0 (0.0) | 0 (0.0) | 0 (0.0) |
| | Stage 1 d6-DM/Q (N = 48) | 0 | 44 (93.6) | 1 (2.1) | 0 (0.0) | 0 (0.0) | 0 (0.0) | 45 (95.7) |
| | Stage 1 d6-DM/Q (N = 48) | 1 | 1 (2.1) | 1 (2.1) | 0 (0.0) | 0 (0.0) | 0 (0.0) | 2 (4.3) |
| | Stage 1 d6-DM/Q (N = 48) | 2 | 0 (0.0) | 0 (0.0) | 0 (0.0) | 0 (0.0) | 0 (0.0) | 0 (0.0) |
| | Stage 1 d6-DM/Q (N = 48) | 3 | 0 (0.0) | 0 (0.0) | 0 (0.0) | 0 (0.0) | 0 (0.0) | 0 (0.0) |
| | Stage 1 d6-DM/Q (N = 48) | 4 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 0 | 38 (97.4) | 0 (0.0) | 0 (0.0) | 0 (0.0) | 0 (0.0) | 38 (97.4) |
| Stage 2 | Stage 1 Placebo Re-randomized to Placebo (N = 40) | 1 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 2 | 0 (0.0) | 1 (2.6) | 0 (0.0) | 0 (0.0) | 0 (0.0) | 1 (2.6) |
| Stage 2 | Stage 1 Placebo Re-randomized to Placebo (N = 40) | 3 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 4 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 0 | 35 (89.7) | 2 (5.1) | 0 (0.0) | 0 (0.0) | 0 (0.0) | 37 (94.9) |
| | Stage 1 Placebo Re-randomized to d6-DM/Q (N = 40) | 1 | 1 (2.6) | 1 (2.6) | 0 (0.0) | 0 (0.0) | 0 (0.0) | 2 (5.1) |
| | Stage 1 Placebo Re-randomized to d6-DM/Q (N = 40) | 2 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 3 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 4 | 0 (0.0) | 0 (0.0) | 0 (0.0) | 0 (0.0) | 0 (0.0) | 0 (0.0) |

Item: Elbow Rigidity

| | | | | | | | | |
|---|---|---|---|---|---|---|---|---|
| Stage 1 | Stage 1 Placebo (N = 96) | 0 | 88 (96.7) | 1 (1.1) | 0 (0.0) | 0 (0.0) | 0 (0.0) | 89 (97.8) |
| Stage 1 | Stage 1 Placebo (N = 96) | 1 | 1 (1.1) | 0 (0.0) | 0 (0.0) | 0 (0.0) | 0 (0.0) | 1 (1.1) |
| Stage 1 | Stage 1 Placebo (N = 96) | 2 | 0 (0.0) | 1 (1.1) | 0 (0.0) | 0 (0.0) | 0 (0.0) | 1 (1.1) |
| Stage 1 | Stage 1 Placebo (N = 96) | 3 | 0 (0.0) | 0 (0.0) | 0 (0.0) | 0 (0.0) | 0 (0.0) | 0 (0.0) |
| Stage 1 | Stage 1 Placebo (N = 96) | 4 | 0 (0.0) | 0 (0.0) | 0 (0.0) | 0 (0.0) | 0 (0.0) | 0 (0.0) |
| | Stage 1 d6-DM/Q (N = 48) | 0 | 46 (97.9) | 0 (0.0) | 0 (0.0) | 0 (0.0) | 0 (0.0) | 46 (97.9) |
| | Stage 1 d6-DM/Q (N = 48) | 1 | 0 (0.0) | 1 (2.1) | 0 (0.0) | 0 (0.0) | 0 (0.0) | 1 (2.1) |
| | Stage 1 d6-DM/Q (N = 48) | 2 | 0 (0.0) | 0 (0.0) | 0 (0.0) | 0 (0.0) | 0 (0.0) | 0 (0.0) |
| | Stage 1 d6-DM/Q (N = 48) | 3 | 0 (0.0) | 0 (0.0) | 0 (0.0) | 0 (0.0) | 0 (0.0) | 0 (0.0) |
| | Stage 1 d6-DM/Q (N = 48) | 4 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 0 | 38 (97.4) | 0 (0.0) | 0 (0.0) | 0 (0.0) | 0 (0.0) | 38 (97.4) |
| Stage 2 | Stage 1 Placebo Re-randomized to Placebo (N = 40) | 1 | 0 (0.0) | 0 (0.0) | 1 (2.6) | 0 (0.0) | 0 (0.0) | 1 (2.6) |
| Stage 2 | Stage 1 Placebo Re-randomized to Placebo (N = 40) | 2 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 3 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 4 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 0 | 35 (89.7) | 1 (2.6) | 0 (0.0) | 0 (0.0) | 0 (0.0) | 36 (92.3) |
| | Stage 1 Placebo Re-randomized to d6-DM/Q (N = 40) | 1 | 3 (7.7) | 0 (0.0) | 0 (0.0) | 0 (0.0) | 0 (0.0) | 3 (7.7) |
| | Stage 1 Placebo Re-randomized to d6-DM/Q (N = 40) | 2 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 3 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 4 | 0 (0.0) | 0 (0.0) | 0 (0.0) | 0 (0.0) | 0 (0.0) | 0 (0.0) |

Item: Wrist Rigid/Fixation of Position

| | | | | | | | | |
|---|---|---|---|---|---|---|---|---|
| Stage 1 | Stage 1 Placebo (N = 96) | 0 | 90 (98.9) | 0 (0.0) | 0 (0.0) | 0 (0.0) | 0 (0.0) | 90 (98.9) |
| Stage 1 | Stage 1 Placebo (N = 96) | 1 | 0 (0.0) | 0 (0.0) | 0 (0.0) | 0 (0.0) | 0 (0.0) | 0 (0.0) |
| Stage 1 | Stage 1 Placebo (N = 96) | 2 | 0 (0.0) | 0 (0.0) | 1 (1.1) | 0 (0.0) | 0 (0.0) | 1 (1.1) |
| Stage 1 | Stage 1 Placebo (N = 96) | 3 | 0 (0.0) | 0 (0.0) | 0 (0.0) | 0 (0.0) | 0 (0.0) | 0 (0.0) |
| Stage 1 | Stage 1 Placebo (N = 96) | 4 | 0 (0.0) | 0 (0.0) | 0 (0.0) | 0 (0.0) | 0 (0.0) | 0 (0.0) |
| | Stage 1 d6-DM/Q (N = 48) | 0 | 45 (95.7) | 0 (0.0) | 0 (0.0) | 0 (0.0) | 0 (0.0) | 45 (95.7) |
| | Stage 1 d6-DM/Q (N = 48) | 1 | 1 (2.1) | 1 (2.1) | 0 (0.0) | 0 (0.0) | 0 (0.0) | 2 (4.3) |
| | Stage 1 d6-DM/Q (N = 48) | 2 | 0 (0.0) | 0 (0.0) | 0 (0.0) | 0 (0.0) | 0 (0.0) | 0 (0.0) |
| | Stage 1 d6-DM/Q (N = 48) | 3 | 0 (0.0) | 0 (0.0) | 0 (0.0) | 0 (0.0) | 0 (0.0) | 0 (0.0) |
| | Stage 1 d6-DM/Q (N = 48) | 4 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 0 | 38 (97.4) | 0 (0.0) | 1 (2.6) | 0 (0.0) | 0 (0.0) | 39 (100.0) |
| Stage 2 | Stage 1 Placebo Re-randomized to Placebo (N = 40) | 1 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 2 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 3 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 4 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 0 | 36 (92.3) | 0 (0.0) | 0 (0.0) | 0 (0.0) | 0 (0.0) | 36 (92.3) |
| | Stage 1 Placebo Re-randomized to d6-DM/Q (N = 40) | 1 | 3 (7.7) | 0 (0.0) | 0 (0.0) | 0 (0.0) | 0 (0.0) | 3 (7.7) |
| | Stage 1 Placebo Re-randomized to d6-DM/Q (N = 40) | 2 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 3 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 4 | 0 (0.0) | 0 (0.0) | 0 (0.0) | 0 (0.0) | 0 (0.0) | 0 (0.0) |

Item: Leg Pendulousness

| | | | | | | | | |
|---|---|---|---|---|---|---|---|---|
| Stage 1 | Stage 1 Placebo (N = 96) | 0 | 88 (96.7) | 1 (1.1) | 0 (0.0) | 0 (0.0) | 0 (0.0) | 89 (97.8) |
| Stage 1 | Stage 1 Placebo (N = 96) | 1 | 1 (1.1) | 0 (0.0) | 1 (1.1) | 0 (0.0) | 0 (0.0) | 2 (2.2) |
| Stage 1 | Stage 1 Placebo (N = 96) | 2 | 0 (0.0) | 0 (0.0) | 0 (0.0) | 0 (0.0) | 0 (0.0) | 0 (0.0) |
| Stage 1 | Stage 1 Placebo (N = 96) | 3 | 0 (0.0) | 0 (0.0) | 0 (0.0) | 0 (0.0) | 0 (0.0) | 0 (0.0) |
| Stage 1 | Stage 1 Placebo (N = 96) | 4 | 0 (0.0) | 0 (0.0) | 0 (0.0) | 0 (0.0) | 0 (0.0) | 0 (0.0) |
| | Stage 1 d6-DM/Q (N = 48) | 0 | 46 (97.9) | 0 (0.0) | 0 (0.0) | 0 (0.0) | 0 (0.0) | 46 (97.9) |
| | Stage 1 d6-DM/Q (N = 48) | 1 | 0 (0.0) | 1 (2.1) | 0 (0.0) | 0 (0.0) | 0 (0.0) | 1 (2.1) |
| | Stage 1 d6-DM/Q (N = 48) | 2 | 0 (0.0) | 0 (0.0) | 0 (0.0) | 0 (0.0) | 0 (0.0) | 0 (0.0) |
| | Stage 1 d6-DM/Q (N = 48) | 3 | 0 (0.0) | 0 (0.0) | 0 (0.0) | 0 (0.0) | 0 (0.0) | 0 (0.0) |
| | Stage 1 d6-DM/Q (N = 48) | 4 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 0 | 39 (100.0) | 0 (0.0) | 0 (0.0) | 0 (0.0) | 0 (0.0) | 39 (100.0) |
| Stage 2 | Stage 1 Placebo Re-randomized to Placebo (N = 40) | 1 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 2 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 3 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 4 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 0 | 36 (92.3) | 1 (2.6) | 0 (0.0) | 0 (0.0) | 0 (0.0) | 37 (94.9) |
| | Stage 1 Placebo Re-randomized to d6-DM/Q (N = 40) | 1 | 1 (2.6) | 0 (0.0) | 0 (0.0) | 0 (0.0) | 0 (0.0) | 1 (2.6) |
| | Stage 1 Placebo Re-randomized to d6-DM/Q (N = 40) | 2 | 0 (0.0) | 1 (2.6) | 0 (0.0) | 0 (0.0) | 0 (0.0) | 1 (2.6) |
| | Stage 1 Placebo Re-randomized to d6-DM/Q (N = 40) | 3 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 4 | 0 (0.0) | 0 (0.0) | 0 (0.0) | 0 (0.0) | 0 (0.0) | 0 (0.0) |

Item: Head Rotation

| | | | | | | | | |
|---|---|---|---|---|---|---|---|---|
| Stage 1 | Stage 1 Placebo (N = 96) | 0 | 84 (92.3) | 4 (4.4) | 0 (0.0) | 0 (0.0) | 0 (0.0) | 88 (96.7) |
| Stage 1 | Stage 1 Placebo (N = 96) | 1 | 2 (2.2) | 1 (1.1) | 0 (0.0) | 0 (0.0) | 0 (0.0) | 3 (3.3) |
| Stage 1 | Stage 1 Placebo (N = 96) | 2 | 0 (0.0) | 0 (0.0) | 0 (0.0) | 0 (0.0) | 0 (0.0) | 0 (0.0) |
| Stage 1 | Stage 1 Placebo (N = 96) | 3 | 0 (0.0) | 0 (0.0) | 0 (0.0) | 0 (0.0) | 0 (0.0) | 0 (0.0) |
| Stage 1 | Stage 1 Placebo (N = 96) | 4 | 0 (0.0) | 0 (0.0) | 0 (0.0) | 0 (0.0) | 0 (0.0) | 0 (0.0) |
| | Stage 1 d6-DM/Q (N = 48) | 0 | 45 (95.7) | 1 (2.1) | 0 (0.0) | 0 (0.0) | 0 (0.0) | 46 (97.9) |
| | Stage 1 d6-DM/Q (N = 48) | 1 | 1 (2.1) | 0 (0.0) | 0 (0.0) | 0 (0.0) | 0 (0.0) | 1 (2.1) |
| | Stage 1 d6-DM/Q (N = 48) | 2 | 0 (0.0) | 0 (0.0) | 0 (0.0) | 0 (0.0) | 0 (0.0) | 0 (0.0) |
| | Stage 1 d6-DM/Q (N = 48) | 3 | 0 (0.0) | 0 (0.0) | 0 (0.0) | 0 (0.0) | 0 (0.0) | 0 (0.0) |
| | Stage 1 d6-DM/Q (N = 48) | 4 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 0 | 38 (97.4) | 0 (0.0) | 0 (0.0) | 0 (0.0) | 0 (0.0) | 38 (97.4) |
| Stage 2 | Stage 1 Placebo Re-randomized to Placebo (N = 40) | 1 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 2 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 3 | 0 (0.0) | 1 (2.6) | 0 (0.0) | 0 (0.0) | 0 (0.0) | 1 (2.6) |
| Stage 2 | Stage 1 Placebo Re-randomized to Placebo (N = 40) | 4 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 0 | 36 (92.3) | 0 (0.0) | 0 (0.0) | 0 (0.0) | 0 (0.0) | 36 (92.3) |
| | Stage 1 Placebo Re-randomized to d6-DM/Q (N = 40) | 1 | 2 (5.1) | 1 (2.6) | 0 (0.0) | 0 (0.0) | 0 (0.0) | 3 (7.7) |
| | Stage 1 Placebo Re-randomized to d6-DM/Q (N = 40) | 2 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 3 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 4 | 0 (0.0) | 0 (0.0) | 0 (0.0) | 0 (0.0) | 0 (0.0) | 0 (0.0) |

Item: Glabella Tap

| | | | | | | | | |
|---|---|---|---|---|---|---|---|---|
| Stage 1 | Stage 1 Placebo (N = 96) | 0 | 87 (95.6) | 1 (1.1) | 0 (0.0) | 0 (0.0) | 0 (0.0) | 88 (96.7) |
| Stage 1 | Stage 1 Placebo (N = 96) | 1 | 1 (1.1) | 1 (1.1) | 0 (0.0) | 0 (0.0) | 0 (0.0) | 2 (2.2) |
| Stage 1 | Stage 1 Placebo (N = 96) | 2 | 0 (0.0) | 0 (0.0) | 0 (0.0) | 0 (0.0) | 0 (0.0) | 0 (0.0) |
| Stage 1 | Stage 1 Placebo (N = 96) | 3 | 0 (0.0) | 0 (0.0) | 0 (0.0) | 0 (0.0) | 0 (0.0) | 0 (0.0) |
| Stage 1 | Stage 1 Placebo (N = 96) | 4 | 1 (1.1) | 0 (0.0) | 0 (0.0) | 0 (0.0) | 0 (0.0) | 1 (1.1) |
| | Stage 1 d6-DM/Q (N = 48) | 0 | 44 (93.6) | 2 (4.3) | 0 (0.0) | 0 (0.0) | 0 (0.0) | 46 (97.9) |
| | Stage 1 d6-DM/Q (N = 48) | 1 | 1 (2.1) | 0 (0.0) | 0 (0.0) | 0 (0.0) | 0 (0.0) | 1 (2.1) |
| | Stage 1 d6-DM/Q (N = 48) | 2 | 0 (0.0) | 0 (0.0) | 0 (0.0) | 0 (0.0) | 0 (0.0) | 0 (0.0) |
| | Stage 1 d6-DM/Q (N = 48) | 3 | 0 (0.0) | 0 (0.0) | 0 (0.0) | 0 (0.0) | 0 (0.0) | 0 (0.0) |
| | Stage 1 d6-DM/Q (N = 48) | 4 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 0 | 38 (97.4) | 1 (2.6) | 0 (0.0) | 0 (0.0) | 0 (0.0) | 39 (100.0) |
| Stage 2 | Stage 1 Placebo Re-randomized to Placebo (N = 40) | 1 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 2 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 3 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 4 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 0 | 37 (94.9) | 0 (0.0) | 0 (0.0) | 0 (0.0) | 0 (0.0) | 38 (97.4) |
| | Stage 1 Placebo Re-randomized to d6-DM/Q (N = 40) | 1 | 1 (2.6) | 0 (0.0) | 0 (0.0) | 0 (0.0) | 0 (0.0) | 1 (2.6) |
| | Stage 1 Placebo Re-randomized to d6-DM/Q (N = 40) | 2 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 3 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 4 | 0 (0.0) | 0 (0.0) | 0 (0.0) | 0 (0.0) | 0 (0.0) | 0 (0.0) |

Item: Tremor

| | | | | | | | | |
|---|---|---|---|---|---|---|---|---|
| Stage 1 | Stage 1 Placebo (N = 96) | 0 | 85 (93.4) | 1 (1.1) | 0 (0.0) | 0 (0.0) | 0 (0.0) | 86 (94.5) |
| Stage 1 | Stage 1 Placebo (N = 96) | 1 | 4 (4.4) | 1 (1.1) | 0 (0.0) | 0 (0.0) | 0 (0.0) | 5 (5.5) |
| Stage 1 | Stage 1 Placebo (N = 96) | 2 | 0 (0.0) | 0 (0.0) | 0 (0.0) | 0 (0.0) | 0 (0.0) | 0 (0.0) |
| Stage 1 | Stage 1 Placebo (N = 96) | 3 | 0 (0.0) | 0 (0.0) | 0 (0.0) | 0 (0.0) | 0 (0.0) | 0 (0.0) |
| Stage 1 | Stage 1 Placebo (N = 96) | 4 | 0 (0.0) | 0 (0.0) | 0 (0.0) | 0 (0.0) | 0 (0.0) | 0 (0.0) |
| | Stage 1 d6-DM/Q (N = 48) | 0 | 40 (85.1) | 4 (8.5) | 0 (0.0) | 0 (0.0) | 0 (0.0) | 44 (93.6) |
| | Stage 1 d6-DM/Q (N = 48) | 1 | 1 (2.1) | 2 (4.3) | 0 (0.0) | 0 (0.0) | 0 (0.0) | 3 (6.4) |
| | Stage 1 d6-DM/Q (N = 48) | 2 | 0 (0.0) | 0 (0.0) | 0 (0.0) | 0 (0.0) | 0 (0.0) | 0 (0.0) |
| | Stage 1 d6-DM/Q (N = 48) | 3 | 0 (0.0) | 0 (0.0) | 0 (0.0) | 0 (0.0) | 0 (0.0) | 0 (0.0) |
| | Stage 1 d6-DM/Q (N = 48) | 4 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 0 | 37 (94.9) | 1 (2.6) | 0 (0.0) | 0 (0.0) | 0 (0.0) | 38 (97.4) |
| Stage 2 | Stage 1 Placebo Re-randomized to Placebo (N = 40) | 1 | 1 (2.6) | 0 (0.0) | 0 (0.0) | 0 (0.0) | 0 (0.0) | 1 (2.6) |
| Stage 2 | Stage 1 Placebo Re-randomized to Placebo (N = 40) | 2 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 3 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 4 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 0 | 34 (87.2) | 3 (7.7) | 0 (0.0) | 0 (0.0) | 0 (0.0) | 37 (94.9) |
| | Stage 1 Placebo Re-randomized to d6-DM/Q (N = 40) | 1 | 1 (2.6) | 1 (2.6) | 0 (0.0) | 0 (0.0) | 0 (0.0) | 2 (5.1) |
| | Stage 1 Placebo Re-randomized to d6-DM/Q (N = 40) | 2 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 3 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 4 | 0 (0.0) | 0 (0.0) | 0 (0.0) | 0 (0.0) | 0 (0.0) | 0 (0.0) |

Item: Salivation

| | | | | | | | | |
|---|---|---|---|---|---|---|---|---|
| Stage 1 | Stage 1 Placebo (N = 96) | 0 | 88 (96.7) | 1 (1.1) | 1 (1.1) | 0 (0.0) | 0 (0.0) | 90 (98.9) |
| Stage 1 | Stage 1 Placebo (N = 96) | 1 | 0 (0.0) | 0 (0.0) | 1 (1.1) | 0 (0.0) | 0 (0.0) | 1 (1.1) |
| Stage 1 | Stage 1 Placebo (N = 96) | 2 | 0 (0.0) | 0 (0.0) | 0 (0.0) | 0 (0.0) | 0 (0.0) | 0 (0.0) |
| Stage 1 | Stage 1 Placebo (N = 96) | 3 | 0 (0.0) | 0 (0.0) | 0 (0.0) | 0 (0.0) | 0 (0.0) | 0 (0.0) |
| Stage 1 | Stage 1 Placebo (N = 96) | 4 | 0 (0.0) | 0 (0.0) | 0 (0.0) | 0 (0.0) | 0 (0.0) | 0 (0.0) |
| | Stage 1 d6-DM/Q (N = 48) | 0 | 47 (100.0) | 0 (0.0) | 0 (0.0) | 0 (0.0) | 0 (0.0) | 47 (100.0) |
| | Stage 1 d6-DM/Q (N = 48) | 1 | 0 (0.0) | 0 (0.0) | 0 (0.0) | 0 (0.0) | 0 (0.0) | 0 (0.0) |
| | Stage 1 d6-DM/Q (N = 48) | 2 | 0 (0.0) | 0 (0.0) | 0 (0.0) | 0 (0.0) | 0 (0.0) | 0 (0.0) |
| | Stage 1 d6-DM/Q (N = 48) | 3 | 0 (0.0) | 0 (0.0) | 0 (0.0) | 0 (0.0) | 0 (0.0) | 0 (0.0) |
| | Stage 1 d6-DM/Q (N = 48) | 4 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 0 | 39 (100.0) | 0 (0.0) | 0 (0.0) | 0 (0.0) | 0 (0.0) | 39 (100.0) |
| Stage 2 | Stage 1 Placebo Re-randomized to Placebo (N = 40) | 1 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 2 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 3 | 0 (0.0) | 0 (0.0) | 0 (0.0) | 0 (0.0) | 0 (0.0) | 0 (0.0) |
| Stage 2 | Stage 1 Placebo Re-randomized to Placebo (N = 40) | 4 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 0 | 35 (89.7) | 1 (2.6) | 0 (0.0) | 0 (0.0) | 0 (0.0) | 36 (92.3) |
| | Stage 1 Placebo Re-randomized to d6-DM/Q (N = 40) | 1 | 3 (7.7) | 0 (0.0) | 0 (0.0) | 0 (0.0) | 0 (0.0) | 3 (7.7) |
| | Stage 1 Placebo Re-randomized to d6-DM/Q (N = 40) | 2 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 3 | 0 (0.0) | 0 (0.0) | 0 (0.0) | 0 (0.0) | 0 (0.0) | 0 (0.0) |
| | Stage 1 Placebo Re-randomized to d6-DM/Q (N = 40) | 4 | 0 (0.0) | 0 (0.0) | 0 (0.0) | 0 (0.0) | 0 (0.0) | 0 (0.0) |

Note:
Percentages are based on the number of patients in each treatment with a baseline and post-baseline value within a stage. Stage 1 compares the Stage 1 Baseline to the last post-baseline visit in Stage 1, including Week 6. Stage 1 re-randomized compares Stage 2 Baseline to the last assessment, whenever it occurred.

TABLE 63
MCCB Composite Score: Change from Baseline SPCD ANCOVA, LOCF Data
mITT Population (N = 127)

| | Visit | | | | |
|---|---|---|---|---|---|
| Stage | Statistics | Placebo | | d6-DM/Q | |
| Stage 1 | Baseline: N, Mean (SD) | 80, 28.8 | (13.09) | 47, 32.5 | (11.37) |
| | Week 6: N, Mean (SD) | 75, 30.1 | (12.44) | 46, 33.6 | (12.73) |
| | Change from Baseline: | 75, 1.6 | (4.55) | 46, 1.2 | (5.11) |
| | N, Mean (SD) | | | | |
| | Standard Effect Size | | | −0.083 | |
| | % Change from Baseline: | 75, 15.7 | (50.64) | 46, 4.7 | (17.95) |
| | N, Mean (SD) | | | | |
| | Treatment Difference vs. Placebo: | | | −0.12 | (−1.88, 1.64) |
| | LS Mean Difference, 95% CI [1] | | | | |
| | p-value [1] | | | 0.893 | |
| Stage 2 | Baseline [2]: N, Mean (SD) | 27, 31.7 | (14.99) | 30, 28.9 | (10.69) |
| (Stage 1 | Week 12: N, Mean (SD) | 27, 30.0 | (15.60) | 30, 30.5 | (10.99) |
| Placebo | Change from Baseline [2]: | 27, −1.6 | (4.06) | 30, 1.6 | (3.71) |
| Non- | N, Mean (SD) | | | | |
| re- | Standard Effect Size | | | 0.833 | |
| sponders) | % Change from Baseline [2]: | 27, −7.9 | (23.15) | 30, 6.6 | (15.50) |
| | N, Mean (SD) | | | | |
| | Treatment Difference vs. Placebo: | | | 3.21 | (1.11, 5.30) |
| | LS Mean Difference, 95% CI [1] | | | | |
| | p-value [1] | | | 0.003 | |
| | SPCD Weighted OLS z-statistic, | | | 1.78, | 0.074 |
| | overall p-value [3] | | | | |

Note:
MCCB Composite Score with higher scores indicating less clinical severity of symptoms.
Standard Effect Size is defined as (mean change in d6-DM/Q-mean change in Placebo)/Change from Baseline Pooled SD.
[1] Change from Baseline was analyzed at each stage by ANCOVA with treatment as fixed effect and baseline value as covariate. Missing values were imputed by LOCF within each stage.
[2] Stage 2 Baseline is the last non-missing assessment prior to re-randomization into Stage 2 (re-randomization visit).
[3] SPCD Weighted OLS z-statistic was calculated using Stage 1 weight = 0.6 and Stage 2 weight = 0.4.

TABLE 64
MCCB Composite Score: Change from Baseline SPCD ANCOVA, LOCF Data
Per Protocol Population (N = 110)

| | Visit | | | | |
|---|---|---|---|---|---|
| Stage | Statistics | Placebo | | d6-DM/Q | |
| Stage 1 | Baseline: N, Mean (SD) | 73, 28.4 | (13.06) | 37, 32.6 | (10.94) |
| | Week 6: N, Mean (SD) | 69, 29.6 | (12.14) | 36, 33.6 | (11.66) |
| | Change from Baseline: | 69, 1.7 | (4.51) | 36, 1.1 | (4.76) |
| | N, Mean (SD) | | | | |
| | Standard Effect Size | | | −0.112 | |
| | % Change from Baseline: | 69, 16.8 | (52.49) | 36, 5.2 | (17.35) |
| | N, Mean (SD) | | | | |
| | Treatment Difference vs. Placebo: | | | −0.04 | (−1.87, 1.80) |
| | LS Mean Difference, 95% CI [1] | | | | |
| | p-value [1] | | | 0.967 | |
| Stage 2 | Baseline 2; N, Mean (SD) | 24, 3 16 | (14.75) | 28, 28.4 | (0.88) |
| (Stage 1 | Week 12: N, Mean (SD) | 24, 29.8 | (15.29) | 28, 30.3 | (11.34) |
| Placebo | Change from Baseline [2]: | 24, −1.8 | (4.23) | 28, 1.9 | (3.70) |
| Non- | N, Mean (SD) | | | | |
| re- | Standard Effect Size | | | 0.923 | |
| sponders) | % Change from Baseline [2]: | 24, −8.6 | (24.33) | 28, 7.5 | (15.69) |
| | N, Mean (SD) | | | | |
| | Treatment Difference vs. Placebo: | | | 3.62 | (1.37, 5.87) |
| | LS Mean Difference, 95% CI [1] | | | | |
| | p-value [1] | | | 0.002 | |
| | SPCD Weighted OLS z-statistic, | | | 2.00, | 0.046 |
| | overall p-value [3] | | | | |

Note:
MCCB Composite Score ranges from 1 to 70, with higher scores indicating less clinical severity of symptoms.
Standard Effect Size is defined as (mean change in d6-DM/Q-mean change in Placebo)/Change front Baseline Pooled SD.
[1] Change from Baseline was analyzed at each stage by ANCOVA with treatment as fixed effect and baseline value as covariate. Missing values were imputed by LOCF within each stage.
[2] Stage 2 Baseline is the last non-missing assessment prior to re-randomization into Stage 2 (re-randomization visit).
[3] SPCD Weighted OLS z-statistic was calculated using Stage 1 weight = 0.6 and Stage 2 weight = 0.4.

TABLE 65
MCCB Composite Score: Change from Baseline
Parallel Group MMRM Analysis, Observed Data
mITT 12-Week Parallel Group (N = 87)

| | Placebo/ | | | |
|---|---|---|---|---|
| Visit/Statistics | Placebo | | d6-DM/Q/d6-DM/Q | |
| Baseline: N, Mean (SD) | 40, 30.8 | (14.26) | 47, 32.5 | (11.37) |
| Week 6 Change from Baseline: N, Mean (SD) | 36, 0.6 | (4.03) | 46, 1.2 | (5.11) |
| Week 12 Change from Baseline: N, Mean (SD) | 31, −1.2 | (4.59) | 42, −0.0 | (6.22) |
| Week 12 Treatment Difference vs. | | | 0.498, 0.91 | (−1.76, 3.59) |
| Placebo: p-value, LS Mean Difference (95% CI) | | | | |

Note:
MCCB Composite Score ranges from 1 to 70, with higher scores indicating less clinical severity of symptoms.
Patients within each treatment group received the same treatment throughout their participation in the study.
Repeated measures model includes fixed effect for treatment, visit, treatment-by-visit interaction, baseline value, and baseline value-by-visit interaction. An unstructured covariance matrix was used.

TABLE 66
MCCB Composite Score: Change from
Baseline Parallel Group MMRM Analysis, Observed Data
Per Protocol 12-Week Parallel Group (N = 74)

| | Placebo/ | | d6-DM/Q/ | |
|---|---|---|---|---|
| Visit/Statistics | Placebo | | d6-DM/Q | |
| Baseline: N, Mean (SD) | 37, 30.5 | (14.20) | 37, 32.6 | (10.94) |
| Week 6 Change from Baseline: | 33, 0.8 | (4.04) | 36, 1.1 | (4.76) |
| N, Mean (SD) | | | | |
| Week 12 Change from Baseline: | 28, −1.1 | (4.51) | 35, 0.1 | (6.48) |
| N, Mean (SD) | | | | |

| | |
|---|---|
| Week 12 Treatment Difference | 0.524, 0.93 |
| vs. Placebo: p-value, LS Mean | (−1.97, 3.84) |
| Difference (95% CI) | |

Note:
MCCB Composite Score ranges from 1 to 70, with higher scores indicating less clinical severity of symptoms.
Patients within each treatment group received the same treatment throughout their participation in the study.
Repeated measures model includes fixed effect for treatment, visit, treatment-by-visit interaction, baseline value, and baseline value-by-visit interaction An unstructured covariance matrix was used.

TABLE 67
Prior Medications
Safety Population (N = 144)

| Anatomical Therapeutic Subgroup | Stage 1 | Stage 1 |
|---|---|---|
| (ATC Level 2), n (%) | Placebo | d6-DM/Q |
| Preferred Term, n (%) | (N = 96) | (N = 48) |
| Overall | 12 (12.5) | 7 (14.6) |
| ANALGESICS | 1 (1.0) | 2 (4.2) |
| Lenoltec With Codeine No 1 | 0 (0.0) | 1 (2.1) |
| Oxycocet | 1 (1.0) | 0 (0.0) |
| Paracetamol | 0 (0.0) | 1 (2.1) |
| ANTI-PARKINSON DRUGS | 0 (0.0) | 1 (2.1) |
| Benzatropine Mesilate | 0 (0.0) | 1 (2.1) |
| ANTIANEMIC PREPARATIONS | 1 (1.0) | 0 (0.0) |
| Mecobalamin | 1 (1.0) | 0 (0.0) |
| ANTIBACTERIALS FOR SYSTEMIC USE | 1 (1.0) | 0 (0.0) |
| Cefadroxil | 1 (1.0) | 0 (0.0) |
| ANTIEMETICS AND ANTINAUSEANTS | 0 (0.0) | 1 (2.1) |
| Ondansetron | 0 (0.0) | 1 (2.1) |
| ANTIINFLAMMATORY AND | 1 (1.0) | 0 (0.0) |
| ANTIRHEUMATIC PRODUCTS | | |
| Ibuprofen | 1 (1.0) | 0 (0.0) |
| DRUGS FOR OBSTRUCTIVE | 1 (1.0) | 0 (0.0) |
| AIRWAY DISEASES | | |
| Seretide | 1 (1.0) | 0 (0.0) |
| DRUGS USED IN DIABETES | 1 (1.0) | 0 (0.0) |
| Metformin | 1 (1.0) | 0 (0.0) |
| OTHER ALIMENTARY TRACT AND | 1 (1.0) | 0 (0.0) |
| METABOLISM PRODUCTS | | |
| Methionine | 1 (1.0) | 0 (0.0) |
| PSYCHOANALEPTICS | 1 (1.0) | 1 (2.1) |
| Fluoxetine | 0 (0.0) | 1 (2.1) |
| Trazodone | 1 (1.0) | 0 (0.0) |
| PSYCHOLEPTICS | 7 (7.3) | 5 (10.4) |
| Aripiprazole | 0 (0.0) | 2 (4.2) |
| Diphenhydramine | 0 (0.0) | 1 (2.1) |
| Diphenhydramine Hydrochloride | 0 (0.01 | 1 (2.1) |
| Hydroxyzine | 0 (0.01 | 1 (2.1) |
| Lithium Carbonate | 1 (1.0) | 0 (0.0) |
| Olanzapine | 0 (0.0) | 1 (2.1) |
| Paliperidone Palmitate | 1 (1.0) | 0 (0.0) |
| Quetiapine Fumarate | 1 (1.0) | 0 (0.0) |
| Risperidone | 4 (4.2) | 0 (0.0) |
| VITAMINS | 1 (1.0) | 0 (0.0) |
| Inositol | 1 (1.0) | 0 (0.0) |

Note:
Prior medications are defined as medications with a stop date prior to the first dose date of randomized study medication. Medications are coded using WHO Drug Dictionary Version September 2015
Treatment allocation based on Stage 1 randomization.

TABLE 68
Concomitant Medications
Safety Population (N = 144)

| Anatomical Therapeutic | | | d6-DM/ | | Placebo/ | |
|---|---|---|---|---|---|---|
| Subgroup (ATC | Placebo/ | | Q/d6- | | d6- | |
| Level 2), n (%) | Placebo | | DM/Q | | DM/Q | |
| Preferred Term, n (%) | (N = 56) | | (N = 48) | | (N = 40) | |
| Overall | 56 | (100.0) | 48 | (100.0) | 40 | (100.0) |
| AGENTS ACTING ON | 13 | (23.2) | 7 | (14.6) | 7 | (17.5) |
| THE RENIN- | | | | | | |
| ANGIOTENSIN SYSTEM | | | | | | |
| Benazepril | 1 | (1.8) | 1 | (2.1) | 0 | (0.0) |
| Enalapril | 2 | (3.6) | 1 | (2.1) | 0 | (0.0) |
| Lisinopril | 6 | (10.7) | 3 | (6.3) | 3 | (7.5) |
| Losartan | 1 | (1.8) | 1 | (2.1) | 0 | (0.0) |
| Quinapril | 0 | (0.0) | 1 | (2.1) | 0 | (0.0) |
| Quinapril Hydrochloride | 0 | (0.0) | 0 | (0.0) | 1 | (2.5) |
| Ramipril | 1 | (1.8) | 0 | (0.0) | 0 | (0.0) |
| Valsartan | 0 | (0.0) | 0 | (0.0) | 1 | (2.5) |
| Zestoretic | 2 | (3.6) | 0 | (0.0) | 2 | (5.0) |
| ANABOLIC AGENTS FOR | 0 | (0.0) | 0 | (0.0) | 1 | (2.5) |
| SYSTEMIC USE | | | | | | |
| Prasterone | 0 | (0.0) | 0 | (0.0) | 1 | (2.5) |
| ANALGESICS | 8 | (14.3) | 6 | (12.5) | 5 | (12.5) |
| Gabapentin | 4 | (7.1) | 0 | (0.0) | 0 | (0.0) |
| Medinite | 0 | (0.0) | 0 | (0.0) | 1 | (2.5) |
| Panadeine Co | 1 | (1.8) | 0 | (0.0) | 0 | (0.0) |
| Paracetamol | 5 | (8.9) | 1 | (2.1) | 3 | (7.5) |
| Safapryn | 0 | (0.0) | 1 | (2.1) | 0 | (0.0) |
| Singlet | 0 | (0.0) | 1 | (2.1) | 1 | (2.5) |
| Topiramate | 0 | (0.0) | 1 | (2.1) | 0 | (0.0) |
| Tramadol | 0 | (0.0) | 1 | (2.1) | 0 | (0.0) |
| Vicks Formula 44m | 0 | (0.0) | 0 | (0.0) | 1 | (2.5) |
| Vicodin | 0 | (0.0) | 1 | (2.1) | 0 | (0.0) |
| ANESTHETICS | 1 | (1.8) | 0 | (0.0) | 0 | (0.0) |
| Anaesthetics, Local | 1 | (1.8) | 0 | (0.0) | 0 | (0.0) |
| ANTI-PARKINSON DRUGS | 1 | (1.8) | 1 | (2.1) | 0 | (0.0) |
| Benzatropine Mesilate | 1 | (1.8) | 1 | (2.1) | 0 | (0.0) |
| ANTIANEMIC | 3 | (5.4) | 2 | (4.2) | 2 | (5.0) |
| PREPARATIONS | | | | | | |
| Cyanocobalamin | 0 | (0.0) | 1 | (2.1) | 1 | (2.5) |
| Ferrous Sulfate | 2 | (3.6) | 1 | (2.1) | 0 | (0.0) |
| Folic Acid | 1 | (1.8) | 0 | (0.0) | 0 | (0.0) |
| Iron | 0 | (0.0) | 0 | (0.0) | 1 | (2.5) |
| ANTIBACTERIALS | 4 | (7.1) | 1 | (2.1) | 2 | (5.0) |
| FOR SYSTEMIC USE | | | | | | |
| Amoxicillin | 2 | (3.6) | 1 | (2.1) | 0 | (0.0) |
| Azithromycin | 1 | (1.8) | 0 | (0.0) | 0 | (0.0) |
| Bactrim | 1 | (1.8) | 0 | (0.0) | 0 | (0.0) |
| Cefalexin | 0 | (0.0) | 0 | (0.0) | 1 | (2.5) |
| Metronidazole | 0 | (0.0) | 0 | (0.0) | 1 | (2.5) |
| ANTIDIARRHEALS | 0 | (0.0) | 0 | (0.0) | 1 | (2.5) |
| INTESTINAL | | | | | | |
| ANTIINFLAMMATORY/ | | | | | | |
| ANTIINTECTIVE AGENTS | | | | | | |
| Loperamide Hydrochloride | 0 | (0.0) | 0 | (0.0) | 1 | (2.5) |
| ANTIEMETICS AND | 0 | (0.0) | 1 | (2.1) | 0 | (0.0) |
| ANTINAUSEANTS | | | | | | |
| Ondansetron | 0 | (0.0) | 1 | (2.1) | 0 | (0.0) |
| ANTIEPILEPT1CS | 3 | (5.4) | 2 | (4.2) | 2 | (5.0) |
| Valproate Semisodium | 3 | (5.4) | 2 | (4.2) | 2 | (5.0) |
| ANTIFUNGALS FOR | 0 | (0.0) | 1 | (2.1) | 0 | (0.0) |
| DERMATOLOGICAL USE | | | | | | |
| Nystatin | 0 | (0.0) | 1 | (2.1) | 0 | (0.0) |
| ANTIGOUT | 0 | (0.0) | 0 | (0.0) | 1 | (2.5) |
| PREPARATIONS | | | | | | |
| Allopurinol | 0 | (0.0) | 0 | (0.0) | 1 | (2.5) |
| ANTIHISTAMINES FOR | 1 | (1.8) | 3 | (6.3) | 4 | (10.0) |
| SYSTEMIC USE | | | | | | |
| Cetirizine Hydrochloride | 0 | (0.0) | 0 | (0.0) | 1 | (2.5) |
| Diphenhydramine | 0 | (0.0) | 1 | (2.1) | 1 | (2.5) |
| Loratadine | 1 | (1.8) | 1 | (2.1) | 2 | (50) |
| Meclozine | 0 | (0.0) | 1 | (2.1) | 0 | (0.0) |
| ANTIHYPERTENSIVES | 0 | (0.0) | 1 | (2.1) | 0 | (0.0) |
| Clonidine | 0 | (0.0) | 1 | (2.1) | 0 | (0.0) |
| ANTIINFLAMMATORY | 5 | (8.9) | 8 | (16.7) | 8 | (20.0) |
| AND ANTIRHEUMATIC | | | | | | |
| PRODUCTS | | | | | | |
| Advil Pm | 0 | (0.0) | 1 | (2.1) | 0 | (0.0) |
| Ibuprofen | 3 | (5.4) | 5 | (10.4) | 5 | (12.5) |
| Naproxen | 2 | (3.6) | 3 | (6.3) | 3 | (7.5) |
| Naproxen Sodium | 0 | (0.0) | 1 | (2.1) | 0 | (0.0) |
| ANTIMYCOTICS FOR | 1 | (1.8) | 0 | (0.0) | 0 | (0.0) |
| SYSTEMIC USE | | | | | | |
| Antibiotics | 1 | (1.8) | 0 | (0.0) | 0 | (0.0) |
| ANTITHROMBOTIC | 6 | (10.7) | 3 | (6.3) | 1 | (2.5) |
| AGENTS | | | | | | |
| Acetylsalicylic Acid | 6 | (10.7) | 3 | (6.3) | 1 | (2.5) |
| BETA BLOCKING AGENTS | 4 | (7.1) | 1 | (2.1) | 3 | (7.5) |
| Atenolol | 0 | (0.0) | 1 | (2.1) | 0 | (0.0) |
| Metoprolol | 3 | (5.4) | 0 | (0.0) | 1 | (2.5) |
| Metoprolol Tartrate | 1 | (1.8) | 0 | (0.0) | 0 | (0.0) |
| Propranolol | 0 | (0.0) | 0 | (0.0) | 2 | (5.0) |
| CALCIUM CHANNEL | 9 | (16.1) | 4 | (8.3) | 7 | (17.5) |
| BLOCKERS | | | | | | |
| Amlodipine | 4 | (7.1) | 3 | (6.3) | 5 | (12.5) |
| Amlodipine Besilate | 5 | (8.9) | 1 | (2.1) | 1 | (2.5) |
| Nifedipine | 0 | (0.0) | 0 | (0.0) | 1 | (2.5) |
| CORTICOSTEROIDS. | 1 | (1.8) | 0 | (0.0) | 1 | (2.5) |
| DERMATOLOGICAL | | | | | | |
| PREPARATIONS | | | | | | |
| Hydrocortisone | 1 | (1.8) | 0 | (0.0) | 0 | (0.0) |
| Triamcinolone Acetonide | 0 | (0.0) | 0 | (0.0) | 1 | (2.5) |
| COUGH AND COLD | 1 | (1.8) | 0 | (0.0) | 1 | (2.5) |
| PREPARATIONS | | | | | | |
| Dextromethorphan | 0 | (0.0) | 0 | (0.0) | 1 | (2.5) |
| Hydrobromide | | | | | | |
| Tussin Dm | 1 | (18) | 0 | (0.0) | 0 | (0.0) |
| DIURETICS | 4 | (7.1) | 7 | (14.6) | 3 | (7.5) |
| Chlortalidone | 0 | (0.0) | 0 | (0.0) | 1 | (2.5) |
| Furosemide | 1 | (1.8) | 0 | (0.0) | 0 | (0.0) |
| Hydrochlorothiazide | 3 | (5.4) | 7 | (14.6) | 2 | (5.0) |
| DRUGS FOR ACID | 5 | (8.9) | 7 | (14.6) | 3 | (7.5) |
| RELATED | | | | | | |
| DISORDERS | | | | | | |
| Calcium Carbonate | 0 | (0.0) | 0 | (0.0) | 1 | (25) |
| Novalucol Novum | 0 | (0.0) | 0 | (0.0) | 1 | (2.5) |
| Omeprazole | 4 | (7.1) | 5 | (10.4) | 1 | (2.5) |
| Ranitidine | 1 | (1.8) | 2 | (4.2) | 0 | (0.0) |
| DRUGS FOR | 5 | (8.9) | 1 | (2.1) | 4 | (10.0) |
| CONSTIPATION | | | | | | |
| Bisacodyl | 0 | (0.0) | 0 | (0.0) | 1 | (2.5) |
| Docusate | 0 | (0.0) | 0 | (0.0) | 1 | (2.5) |
| Docusate Sodium | 4 | (7.1) | 0 | (0.0) | 0 | (0.0) |
| Macrogol | 0 | (0.0) | 0 | (0.0) | 1 | (2.5) |
| Magnesium Hydroxide | 0 | (0.0) | 1 | (2.1) | 0 | (0.0) |
| Psyllium Hydrophilic | 0 | (0.0) | 0 | (0.0) | 1 | (2.5) |
| Mucilloid | | | | | | |
| Senokot-S | 1 | (1.8) | 0 | (0.0) | 0 | (0.0) |
| DRUGS FOR | 5 | (8.9) | 3 | (6.3) | 2 | (5.0) |
| OBSTRUCTIVE | | | | | | |
| AIRWAY DISEASES | | | | | | |
| Budesonide W/ | 1 | (1.8) | 0 | (0.0) | 2 | (5.0) |
| Formoterol Fumarate | | | | | | |
| Combivent | 0 | (0.0) | 2 | (4.2) | 0 | (0.0) |
| Montelukast | 1 | (1.8) | 0 | (0.0) | 0 | (0.0) |
| Montelukast Sodium | 1 | (1.8) | 1 | (2.1) | 0 | (0.0) |
| Salbutamol | 3 | (54) | 1 | (2.1) | 1 | (2.5) |
| Salbutamol Sulfate | 0 | (0.0) | 0 | (0.0) | 1 | (2.5) |
| Seretide | 1 | (1.8) | 0 | (0.0) | 0 | (0.0) |
| Tiotropium Bromide | 0 | (0.0) | 1 | (2.1) | 0 | (0.0) |
| DRUGS FOR TREATMENT | 0 | (0.0) | 0 | (0.0) | 1 | (2.5) |
| OF BONE DISEASES | | | | | | |
| Alendronate Sodium | 0 | (0.0) | 0 | (0.0) | 1 | (2.5) |
| DRUGS USED IN | 10 | (17.9) | 12 | (25.0) | 9 | (22.5) |
| DIABETES | | | | | | |
| Glibenclamide | 1 | (1.8) | 0 | (0.0) | 0 | (0.0) |
| Glimepiride | 1 | (1.8) | 1 | (2.1) | 0 | (0.0) |
| Glipizide | 0 | (0.0) | 0 | (0.0) | 4 | (10.0) |
| Insulin Gilargine | 1 | (1.8) | 1 | (2.1) | 1 | (2.5) |
| Linagliptin | 1 | (1.8) | 0 | (0.0) | 0 | (0.0) |
| Metformin | 10 | (17.9) | 10 | (20.8) | 8 | (20.0) |
| Metformin Hydrochloride | 0 | (0.0) | 2 | (4.2) | 0 | (0.0) |
| Sitagliptin | 0 | (0.0) | 1 | (2.1) | 0 | (0.0) |
| EMOLLIENTS AND | 1 | (1.8) | 0 | (0.0) | 1 | (2.5) |
| PROTECTIVES | | | | | | |
| Emollients And Protectives | 1 | (1.8) | 0 | (0.0) | 0 | (0.0) |
| Magnesium Stearate | 0 | (0.0) | 0 | (0.0) | 1 | (2.5) |
| LIPID MODIFYING | 16 | (28.6) | 12 | (25.0) | 12 | (30.0) |
| AGENTS | | | | | | |
| Atorvastatin | 4 | (7.1) | 0 | (0.0) | 4 | (10.0) |
| Atorvastatin Calcium | 1 | (1.8) | 0 | (0.0) | 0 | (0.0) |
| Coleaciferol W/Fish Oil | 1 | (1.8) | 0 | (0.0) | 0 | (0.0) |
| Fenofibrate | 2 | (3.6) | 0 | (0.0) | 0 | (0.0) |
| Fish Oil | 0 | (0.0) | 2 | (4.2) | 2 | (5.0) |
| Gemfibrozil | 0 | (0.0) | 1 | (2.1) | 1 | (2.5) |
| Inegy | 0 | (0.0) | 1 | (2.1) | 0 | (0.0) |
| Lovastatin | 1 | (1.8) | 0 | (0.0) | 0 | (0.0) |
| Pravastatin | 1 | (1.8) | 1 | (2.1) | 2 | (5.0) |
| Rosuvastatin | 1 | (1.8) | 1 | (2.1) | 0 | (0.0) |
| Simvastatin | 9 | (16.1) | 6 | (12.5) | 4 | (10.0) |
| MINERAL SUPPLEMENTS | 3 | (5.4) | 0 | (0.0) | 2 | (5.0) |
| Calcium | 1 | (1.8) | 0 | (0.0) | 1 | (2.5) |
| Calcium Carbonate | 1 | (1.8) | 0 | (0.0) | 0 | (0.0) |
| Magnesium | 0 | (0.0) | 0 | (0.0) | 1 | (2.5) |
| Potassium | 0 | (0.0) | 0 | (0.0) | 1 | (2.5) |
| Potassium Chloride | 2 | (3.6) | 0 | (0.0) | 0 | (0.0) |
| MUSCLE RELAXANTS | 0 | (0.0) | 1 | (2.1) | 0 | (0.0) |
| Baclofen | 0 | (0.0) | 1 | (2.1) | 0 | (0.0) |
| NASAL PREPARATIONS | 1 | (1.8) | 0 | (0.0) | 3 | (7.5) |
| Fluticasone | 0 | (0.0) | 0 | (0.0) | 2 | (5.0) |
| Mometasone Furoate | 1 | (1.8) | 0 | (0.0) | 0 | (0.0) |
| Oxymetazoline Hydrochloride | 0 | (0.0) | 0 | (0.0) | 1 | (2.5) |
| Phenylephrine Hydrochloride | 0 | (0.0) | 0 | (0.0) | 1 | (2.5) |
| Sodium Chloride | 0 | (0.0) | 0 | (0.0) | 1 | (2.5) |
| OPHTHALMOLOGICALS | 0 | (0.0) | 0 | (0.0) | 2 | (5.0) |
| Bimatoprost | 0 | (0.0) | 0 | (0.0) | 1 | (2.5) |
| Dorzolamide W/Timolol | 0 | (0.0) | 0 | (0.0) | 1 | (2.5) |
| OTHER ALIMENTARY | 0 | (0.0) | 0 | (0.0) | 1 | (2.5) |
| TRACT AND | | | | | | |
| METABOLISM | | | | | | |
| PRODUCTS | | | | | | |
| Probiotics Nos | 0 | (0.0) | 0 | (0.0) | 1 | (2.5) |
| OTHER NERVOUS | 1 | (1.8) | 1 | (2.1) | 1 | (2.5) |
| SYSTEM DRUGS | | | | | | |
| Bupropion Hydrochloride | 1 | (1.8) | 1 | (2.1) | 0 | (0.0) |
| Naltrexone | 0 | (0.0) | 0 | (0.0) | 1 | (2.5) |
| PITUITARY AND | 1 | (1.8) | 0 | (0.0) | 0 | (0.0) |
| HYPOTHALAMIC | | | | | | |
| HORMONES AND | | | | | | |
| ANALOGUES | | | | | | |
| Desmopressin | 1 | (1.8) | 0 | (0.0) | 0 | (0.0) |
| PSYCHOANALEPTICS | 21 | (37.5) | 12 | (25.0) | 9 | (22.5) |
| Bupropion | 1 | (1.8) | 1 | (2.1) | 0 | (0.0) |
| Bupropion Hydrochloride | 1 | (1.8) | 0 | (0.0) | 0 | (0.0) |
| Citalopram Hydrobromide | 1 | (1.8) | 0 | (0.0) | 1 | (2.5) |
| Escitalopram | 0 | (0.0) | 0 | (0.0) | 1 | (2.5) |
| Escitalopram Oxalate | 2 | (3.6) | 1 | (2.1) | 1 | (2.5) |
| Fluoxetine | 1 | (1.8) | 2 | (4.2) | 0 | (0.0) |
| Fluoxetine Hydrochloride | 1 | (1.8) | 1 | (2.1) | 2 | (5.0) |
| Fluvoxamine | 1 | (1.8) | 0 | (0.0) | 0 | (0.0) |
| Memantine | 1 | (1.8) | 0 | (0.0) | 0 | (0.0) |
| Mirtazapine | 1 | (1.8) | 2 | (4.2) | 0 | (0.0) |
| Paroxetine | 2 | (3.6) | 0 | (0.0) | 0 | (0.0) |
| Paroxetine Hydrochloride | 1 | (1.8) | 0 | (0.0) | 0 | (0.0) |
| Sertraline | 2 | (3.6) | 1 | (2.1) | 2 | (5.0) |
| Sertraline Hydrochloride | 3 | (5.4) | 2 | (4.2) | 2 | (5.0) |
| Trazodone | 7 | (12.5) | 2 | (4.2) | 0 | (0.0) |
| Trazodone Hydrochloride | 0 | (0.0) | 0 | (0.0) | 1 | (2.5) |
| Venlafaxine | 2 | (3.6) | 1 | (2.1) | 0 | (0.0) |
| PSYCHOLEPTICS | 56 | (100.0) | 48 | (100.0) | 40 | (100.0) |
| Aripiprazole | 11 | (19.6) | 16 | (33.3) | 5 | (12.5) |
| Buspirone | 1 | (1.8) | 0 | (0.0) | 0 | (0.0) |
| Buspirone Hydrochloride | 0 | (0.0) | 0 | (0.0) | 2 | (5.0) |
| Clonazepam | 0 | (0.0) | 1 | (2.1) | 0 | (0.0) |
| Diphenhydramine | 0 | (0.0) | 2 | (4.2) | 0 | (0.0) |
| Hydroxyzine | 2 | (3.6) | 0 | (0.0) | 1 | (2.5) |
| Lithium | 1 | (1.8) | 0 | (0.0) | 0 | (0.0) |
| Lorazepam | 3 | (5.4) | 0 | (0.0) | 3 | (7.5) |
| Lurasidone | 2 | (3.6) | 1 | (2.1) | 0 | (0.0) |
| Lurasidone Hydrochloride | 1 | (1.8) | 3 | (6.3) | 2 | (5.0) |
| Olanzapine | 13 | (23.2) | 12 | (25.0) | 13 | (32.5) |
| Paliperidone | 3 | (5.4) | 3 | (6.3) | 0 | (0.0) |
| Paliperidone Palmitate | 7 | (12.5) | 2 | (4.2) | 4 | (10.0) |
| Quetiapine | 1 | (1.8) | 2 | (4.2) | 1 | (2.5) |
| Quetiapine Fumarate | 9 | (16.1) | 7 | (14.6) | 5 | (12.5) |
| Risperidone | 13 | (23.2) | 10 | (20.8) | 12 | (30.0) |
| Ziprasidone | 1 | (1.8) | 1 | (2.1) | 1 | (2.5) |
| Zolpidem | 3 | (5.4) | 1 | (2.1) | 1 | (2.5) |
| Zolpidem Tartrate | 1 | (1.8) | 1 | (2.1) | 1 | (2.5) |
| SEX HORMONES AND | 1 | (1.8) | 0 | (0.0) | 0 | (0.0) |
| MODULATORS OF THE | | | | | | |
| GENITAL SYSTEM | | | | | | |
| Anovlar | 1 | (1.8) | 0 | (0.0) | 0 | (0.0) |
| THYROID THERAPY | 6 | (10.7) | 4 | (8.3) | 3 | (7.5) |
| Levothyroxine | 4 | (7.1) | 2 | (4.2) | 3 | (7.5) |
| Levothyroxine Sodium | 2 | (3.6) | 2 | (4.2) | 0 | (0.0) |
| UNSPECIFIED HERBAL | 0 | (0.0) | 0 | (0.0) | 1 | (2.5) |
| AND TRADITIONAL | | | | | | |
| MEDICINE | | | | | | |
| Berberis Vulgaris | 0 | (0.0) | 0 | (0.0) | 1 | (2.5) |
| Linum Usitatissimum Seed Oil | 0 | (0.0) | 0 | (0.0) | 1 | (2.5) |
| UROLOGICALS | 3 | (5.4) | 0 | (0.0) | 1 | (2.5) |
| Mirabegron | 1 | (1.8) | 0 | (0.0) | 0 | (0.0) |
| Oxybutynin Hydrochloride | 0 | (0.0) | 0 | (0.0) | 1 | (2.5) |
| Tamsulosin | 3 | (5.4) | 0 | (0.0) | 0 | (0.0) |
| VITAMINS | 7 | (12.5) | 8 | (16.7) | 8 | (20.0) |
| Ascorbic Acid | 0 | (0.0) | 0 | (0.0) | 1 | (2.5) |
| Cod-Liver Oil | 0 | (0.0) | 1 | (2.1) | 0 | (0.0) |
| Colecalciferol | 3 | (5.4) | 2 | (4.2) | 2 | (5.0) |
| Ergocalciferol | 0 | (0.0) | 1 | (2.1) | 0 | (0.0) |
| Herbal Nos W/Vitamins Nos | 0 | (0.0) | 0 | (0.0) | 1 | (2.5) |
| Minerals Nos W/Vitamins Nos | 0 | (0.0) | 0 | (0.0) | 1 | (2.5) |
| Multivitamins, Plain | 4 | (7.1) | 5 | (10.4) | 4 | (10.0) |
| Thiamine Hydrochloride | 1 | (1.8) | 0 | (0.0) | 0 | (0.0) |
| Vitamin B Complex | 0 | (0.0) | 1 | (2.1) | 1 | (2.5) |
| Vitamin D Nos | 1 | (1.8) | 0 | (0.0) | 2 | (5.0) |

Note:
Concomitant medications are defined as medications with a start date on or before the last dose date of randomized study medication. and a stop date on or after the first dose date of randomized study medication. Concomitant medications are coded using WHO Drug Dictionary Version September 2015.

TABLE 69
NSA-16 Total Score: Change from Baseline SPCD ANCOVA, LOCF Data
mITT Population (N = 127) Subgroup: Baseline Conmeds Benzodiazepine Use

| | Visit | | | | |
|---|---|---|---|---|---|
| Stage | Statistics | Placebo | | d6-DM/Q | |
| Stage 1 | Baseline: N, Mean (SD) | 10, 62.8 | (5.96) | 3, 64.7 | (6.03) |
| | Week 6: N, Mean (SD) | 10, 59.3 | (6.53) | 3, 56.7 | (8.62) |
| | Change from Baseline: N, Mean (SD) | 10, −3.5 | (6.10) | 3, −8.0 | (6.08) |
| | Standard Effect Size | | | −0.738 | |
| | % Change from Baseline: N, Mean (SD) | 10, −5.3 | (9.22) | 3, −12.4 | (9.54) |
| | Treatment Difference vs. Placebo: | | | −3.86 | (−12.79, 5.06) |
| | LS Mean Difference, 95% CI [1] | | | | |
| | p-value [1] | | | 0.358 | |
| Stage 2 | Baseline [2]: N. Mean (SD) | 4, 59.5 | (5.51) | 3, 54.3 | (3.06) |
| (Stage 1 | Week 12: N, Mean (SD) | 4, 57.5 | (5.97) | 3, 46.3 | (1.53) |
| Placebo | Change from Baseline [2]: N, Mean (SD) | 4, −2.0 | (5.66) | 3, −8.0 | (2.65) |
| Non- | Standard Effect Size | | | −1.279 | |
| re- | % Change from Baseline [2]: N. Mean (SD) | 4, −3.1 | (8.84) | 3,−14.6 | (4.10) |
| sponders) | Treatment Difference vs. Placebo: | | | −8.54 | (−20.07, 2.98) |
| | LS Mean Difference, 95% CI [1] | | | | |
| | p-value [1] | | | 0.109 | |
| | SPCD Weighted OLS z-statistic, | | | −1.96, | 0.050 |
| | overall p-value [3] | | | | |

Note:
NSA-16 Total Score ranges from 16 to 96, with higher scores indicating greater clinical severity of symptoms.
Standard Effect Size is defined as (mean change in d6-DM/Q-mean change in Placebo/Change from Baseline Pooled SD.
[1] Change from Baseline was analyzed at each stage by ANCOVA with treatment as fixed effect and baseline value as covariate. Missing values were imputed by LOCF within each stage.
[2] Stage 2 Baseline is the last non-missing assessment prior to re-randomization into Stage 2 (re-randomization visit).
[3] SPCD Weighted OLS z-statistic was calculated using Stage 1 weight = 0.6 and Stage 2 weight = 0.4

TABLE 70
NSA-16 Total Score: Change from Baseline SPCD ANCOVA,
LOCF Data mITT Population (N = 127)
Subgroup: Baseline Conmeds SNRI Use

| | Visit | | | | |
|---|---|---|---|---|---|
| Stage | Statistics | Placebo | | d6-DM/Q | |
| Stage 1 | Baseline: N, Mean (SD) | 2, 67.0 | (1.41) | 1, 54.0 | ( ) |
| | Week 6: N, Mean (SD) | 2, 63.0 | (2.83) | 1, 51.0 | ( ) |
| | Change from Baseline: | 2, −4.0 | (4.24) | 1, −3.0 | ( ) |
| | N, Mean (SD) | | | | |
| | Standard Effect Size | | | | |
| | % Change from Baseline: | 2, −5.9 | (6.21) | 1, −5.6 | ( ) |
| | N, Mean (SD) | | | | |
| | Treatment Difference vs. Placebo: | | | −38.00 | ( ) |
| | LS Mean Difference, 95% CI [1] | | | | |
| | p-value [1] | | | | |
| Stage 2 | Baseline [2]: N, Mean (SD) | 2, 63.0 | (2.83) | | |
| (Stage 1 | Week 12: N, Mean (SD) | 2, 55.0 | (0.00) | | |
| Placebo | Change from Baseline [2]: | 2, −8.0 | (2.83) | | |
| Non- | N, Mean (SD) | | | | |
| re- | Standard Effect Size | | | | |
| sponders) | % Change from Baseline [2]: | 2, −12.6 | (3.92) | | |
| | N, Mean (SD) | | | | |
| | Treatment Difference vs. Placebo: | | | | |
| | LS Mean Difference, 95% CI [1] | | | | |
| | p-value [1] | | | | |

Note:
NSA-16 Total Score ranges from 16 to 96, with higher scores indicating greater clinical severity of symptoms.
Standard Effect Size is defined as (mean change in d6-DM/Q-mean change in Placebo)/Change from Baseline Pooled SD.
[1] Change from Baseline was analyzed at each stage by ANCOVA with treatment as fixed effect and baseline value as covariate. Missing values wereimputed by LOCF within each stage.
[2] Stage 2 Baseline is the last non-missing assessment prior to re-randomization into Stage 2 (re-randomization visit).
[3] SPCD Weighted OLS z-statistic was calculated using Stage 1 weight = 0.6 and Stage 2 weight = 0.4.

TABLE 71
NSA-16 Total Score: Change from Baseline SPCD ANCOVA, LOCF Data
mITT Population (N = 127)
Subgroup: Baseline Conmeds SSRI Use

| | Visit | | | | |
|---|---|---|---|---|---|
| Stage | Statistics | Placebo | | d6-DM/Q | |
| Stage 1 | Baseline: N, Mean (SD) | 22, 61.7 | (8.51) | 7, 63.4 | (8.98) |
| | Week 6: N, Mean (SD) | 22, 58.7 | (11.24) | 7, 60.3 | (9.14) |
| | Change from Baseline: N, Mean (SD) | 22, −3.0 | (5.99) | 7, −3.1 | (4.06) |
| | Standard Effect Size | | | −0.025 | |
| | % Change from Baseline: N, Mean (SD) | 22, −5.1 | (9.97) | 7, −4.9 | (6.08) |
| | Treatment Difference vs. Placebo: | | | −0.27 | (−5.36, 4.82) |
| | LS Mean Difference, 95% CI [1] | | | | |
| | p-value [1] | | | 0.914 | |
| Stage 2 | Baseline [2]: N, Mean (SD) | 10, 58.1 | (11.61) | 8, 60.6 | (12.58) |
| (Stage 1 | Week 12: N, Mean (SD) | 10, 57.3 | (11.86) | 8, 55.9 | (11.80) |
| Placebo | Change from Baseline [2]: N, Mean (SD) | 10, −0.8 | (4.37) | 8, −4.8 | (6.98) |
| Non- | Standard Effect Size | | | −0.698 | |
| re- | % Change from Baseline [2]: N, Mean (SD) | 10, −1.4 | (7.32) | 8, −7.3 | (10.16) |
| sponders) | Treatment Difference vs. Placebo: | | | −3.63 | (−9.35, 2.10) |
| | LS Mean Difference, 95% CI [1] | | | | |
| | p-value [1] | | | 0.197 | |
| | SPCD Weighted OLS z-statistic, | | | −0.88, | 0.379 |
| | overall p-value [3] | | | | |

Note:
NSA-16 Total Score ranges from 16 to 96. with higher scores indicating greater clinical severity of symptoms.
Standard Effect Size is defined as (mean change in d6-DM/Q-mean change in Placebo)/Change from Baseline Pooled SD.
[1] Change from Baseline was analyzed at each stage by ANCOVA with treatment as fixed effect and baseline value as covariate. Missing values were imputed by LOCF within each stage.
[2] Stage 2 Baseline is the last non-missing assessment prior to re-randomization into Stage 2 (re-randomization visit).
[3] SPCD Weighted OLS z-statistic was calculated using Stage 1 weight = 0.6 and Stage 2 weight = 0.4.

TABLE 72
NSA-16 Total Score: Change from Baseline SPCD ANCOVA, LOCF Data
mITT Population (N = 127)
Subgroup: Patients Who Took Baseline Concomitant Psychotropic
Medications with Major CYP2D6 Substrate = Yes

| | Visit | | | | |
|---|---|---|---|---|---|
| Stage | Statistics | Placebo | | d6-DM/Q | |
| Stage 1 | Baseline: N, Mean (SD) | 36, 61.8 | (8.11) | 25, 59.8 | (7.27) |
| | Week 6: N, Mean (SD) | 36, 58.6 | (9.78) | 25, 55.0 | (8.45) |
| | Change from Baseline: N, Mean (SD) | 36, −3.2 | (4.63) | 25, −4.8 | (4.40) |
| | Standard Effect Size | | | −0.356 | |
| | % Chane from Baseline: N, Mean (SD) | 36, −5.4 | (7.50) | 25, 8.2 | (7.41) |
| | Treatment Difference vs. Placebo: | | | −1.54 | (−3.94, 0.86) |
| | LS Mean Difference, 95% CI [1] | | | | |
| | p-value [1] | | | 0.203 | |
| Stage 2 | Baseline 2: N, Mean (SD) | 16, 59.8 | (10.23) | 12, 59.7 | (10.00) |
| (Stage 1 | Week 12: N, Mean (SD) | 16, 55.7 | (11.86) | 12, 56.7 | (9.94) |
| Placebo | Change from Baseline [2]: N, Mean (SD) | 16, −4.1 | (6.74) | 12, −3.0 | (5.97) |
| Non- | Standard Effect Size | | | 0.175 | |
| re- | % Change from Baseline [2]: N, | 16, −6.9 | (10.87) | 12, −4.8 | (8.67) |
| sponders) | Mean (SD) | | | | |
| | Treatment Difference vs. Placebo: | | | 1.11 | (−3.98, 6.20) |
| | LS Mean Difference, 95% CI [1] | | | | |
| | p-value [1] | | | 0.657 | |
| | SPCD Weighted OLS z-statistic, | | | −0.39, | 0.693 |
| | overall p-value [3] | | | | |

Note:
NSA-16 Total Score ranges from 16 to 96, with higher scores indicating greater clinical severity of symptoms.
Standard Effect Size is defined as (mean change in d6-DM/Q-mean change in Placebo)/Change from Baseline Pooled SD.
[1] Change from Baseline was analyzed at each stage by ANCOVA with treatment as fixed effect and baseline value as covariate. Missing values were imputed by LOCF within each stage.
[2] Stage 2 Baseline is the last non-missing assessment prior to re-randomization into Stage 2 (re-randomization visit).
[3] SPCD Weighted OLS z-statistic was calculated using Stage 1 weight =0.6 and Stage 2 weight = 0.4.

TABLE 73
NSA-16 Total Score: Change from Baseline SPCD ANCOVA, LOCF Data
mITT Population (N = 127)
Subgroup: Patients Who Took Baseline Concomitant Psychotropic
Medications with Major CYP2D6 Substrate = No

| | Visit | | | | |
|---|---|---|---|---|---|
| Stage | Statistics | Placebo | | d6-DM/Q | |
| Stage 1 | Baseline: N, Mean (SD) | 44, 59.2 | (7.25) | 22, 62.2 | (7.78) |
| | Week 6: N, Mean (SD) | 44, 56.5 | (8.72) | 22, 57.0 | (9.16) |
| | Change from Baseline: N, Mean (SD) | 44, −2.7 | (6.62) | 22, −5.3 | (6.89) |
| | Standard Effect Size | | | −0.379 | |
| | % Chane from Baseline: N, Mean (SD) | 44, −4.4 | (10.88) | 22, −8.3 | (11.38) |
| | Treatment Difference vs. Placebo: | | | −1.96 | (−5.47, 1.55) |
| | LS Mean Difference, 95% CI [1] | | | | |
| | p-value [1] | | | 0.269 | |
| Stage 2 | Baseline [2]: N, Mean (SD) | 14, 55.0 | (7.91) | 21, 56.4 | (8.55) |
| (Stage 1 | Week 12: N, Mean (SD) | 14, 55.1 | 1.01) | 21, 52.5 | (7.64) |
| Placebo | Change from Baseline [2]: N, Mean (SD) | 14, 0.1 | (3.85) | 21, −3.9 | (6.67) |
| Non- | Standard Effect Size | | | −0.694 | |
| re- | % Change from Baseline [2]: N, | 14, −0.6 | (7.45) | 21, −6.1 | (11.87) |
| sponders) | Mean (SD) | | | | |
| | Treatment Difference vs. | | | −3.78 | (−7.81, 0.24) |
| | Placebo: LS Mean Difference, 95% CI [1] | | | | |
| | p-value [1] | | | 0.064 | |
| | SPCD Weighted OLS z-statistic, | | | −2.04, | 0.041 |
| | overall p-value [3] | | | | |

Note:
NSA-16 Total Score ranges from 16 to 96, with higher scores indicating greater clinical severity of symptoms.
Standard Effect Size is defined as (mean change in d6-DM/Q-mean change in Placebo)/Change from Baseline Pooled SD.
[1] Change from Baseline was analyzed at each stage by ANCOVA with treatment as fixed effect and baseline value as covariate. Missing values were imputed by LOCF within each stage.
[2] Stage 2 Baseline is the last non-missing assessment prior to re-randomization into Stage 2 (re-randomization visit).
[3] SPCD Weighted OLS z-statistic was calculated using Stage 1 weight = 0.6 and Stage 2 weight = 0.4.

TABLE 74
NSA-16 Total Score: Change from Baseline Parallel Group ANCOVA by
Visit, LOCF Data mITT 12-Week Parallel Group Population (N = 87)
Subgroup: Patients Who Took Baseline Concomitant Psychotropic
Medications with Major CYP2D6 Substrate = Yes

| | | Placebo/ | | d6-DM/ | |
|---|---|---|---|---|---|
| Visit | Result/Statistics | Placebo | | Q/d6-DM/Q | |
| Baseline | N, Mean (SD) | 23, 62.2 | (8.38) | 25, 59.8 | (7.27) |
| Week 3 | N, Mean (SD) | 23, 58.3 | (8.36) | 24, 57.7 | (8.38) |
| Week 3 | Change from Baseline: N, Mean (SD) | 23, −3.9 | (4.78) | 24, −2.6 | (5.07) |
| Week 3 | Standard Effect Size | | | 0.270 | |
| Week 3 | Treatment Difference versus Placebo: | | | 0.446, 1.11 | |
| | p-value, LS Mean Difference (95% CI) | | | (−1.80, 4.01) | |
| Week 6 | N, Mean (SD) | 23, 58.5 | (9.71) | 25, 55.0 | (8.45) |
| Week 6 | Change from Baseline: N, Mean (SD) | 23, −3.7 | (4.03) | 25, −4.8 | (4.40) |
| Week 6 | Standard Effect Size | | | −0.281 | |
| Week 6 | Treatment Difference versus Placebo: | | | 0.372, −1.12 | |
| | p-value, LS Mean Difference (95% CI) | | | (−3.64, 1.39) | |
| Week 9 | N, Mean (SD) | 23, 57.6 | (11.04) | 25, 55.0 | (9.82) |
| Week 9 | Change from Baseline: N, Mean (SD) | 23, −4.6 | (6.77) | 25, −4.9 | (5.08) |
| Week 9 | Standard Effect Size | | | −0.053 | |
| Week 9 | Treatment Difference versus Placebo: | | | 0.960, −0.09 | |
| | p-value, LS Mean Difference (95% CI) | | | (−3.60, 3.42) | |
| Week 12 | N, Mean (SD) | 23, 55.1 | (11.21) | 25, 53.1 | (9.76) |
| Week 12 | Change from Baseline: N, Mean (SD) | 23, −7.0 | (7.46) | 25, −6.7 | (7.10) |

| | | |
|---|---|---|
| Week 12 | Standard Effect Size | 0.044 |
| Week 12 | Treatment Difference versus Placebo: | 0.911, 0.24 |
| | p-value, LS Mean Difference (95% CI) | (−4.08, 4.57) |

Note:
NSA-16 Total Score ranges from 16 to 96, with higher scores indicating greater clinical severity of symptoms.
Standard Effect Size is defined as (mean change in d6-DM/Q-mean change in Placebo)/Change from Baseline Pooled SD.
Missing values were imputed by LOCF and visit windows were used to classify unscheduled or ET visits.
LS mean difference and p-values were from ANCOVA with treatment as fixed effect and Stage 1 Baseline value as a covariate.
Patients within each treatment group received the same treatment throughout their participation in the study.

TABLE 75
NSA-16 Total Score: Change from Baseline Parallel Group ANCOVA by Visit,
LOCF Data mITT 12-Week Parallel Group Population (N =87)
Subgroup: Patients Who Took Baseline Concomitant Psychotropic
Medications with Major CYP2D6 Substrate = No

| Visit | Result/Statistics | Placebo/Placebo | | d6-DM/Q/d6-DM/Q | |
|---|---|---|---|---|---|
| Baseline | N, Mean (SD) | 17, 59.5 | (7.12) | 22, 62.2 | (7.78) |
| Week 3 | N, Mean (SD) | 17, 56.8 | (10.02) | 21, 59.3 | (8.20) |
| Week 3 | Change from Baseline: N, Mean (SD) | 17, −2.8 | (9.56) | 21, −2.5 | (6.58) |
| Week 3 | Standard Effect Size | | | 0.030 | |
| Week 3 | Treatment Difference versus Placebo: | | | 0.684, 1.05 | |
| | p-value, LS Mean Difference (95% CI) | | | (−4.13, 6.23) | |
| Week 6 | N, Mean (SD) | 17, 55.6 | (8.41) | 22, 57.0 | (9.16) |
| Week 6 | Change from Baseline: N, Mean (SD) | 17, −3.9 | (5.58) | 22, −5.3 | (6.89) |
| Week 6 | Standard Effect Size | | | −0.209 | |
| Week 6 | Treatment Difference versus Placebo: | | | 0.672, −0.89 | |
| | p-value, LS Mean Difference (95% CI) | | | (−5.10, 3.32) | |
| Week 9 | N, Mean (SD) | 17, 56.1 | (11.52) | 22, 53.2 | (11.24) |
| Week 9 | Change from Baseline: N, Mean (SD) | 17, −3.4 | (7.86) | 22, −9.0 | (8.57) |
| Week 9 | Standard Effect Size | | | −0.681 | |
| Week 9 | Treatment Difference versus Placebo: | | | 0.044, −5.74 | |
| | p-value, LS Mean Difference (95% CI) | | | (−11.32, −0.16) | |
| Week 12 | N, Mean (SD) | 17, 55.6 | (10.88) | 22, 56.9 | (11.93) |
| Week 12 | Change from Baseline: N, Mean (SD) | 17, −3.9 | (7.37) | 22, −5.4 | (8.57) |

| | | |
|---|---|---|
| Week 12 | Standard Effect Size | −0.183 |
| Week 12 | Treatment Difference versus Placebo: | 0.522, −1.73 |
| | p-value, LS Mean Difference (95% CI) | (−7.16, 3.70) |

Note:
NSA-16 Total Score ranges from 16 to 96. with higher scores indicating greater clinical severity of symptoms.
Standard Effect Size is defined as (mean change in d6-DM/Q-mean change in Placebo)/Change from Baseline Pooled SD.
Missing values were imputed by LOCF and visit windows were used to classify unscheduled or ET visits.
LS mean difference and p-values were from ANCOVA with treatment as fixed effect and Stage 1 Baseline value as a covariate.
Patients within each treatment group received the same treatment throughout their participation in the study.

TABLE 76
PANSS Total Score: Change from Baseline Parallel
Group MMRM Analysis. Observed Data
mITT 12-Week Parallel Group (N = 87)

| | Placebo/ | d6-DM/ |
|---|---|---|
| Visit/Statistics | Placebo | Q/d6-DM/Q |
| Baseline: N, Mean (SD) | 40, 69.4 | 47, 67.4 |
| | (8.18) | (8.26) |
| Week 3 Change from Baseline: | 40, −2.8 | 45, −3.2 |
| N, Mean (SD) | (7.56) | (6.84) |
| Week 6 Change from Baseline: | 35, −3.0 | 47, −4.7 |
| N, Mean (SD) | (6.27) | (6.98) |
| Week 9 Change from Baseline: | 32, −3.0 | 42, −7.3 |
| N, Mean (SD) | (8.43) | (6.91) |
| Week 12 Change from Baseline: N, | 31, −4.3 | 42, −7.4 |
| Mean (SD) | (9.82) | (7.66) |
| Week 12 Treatment Difference vs. Placebo: | | 0.141, −2.90 |
| p-value. LS Mean Difference. (95% CI) | | (−6.78, 0.98) |

Note:
PANSS Total Score ranges from 30 to 210, with higher scores indicating greater clinical severity of symptoms.
Patients within each treatment group received the same treatment throughout their participation in the study.
Repeated measures model includes fixed effect for treatment, visit, treatment-by-visit interaction, baseline value, and baseline value-by-vsit interaction. An unstructured covariance matrix was used.

TABLE 77
Association between NSA-16 Total Score Change from Baseline (LOCF) and Deuterated
(d6)-dextromethorphan (d6-DM) Cmax in Stage 1 mITT Population (N = 127)

| | Actual | | | Change from Baseline | | | |
|---|---|---|---|---|---|---|---|
| | | | Median | | | Median | |
| Treatment | N | Mean (SD) | (Min, Max) | N | Mean (SD) | (Min, Max) | |
| NSA-16 Total Score at Week 6 | | | | | | | |
| d6-DM/Q (N = 47) | 47 | 55.9 (8.75) | 55.0 (37, 72) | 47 | −5.0 (5.64) | −5.0 (−17, 10) | |
| Placebo (N = 80) | 80 | 57.4 (9.21) | 57.0 (43, 86) | 80 | −3.0 (5.78) | −2.5 (−15, 10) | |
| d6-DM Cmax at Week 6 | | | | | | | |
| d6-DM/Q (N = 47) | 43 | 51.6 (38.53) | 40.0 (8, 192) | | | | |
| Pearson correlation | | | | | | | −0.132, 0.4001 |
| coefficient, p-value [1]) | | | | | | | |

Note:
Missing values for the NSA-16 were imputed by LOCF within each stage.
[1] Test of whether the correlation between change in NSA-16 total score and the PK parameter is significantly different from 0.

TABLE 78
Association between NSA-16 Total Score Change from Baseline (LOCF) and Deuterated
(d6)-dextromethorphan (d6-DM) Cmax in Stage 2 Stage 1 Placebo Non-Responders (N = 77)

| | Actual | | | Change from Baseline | | | |
|---|---|---|---|---|---|---|---|
| | | | Median | | | Median | |
| Treatment | N | Mean (SD) | (Min, Max) | N | Mean (SD) | (Min, Max) | |
| NSA-16 Total Score at Week 12 | | | | | | | |
| d6-DM/Q (N = 33) | 33 | 54.0 (8.63) | 55.0 (42, 75) | 33 | −3.6 (6.34) | −2.0 (−19, 11) | |
| Placebo (N = 31) | 30 | 55.4 (11.28) | 55.0 (37, 81) | 30 | −2.2 (5.89) | −1.0 (−23, 7) | |
| d6-DM Cmax at Week 12 | | | | | | | |
| d6-DM/Q (N = 33) | 31 | 48.9 (28.34) | 44.3 (7, 121) | | | | |
| Pearson correlation | | | | | | | −0.249, 0.1774 |
| coefficient, p-value [1]) | | | | | | | |

Note:
Missing values for the NSA-16 were imputed by LOCF within each stage.
[1] Test of whether the correlation between change in NSA-16 total score and the PK parameter is significantly different from 0.

TABLE 79
Association between NSA-16 Total Score Change from Baseline (LOCF) and Deuterated
(d6)-dextromethorphan (d6-DM) Cmax at Week 12 mITT 12-Week Parallel Group (N = 87)

| | Actual | | | Change from Baseline | | | |
|---|---|---|---|---|---|---|---|
| | | | Median | | | Median | |
| Treatment | N | Mean (SD) | (Min, Max) | N | Mean (SD) | (Min, Max) | |
| NSA-16 Total Score at Week 12 | | | | | | | |
| d6-DM/Q (N = 47) | 47 | 54.9 (10.87) | 53.0 (31, 76) | 47 | −6.1 (7.77) | −5.0 (−23, 10) | |
| Placebo (N = 40) | 40 | 55.4 (10.93) | 55.0 (37, 81) | 40 | −5.7 (7.50) | −4.5 (−24, 9) | |
| d6-DM Cmax at Week 12 | | | | | | | |
| d6-DM/Q (N = 47) | 40 | 53.4 (40.09) | 42.3 (8, 194) | | | | |
| Pearson correlation | | | | | | | 0.140, 0.3887 |
| coefficient, p-value [1]) | | | | | | | |

Note:
Missing values for the NSA-16 were imputed by LOCF within each stage.
[1] Test of whether the correlation between change in NSA-16 total score and the PK parameter is significantly different from 0.

TABLE 80
Association between NSA-16 Total Score Change from Baseline (LOCF) and d3-dextrorphan (d3-DX) Cmax in Stage 1.
mITT Population (N = 127)

| | Actual | | | Change from Baseline | | | |
|---|---|---|---|---|---|---|---|
| Treatment | N | Mean (SD) | Median (Min, Max) | N | Mean (SD) | Median (Min, Max) | |
| NSA-16 Total Score at Week 6 | | | | | | | |
| d6-DM/Q (N = 47) | 47 | 55.9 (8.75) | 55.0 (37, 72) | 47 | −5.0 (5.64) | −5.0 (−17, 10) | |
| Placebo (N = 80) | 80 | 57.4 (9.21) | 57.0 (43, 86) | 80 | −3.0 (5.78) | −2.5 (−15, 10) | |
| d3-DX Cmax at Week 6 | | | | | | | |
| d6-DM/Q (N = 47) | 43 | 127.4 (50.25) | 121.6 (56, 332) | | | | |
| Pearson correlation coefficient, p-value [1]) | | | | | | | −0.158, 0.3124 |

Note:
Missing values for the NSA-16 were imputed by LOCF within each stage.
[1] Test of whether the correlation between change in NSA-16 total score and the PK parameter is significantly different from 0.

TABLE 81
Association between NSA-16 Total Score Change from Baseline (LOCF)
and d3-dextrorphan (d3-DX) Cmax in Stage 2 Stage 1 Placebo Non-Responders (N = 77)

| | Actual | | | Change from Baseline | | | |
|---|---|---|---|---|---|---|---|
| Treatment | N | Mean (SD) | Median (Min, Max) | N | Mean (SD) | Median (Min, Max) | |
| NSA-16 Total Score at Week 12 | | | | | | | |
| d6-DM/Q (N = 33) | 33 | 54.0 (8.63) | 55.0 (42, 75) | 33 | −3.6 (6.34) | −2.0 (−19, 11) | |
| Placebo (N = 31) | 30 | 55.4 (11.28) | 55.0 (37, 81) | 30 | −2.2 (5.89) | −1.0 (−23, 7) | |
| d3-DX Cmax at Week 12 | | | | | | | |
| d6-DM/Q (N = 33) | 31 | 127.1 (50.06) | 116.4 (65, 318) | | | | |
| Pearson correlation coefficient, | | | | | | | −0.026, 0.8876 |
| p-value [1]) | | | | | | | |

Note:
Missing values for the NSA-16 were imputed by LOCF within each stage.
[1] Test of whether the correlation between change in NSA-16 total score and the PK parameter is significantly different from 0.

TABLE 82
Association between NSA-16 Total Score Change from Baseline (LOCF)
and d3-dextrorphan (d3-DX) Cmax at Week 12 mITT 12-Week Parallel Group (N = 87)

| | Actual | | | Change from Baseline | | | |
|---|---|---|---|---|---|---|---|
| Treatment | N | Mean (SD) | Median (Min, Max) | N | Mean (SD) | Median (Min, Max) | |
| NSA-16 Total Score at Week 12 | | | | | | | |
| d6-DM/Q (N = 47) | 47 | 54.9 (10.87) | 53.0 (31, 76) | 47 | −6.1 (7.77) | −5.0 (−23, 10) | |
| Placebo (N = 40) | 40 | 55.4 (10.93) | 55.0 (37, 81) | 40 | −5.7 (7.50) | −4.5 (−24, 9) | |
| d3-DX Cmax at Week 12 | | | | | | | |
| d6-DM/Q (N = 47) | 40 | 126.8 (50.32) | 123.0 (57, 337) | | | | |
| Pearson correlation coefficient, | | | | | | | −0.036, 0.8243 |
| p-value [1]) | | | | | | | |

Note:
Missing values for the NSA-16 were imputed by LOCF within each stage.
[1] Test of whether the correlation between change in NSA-16 total score and the PK parameter is significantly different from 0.

TABLE 83
Association between NSA-16 Total Score Change from Baseline
(LOCF) and Quinidine (Q) Cmax in Stage 1 mITT Population (N = 127)

| | Actual | | | Change from Baseline | | | |
|---|---|---|---|---|---|---|---|
| Treatment | N | Mean (SD) | Median (Min, Max) | N | Mean (SD) | Median (Min, Max) | |
| NSA-16 Total Score at Week 6 | | | | | | | |
| d6-DM/Q (N = 47) | 47 | 55.9 (8.75) | 55.0 (37, 72) | 47 | −5.0 (5.64) | −5.0 (−17, 10) | |
| Placebo (N = 80) | 80 | 57.4 (9.21) | 57.0 (43, 86) | 80 | −3.0 (5.78) | −2.5 (−15, 10) | |
| Quinidine Cmax at Week 6 | | | | | | | |
| d6-DM/Q (N = 47) | 43 | 20.0 (8.21) | 17.7 (9, 43) | | | | |
| Pearson correlation coefficient, | | | | | | | −0.231, 0.1367 |
| p-value [1]) | | | | | | | |

Note:
Missing values for the NSA-16 were imputed by LOCF within each stage.
[1] Test of whether the correlation between change in NSA-16 total score and the PK parameter is significantly different from 0.

TABLE 84
Association between NSA-16 Total Score Change from Baseline
(LOCF) and Quinidine (Q) Cmax in Stage 2 Stage 1 Placebo Non-Responders (N = 77)

| | Actual | | | Change from Baseline | | | |
|---|---|---|---|---|---|---|---|
| Treatment | N | Mean (SD) | Median (Min, Max) | N | Mean (SD) | Median (Min, Max) | |
| NSA-16 Total Score at Week 12 | | | | | | | |
| d6-DM/Q (N = 33) | 33 | 54.0 (8.63) | 55.0 (42, 75) | 33 | −3.6 (6.34) | −2.0 (−19, 11) | |
| Placebo (N = 31) | 30 | 55.4 (11.28) | 55.0 (37, 81) | 30 | −2.2 (5.89) | −1.0 (−23, 7) | |
| Quinidine Cmax at Week 12 | | | | | | | |
| d6-DM/Q (N = 33) | 31 | 21.4 (9.05) | 19.5 (10, 45) | | | | |
| Pearson correlation coefficient, | | | | | | | 0.013, 0.9436 |
| p-value [1]) | | | | | | | |

Note:
Missing values for the NSA-16 were imputed by LOCF within each stage.
[1] Test of whether the correlation between change in NSA-16 total score and the PK parameter is significantly different from 0.

TABLE 85
Association between NSA-16 Total Score Change from Baseline (LOCF)
and Quinidine (Q) Cmax at Week 12 mITT 12-Week Parallel Group (N = 87)

| | Actual | | | Change from Baseline | | | |
|---|---|---|---|---|---|---|---|
| Treatment | N | Mean(SD) | Median (Min, Max) | N | Mean (SD) | Median (Min, Max) | |
| NSA-16 Total Score at Week 12 | | | | | | | |
| d6-DM/Q (N = 47) | 47 | 54.9 (10.87) | 53.0 (31, 76) | 47 | −6.1 (7.77) | −5.0 (−23, 10) | |
| Placebo (N = 40) | 40 | 55.4 (10.93) | 55.0 (37, 81) | 40 | −5.7 (7.50) | −4.5 (−24, 9) | |
| Quinidine Cmax at Week 12 | | | | | | | |
| d6-DM/Q (N = 47) | 40 | 20.6 (8.73) | 18.8 (10, 45) | | | | |
| Pearson correlation coefficient, | | | | | | | 0.147, 0.3667 |
| p-value [1]) | | | | | | | |

Note:
Missing values for the NSA-16 were imputed by LOCF within each stage.
[1] Test of whether the correlation between change in NSA-16 total score and the PK parameter is significantly different from 0.

TABLE 86
Association between NSA-16 Total Score Change from Baseline (LOCF)
and Deuterated (d6)-dextromethorphan (d6-DM) AUC in Stage 1 mITT Population (N = 127)

| | Actual | | | Change from Baseline | | | |
|---|---|---|---|---|---|---|---|
| Treatment | N | Mean (SD) | Median (Min, Max) | N | Mean (SD) | Median (Min, Max) | |
| NSA-16 Total Score at Week 6 | | | | | | | |
| d6-DM/Q (N = 47) | 47 | 55.9 (8.75) | 55.0 (37, 72) | 47 | −5.0 (5.64) | −5.0 (−17, 10) | |
| Placebo (N = 80) | 80 | 57.4 (9.21) | 57.0 (43, 86) | 80 | −3.0 (5.78) | −2.5 (−15, 10) | |
| d6-DM AUC at Week 6 | | | | | | | |
| d6-DM/Q (N = 47) | 43 | 456.2 (362.05) | 355.4 (73, 1793) | | | | |
| Pearson correlation coefficient, | | | | | | | −0.131, 0.4008 |
| p-value [1]) | | | | | | | |

Note:
Missing values for the NSA-16 were imputed by LOCF within each stage.
[1] Test of whether the correlation between change in NSA-16 total score and the PK parameter is significantly different from 0.

TABLE 87
Association between NSA-16 Total Score Change from Baseline (LOCF)
and Deuterated (d6)-dextromethorphan (d6-DM) AUC in Stage 2 Stage 1 Placebo Non-Responders (N = 77)

| | Actual | | | Change from Baseline | | | |
|---|---|---|---|---|---|---|---|
| Treatment | N | Mean (SD) | Median (Min, Max) | N | Mean (SD) | Median (Min, Max) | |
| NSA-16 Total Score at Week 12 | | | | | | | |
| d6-DM/Q (N = 33) | 33 | 54.0 (8.63) | 55.0 (42, 75) | 33 | −3.6 (6.34) | −2.0 (−19, 11) | |
| Placebo (N = 31) | 30 | 55.4 (11.28) | 55.0 (37, 81) | 30 | −2.2 (5.89) | −1.0 (−23, 7) | |
| d6-DM AUC at Week 12 | | | | | | | |
| d6-DM/Q (N = 33) | 31 | 422.8 (255.94) | 374.8 (53, 1081) | | | | |
| Pearson correlation coefficient, | | | | | | | −0.239, 0.1955 |
| p-value [1]) | | | | | | | |

Note:
Missing values for the NSA-16 were imputed by LOCF within each stage.
[1] Test of whether the correlation between change in NSA-16 total score and the PK parameter is significantly different from 0.

TABLE 88
Association between NSA-16 Total Score Change from Baseline (LOCF)
and Deuterated (d6)-dextromethorphan (d6-DM) AUC at Week 12 mITT 12-Week Parallel Group (N = 87)

| | Actual | | | Change from Baseline | | | |
|---|---|---|---|---|---|---|---|
| Treatment | N | Mean (SD) | Median (Min, Max) | N | Mean (SD) | Median (Min, Max) | |
| NSA-16 Total Score at Week 12 | | | | | | | |
| d6-DM/Q (N = 47) | 47 | 54.9 (10.87) | 53.0 (31, 76) | 47 | −6.1 (7.77) | −5.0 (−23, 10) | |
| Placebo (N = 40) | 40 | 55.4 (10.93) | 55.0 (37, 81) | 40 | −5.7 (7.50) | −4.5 (−24, 9) | |
| d6-DM AUC at Week 12 | | | | | | | |
| d6-DM/Q (N = 47) | 40 | 473.4 (375.01) | 369.4 (70, 1810) | | | | |
| Pearson correlation coefficient, | | | | | | | 0.129, 0.4269 |
| p-value [1]) | | | | | | | |

Note:
Missing values for the NSA-16 were imputed by LOCF within each stage.
[1] Test of whether the correlation between change in NSA-16 total score and the PK parameter is significantly different from 0.

TABLE 89
Association between NSA-16 Total Score Change from Baseline
(LOCF) and d3-dextrorphan (d3-DX) AUC in Stage 1 mITT Population (N = 127)

| | Actual | | | Change from Baseline | | | |
|---|---|---|---|---|---|---|---|
| Treatment | N | Mean (SD) | Median (Min, Max) | N | Mean (SD) | Median (Min, Max) | |
| NSA-16 Total Score at Week 6 | | | | | | | |
| d6-DM/Q (N = 47) | 47 | 55.9 (8.75) | 55.0 (37, 72) | 47 | −5.0 (5.64) | −5.0 (−17, 10) | |
| Placebo (N = 80) | 80 | 57.4 (9.21) | 57.0 (43, 86) | 80 | −3.0 (5.78) | −2.5 (−15, 10) | |
| d3-DX AUC at Week 6 | | | | | | | |
| d6-DM/Q (N = 47) | 43 | 1142.3 (378.08) | 1083.8 (506, 2091) | | | | |
| Pearson correlation coefficient, | | | | | | | −0.207, 0.1834 |
| p-value [1]) | | | | | | | |

Note:
Missing values for the NSA-16 were imputed by LOCF within each stage.
[1] Test of whether the correlation between change in NSA-16 total score and the PK parameter is significantly different from 0.

TABLE 90
Association between NSA-16 Total Score Change from Baseline (LOCF)
and d3-dextrorphan (d3-DX) AUC in Stage 2 Stage 1 Placebo Non-Responders (N = 77)

| | Actual | | | Change from Baseline | | | |
|---|---|---|---|---|---|---|---|
| Treatment | N | Mean (SD) | Median (Min, Max) | N | Mean (SD) | Median (Min, Max) | |
| NSA-16 Total Score at Week 12 | | | | | | | |
| d6-DM/Q (N = 33) | 33 | 54.0 (8.63) | 55.0 (42, 75) | 33 | −3.6 (6.34) | −2.0 (−19, 11) | |
| Placebo (N = 31) | 30 | 55.4 (11.28) | 55.0 (37, 81) | 30 | −2.2 (5.89) | −1.0 (−23, 7) | |
| d3-DX AUC at Week 12 | | | | | | | |
| d6-DM/Q (N = 33) | 31 | 1173.1 (537.52) | 1072.1 (620, 3622) | | | | |
| Pearson correlation coefficient, | | | | | | | −0.126, 0.4993 |
| p-value [1]) | | | | | | | |

Note:
Missing values for the NSA-16 were imputed by LOCF within each stage.
[1] Test of whether the correlation between change in NSA-16 total score and the PK parameter is significantly different from 0.

TABLE 91
Association between NSA-16 Total Score Change from Baseline (LOCF)
and d3-dextrorphan (d3-DX) AUG at Week 12 mITT 12-Week Parallel Group (N = 87)

| | Actual | | | Change from Baseline | | | |
|---|---|---|---|---|---|---|---|
| Treatment | N | Mean (SD) | Median (Min, Max) | N | Mean (SD) | Median (Min, Max) | |
| NSA-16 Total Score at Week 12 | | | | | | | |
| d6-DM/Q (N = 47) | 47 | 54.9 (10.87) | 53.0 (31, 76) | 47 | −6.1 (7.77) | −5.0 (−23, 10) | |
| Placebo (N = 40) | 40 | 55.4 (10.93) | 55.0 (37, 81) | 40 | −5.7 (7.50) | −4.5 (−24, 9) | |
| d3-DX AUC at Week 12 | | | | | | | |
| d6-DM/Q (N = 47) | 40 | 1142.8 (378.46) | 1087.1 (511, 2115) | | | | |
| Pearson correlation coefficient, | | | | | | | −0.141, 0.3847 |
| p-value [1]) | | | | | | | |

Note:
Missing values for the NSA-16 were imputed by LOCF within each stage.
[1] Test of whether the correlation between change in NSA-16 total score and the PK parameter is significantly different from 0.

TABLE 92
Association between NSA-16 Total Score Change from Baseline
(LOCF) and Quinidine (Q) AUC in Stage 1 mITT Population (N = 127)

| | Actual | | | Change from Baseline | | | |
|---|---|---|---|---|---|---|---|
| Treatment | N | Mean (SD) | Median (Min, Max) | N | Mean (SD) | Median (Min, Max) | |
| NSA-16 Total Score at Week 6 | | | | | | | |
| d6-DM/Q (N = 47) | 47 | 55.9 (8.75) | 55.0 (37, 72) | 47 | −5.0 (5.64) | −5.0 (−17, 10) | |
| Placebo (N = 80) | 80 | 57.4 (9.21) | 57.0 (43, 86) | 80 | −3.0 (5.78) | −2.5 (−15, 10) | |
| Quinidine AUC at Week 6 | | | | | | | |
| d6-DM/Q (N = 47) | 43 | 159.1 (63.81) | 142.1 (75, 325) | | | | |
| Pearson correlation coefficient, | | | | | | | −0.224, 0.1490 |
| p-value [1]) | | | | | | | |

Note:
Missing values for the NSA-16 were imputed by LOCF within each stage.
[1] Test of whether the correlation between change in NSA-16 total score and the PK parameter is significantly different from 0.

TABLE 93
Association between NSA-16 Total Score Change from Baseline (LOCF)
and Quinidine (Q) AUC in Stage 2 Stage 1 Placebo Non-Responders (N = 77)

| | Actual | | | Change from Baseline | | | |
|---|---|---|---|---|---|---|---|
| Treatment | N | Mean (SD) | Median (Min, Max) | N | Mean (SD) | Median (Min, Max) | |
| NSA-16 Total Score at Week 12 | | | | | | | |
| d6-DM/Q (N = 33) | 33 | 54.0 (8.63) | 55.0 (42, 75) | 33 | −3.6 (6.34) | −2.0 (−19, 11) | |
| Placebo (N = 31) | 30 | 55.4 (11.28) | 55.0 (37, 81) | 30 | −2.2 (5.89) | −1.0 (−23, 7) | |
| Quinidine AUC at Week 12 | | | | | | | |
| d6-DM/Q (N = 33) | 31 | 167.1 (69.68) | 155.5 (84, 342) | | | | |
| Pearson correlation coefficient, | | | | | | | 0.017, 0.9291 |
| p-value [1]) | | | | | | | |

Note:
Missing values for the NSA-16 were imputed by LOCF within each stage.
[1] Test of whether the correlation between change in NSA-16 total score and the PK parameter is significantly different from 0.

TABLE 94
Association between NSA-16 Total Score Change from Baseline
(LOCF) and Quinidine (Q) AUC at Week 12 mITT 12-Week Parallel Group (N = 87)

| | Actual | | | Change from Baseline | | | |
|---|---|---|---|---|---|---|---|
| Treatment | N | Mean (SD) | Median (Min, Max) | N | Mean (SD) | Median (Min, Max) | |
| NSA-16 Total Score at Week 12 | | | | | | | |
| d6-DM/Q (N = 47) | 47 | 54.9 (10.87) | 53.0 (31, 76) | 47 | −6.1 (7.77) | −5.0 (−23, 10) | |
| Placebo (N = 40) | 40 | 55.4 (10.93) | 55.0 (37, 81) | 40 | −5.7 (7.50) | −4.5 (−24, 9) | |
| Quinidine AUC at Week 12 | | | | | | | |
| d6-DM/Q (N = 47) | 40 | 163.7 (68.42) | 146.5 (81, 347) | | | | |
| Pearson correlation coefficient, | | | | | | | 0.151, 0.3511 |
| p-value [1]) | | | | | | | |

Note:
Missing values for the NSA-16 were imputed by LOCF within each stage.
[1] Test of whether the correlation between change in NSA-16 total score and the PK parameter is significantly different from 0.

1. A method of treating negative symptoms of schizophrenia in a patient having schizophrenia and as having clinically stable positive symptoms, comprising administering to the patient therapeutically effective amounts of deuterated [d6]-dextromethorphan hydrobromide (d6-DM) and quinidine sulfate (Q).

2. The method according to claim 1, wherein the patient is administered d6-DM in a 27 mg to 54 mg dose twice daily and Q in a 4 mg to 7.5 mg dose twice daily.

3. The method according to claim 1, wherein the patient is administered d6-DM in a 30 mg to 45 mg dose twice daily and Q in a 4 mg to 6 mg dose twice daily.

4. The method according to claim 1, wherein the patient is administered d6-DM in a 34 mg to 42.63 mg dose twice daily and Q in a 4.9 mg dose twice daily.

5-6. (canceled)

7. A method of treating negative symptoms of schizophrenia in a patient having schizophrenia, comprising administering to the patient therapeutically effective amounts of d6-DM and Q, wherein the patient has been assessed as having a score of less than or equal to 4 on the Positive and Negative Syndrome Scale (PANSS) items of delusions, hallucinations, and hostility, or wherein the patient has been assessed as having a score of less than or equal to 4 on the Positive and Negative Syndrome Scale (PANSS) items of delusions, hallucinations, suspiciousness/persecution, and hostility.

8. The method of claim 7, wherein the patient has been assessed as having a score of greater than or equal to 4 on any two, or greater than or equal to 5 on any one, of the PANSS items of blunted affect (N1), emotional withdrawal (N2), passive/apathetic social withdrawal (N4), and lack of spontaneity/flow of conversation (N6).

9. The method of claim 7, wherein the patient has been assessed as having a total PANSS negative subscale score (N1 to N7) of greater than or equal to 18.

10-11. (canceled)

12. The method of claim 7, wherein the patient has been assessed as having a total PANSS Marder negative factors score of greater than or equal to 20.

13. A method of specifically treating negative symptoms of schizophrenia in a patient having schizophrenia, comprising administering to the patient therapeutically effective amounts of d6-DM and Q,
wherein the patient is being treated with an atypical antipsychotic, wherein the patient has been treated with the atypical antipsychotic for at least 3 months prior to treatment with d6-DM and Q and the dose of the atypical antipsychotic has been stable for at least 1 month prior to treatment with d6-DM and Q, and/or
wherein the patient is being treated with an antidepressant, wherein the patient has been treated with the antidepressant for at least 3 months, and the dose of the antidepressant has been stable for at least 1 month, prior to treatment with d6-DM and Q.

14-37. (canceled)

38. A method of specifically treating negative symptoms of schizophrenia in a patient having schizophrenia, comprising administering to the patient therapeutically effective amounts of d6-DM and Q, wherein
during the first week of treatment, the d6-DM is administered in a 24 mg dose once daily and the Q is administered in a 4.9 mg dose once daily;
during the second week of treatment, the d6-DM is administered in a 24 mg dose twice daily and the Q is administered in a 4.9 mg dose twice daily; and
during the remainder of the treatment, the d6-DM is administered in a 34 mg dose twice daily and the Q is administered in a 4.9 mg dose twice daily, or wherein
during the first three days of treatment, the d6-DM is administered in a 28 mg dose once daily and the Q is administered in a 4.9 mg dose once daily;
during the next four days of treatment, the d6-DM is administered in a 28 mg dose twice daily and the Q is administered in a 4.9 mg dose twice daily; and
during the remainder of the treatment, the d6-DM is administered in a 42.63 mg dose twice daily and the Q is administered in a 4.9 mg dose twice daily.

39. (canceled)

40. The method of claim 1, wherein the patient is further administered an atypical antipsychotic other than clozapine.

41-46. (canceled)

47. The method of claim 1, wherein the patient has been diagnosed as having schizophrenia based on the Diagnostic and Statistical Manual of Mental Disorders (DSM) criteria for schizophrenia.

48. The method of claim 47, wherein the diagnosis based on the DSM criteria has been confirmed by the Mini International Neuropsychiatric Interview (M.I.N.I.).

49. The method of claim 1, wherein the treatment results in an at least 20% decrease in the PANSS Marder Negative Factor Score from baseline prior to treatment.

50. The method of claim 1, wherein the treatment results in an at least 2 point decrease in the PANSS Marder Negative Factor Score from baseline prior to treatment.

51. The method of claim 1, wherein the d6-DM is administered in a dose of 34 mg to 42.63 mg twice daily and the Q is administered in a dose of 4.9 mg twice daily.

52. The method of claim 4, wherein the d6-DM is administered in a 34 mg dose twice daily.

53. The method of claim 4, wherein the d6-DM is administered in a 42.63 mg dose twice daily.

54. The method of claim 1, further comprising treating prosocial factors by the administration of the d6-DM and the Q.
